(19) United States
(12) Patent Application Publication
(10) Pub. No.: US 2007/0031951 A1
(43) Pub. Date: Feb. 8, 2007

(54) Method for the production of resveratrol in a recombinant bacterial host cell

(76) Inventors: Lixuan Lisa Huang, Hockessin, DE (US); Zhixiong Xue, Chadds Ford, PA (US); Quinn Qun Zhu, West Chester, PA (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 11/436,160

(22) Filed: May 17, 2006

Related U.S. Application Data
(60) Provisional application No. 60/682,556, filed on May 19, 2005.

(51) Int. Cl. C12P 7/22; C12N 9/88; C12N 1/21; C12N 15/74

(52) U.S. Cl. 435/156; 435/252.3; 435/232; 435/471

(57) ABSTRACT

A method to produce resveratrol in a recombinant bacterial host cell is provided. Expression of a resveratrol synthase gene in combination with genes involved in the phenylpropanoid pathway enabled recombinant microbial production of resveratrol.

This application claims the benefit of U.S. Provisional Application No. 60/682,556 filed May 19, 2005.

### FIELD OF THE INVENTION

The invention is in the field of molecular biology and microbiology. Specifically, the invention relates to a method for producing resveratrol in a recombinant bacterial microorganism. Recombinant expression of genes involved in the phenylpropanoid pathway along with a resveratrol synthase gene enabled production of resveratrol.

### BACKGROUND OF THE INVENTION

Resveratrol (trans-3,4′,5-trihydroxystilbene) and/or its corresponding glucoside (piceid) are stilbene compounds reported to have many beneficial health effects. Resveratrol is a potent antioxidant that decreases low density lipid (LDL) oxidation, a factor associated with the development of atherosclerosis (Manna et al., J. Immunol., 164:6509-6519 (2000)). It is also reported to lower serum cholesterol levels and the incidents of heart disease. This effect as been attributed to a phenomenon known as the “French Paradox”. French citizens that regularly consume red wine tend to have lower incidents of heart disease and serum cholesterol levels even though this same group tends to consume foods high in both fat and cholesterol. There is also evidence that resveratrol may have other cardiovascular protective effects including modulation of vascular cell function, suppression of platelet aggregation, and reduction of myocardial damage during ischemia-reperfusion (Bradamante et al., Cardiovasc. Drug. Rev., 22(3):169-188 (2004)). Resveratrol is reported to have anti-inflammatory effects associated with the inhibition of the cyclooxygenase-1 (Cox-1), an enzyme associated with the conversion of arachidonic acid to pro-inflammatory mediators. It may also aid in the inhibition of carcinogenesis (Schultz, J., J Natl Cancer Inst., 96(20):1497-1498 (2004); Scifo et al., Oncol Res., 14(9):415-426 (2004); and Kundu, J. and Surh, Y., Mutat Res., 555(1-2):65-80 (2004)).

Resveratrol is classified as a phytoalexin due to its antifungal properties. It appears that some plants produce resveratrol as natural defense mechanism against fungal infections. For example, red grapes have been reported to produce resveratrol in response to fungal infections (fungal cell wall components can stimulate local expression of the resveratrol synthase gene in grapes). The antifungal property of resveratrol has been applied to plants that do not naturally produce the compound. Transgenic plants modified to express the resveratrol synthase gene exhibit improved resistance to fungal infections. Furthermore, it has been reported that treatment of fresh fruits and vegetables with effective amounts resveratrol will significantly increase shelf life (Gonzalez-Urena et al., J. Agric. Food Chem., 51:82-89 (2003)).

Use of resveratrol in commercial products (e.g., pharmaceuticals, personal care products, antifungal compositions, antioxidant compositions, dietary supplements, etc.) is limited due to the current market price of the compound. Methods to extract resveratrol from plant tissues such as red grape skins, peanuts or the root tissue of Polygonum cuspidatum are not economical. Means to produce resveratrol by chemical synthesis are difficult, inefficient, and expensive. There is a need for an efficient and cost-effective method to synthesize resveratrol.

Resveratrol (and/or resveratrol glucoside) is naturally produced in a variety of herbaceous plants (Vitaceae, Myrtaceae, and Leguminosae). The resveratrol biosynthesis pathway is well known. In plants, a single type III polyketide synthase (resveratrol synthase; E.C. 2.3.1.95) catalyzes three consecutive Claisen condensations of the acetate unit from malonyl CoA with the phenylpropanoid compound p-coumaroyl CoA, which is succeeded by (1) an aldol reaction that forms the second aromatic ring, (2) cleavage of the thioester, and (3) decarboxylation to form resveratrol.

Industrial microbial production offers a possible means to economically produce commercial quantities of resveratrol. Microbial production requires functional expression of the resveratrol synthase gene in the presence of suitable quantities of malonyl CoA and p-coumaroyl CoA. Cost-effective microbial production generally requires host cells having the ability to produce both malonyl CoA and p-coumaroyl CoA in suitable quantities. Preferably, the microbial host cell has the ability to product both substrates in suitable amounts when grown on an inexpensive carbon source, such as glusose. However, supplementation of one or more phenylpropanoid intermediates may also be required to achieve resveratrol production in commercially-suitable amounts.

Many naturally occurring microorganisms, such as E. coli and Saccharomyces cerevisiae, produce malonyl CoA (Davis et al., J Biol. Chem., 275:28593-28598 (2000) and Subrahmanyam, S. and Cronan, J., J. Bacteriol., 180:45964602 (1998)). However, many bacterial host cells do not make suitable amounts of malonyl CoA. As such, recombinant microbial production of resveratrol may require a cell engineered for increased malonyl CoA production.

Recombinant microbial production of resveratrol also requires the substrate p-coumaroyl CoA. This phenylpropanoid compound is ubiquitously produced in plants, but is found in relatively low quantitities (if at all) in many microbial host cells. As such, the resveratrol-producing microbial cell should be engineered to produce suitable amounts of p-coumaroyl CoA.

The enzyme coumaroyl CoA ligase (4CL; E.C. 6.2.1.12) converts p-hydroxycinnamic acid (pHCA) into p-coumaroyl CoA. In the past, coumaroyl CoA ligases were generally considered to only exist in plants, however a coumaroyl CoA ligase was recently reported in the filamentous bacterium Streptomyces coelicolor (Kaneko et al., J. Bacteriol., 185(1):20-27 (2003)). Recombinant microbial expression of coumaroyl CoA ligase has been reported (Becker et al., FEMS Yeast Research, 4(1):79-85 (2003)); Keneko et al., supra; Watts et al., Chembiochem, 5:500-507 (2004); and Hwang et al., Appl. Environ. Microbiol., 69(5):2699-2706 (2003)).

Recombinant biosynthesis of coumaroyl CoA require a suitable source of pHCA. The source of pHCA may be supplied exogenously to the host cell or it may be produced within the host cell. Preferably, the host cell can be engineered to produce suitable amounts of pHCA when grown on an inexpensive carbon source, such as glucose. Recombinant microbial host cells engineered to produce and/or accumulate phenylpropanoid-derived compounds (i.e., p-hydroxycinnamic acid) have previously been reported (U.S. Pat. No. 6,368,837, U.S. Pat. No. 6,521,748, U.S. Ser. No. 10/138970, U.S. Ser. No. 10/439,479, U.S. Ser. No. 10/621,826; and Schroder, J. and Schroder, G., Z. Naturforsch, 45:1-8 (1990)). Recombinant expression of a coumaroyl CoA ligase gene in cells engineered to produce p-hydroxycinnamic acid results in the production of p-coumaroyl CoA.

Microbial expression of genes encoding enzymes involved in the phenylpropanoid pathway for the production of the flavanone narigenin has been described by Watts et al. (supra) and Hwang et al. (supra). Specifically, Watts et al. describe the simultaneous expression of a phenylalanine ammonia lyase, a tyrosine ammonia lyase, a cinnamate 4-hydroxylase (C4H), a coumaroyl CoA ligase, and a chalcone synthase in E. coli to produce narigenin and phloretin up to 20.8 mg/L. However, Watts et al. were not able to actively express cinnamate-4-hydroxylase (C4H) in E. coli and had to supply exogenous p-coumaric acid or 3-(4-hydroxyphenyl)propionic acid to obtain significant concentrations of the desired products. Watts et al. do not describe recombinant microbial production of resveratrol.

Hwang et al. describe recombinant bacterial (i.e., E. coli) production of the flavanones pinocembrin and narigenin by simultaneously expressing phenylalanine ammonia lyase, coumaroyl CoA ligase, and a chalcone synthase. The bacterial coumaroyl CoA ligase used by Hwang et al. was able to convert both cinnamic acid to cinnamoyl CoA and p-coumaric acid to p-coumaroyl CoA, resulting in the production of pinocembrin (from phenylalanine) and naringenin (from tyrosine) as the PAL used also exhibited tyrosine ammonia lyase activity, resulting in the production of pHCA. In the absence of exogenously supplementing the medium with excess L-phenylalanine and/or L-tyrosine, only small amounts of each flavanone were produced (<0.3 μg/L). Hwang et al. do not describe recombinant microbial production of resveratrol.

Becker et al. (supra) recombinantly expressed several phenylpropanoid pathway genes in the yeast Saccharomyces cerevisiae FY23 for the production of resveratrol. Genes encoding a coumaroyl CoA ligase and a resveratrol synthase were recombinantly expressed in S. cerevisiae in a culture medium supplemented with pHCA, producing resveratrol in amounts up to 1.45 μg/L in the culture volume. Becker et al. reported that experiments supplementing the culture medium with additional precursors necessary for resveratrol production did not produce significantly more resveratrol. Becker et al. do not describe a method to produce resveratrol in a recombinant bacterial host cell.

The problem to be solved is to provide a method for recombinant bacterial production of resveratrol.

### SUMMARY OF THE INVENTION

The stated problem has been solved by providing a method to produce resveratrol in a recombinant bacterial host cell. The recombinant bacterial host cell was engineered to express at least one coumaroyl CoA ligase gene in combination with at least one resveratrol synthase gene. Para-hydroxycinnamic acid was supplemented to the culture medium, enabling production of resveratrol. Reseveratrol production was further enhanced by recombinantly expressing at least one malonyl CoA synthetase gene and at least one gene providing dicarboxylate or malonate transport protein activity (i.e., enhances malonate transport across the plasma membrane). Supplementation of malonic acid/malonate and p-hydroxycinnamic acid to the culture medium increased resveratrol production in the recombinant bacterial cell.

It has been shown in the art that bacterial host cells can be engineered to produce p-hydroxycinnamic acid from L-phenylalanine and/or L-tyrosine by recombinantly expressing a gene encoding an enzyme having phenylalanine/tyrosine ammonia lyase activity. In another aspect, the recombinant host cell is engineered to produce suitable quantities of p-coumaroyl CoA by recombinantly expressing at least one gene encoding an enzyme having phenylalanine/tyrosine ammonia lyase activity.

Accordingly the invention provides a method for the production of resveratrol comprising:

a) providing a bacterial host cell comprising:
1) at least one nucleic acid molecule encoding an enzyme having resveratrol synthase activity;
2) a source of malonyl CoA and coumaroyl CoA;

b) growing the bacterial host of (a) under conditions where malonyl CoA and coumaroyl CoA are reacted to resveratrol; and

c) optionally recovering the resveratrol of step (b).

In alternated embodiments the invention provides methods for resveratrol production using bacterial host cells additionally expressing nucleic acid molecules encoding various polypeptides, including a malonyl transporter protein; coumaroyl CoA ligase, tyrosine ammonium lyase, cinnamate-4-hydroxylase, and phenylalanine ammonium lyase. Various intermediates in the production of resveratrol may also be provided including malonyl CoA, p-hydroxycinnamic acid, tyrosine, cinnamic acid and phenylalanine.

In another embodiment the invention provides a recombinant bacterial host cell comprising at least one nucleic acid molecule encoding an enzyme having resveratrol synthase acivity which produces resveratrol. Preferred recombinant bacterial host cells of the invention may optionally express least one nucleic acid molecule encoding a polypeptide selected from the group consisting of; malonyl CoA synthetase, malonate transporter protein, coumaroyl CoA ligase, tyrosine ammonium lyase, cinnamate-4-hydroxylase and phenylalanine ammonium lyase.

In another embodiment the invention provides an animal feed, pharmaceutical composition, antifungal composition, or a dietary supplement comprising at least 0.1 wt % of the transformed bacterial biomass having at least 0.2% dry cell weight resveratrol.

### BRIEF DESCRIPTION OF THE FIGURES SEQUENCE DESCRIPTIONS

The invention can be more fully understood from the following detailed description, the figures, and the accompanying sequence descriptions which form a part of this application.

FIG. 1. The resveratrol biosynthetic pathway. L-Phenylalanine (Phe) and/or L-tyrosine (Tyr) can be converted into para-hydroxycinnamic acid (pHCA). Phenylalanine is converted into L-tyrosine using an enzyme having phenylalanine hydroxylase activity. The tyrosine is converted into pHCA using an enzyme have PAL/TAL activity. In another aspect, phenylalanine can be converted into trans-cinnamic acid (CA) using an enzyme having PAL/TAL activity. A cytochrome P450/P450 reductase system (cinnamate 4-hydroxylase) converts trans-cinnamic acid to pHCA. pHCA is converted into p-coumaroyl CoA by coumaroyl CoA ligase. Malonyl CoA and p-coumaroyl CoA are converted into resveratrol by an enzyme having resveratrol synthase activity (stilbene synthase).

FIG. 2. Plasmid maps for pETDuet™-1, pCCL-ET-D3, and pET-ESTS-CCL.

FIG. 3. Plasmid map for pACYC.matBC.

FIG. 4. Plasmid map for pACYC.PCCL.matBC.

FIG. 5. Mass spec analysis of resveratrol produced by recombinant E. coli from sample Res2. Using negative ion electrospray mass spectroscopy, a peak at 11.04 min contains the molecular ion of 227 that matches the molecular weight of resveratrol (top). The peak at 7.84 min contains the molecular ion of 163 that matches the molecular weight of pHCA (bottom).

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the European Patent Convention (EPC) and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

A Sequence Listing is provided herewith on Compact Disk. The contents of the Compact Disk containing the Sequence Listing are hereby incorporated by reference in compliance with 37 CFR 1.52(e). The Compact Discs are submitted in duplicate and are identical to one another. The discs are labeld “Copy 1—Sequence Listing” and “Copy 2 Sequence listing” The discs contain the following file: CL3059 US NA.ST25 having the following size: 341,000 bytes and which was created May 17, 2006.

SEQ ID NO:1 is the nucleotide sequence of primer OT452.

SEQ ID NO:2 is the nucleotide sequence of primer OT453.

SEQ ID NO:3 is the nucleotide sequence of the Streptomyces coelicolor (ATCC® BAA-471 D™) coumaroyl CoA ligase.

SEQ ID NO:4 is the deduced amino acid sequence of the Streptomyces coelicolor (ATCC® BAA471 D™) coumaroyl CoA ligase.

SEQ ID NO: 5 is the nucleotide sequence of plasmid pET-Duet™-1 (Novagen-EMB Biosciences, Darmstadt, Germany).

SEQ ID NO: 6 is the nucleotide sequence of plasmid pCCL-ET-D3.

SEQ ID NO: 7 is the nucleotide sequence of a stilbene synthase coding sequence from Vitis sp.

SEQ ID NO: 8 is the deduced amino acid sequence of the resveratrol synthase polypeptide encoded by SEQ ID NO: 7.

SEQ ID NO: 9 is the nucleotide sequence of a resveratrol synthase coding sequence codon optimized for expression in E. coli.

SEQ ID NO: 10 is the nucleotide sequence of plasmid pET-ESTS-CCL.

SEQ ID NO: 11 is the nucleotide sequence of the phenylalanine ammonia lyase coding sequence from Rhodosporidium toruloides (GenBank® Accession No. X12702).

SEQ ID NO: 12 is the deduced amino acid sequence of the phenylalanine ammonia lyase encoded by SEQ ID NO: 11 isolated from Rhodosporidium toruloides (GenBank® Accession No. X12702).

SEQ ID NO: 13 is the nucleotide sequence of the malonyl CoA synthetase coding sequence from Rhizobium leguminosarum bv. Trifolii.

SEQ ID NO: 14 is the deduced amino acid sequence of the malonyl CoA synthetase from Rhizobium leguminosarum bv. Trifolii.

SEQ ID NO: 15 is the nucleotide sequence of the dicarboxylate transporter protein (the “malonate transporter MatC”) coding sequence from Rhizobium leguminosarum bv. Trifolii.

SEQ ID NO: 16 is the deduced amino acid sequence of the dicarboxylate transporter protein (the “malonate transporter MatC”) from Rhizobium leguminosarum bv. Trifolii.

SEQ ID NO: 17 is the nucleotide sequence of primer OT628.

SEQ ID NO: 18 is the nucleotide sequence of primer OT648.

SEQ ID NO: 19 is the nucleotide sequence of plasmid pACYC.matBC.

SEQ ID NO: 20 is the nucleotide seuqence of the coumaroyl CoA ligase coding sequence from Petroselineum crispum.

SEQ ID NO: 21 is the deduced amino acid sequence of coumaroyl CoA ligase from Petroselineum crispum.

SEQ ID NO: 22 is the nucleotide sequence of plasmid pACYC.PCCL.matBC.

SEQ ID NO: 23 is the nucleotide sequence comprising a phenylalanine ammonia lyase coding sequence from Rhodotorula mucilaginosa.

SEQ ID NO: 24 is the nucleotide sequence comprising a phenylalanine ammonia lyase coding sequence from Amanita muscaria.

SEQ ID NO: 25 is the nucleotide sequence comprising a phenylalanine ammonia lyase coding sequence from Ustilago maydis.

SEQ ID NO: 26 is the nucleotide sequence comprising a phenylalanine ammonia lyase coding sequence from Arabidopsis thaliana.

SEQ ID NO: 27 is the nucleotide sequence comprising a phenylalanine ammonia lyase coding sequence from Rubus idaeus.

SEQ ID NO: 28 is the nucleotide sequence comprising a phenylalanine ammonia lyase coding sequence from Medicago sativa.

SEQ ID NO: 29 is the nucleotide sequence comprising a phenylalanine ammonia lyase coding sequence from Rehmannia glutinosa.

SEQ ID NO: 30 is the nucleotide sequence comprising a phenylalanine ammonia lyase coding sequence from Lactuca savita.

SEQ ID NO: 31 is the nucleotide sequence comprising a phenylalanine ammonia lyase coding sequence from Petroselinium crispum.

SEQ ID NO: 32 is the nucleotide sequence comprising a phenylalanine ammonia lyase coding sequence from Prunus avium.

SEQ ID NO: 33 is the nucleotide sequence comprising a phenylalanine ammonia lyase coding sequence from Lithospernum erythrorhizon.

SEQ ID NO: 34 is the nucleotide sequence comprising a phenylalanine ammonia lyase coding sequence from Citrus limon.

SEQ ID NO: 35 is the nucleotide sequence comprising a tyrosine ammonia lyase coding sequence from Rhodotorula glutinis.

SEQ ID NO: 36 is the nucleotide sequence comprising a phenylalanine ammonia lyase coding sequence from Rhodobacter sphaeroides.

SEQ ID NO: 37 is the nucleotide sequence comprising a phenylalanine ammonia lyase coding sequence from Trichosporon cutaneum (U.S. Pat. No. 6,951,751).

SEQ ID NO: 38 is the nucleotide sequence comprising a coumaroyl CoA ligase coding sequence from Streptomyces coelicolor.

SEQ ID NO: 39 is the nucleotide sequence comprising a coumaroyl CoA ligase coding sequence from Allium cepa.

SEQ ID NO: 40 is the nucleotide sequence comprising a coumaroyl CoA ligase coding sequence from Streptomyces avermitilis.

SEQ ID NO: 41 is the nucleotide sequence comprising a coumaroyl CoA ligase coding sequence from Populus tremuloides.

SEQ ID NO: 42 is the nucleotide sequence comprising a coumaroyl CoA ligase coding sequence from Oryza sativa.

SEQ ID NO: 43 is the nucleotide sequence comprising a coumaroyl CoA ligase coding sequence from Amorpha fruticosa.

SEQ ID NO: 44 is the nucleotide sequence comprising a coumaroyl CoA ligase coding sequence from Populus tomentosa.

SEQ ID NO: 45 is the nucleotide sequence comprising a coumaroyl CoA ligase coding sequence from Nicotiana tabacum.

SEQ ID NO: 46 is the nucleotide sequence comprising a coumaroyl CoA ligase coding sequence from Pinus taeda.

SEQ ID NO: 47 is the nucleotide sequence comprising a coumaroyl CoA ligase coding sequence from Glycine max.

SEQ ID NO: 48 is the nucleotide sequence comprising a coumaroyl CoA ligase coding sequence from Arabidopsis thaliana.

SEQ ID NO: 49 is the nucleotide sequence comprising a coumaroyl CoA ligase coding sequence from Arabidopsis thaliana.

SEQ ID NO: 50 is the nucleotide sequence comprising a coumaroyl CoA ligase coding sequence from Rubus idaeus.

SEQ ID NO: 51 is the nucleotide sequence comprising a coumaroyl CoA ligase coding sequence from Lithospermum erythrorhizon.

SEQ ID NO: 52 is the nucleotide sequence comprising a coumaroyl CoA ligase coding sequence from Zea mays.

SEQ ID NO: 53 is the nucleotide sequence comprising a resveratrol synthase (stilbene synthase) coding sequence from Vitis sp.

SEQ ID NO: 54 is the nucleotide sequence comprising a resveratrol synthase (stilbene synthase) coding sequence from Vitis vinifera.

SEQ ID NO: 55 is the nucleotide sequence comprising a resveratrol synthase (stilbene synthase) coding sequence from Vitis vinifera.

SEQ ID NO: 56 is the nucleotide sequence comprising a resveratrol synthase (stilbene synthase) coding sequence from Arachis hypogaea.

SEQ ID NO: 57 is the nucleotide sequence comprising a resveratrol synthase (stilbene synthase) coding sequence from Cissus rhombifolia.

SEQ ID NO: 58 is the nucleotide sequence comprising a resveratrol synthase (stilbene synthase) coding sequence from Parthenocissus henryana.

SEQ ID NO: 59 is the nucleotide sequence comprising a resveratrol synthase (stilbene synthase) coding sequence from Parthenocissus quinquefolia.

SEQ ID NO: 60 is the nucleotide sequence comprising a resveratrol synthase (stilbene synthase) coding sequence from Vitis riparia.

SEQ ID NO: 61 is the nucleotide sequence comprising a resveratrol synthase (stilbene synthase) coding sequence from Vitis labrusca.

SEQ ID NO: 62 is the nucleotide sequence comprising a resveratrol synthase (stilbene synthase) coding sequence from Vitis sp. cv. “Norton”.

SEQ ID NO: 63 is the nucleotide sequence comprising a cinnamate 4-hydroxylase coding sequence from Cicer arietinum.

SEQ ID NO: 64 is the nucleotide sequence comprising a cinnamate 4-hydroxylase coding sequence from Populus tremuloides.

SEQ ID NO: 65 is the nucleotide sequence comprising a cinnamate 4-hydroxylase coding sequence from Oryza sativa.

SEQ ID NO: 66 is the nucleotide sequence comprising a cinnamate 4-hydroxylase coding sequence from Camellia sinensis.

SEQ ID NO: 67 is the nucleotide sequence comprising a cinnamate 4-hydroxylase coding sequence from Vigna radiata.

SEQ ID NO: 68 is the nucleotide sequence comprising a cinnamate 4-hydroxylase coding sequence from Helianthus tuberosus.

SEQ ID NO: 69 is the nucleotide sequence comprising a cinnamate 4-hydroxylase coding sequence from Camptotheca acuminata.

SEQ ID NO: 70 is the nucleotide sequence comprising a cinnamate 4-hydroxylase coding sequence from Arabidopsis thaliana.

SEQ ID NO: 71 is the nucleotide sequence comprising a cinnamate 4-hydroxylase coding sequence from Ruta graveolens.

SEQ ID NO: 72 is the nucleotide sequence comprising a cinnamate 4-hydroxylase coding sequence from Glycine max.

SEQ ID NO: 73 is the nucleotide sequence comprising a cinnamate 4-hydroxylase coding sequence from Citrus sinensis.

SEQ ID NO: 74 is the nucleotide sequence comprising a phenylalanine hydroxylase coding sequence from Chromobacterium violaceum.

SEQ ID NO: 75 is the nucleotide sequence comprising a phenylalanine hydroxylase coding sequence from Pseudomonas aeruginosa.

SEQ ID NO: 76 is the nucleotide sequence comprising a phenylalanine hydroxylase coding sequence from Geodia cydonium.

SEQ ID NO: 77 is the nucleotide sequence comprising a phenylalanine hydroxylase coding sequence from Xanthomonas axonopodis.

SEQ ID NO: 78 is the nucleotide sequence comprising a phenylalanine hydroxylase coding sequence from Xanthomonas campestris.

SEQ ID NO: 79 is the nucleotide sequence comprising a phenylalanine hydroxylase coding sequence from Nocardia farcinica.

SEQ ID NO: 80 is the nucleotide sequence comprising a phenylalanine hydroxylase coding sequence from Gallus gallus.

SEQ ID NO: 81 is the nucleotide sequence comprising a acetyl CoA carboxylase coding sequence from Saccharomyces cerevisiae.

SEQ ID NO: 82 is the nucleotide sequence comprising a acetyl CoA carboxylase coding sequence from Saccharomyces cerevisiae.

SEQ ID NO: 83 is the nucleotide sequence comprising a acetyl CoA carboxylase coding sequence from Kluyveromyces lactis.

SEQ ID NO: 84 is the nucleotide sequence comprising a acetyl CoA carboxylase coding sequence from Debaryomyces hansenii.

SEQ ID NO: 85 is the nucleotide sequence comprising a acetyl CoA carboxylase coding sequence from Yarrowia lipolytica.

SEQ ID NO: 86 is the nucleotide sequence comprising a acetyl CoA carboxylase coding sequence from Aspergillus nidulans.

SEQ ID NO: 87 is the nucleotide sequence comprising a acetyl CoA carboxylase coding sequence from Schizosaccharomyces pombe.

SEQ ID NO: 88 is the nucleotide sequence comprising a acetyl CoA carboxylase coding sequence from Ustilago maydis.

SEQ ID NO: 89 is the nucleotide sequence comprising a acetyl CoA carboxylase coding sequence from Gallus gallus.

SEQ ID NO: 90 is the nucleotide sequence comprising a β-glucosidase coding sequence from Mesoplasma florum.

SEQ ID NO: 91 is the nucleotide sequence comprising a β-glucosidase coding sequence from Oryza sativa.

SEQ ID NO: 92 is the nucleotide sequence comprising a β-glucosidase coding sequence from Pseudomonas putida.

SEQ ID NO: 93 is the nucleotide sequence comprising a β-glucosidase coding sequence from Pseudomonas syringae.

SEQ ID NO: 94 is the nucleotide sequence comprising a β-glucosidase coding sequence from Streptomyces coelicolor.

SEQ ID NO: 95 is the nucleotide sequence comprising a β-glucosidase coding sequence from Caulobacter crescentus.

SEQ ID NO: 96 is the nucleotide sequence comprising a β-glucosidase coding sequence from Candida wickerhamii.

SEQ ID NO: 97 is the nucleotide sequence comprising a malonyl CoA synthetase coding sequence from Bradyrhizobium japonicum.

SEQ ID NO: 98 is the nucleotide sequence comprising a malonyl CoA synthetase coding sequence from Bradyrhizobium sp. BTAi1.

SEQ ID NO: 99 is the nucleotide sequence comprising a malonyl CoA synthetase coding sequence from Rhodopseudomonas palustris.

SEQ ID NO: 100 is the nucleotide sequence comprising a malonyl CoA synthetase coding sequence from Mesorhizobium loti.

SEQ ID NO: 101 is the nucleotide sequence comprising a dicarboxylate transport protein coding sequence from Rhizobium etli.

SEQ ID NO: 102 is the nucleotide sequence comprising a dicarboxylate transport protein coding sequence from Xanthomonas campestris pv. vesicatoria str.

SEQ ID NO: 103 is the nucleotide sequence comprising a dicarboxylate transport protein coding sequence from Xanthomonas campestris pv. Campestris.

### DETAILED DESCRIPTION OF THE INVENTION

A method is provided for the production of resveratrol in a recombinant bacterial host cell. The method is exemplified by producing resveratrol in E. coli. Genes from the phenylpropanoid pathway were recombinantly expressed in combination with a codon optimized resveratrol synthase gene for the production of resveratrol. In one embodiment, the recombinant bacterial biosynthesis occurs in the presence of at least one exogenously supplemented product intermediate, such as p-hydroxycinnamic acid, L-tyrosine, and malonate (typically supplied as malonic acid). The resveratrol produced using the present method can be optionally isolated and/or purified.

The present invetion also provides the corresponding recombinant bacterial strains as well as resveratrol-containing bacterial biomass. In a further embodiment, the resveratrol-containing recombinant biomass can be used an ingredient in a variety of compositions.

In the following disclosure, a number of terms and abbreviations are used. The following definitions are provided:

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably with 5% of the reported numerical value.

The term “invention” or “present invention” as used herein is not meant to be limiting to any specific embodiment of the invention but refers to all aspects of the invention as described in the claims and the specification.

As used herein, the term “comprising” means the presence of the stated features, integers, steps, or components as referred to in the claims, but that it does not preclude the presence or addition of one or more other features, integers, steps, components or groups thereof.

As used herein, the terms “para-hydroxycinnamic acid”, “p-hydroxycinnamic acid”, and “4-hydroxycinnamic acid” are used interchangeably and are abbreviated as “pHCA”.

As used herein, the terms “phenylalanine” and “L-phenylalanine” are used interchangeably.

As used herein, the terms “tyrosine” and “L-tyrosine” are used interchangeably.

As used herein, the terms “trans-cinnamate” and “cinnamic acid” are used interchangeably.

As used herein, the term “resveratrol” is used to describe the compound trans-3,4′,5-trihydroxystilbene as shown below.

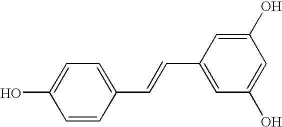

### Resveratrol (3,4′,5-trihydroxystilbene)

As used herein the terms “cinnamic acid” and “cinnamate” are used interchangeably.

As used herein, the term “stilbene synthase” and “resveratrol synthase” are used interchangeably and are abbreviated as RS. Resveratrol synthase is a type III polyketide synthase (E.C. 2.3.1.95) that condenses one molecule of p-coumaroyl CoA with 3 molecules of malonyl CoA to produce 1 molecule of resveratrol (trans-3,4′,5-trihydroxystilbene).

As used herein, the terms “para-coumaroyl-CoA” and “p-coumaroyl CoA” are used interchangeably.

As used herein, the term “coumaroyl CoA ligase” is used to described an enzyme (E.C. 6.2.1.12) that converts pHCA into p-coumaroyl CoA.

As used herein, the term “phenylalanine hydroxylase” is abbreviated PAH (E.C. 1.14.16.1). The term “PAH” activity” or “PAH enzyme” refers to an enzyme that hydroxylates phenylalanine to produce tyrosine.

As used herein, the term “cinnamate 4-hydroxylase” is used to describe one or more enzymes having an enzyme activity (E.C. 1.14.13.1 1) that converts trans-cinnamic acid to p-hydroxycinnamic acid and is abbreviated C4H.

As used herien, the term “product intermediate” refers to a compound selected from the group consisting of p-hydroxycinnamic acid, trans-cinnamic acid, malonate, malonic acid, L-tyrosine, L-phenylalanine, and mixtures thereof. In one embodiment, the product intermediate is selected from the group consisting of p-hydroxycinnamic acid, malonate, malonic acid, L-tyrosine, and mixtures thereof. In a further embodiment, the product intermediate is selected from the group consisting of p-hydroxycinnamic acid, malonate, L-tyrosine, and mixtures thereof. The product intermediate is typically added (“supplemented”) to the culture medium as part of the suitable growth conditions for resveratrol production. Supplementation of the culture medium with a product intermediate is optional, however supplmentation of at least one culture intermediate is preferred.

As used herein, the term “phenylalanine ammonia-lyase” is abbreviated PAL (EC 4.3.1.5). As used herein, the term “PAL activity” or “PAL enzyme” refers to the ability of a protein to catalyze the conversion of phenylalanine to cinnamic acid. “pal” represents a gene that encodes an enzyme with PAL activity. PAL enzymes normally have some TAL activity (E.C. 4.3.1.-). As such, phenylalanine ammonia lyases (especially those with significant TAL activity) will also be referred to herein as “phenylalanine/tyrosine ammonia lyases” or “PAL/TAL enzymes”.

As used herein, the term “tyrosine ammonia lyase” is abbreviated TAL (EC 4.3.1.-). As used herein, the term “TAL activity” or “TAL enzyme” refers to the ability of a protein to catalyze the direct conversion of tyrosine to p-hydroxycinnamic acid (pHCA). “tal” represents a gene that encodes an enzyme with TAL activity. TAL enzymes typically have some PAL activity (E.C. 4/3/1/5). As such, TAL enzymes may also be referred to herein as “phenylalanine/tyrosine ammonia lyases” or “PAL/TAL enyzmes”.

As used herein, the term “PAL/TAL activity” or “PAL/TAL enzyme” refers to a protein which contains both PAL and TAL activity. Such a protein has at least some specificity for both tyrosine and phenylalanine as an enzymatic substrate. The term “modified PAL/TAL” or “mutant PAL/TAL” refers to a protein that has been derived from a wild type PAL enzyme which has greater TAL activity than PAL activity (U.S. Pat. No. 6,368,837). As such, a modified PAL/TAL protein has a greater substrate specificity (or at least greatly improved in comparison to the non-modified enzyme from which is was derived) for tyrosine than for phenylalanine.

As used herein, “pETDuet™-1” is a commercially available expression plasmid from Novagen (Madison, Wis.; SEQ ID NO: 5).

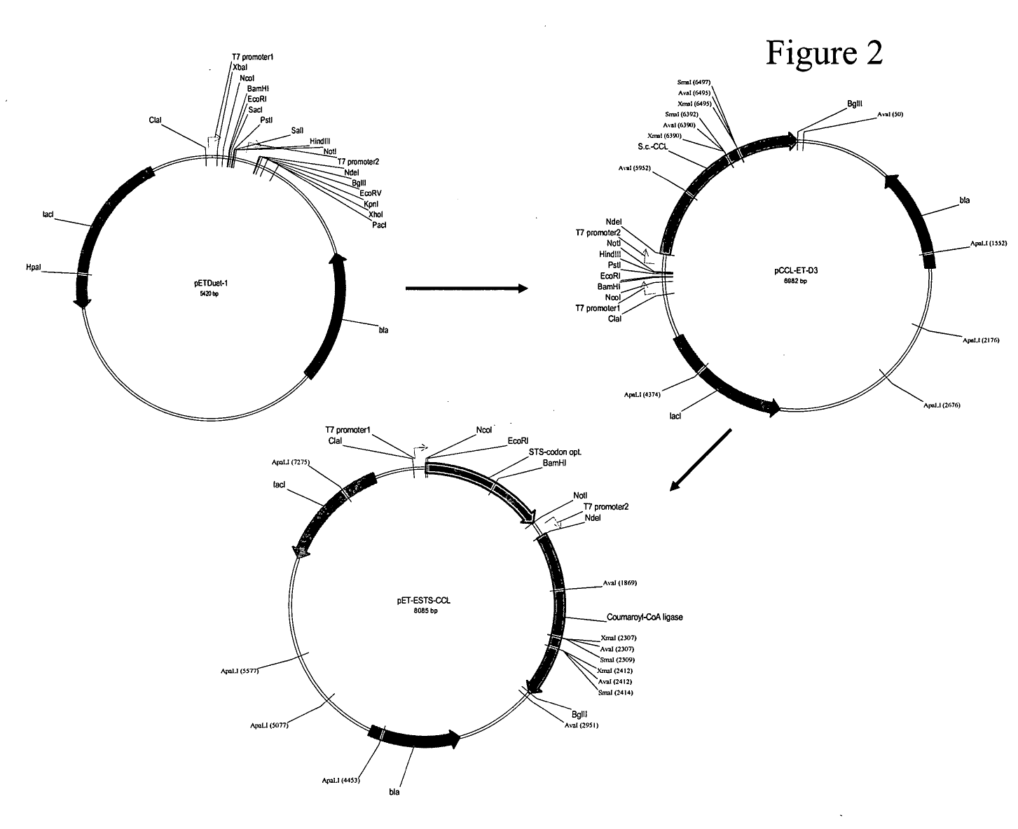

As used herein, “pCCL-ET-D3” is a plasmid (SEQ ID NO: 6) created by cloning the coumaroyl CoA ligase gene (SEQ ID NO: 3) from Streptomyces coelicolor (ATCC® BAA-471D™) into the commercial expression vector pETDuet™-1 (FIG. 2)

As used herein, “pET-ESTS-CCL” is used to described the plasmid (SEQ ID NO: 10) created by cloning a codon optimized version of a resveratrol synthase gene from Vitis sp. (SEQ ID NO: 9) into plasmid pCCL-ET-D3 (FIG. 2).

As used herein, the terms “significant amount” and “significant amount of resveratrol” are used to describe the amount of resveratrol produced using the present method (recombinant bacterial production of resveratrol). In one aspect, a significant amount produced by the present method is a resveratrol titer of at least 0.5 mg/L within the culture volume, preferably at least 1.5 mg/L within the culture volume, and most preferably at least 3 mg/L within the culture volume. In one aspect, “significant amount” is defined as at least 0.1% dry cell weight (dcw), preferably at least 0.2% (dcw), more preferably at least 1% (dcw), and most preferably at least 2% (dcw) resveratrol produced by the recombinant bacterial cell.

As used herein, the terms “suitable amount” and “suitable substrate amount” are used to describe an amount of available substrate that enables recombinant microbial production of resveratrol using the present method. In one aspect, the recombinant microbial host cell can produce suitable amounts of the necessary substrates for resveratrol production from the fermentable carbon source supplied to the fermentation media. In another aspect, one or more substrates (product intermediates) useful for the biosynthesis of resveratrol may be exogenously supplemented to the fermentation media to enable production resveratrol. In yet another aspect, the exogenously supplied substrate is selected from the group consisting of malonic acid (including salts of malonic acid), L-phenylalanine, L-tyrosine, p-hydroxycinnamic acid, and trans-cinnamic acid. In a preferred aspect, the exogenously supplied substrate is selected from the group consisting of p-hydroxycinnamic acid, malonic acid, and mixtures thereof.

As used herein, the terms “P450/P-450 reductase system” and “cytochrome P450/P450 reductase system” refers to a protein system responsible for the catalytic conversion of trans-cinnamic acid to pHCA. The P-450/P-450 reductase system is one of several enzymes or enzyme systems known in the art that performs a cinnamate 4-hydroxylase function. As used herein, the term “cinnamate 4-hydroxylase” (E.C. 1.14.13.11) will refer to the general enzymatic activity that results in the conversion of trans-cinnamic acid to pHCA, whereas the term “P450/P-450 reductase system” will refer to a specific binary protein system that has cinnamate 4-hydroxylase activity.

As used herein, the term “aromatic amino acid biosynthesis” means the biological processes and enzymatic pathways internal to a cell needed for the production of an aromatic amino acid (i.e., L-phenylalanine and/or L-tyrosine).

As used herein, the term “fermentable carbon substrate” refers to a carbon source capable of being metabolized by host organisms of the present invention and particularly carbon sources selected from the group consisting of monosaccharides (e.g., glucose, fructose), disaccharides (e.g., lactose, sucrose), oligosaccharides, polysaccharides (e.g., starch, cellulose or mixtures thereof), sugar alcohols (e.g., glycerol) or mixtures from renewable feedstocks (e.g., cheese whey permeate, cornsteep liquor, sugar beet molasses, barley malt). Additionally, carbon sources may include alkanes, fatty acids, esters of fatty acids, monoglycerides, diglycerides, triglycerides, phospholipids and various commercial sources of fatty acids including vegetable oils (e.g., soybean oil) and animal fats. Additionally, the carbon source may include one-carbon sources (e.g., carbon dioxide, methanol, formaldehyde, formate, carbon-containing amines) for which metabolic conversion into key biochemical intermediates has been demonstrated. In one aspect, the carbon source is a methylotrophic bacteria grown on methane and/or methanol. In a further aspect, the carbon source is a methanotrophic bacteria grown on methane and/or methanol. Hence, it is contemplated that the source of carbon utilized in the present invention may encompass a wide variety of carbon-containing sources and will only be limited by the choice of the host organism. Although all of the above mentioned carbon sources and mixtures thereof are expected to be suitable in the present invention, preferred carbon sources are sugars, single carbon sources such as methane and/or methanol, and/or fatty acids. Most preferred is glucose and/or fatty acids containing between 10-22 carbons.

As used herein, the term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing as well as those substantially similar nucleic acid sequences. In one aspect, substantially similar nucleic acid sequence sequences are those having at least 90% sequence identity.

As used herein, “gene” refers to a nucleic acid fragment that expresses a specific protein, including regulatory sequences preceding (5′ non-coding sequences) and following (3′ non-coding sequences) the coding sequence. “Native gene” or “wild type gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes.

As used herein, “coding sequence” refers to a DNA sequence that codes for a specific amino acid sequence.

As used herein, “suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, and polyadenylation recognition sequences.

As used herein, “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

As used herein, the term “promoter activity” will refer to an assessment of the transcriptional efficiency of a promoter. This may, for instance, be determined directly by measurement of the amount of mRNA transcription from the promoter (e.g., by Northern blotting or primer extension methods) or indirectly by measuring the amount of gene product expressed from the promoter.

As used herein, the term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

As used herein, the term “expression”, as used herein, refers to the transcription and stable accumulation of sense (mRNA) or antisense RNA derived from the nucleic acid fragment of the invention. Expression may also refer to translation of mRNA into a polypeptide. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression of identical or substantially similar foreign or endogenous genes (U.S. Pat. No. 5,231,020).

As used herein, “transformation” refers to the transfer of a nucleic acid molecule into the genome of a host organism, resulting in genetically stable inheritance. In the present invention, the host cell's genome includes chromosomal and extrachromosomal (e.g. plasmid) genes. Host organisms containing the transformed nucleic acid fragments are referred to as “transgenic”, “recombinant” or “transformed” organisms. In the present application, the nucleic acid molecule(s) transferred into the genome of host organism are operably linked to suitable regulatory sequences (e.g., promoters, terminators, etc.) that facilitate expression (i.e., a chimeric gene) in the host. The present genes may be chromosomally or extrachromosomally expressed.

As used herein, the terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, the term “amino acid” will refer to the basic chemical structural unit of a protein or polypeptide. The following abbreviations will be used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Asparagine or aspartic acid | Asx | B |
| Cysteine | Cys | C |
| Glutamine | Gln | Q |
| Glutamine acid | Glu | E |
| Glutamine or glutamic acid | Glx | Z |
| Glycine | Gly | G |
| Histidine | His | H |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

As used herein, the term “chemically equivalent amino acid” will refer to an amino acid that may be substituted for another in a given protein without altering the chemical or functional nature of that protein. For example, it is well known in the art that alterations in a gene which result in the production of a chemically equivalent amino acid at a given site, but do not effect the functional properties of the encoded protein are common. For the purposes of the present invention substitutions are defined as exchanges within one of the following five groups:
1. Small aliphatic, nonpolar or slightly polar residues: Ala, Ser, Thr (Pro, Gly);
2. Polar, negatively charged residues and their amides: Asp, Asn, Glu, Gin;
3. Polar, positively charged residues: His, Arg, Lys;
4. Large aliphatic, nonpolar residues: Met, Leu, lle, Val (Cys); and
5. Large aromatic residues: Phe, Tyr, Trp.

Thus, alanine, a hydrophobic amino acid, may be substituted by another less hydrophobic residue (such as glycine) or a more hydrophobic residue (such as valine, leucine, or isoleucine). Similarly, changes which result in substitution of one negatively charged residue for another (such as aspartic acid for glutamic acid) or one positively charged residue for another (such as lysine for arginine) can also be expected to produce a functionally equivalent product. Additionally, in many cases, alterations of the N-terminal and C-terminal portions of the protein molecule would also not be expected to alter the activity of the protein.

A nucleic acid molecule is “hybridizable” to another nucleic acid molecule, such as a cDNA, genomic DNA, or RNA molecule, when a single-stranded form of the nucleic acid molecule can anneal to the other nucleic acid molecule under the appropriate conditions of temperature and solution ionic strength. Given the nucleic acid sequences described herein, one of skill in the art can identify substantially similar nucleic acid fragments that may encode proteins having similar activity. Hybridization and washing conditions are well known and exemplified in Sambrook, J., Fritsch, E. F. and Maniatis, T. Molecular Cloning: A Laboratory Manual, 2^{nd} ed., Cold Spring Harbor Laboratory: Cold Spring Harbor, N.Y. (1989), particularly Chapter 11 and Table 11.1 therein. The conditions of temperature and ionic strength determine the “stringency” of the hybridization. Stringency conditions can be adjusted to screen for moderately similar fragments (such as homologous sequences from distantly related organisms), to highly similar fragments (such as genes that duplicate functional enzymes from closely related organisms). Post-hybridization washes determine stringency conditions. One set of preferred conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. A more preferred set of stringent conditions uses higher temperatures in which the washes are identical to those above except for the temperature of the final two 30 min washes in 0.2×SSC, 0.5% SDS was increased to 60° C. Another preferred set of highly stringent conditions uses two final washes in 0.1×SSC, 0.1% SDS at 65° C. An additional set of stringent conditions include hybridization at 0.1×SSC, 0.1% SDS, 65° C. and washed with 2×SSC, 0.1% SDS at 65° C. followed by 0.1×SSC, 0.1% SDS at 65° C., for example.

Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tm for hybrids of nucleic acids having those sequences. The relative stability (corresponding to higher Tm) of nucleic acid hybridizations decreases in the following order: RNA:RNA, DNA:RNA, DNA:DNA. For hybrids of greater than 100 nucleotides in length, equations for calculating Tm have been derived (see Sambrook et al., supra, 9.50-9.51). For hybridizations with shorter nucleic acids, i.e., oligonucleotides, the position of mismatches becomes more important, and the length of the oligonucleotide determines its specificity (see Sambrook et al., supra, 11.7-11.8). In one aspect the length for a hybridizable nucleic acid is at least about 10 nucleotides. Preferably a minimum length for a hybridizable nucleic acid is at least about 15 nucleotides; more preferably at least about 20 nucleotides; and most preferably the length is at least about 30 nucleotides. Furthermore, the skilled artisan will recognize that the temperature and wash solution salt concentration may be adjusted as necessary according to factors such as length of the probe.

As used herein, a “substantial portion” of an amino acid or nucleotide sequence is that portion comprising enough of the amino acid sequence of a polypeptide or the nucleotide sequence of a gene and/or a nucleic acid fragment to putatively identify that polypeptide or gene and/or nucleic acid fragment, either by manual evaluation of the sequence by one skilled in the art, or by computer-automated sequence comparison and identification using algorithms such as BLAST (Basic Local Alignment Search Tool; Altschul, S. F., et al., J. Mol. Biol. 215:403-410 (1993)). In general, a sequence of ten or more contiguous amino acids or thirty or more nucleotides is necessary in order to identify putatively a polypeptide or nucleic acid sequence as homologous to a known protein or gene. Moreover, with respect to nucleotide sequences, gene-specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The instant specification teaches partial or complete amino acid and nucleotide sequences encoding one or more particular proteins and promoters. The skilled artisan, having the benefit of the sequences as reported herein, may now use all or a substantial portion of the disclosed sequences for purposes known to those skilled in this art. Accordingly, the instant invention comprises the complete sequences as reported in the accompanying Sequence Listing and Table 1, as well as substantial portions of those sequences as defined above.

As used herein, the term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as reported in the accompanying Sequence Listing, as well as those substantially similar nucleic acid sequences.

As used herein, the term “percent identity”, as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: 1.) Computational Molecular Biology (Lesk, A. M., Ed.) Oxford University: NY (1988); 2.) Biocomputing: Informatics and Genome Projects (Smith, D. W., Ed.) Academic: NY (1993); 3.) Computer Analysis of Sequence Data, Part I (Griffin, A. M., and Griffin, H. G., Eds.) Humana: NJ (1994); 4.) Sequence Analysis in Molecular Biology (von Heinje, G., Ed.) Academic (1987); and 5.) Sequence Analysis Primer (Gribskov, M. and Devereux, J., Eds.) Stockton: NY (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences is performed using the Clustal method of alignment (Higgins and Sharp, CABIOS, 5:151-153 (1989)) with default parameters (GAP PENALTY=10, GAP LENGTH PENALTY=10). Default parameters for pairwise alignments using the Clustal method are: KTUPLE 1, GAP PENALTY=3, WINDOW=5 and DIAGONALS SAVED=5.

In one aspect, suitable nucleic acid molecules encode polypeptides that are at least about 70% identical to the amino acid sequences reported herein. In another aspect, the nucleic acid fragments encode amino acid sequences that are about 85% identical to the amino acid sequences reported herein. In a further aspect, the nucleic acid fragments encode amino acid sequences that are at least about 90% identical to the amino acid sequences reported herein. In yet a further aspect, the nucleic acid fragments encode amino acid sequences that are at least about 95% identical to the amino acid sequences reported herein. In even yet a further aspect, the nucleic acid fragments encode amino acid sequences that are at least 99% identical to the amino acid sequences reported herein. In another embodiment, suitable nucleic acid fragments also include those encoding amino acid sequences that are identical to the amino acid sequences reported herein. Suitable nucleic acid fragments not only have the above homologies but typically encode a polypeptide having at least 50 amino acids, preferably at least 100 amino acids, more preferably at least 150 amino acids, still more preferably at least 200 amino acids, and most preferably at least 250 amino acids.

As used herein, “codon degeneracy” refers to the nature in the genetic code permitting variation of the nucleotide sequence without affecting the amino acid sequence of an encoded polypeptide. The skilled artisan is well aware of the “codon-bias” exhibited by a specific host cell in usage of nucleotide codons to specify a given amino acid. Therefore, when synthesizing a gene for improved expression in a host cell, it is desirable to design the gene such that its frequency of codon usage approaches the frequency of preferred codon usage of the host cell. In one aspect, the recombinantly expressed genes are codon optimized for expression in the bacterial host cell. In another aspect, the recombinantly expressed genes are codon optimized for expression in a bacterial genera selected from the group consisting of Escherichia, Bacillus, and Methylomonas. In yet another aspect, the recombinantly expressed genes are codon optimized for expression in Escherichia coli.

As used herein, the term “sequence analysis software” refers to any computer algorithm or software program that is useful for the analysis of nucleotide or amino acid sequences. “Sequence analysis software” may be commercially available or independently developed. Typical sequence analysis software will include, but is not limited to: 1.) the GCG suite of programs (Wisconsin Package Version 9.0, Genetics Computer Group (GCG), Madison, Wis.); 2.) BLASTP, BLASTN, BLASTX (Altschul et al., J. Mol. Biol. 215:403-410 (1990)); 3.) DNASTAR (DNASTAR, Inc. Madison, Wis.); and 4.) the FASTA program incorporating the Smith-Waterman algorithm (W. R. Pearson, Comput. Methods Genome Res., [Proc. Int. Symp.] (1994), Meeting Date 1992, 111-20. Editor(s): Suhai, Sandor. Plenum: New York, N.Y.). Within the context of this application it will be understood that where sequence analysis software is used for analysis, that the results of the analysis will be based on the “default values” of the program referenced, unless otherwise specified. As used herein “default values” will mean any set of values or parameters (as set by the software manufacturer) which originally load with the software when first initialized.

Standard recombinant DNA and molecular cloning techniques used here are well known in the art and are described by Sambrook, J., Fritsch, E. F. and Maniatis, T. Molecular Cloning: A Laboratory Manual, 2^{nd} ed.; Cold Spring Harbor Laboratory: Cold Spring Harbor, N.Y., 1989 (hereinafter “Maniatis”); and by Silhavy, T. J., Bennan, M. L. and Enquist, L. W. Experiments with Gene Fusions; Cold Spring Harbor Laboratory: Cold Spring Harbor, N.Y., 1984; and by Ausubel, F. M. et al., In Current Protocols in Molecular Biology, published by Greene Publishing and Wiley-lnterscience, 1987.

Engineering p-Hydroxycinnamic Acid Production in a Recombinant Bacterial Host Cell

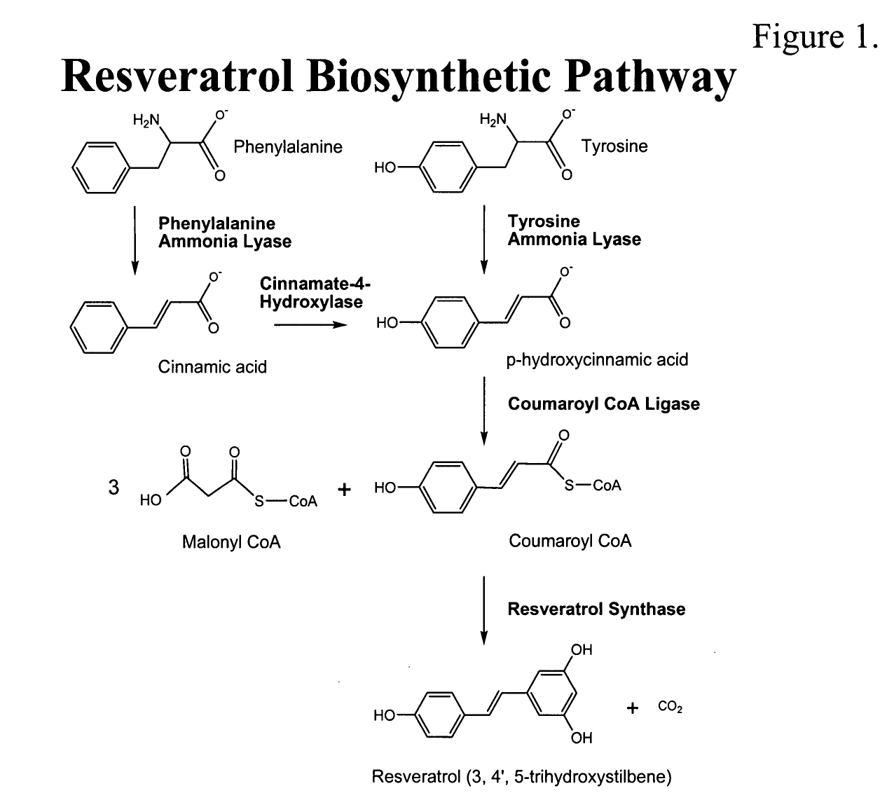

Coumaroyl CoA ligases converts p-hydroxycinnamic acid (pHCA) into p-coumaroyl CoA. In one aspect, the present method uses bacterial host cells engineered to produce pHCA (FIG. 1). In one embodiment, Para-hydroxycinnamic acid is produced by expressing a phenylalanine ammonia lyase in combination with a cinnamate 4-hydroxylase (C4H), harnessing the endogenous production of the aromatic amino acid phenylalanine to produce pHCA. In a preferred embodiment, the host cell endogenously provides cinnamate 4-hydroxylase activity.

In another aspect, L-tyrosine is converted directly to p-hydroxycinnamic acid by expressing a tyrosine ammonia lyase or a phenylalanine ammonia lyase having activity towards tyrosine (i.e., a “PAL/TAL” enzyme). In yet another aspect, pHCA is supplied exogenously and/or synthesized by the recombinant host cell. In a preferred aspect, pHCA is supplemented/added to the culture medium. In a further aspect, L-phenylalanine, L-tyrosine, malonic acid (or salt thereof) and/or trans-cinnamate is exogenously supplied to the recombinant host cell expressing a phenylalanine/tyrosine ammonia lyase and/or a cinnamate 4-hydroxylase.

In one aspect, a phenylalanine hydroxylase (PAH) is recombinantly expressed in a host cell capable of producing phenylalanine to increase tyrosine production (assuming that a tyrosine ammonia lyase activity is present to convert tyrosine into pHCA). In another aspect, the host cell is engineered to recombinantly express genes required to convert a portion of the aromatic amino acids endogenously produced by the host cell (L-phenylalanine and/or L-tyrosine) into pHCA (i.e., introduction of genes in the phenylpropanoid pathway). One of skill in the art will recognize that there is a need to balance the carbon flow from aromatic amino acid production into pHCA production (and eventually resveratrol production) so that a decrease in concentration of the free aromatic amino acids is not detrimental to the viability or health of the recombinant host cell. In a further aspect, phenylalanine and/or tyrosine can be supplemented to the culture medium to increase resveratrol production. In yet a further aspect, the genes involved in aromatic amino acid biosynthesis are upregulated to increase the production of L-phenylalanine and/or L-tyrosine.

Recombinant microbial expression of a nucleic acid molecule encoding an enzymes having phenylalanine/tyrosine ammonia lyase activity for converting L-tyrosine to pHCA has been reported. For example, recombinant expression of the Rhodotorula glutinis PAL (SEQ ID NOs: 11 and 12) has been shown to produce pHCA from L-tyrosine. Other PAL/TAL genes are publicly available and known in the art (for example, see Table 1 for a non-limiting list). One of skill in the art can select and recombinantly express one or more genes encoding enzyme(s) having PAL/TAL activity using the present methods. In another aspect, a gene encoding a polypeptide having PAL/TAL activity is codon optimized according the preferred codon usage frequency of the chosen bacterial host cell.

Production of p-Coumaroyl CoA from pHCA

The pHCA is converted into p-coumaroyl CoA by expressing an enzyme having coumaroyl CoA ligase activity. The coumaroyl CoA ligase can be endogenous to the host cell or can be recombinantly expressed within the host cell to increase p-coumaroyl CoA production. Microbial expression of plant and/or bacterial coumaroyl CoA ligases has previously been reported. In one aspect, the coumaroyl CoA ligase is codon optimized for optimal expression within the chosen bacterial host cell. The coumaroyl CoA ligases presently exemplified was isolated from Streptomyces coelicolor (ATCC® BAA471D™) (SEQ ID NOs: 3 and 4) or from Petroselinium crispum (SEQ ID NOs: 20 and 21). However, one of skill in the art can select and recombinantly expression any of the publicly available coumaroyl CoA ligases (see for example, Table 1 for a non-limiting list). In one aspect, the coumaroyl CoA ligase is chosen based on its ability to convert pHCA into p-coumaroyl CoA. In another aspect, a plurality of coumaroyl CoA ligases are coexpressed to increase the production of p-coumaroyl CoA. In yet another aspect, the coumaroyl CoA ligase activity is derived from Streptomyces coelicolor, Acinectobacter sp. ADP1, or Petroselinum crispum. In a further embodiment, the gene(s) encoding the coumaroyl CoA ligase are overexpressed in the recombinant bacterial cell.

Production of Malonyl CoA

Resveratrol synthase (stilbene synthase) catalyzes the formation of resveratrol (trans-3,4′,5-trihydroxystilbene) by combining 3 molecules of malonyl CoA with 1 molecule p-coumaroyl CoA (FIG. 1). In one aspect, the recombinant bacterial host cell endogenously produces suitable amounts of malonyl CoA.

In another aspect, the bacterial host cell is engineered to produce suitable amounts malonyl CoA by recombinantly expressing acetyl CoA carboxylase (Davis et al., J. Biol. Chem., 275:28593-28598 (2000)). Acetyl CoA carboxylase catalyzes the production of malonyl CoA from acetyl CoA (carboxylates acetyl CoA, creating malonyl CoA). Acetyl CoA carboxylases are known in the art (Table 1; Davis et al., supra). In another aspect, the gene encoding acetyl CoA carboxylase is codon optimized according to the preferred codon usage of the target host cell.

In another embodiment, the recombinant host cell is engineered to recombinantly express an enzyme having malonyl CoA synthetase activity (E.C. 6.2.1.-). Malonyl CoA synthetases catalyzes the synthesis of malonyl CoA from malonate and CoA (Kim and Yang, Biochem. J. 297:327-333 (1994)). Genes encoding enzymes having malonyl CoA synthetase activity are known in the art. Recombinant expression of malonyl CoA synthetases has been reported (An, J. H., and Kim, Y. S., Eur. J. Biochem. 257:395402 (1998)). A non-limiting list of malonyl CoA synthetases is provided in Table 1. In one embodiment, the recombinant host cell expresses at least one malonyl CoA synthetase in order to produce suitable amounts of malonyl CoA when grown on an inexpensive carbon source (i.e., the cell produces malonate and CoA). In another embodiment, a source of malonate (e.g., malonic acid or salt thereof) is supplemented to the fermentation medium to increase resveratrol production.

Uptake of exogenous supplied malonic acid/malonate may be improved by coexpressing at least one nucleic acid molecule encoding an enzyme having dicarboxylate carrier protein activity. Dicarboxylate carrier proteins are membrane bound proteins that facilitate dicarboyxlate transport across the cell membrane. As used herein, “dicarboxylate carrier protein” and “malonyl transport protein” will be used interchangeably and refer to membrance bound proteins that aid in the transport of dicarboxylates (i.e., malonate) into the cell. As used herien, “dicarboxylate carrier protein activity” and “malonyl transport activity” will be used to describe membrance proteins that aid in the transport of dicarboxylates (i.e., malonate) into the cell. In a preferred embodiment it has been found that resveratrol yield may be improved by supplementation of the culture medium with either p-hydroxycinnamic acid, or malonic acid (malonate), or mixtures thereof at a concentration of at least 3 mM, preferably at least 5 mM, and most preferably at least 10 mM.

Interestingly, malonyl CoA biosynthesis operons have been reported to contain coding regions for both malonyl CoA synthetase (matB) and a dicarboxylate carrier protein (malonate transporter; matC), often adjacent to one another in the bacterial genome. Recombinant expression of matB and matC genes has been reported (An, J. H., and Kim, Y. S., supra). A non-limiting list of genes encoding dicarboyxlate transport proteins is provided in Table 1. In one embodiment, host cells grown in the presence of endogenously supplemented malonate/malonic acid recombinantly express at least one nucleic acid molecule encoding a protein having dicarboxylate carrier protein (malonic acid transporter) activitiy.

In one embodiment, the recombinant bacterial host cell engineered for resveratrol production expresses at least one nucleic acid molecule encoding an enzyme having malonyl CoA synthetase activity and at least one nucleic acid molecule encoding a dicarboxylate carrier protein.

Hydrolysis of Resveratrol Glucoside to Free Resveratrol

Although glycosylation activity is typically not observed in bacterial host cells, one can engineer the host cell to produce resveratrol glucoside (piceid). In one aspect, the bacterial host cell may endogenously glycosylate the resveratrol to produce resveratrol glucoside. In another aspect, the bacterial host cell may be engineered to recombinantly express a glucosyl transferase (U.S. Ser. No. 10/359,369; hereby incorporated by reference). Glucose moieties attached to the resveratrol glucoside can be hydrolyzed to produce free resveratrol (i.e., the aglycone or “free” resveratrol). In yet another aspect, the glucose moieties are removed from the piceid using a non-enzymatic process such as acid or base hydrolysis (Jencks, William, P., in Catalysis in Chemistry and Enzymology, Dover Publications, New York, 1987). In a further aspect, the recombinantly produced resveratrol glucoside is treated with a β-glucosidase to release the sugar moieties bound to resveratrol. In yet a further aspect, gene(s) encoding endogenous glucosyltransferase(s) is/are disrupted to block the production of the resveratrol glycoside (assuming this is not detrimental to the growth characteristics and/or viability of the host cell).

In one aspect, the resveratrol and/or resveratrol glycoside is accumulated within the recombinant bacterial host cell. In this instance, the resveratrol and/or resveratrol glycoside is purified from the recombinant host cells. In a further aspect, the recombinant host cell is further modified so that the resveratrol (or resveratrol glucoside) produced is secreted from the host cell into the fermentation medium where it can be purified in batch or continuously removed from the fermentation medium. In yet another aspect, the resveratrol glucoside produced by the recombinant host cell is the desired end product (i.e., for use in personal care products, dietary supplements, antioxidant compositions, antifungal compositions, animal feeds, cometics, and pharmaceutical compositions, to name a few).

Gene Useful for Recombinant Production of Resveratrol

The key enzymatic activities used in the present invention are encoded by a number of genes known in the art. The principal enzymes used in recombinant bacterial biosyntheis typically include, but are not limited to phenylalaine/tyrosine ammonia lyase, cinnamate 4-hydroxylase (when converting phenylalanine to cinnamate using PAL activity), coumaroyl CoA ligase, malonyl CoA synthetase (preferably in combination with a protein having dicarboxylate transport protein activity), and resveratrol synthase (FIG. 1). Additional enzymes useful for the production of resveratrol in the transformed microorganisms may also include acetyl CoA carboxylase (E.C. 6.4.1.2; carboxylates acetyl CoA to make malonyl CoA), phenylalanine hydroxylase (used to convert phenylalanine to tyrosine), and β-glucosidase (used to remove sugar moieties from resveratrol glycoside) (FIG. 1). In one aspect, the genes useful to produce resveratrol are expressed in multiple copies, optionally having divergent amino acid and/or nucleic acid sequences to ensure genetic stability in the production host (i.e., reduce or eliminate the probability of homologous recombination). In one aspect, one or more of the genes used to produce resveratrol are chromosomally-integrated for expression. In yet another aspect, one or more of the genes used to produced resveratrol are expressed extrachromosomally (i.e., on an expression vector).

In one aspect, one or more of the present genes are codon-optimized for expression in the bacterial host cell. Preferred codon usage frequencies for a variety of bacterial host cells are known in the art. In another aspect, one of skill in the art can determine the preferred codon usage frequency of the target bacterial cell by sequencing a plurality of genes endogenously expressed within the host cell and comparing the relative frequency of each codon used. Less frequently used codons are then replaced with codons typically used by the target host cell.

The current methods are exemplified using genes isolated from specific sources. However, one of skill in the art recognizes that homologs for each of the exemplified genes are known in the art as shown (but not limited to) in Table 1.

TABLE 1
Examples of Alternative Sources for Genes Useful for
Recombinant Production of Resveratrol

| | GenBank ® Accession No., | |
|---|---|---|
| Gene | Source Organism | SEQ ID NO.: |
| pal, tal | X13094, Rhodotorula mucilaginosa | 23 |
| (phenylalanine | AAJ10143, Amanita muscaria | 24 |
| ammonia lyases | XM397693, AF306551, Ustilago | 25 |
| and/or | maydis | |
| tyrosine | AY079363, Arabidopsis thaliana | 26 |
| ammonia lyases) | AF237955, Rubus idaeus | 27 |
| | X58180, Medicago sativa | 28 |
| | AF401636, Rehmannia glutinosa | 29 |
| | AF299330, Lactuca savita | 30 |
| | P14913, Petroselinium crispum | 31 |
| | AF036948, Prunus avium | 32 |
| | D83075, Lithospernum | 33 |
| | erythrorhizon | |
| | U43338, Citrus limon | 34 |
| | AAP01719, Rhodotorula glutinis | 35 |
| | from U.S. Pat. No. 6521748 | |
| | ZP_00005404, Rhodobacter | 36 |
| | sphaeroides | |
| | AR722988, Trichosporon cutaneum | 37 |
| | from U.S. Pat. No. 6951751 | |
| Coumaroyl CoA | CAB95894, AL939119, for | 38 |
| ligase (4CL) | Streptomyces coelicolor | |
| | AY541033, Allium cepa | 39 |
| | AP005036, Streptomyces | 40 |
| | avermitilis | |
| | AF041049, Populus tremuloides | 41 |
| | XM_482683, Oryza sativa | 42 |
| | AF435968, Amorpha fruticosa | 43 |
| | AY043495, Populus tomentosa | 44 |
| | D43773, Nicotiana tabacum | 45 |
| | U12013, Pinus taeda | 46 |
| | AF279267, Glycine max | 47 |
| | NM_113019, Arabidopsis thaliana | 48 |
| | AY376731, Arabidopsis thaliana | 49 |
| | AF239687, Rubus idaeus | 50 |
| | D49367, Lithospermum | 51 |
| | erythrorhizon | |
| | AY566301, Zea mays | 52 |
| Resveratrol | S63225, Vitis sp. | 53 |
| Synthase (RS) | AF274281, Vitis vinifera | 54 |
| (Stilbene | X76892.1, Vitis vinifera | 55 |
| synthase) | AB027606, Arachis hypogaea | 56 |
| | AY094616.1, Cissus rhombifolia | 57 |
| | AY094615.1, Parthenocissus | 58 |
| | henryana | |
| | AY094617.1, Parthenocissus | 59 |
| | quinquefolia | |
| | AB046373.1, Vitis riparia | 60 |
| | AB046374.1, Vitis labrusca | 61 |
| | AF418566, Vitis sp. cv. “Norton” | 62 |
| Cinnamate 4- | O81928, AJ007449, Cicer arietinum | 63 |
| hydroxylase | O24312, U47293, Populus | 64 |
| (C4H) | tremuloides | |
| | XP_465542, Oryza sativa | 65 |
| | AAT68775, AY641731, Camellia | 66 |
| | sinensis | |
| | P37115, L07634, Vigna radiata | 67 |
| | Q04468, Z17369, Helianthus | 68 |
| | tuberosus | |
| | AAT39513, AY621152, | |
| | Camptotheca acuminata | 69 |
| | P92994, U71081, Arabidopsis | 70 |
| | thaliana | |
| | AAN63028, AF548370, Ruta | 71 |
| | graveolens | |
| | Q42797, X92437, Glycine max | 72 |
| | AAF66065, AF255013, Citrus | 73 |
| | sinensis | |
| Phenylalanine | AAA23115, M55915, | |
| hydroxylase | Chromobacterium violaceum | 74 |
| (PAH) | AAA25938, M88627, | |
| | Pseudomonas aeruginosa | 75 |
| | CAA76184, Y16353, Geodia | 76 |
| | cydonium | |
| | AAM35066, AE011641, | |
| | Xanthomonas axonopodis | 77 |
| | AAM39475, AE012111, | |
| | Xanthomonas campestris | 78 |
| | BAD55786, AP006618 Nocardia | 79 |
| | farcinica | |
| | NP_001001298, Gallus gallus | 80 |
| Acetyl CoA | NP_014413, NC_001146, | |
| carboxylase | Saccharomyces cerevisiae | 81 |
| | M92156, Saccharomyces | 82 |
| | cerevisiae | |
| | XM_455355, Kluyveromyces lactis | 83 |
| | XM_457211, Debaryomyces | 84 |
| | hansenii | |
| | XM_501721, Yarrowia lipolytica | 85 |
| | Y15996, Aspergillus nidulans | 86 |
| | D78169, Schizosaccharomyces | 87 |
| | pombe | |
| | Z46886, Ustilago maydis | 88 |
| | J03541, Gallus gallus | 89 |
| β-Glucosidase | YP_053668, NC_006055 | |
| | Mesoplasma forum | 90 |
| | AAV32242, AC135927 Oryza sativa | 91 |
| | NP_743562, NC_002947 | |
| | Pseudomonas putida | 92 |
| | NP_793101, NC_004578 | |
| | Pseudomonas syringae | 93 |
| | NP_630676, NC_003888 | |
| | Streptomyces coelicolor A3(2) | 94 |
| | NP_420939, NC_002696 | |
| | Caulobacter crescentus | 95 |
| | 2107160A, U13672, Candida | 96 |
| | wickerhamii | |
| Malonyl CoA | AF118888, Bradyrhizobium | 97 |
| Synthetase | japonicum | |
| (matB) | NZ_AALJ01000002, | |
| | Bradyrhizobium sp. BTAi1 | 98 |
| | BX572593, Rhodopseudomonas | 99 |
| | palustris | |
| | BA000012, Mesorhizobium loti | 100 |
| Dicarboxylate | NC_007761, Rhizobium etli | 101 |
| Transport | NC_007508, Xanthomonas | 102 |
| Protein (i.e., | campestris pv. vesicatoria tr. | |
| malonate | NC_003902, Xanthomonas | 103 |
| transporter) | campestris pv. Campestris | |
| (matC) | | |

In one embodiment, the present method comprising at least one nucleic acid molecule encoding an enzyme providing resveratrol synthase activity selected from the group consisting of SEQ ID NOs: 7, 53, 54, 55, 56, 57, 58, 59, 60, 61, and 62.

In another embodiment, the present method comprises at least one nucleic acid molecule encoding an enzyme providing resveratrol synthase activity is selected from the group consisting of:
(1) a nucleic acid molecule encoding a polypeptide having resveratrol synthase activity, said polypeptide having an amino acid sequence SEQ ID NO: 8;
(2) a nucleic acid molecule encoding a polypeptide having resveratrol synthase activity, said polypeptide having 95% identity to SEQ ID NO: 8; and
(3) a nucleic acid molecule that hybridizes with (a) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In one embodiment, the present method comprising at least one nucleic acid molecule encoding an enzyme providing coumaroyl CoA ligase activity selected from the group consisting of SEQ ID NOs: 3, 20, 38, 39, 40, 41, 42, 43, 44, 45, 46, 47, 48, 49, 50, 51, and 52.

In another embodiment, the present method comprises at least one nucleic acid molecule encoding an enzyme providing coumaroyl CoA ligase activity is selected from the group consisting of:
(1) a nucleic acid molecule encoding a polypeptide having coumaroyl CoA ligase activity, said polypeptide having an amino acid sequence selected from the group consisting of SEQ ID NO: 4 and SEQ ID NO: 21;
(2) a nucleic acid molecule encoding a polypeptide having coumaroyl CoA ligase activity, said polypeptide having 95% identity to an amino acid sequence selected from the group consiting of SEQ ID NO: 4 and SEQ ID NO: 8; and
(3) a nucleic acid molecule that hybridizes with (a) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In one embodiment, the present method comprising at least one nucleic acid molecule encoding an enzyme providing phenylalanine/tyrosine ammonia lyase activity selected from the group consisting of SEQ ID NOs: 11, 23, 24, 25, 26, 27, 28, 29, 30, 31, 32, 33, 34, 35, 36, and 37.

In another embodiment, the present method optionally comprises at least one nucleic acid molecule encoding an enzyme providing phenylalanine/tyrosine ammonia lyase activity is selected from the group consisting of:
(1) a nucleic acid molecule encoding a polypeptide having phenylalanine/tyrosine ammonia lyase activity, said polypeptide having an amino acid sequence SEQ ID NO: 12;
(2) a nucleic acid molecule encoding a polypeptide having phenylalanine/tyrosine ammonia lyase activity, said polypeptide having 95% identity to SEQ ID NO: 12; and
(3) a nucleic acid molecule that hybridizes with (a) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In one embodiment, the present method comprising at least one nucleic acid molecule encoding an enzyme providing Malonyl CoA synthetase activity selected from the group consisting of SEQ ID NOs: 13, 97, 98, 99, and 100.

In another embodiment, the present method includes at least one nucleic acid molecule encoding a malonyl CoA synthetase selected from the group consisting of:
a) an isolated nucleic acid molecule encoding a polypeptide having malonyl CoA synthetase activity; said polypeptide having the amino acid sequence SEQ ID NO: 14;
b) an isolated nucleic acid molecule encoding a polypeptide having malonyl CoA synthetase activity, said polypeptide having 95% amino acid identity to to SEQ ID NO: 14; and
c) an isolated nucleic acid molecule that hybridizes with (a) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In one embodiment, the present method comprising at least one nucleic acid molecule encoding an polypeptide providing dicarboxylate transport protein activity selected from the group consisting of SEQ ID NOs: 15,101, 102, and 103.

In another embodiment, the present method includes at least one nucleic acid molecule encoding a dicarboxylate carrier protein selected from the group consisting of:
a) an isolated nucleic acid molecule encoding a polypeptide having dicarboxylate carrier protein activity; said polypeptide having the amino acid sequence SEQ ID NO: 16;
b) an isolated nucleic acid molecule encoding a polypeptide having dicarboxylate carrier protein activity, said polypeptide having 95% amino acid identity to to SEQ ID NO: 16; and
c) an isolated nucleic acid molecule that hybridizes with (a) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In another embodiment, the present invention provides a resveratrol-producing and/or resveratrol glucoside-producing recombinant bacterial host cell comprising at least one isolated nucleic acid molecule encoding an enzyme having resveratrol synthase activity, at least one isolated nucleic acid molecule encoding an enzyme providing coumaroyl CoA ligase activity, and optionally at least one nucleic acid molecule encoding an enzyme having malonyl CoA synthetase activity.

In another embodiment, the present invention provides a resveratrol-producing and/or resveratrol glucoside-producing recombinant bacterial host cell comprising at least one isolated nucleic acid molecule encoding an enzyme having resveratrol synthase activity, at least one isolated nucleic acid molecule encoding an enzyme providing coumaroyl CoA ligase activity, and at least one nucleic acid molecule encoding an enzyme having malonyl CoA synthetase activity.

In another embodiment, the present invention provides a resveratrol-producing and/or resveratrol glucoside-producing recombinant bacterial host cell comprising at least one isolated nucleic acid molecule encoding an enzyme having resveratrol synthase activity, at least one isolated nucleic acid molecule encoding an enzyme providing coumaroyl CoA ligase activity, and at least one nucleic acid molecule encoding an enzyme having malonyl CoA synthetase activity, and at least one nucleic acid molecule encoding a polypeptide having dicarboxylate carrier protein activity (i.e., transports malonate/malonic acid into the host cell).

In a further embodiment, the present invention provides a recombinant bacterial host cell further comprising at least one nucleic acid molecule encoding an enzyme having phenylalanine/tyrosine ammonia lyase activity. Preferably the enzyme having phenylalanine/tyrosine ammonia lyase activity will have a tyrosine ammonia lyase activity to phenylalanine ammonia lyase activity (TAL specific activity:PAL specific activity) of at least 0.1, more preferably at least 1, even more preferably at least 10, and most preferably at least 1000.

In still a further embodiment, an a resveratrol producing recombinant bacterial host cell is provided comprising:
a) at least one nucleic acid molecule encoding an enzyme having resveratrol synthase activity selected from the group consisting of:
   i) a nucleic acid molecule encoding a polypeptide having an amino acid sequence SEQ ID NO: 8;
   ii) a nucleic acid molecule encoding a polypeptide having having 95% identity to SEQ ID NO: 8; and
   iii) a nucleic acid molecule that hybridizes with (a)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.;
b) at least one nucleic acid molecule encoding an enzyme having coumaroyl CoA ligase activity selected from the group consisting of:
   i) a nucleic acid molecule encoding a polypeptide having an amino acid sequence selected from the group consiting of SEQ ID NO: 4 and SEQ ID NO: 21;
   ii) a nucleic acid molecule encoding a polypeptide having 95% identity to an amino acid sequence selected from the group consiting of SEQ ID NO: 4 and SEQ ID NO: 21; and
   iii) a nucleic acid molecule that hybridizes with (b)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.; and
c) optionally at least one nucleic acid molecule encoding an enzyme having phenylalanine/tyrosine ammonia lyase activity selected from the group consisting of:
   i) a nucleic acid molecule encoding a polypeptide having an amino acid sequence SEQ ID NO:12;
   ii) a nucleic acid molecule encoding a polypeptide having 95% identity to SEQ ID NO: 12; and
   iii) a nucleic acid molecule that hybridizes with (c)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In yet another embodiment, an isolated recombinant bacterial cell capable of producing resveratrol or resveratrol glucoside is provided comprising:
a) at least one nucleic acid molecule encoding an enzyme having resveratrol synthase activity selected from the group consisting of:
   i) a nucleic acid molecule encoding a polypeptide having an amino acid sequence SEQ ID NO: 8;
   ii) a nucleic acid molecule encoding a polypeptide having 95% identity to SEQ ID NO: 8; and
   iii) a nucleic acid molecule that hybridizes with (a)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.;
b) at least one nucleic acid molecule encoding an enzyme having coumaroyl CoA ligase activity selected from the group consisting of:
   i) a nucleic acid molecule encoding a polypeptide having an amino acid sequence selected from the group consisting of SEQ ID NO: 4 and SEQ ID NO: 21;
   ii) a nucleic acid molecule encoding a polypeptide having 95% identity to an amino acid sequence selected from the group consisting of SEQ ID NO: 4 and SEQ ID NO: 21; and
   iii) a nucleic acid molecule that hybridizes with (b)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.; and
c) at least one nucleic acid molecule encoding a polypeptide having malonyl CoA synthetase activity selected from the group consisting of:
   i) a nucleic acid molecule encoding an enzyme having an amino acid sequence SEQ ID NO: 14;
   ii) an isolated nucleic acid molecule encoding a polypeptide having 95% amino acid identity to to SEQ ID NO: 14; and
   iii) an isolated nucleic acid molecule that hybridizes with (c)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.; and
d) at least nucleic acid molecule encoding a polypeptide having dicarboxylate transport protein activity selected from the group consisting of:
   i) an nucleic acid molecule encoding a polypeptide having the amino acid sequence SEQ ID NO: 16;
   ii) an isolated nucleic acid molecule encoding a polypeptide having 95% amino acid identity to to SEQ ID NO: 16; and
   iii) an isolated nucleic acid molecule that hybridizes with (d)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.
e) optionally at least one nucleic acid molecule encoding an enzyme having phenylalanine/tyrosine ammonia lyase activity selected from the group consisting of:
   i) a nucleic acid molecule encoding a polypeptide having amino acid sequence SEQ ID NO: 12;
   ii) a nucleic acid molecule encoding a polypeptide having 95% identity to SEQ ID NO: 12; and
   iii) a nucleic acid molecule that hybridizes with (e)(i) under the following hybridization conditions: 0.1×SSC, 0.1% SDS, 65° C. and washed with 2×SSC, 0.1% SDS, at 65° C.; followed by 0.1×SSC, 0.1% SDS, at 65° C.

In another embodiment, the present invention provides an recombinant bacterial biomass comprising at least 0.1% dry cell weight (dcw), preferably at least 0.2% (dcw), more preferably at least 1% (dcw), and most preferably at least 2% (dcw) resveratrol for inclusion in an animal feed, a pharmaceutical composition, an antioxidant composition, a personal care product, an antifungal composition, or a dietary supplement.

Phenylalanine Ammonia Lyase (PAL) and Cinnamate 4-hydroxylase (C4H)

Phenylalanine ammonia-lyase (PAL) (EC 4.3.1.5) is widely distributed in plants (Koukol et al., J. Biol. Chem., 236:2692-2698 (1961)), fungi (Bandoni et al., Phytochemistry, 7:205-207 (1968)), yeast (Ogata et al., Agric. Biol. Chem., 31:200-206 (1967)), and Streptomyces (Emes et al., Can. J. Biochem., 48:613-622 (1970)), but it has not been found in Escherichia coli or mammalian cells (Hanson and Havir In The Enzymes, 3^{rd} ed.; Boyer, P., Ed.; Academic: New York, 1967; pp 75-167). PAL is the first enzyme of phenylpropanoid metabolism and catalyzes the removal of the (pro-3S)-hydrogen and —NH₃⁺ from L-phenylalanine to form trans-cinnamic acid. In the presence of a P450 enzyme system, trans-cinnamic acid can be converted to para-hydroxycinnamic acid (pHCA) which serves as the common intermediate in plants for production of various secondary metabolites such as lignin and isoflavonoids. In microbes however, cinnamic acid and not pHCA acts as the precursor for secondary metabolite formation. A cinnamate 4-hydroxylase enzyme (C4H) converts cinnamic acid to p-hydroxycinnamic acid.

Tyrosine Ammonia Lyase (TAL) to Convert Tyrosine to pHCA

Another biosynthetic pathway leading to the production of pHCA is based on an enzyme having tyrosine ammonia lyase activity. Instead of the two enzyme reaction to convert phenylalanine to pHCA, tyrosine ammonia lyase converts tyrosine directly into pHCA. A coumaroyl CoA ligase is then be used to convert pHCA into p-coumaroyl CoA. In one aspect, an enzyme classified as a tyrosine ammonia lyase can be recombinantly expressed in the host cell. In another aspect, a phenylalanine ammonia lyase having tyrosine ammonia lyase activity is used to convert tyrosine into pHCA.

Mutating Phenylalanine Ammonia Lyase to Create Tyrosine Ammonia Lyase (TAL)

In nature, genes encoding phenylalanine ammonia-lyase are known to convert phenylalanine to trans-cinnamate, which may be converted to para-hydroxycinnamic acid (pHCA) via a p-450/p-450 reductase enzyme system (FIG. 1). In many instances phenylalanine ammonia lyases will recognize tyrosine as a substrate, catalyzing its conversion directly to pHCA. For example, the PAL enzyme isolated from parsley (Appert et al., Eur. J. Biochem., 225:491 (1994)) and corn ((Havir et al., Plant Physiol., 48:130 (1971)) both demonstrate the ability to use tyrosine as a substrate. Similarly, the PAL enzyme isolated from Rhodosporidium (Hodgins D S, J. Biol. Chem., 246:2977 (1971)) also accepts tyrosine as a substrate. Such enzymes will be referred to herein as “PAL/TAL” enzymes or activities. Where it is desired to create a recombinant organism expressing a wild type gene encoding PAL/TAL activity, genes isolated from maize, wheat, parsley, Rhizoctonia solani, Rhodosporidium, Sporobolomyces pararoseus, and Rhodosporidium may be used as discussed in Hanson and Havir, The Biochemistry of Plants; Academic: New York, 1981; Vol. 7, pp 577-625.

In some instances it is possible to alter the substrate specificity of the PAL/TAL enzyme via various forms of mutagenesis and protein engineering. In one aspect, phenylalanine ammonia lyase is protein engineered to accept tyrosine as a substrate for the production of pHCA (U.S. Pat. No. 6,521,748; hereby incorporated by reference). A variety of approaches may be used for the mutagenesis of the PAL/TAL enzyme. Suitable approaches for mutagenesis include error-prone PCR (Leung et al., Techniques, 1:11-15 (1989) and Zhou et al., Nucleic Acids Res., 19:6052-6052 (1991) and Spee et al., Nucleic Acids Res., 21:777-778 (1993)), in vitro mutagenesis, and in vivo mutagenesis. Protein engineering may be accomplished by the method commonly known as “gene shuffling” (U.S. Pat. No. 5,605,793; U.S. Pat. No. 5,811,238; U.S. Pat. No. 5,830,721; and U.S. Pat. No. 5,837,458), by recombinogenic methods as described in U.S. Ser. No. 10/374,366, or by rationale design methods based on three-dimensional structure and classical protein chemistry.

The process of protein engineering a phenylalanine ammonia lyase into an mutant enzyme capable of using tyrosine as a substrate (hence tyrosine ammonia lyase activity) has previously been reported (U.S. Pat No. 6,368,837; hereby incorporated by reference).

Phenylalanine Hydroxylase (PAH) to Increase Tyrosine Production

In another aspect, phenylalanine hydroxylase (PAH) activity is endogenous to the bacterial host cell or is introduced into the host cell to increase production of tyrosine (FIG. 1). The PAH enzyme hydroxylates phenylalanine to produce tyrosine. This enzyme is well known in the art and has been reported in Proteobacteria (Zhao et al., In Proc. Natl. Acad. Sci. USA., 91:1366 (1994)). For example Pseudomonas aeruginosa possesses a multi-gene operon that includes phenylalanine hydroxylase, which is homologous with mammalian phenylalanine hydroxylase, tryptophan hydroxylase, and tyrosine hydroxylase (Zhao et al., supra). The enzymatic conversion of phenylalanine to tyrosine is known in eukaryotes. Human phenylalanine hydroxylase is expressed in the liver, converting L-phenylalanine to L-tyrosine (Wang et al., J. Biol. Chem., 269 (12): 9137-46 (1994)). Although any gene encoding a PAH activity is useful, genes isolated from Proteobacteria are particularly suitable. A PAH gene has been isolated from Chromobacterium violaceum and recombinantly expressed (U.S. Ser. No. 10/138,970; hereby incorporated by reference).

Coumaroyl CoA Ligase (4CL) for the Synthesis of p-Coumaroyl-CoA from pHCA

Coumaroyl CoA ligase catalyzes the conversion of 4-coumaric acid and other substituted cinnamic acids into the corresponding CoA thiol esters. In the present invention, coumaroyl CoA ligase is used to convert pHCA into p-coumaroyl CoA, one of the substrates used by resveratrol synthase to produce resveratrol. Coumaroyl CoA ligases are well-known in the art and have been recombinantly expressed in microorganisms (Watts et al., supra; Hwang et al., supra; and Kaneko et al., supra). A non-limited list of additional, publicly available, coumaroyl CoA ligase genes is provided in Table 1.

Resveratrol Synthase (Stilbene Synthase)

Resveratrol synthase, also referred to as stilbene synthase, catalyzes the formation of resveratrol from p-coumaroyl CoA and malonyl CoA. Specifically, resveratrol is formed by three consecutive Claisen condensations of the acetate unit from malonyl CoA with p-coumaroyl CoA, which is succeeded by an aldol reaction that forms the second aromatic ring, cleaves the thioester, and decarboxylates to produce resveratrol.

The present methods are exemplified using the resveratrol synthase isolated from Vitis sp. (SEQ ID NOs: 7-9). Resveratrol synthases are highly conserved in both structure and function based on comparisons to publicly available sequences. As such, one of skill in the art would expect that the present methods are not limited to the particular resveratrol synthase exemplified in the present examples. In one preferred aspect, the present method uses one or more resveratrol synthase genes codon optimized for expression in the bacterial host cell. In yet another preferred aspect, the gene is codon optimized for expression in E. coli. A non-limited list of additional, publicly available, resveratrol synthase genes is provided in Table 1.

Synthesis of Malonyl CoA

Synthesis of resveratrol is dependent upon an available pool of malonyl CoA. In one aspect, the bacterial host cell naturally produces suitable amounts of malonyl CoA. In another aspect, the bacterial host cell is genetically modified to increase the amount of available malonyl CoA. In yet a further aspect, the bacterial host cell is modified to increase expression of acetyl CoA carboxylase (Davis et al., supra). A non-limited list of additional, publicly available acetyl CoA carboxylases is provided in Table 1.
a) In another embodiment, the bacterial host cell is engineered to expression at least one nucleic acid molecule encoding an enzyme having malonyl CoA synthetase activity (the enzyme catalyzes the carboxylation of acetyl CoA into malonyl CoA). A non-limited list of additional, publicly available malonyl CoA synthetase genes is provided in Table 1.
   In a preferred embodiment, the malonly CoA synthetase gene is coexpressed
a) with at least one nucleic acid molecule encoding a protein have dicarboxylate transport protein activity (i.e., aids in the transport of extracellular malonate/malonic acid across the cell membrance). A non-limited list of additional, publicly available genes encoding dicarboxylate transport proteins is provided in Table 1
   Recombinant Expression—Microbial

The genes and gene products of the instant sequences may be produced in heterologous host cells, particularly in the cells of microbial hosts. Expression in recombinant microbial cells may be useful for: the expression of various pathway intermediates; the modulation of pathways already existing in the host, or the synthesis of new products heretofore not possible using the host. In one aspect, recombinant expression of the present genes is useful to increase resveratrol production.

Preferred heterologous host cells for expression of the instant genes and nucleic acid molecules are microbial hosts that can be found within bacterial families and which grow over a wide range of temperature, pH values, and solvent tolerances. For example, it is contemplated that any of bacteria may suitably host the expression of the present nucleic acid molecules. Transcription, translation and the protein biosynthetic apparatus remain invariant relative to the cellular feedstock used to generate cellular biomass; functional genes will be expressed regardless Examples of suitable host strains include, but are not limited to bacterial species such as Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. Preferred bacterial host strains include Escherichia, Bacillus, and Methylomonas. A most preferred bacterial host strain is Escherichia coli.

Large-scale microbial growth and functional gene expression may be regulated by certain growth conditions, such as the use of a wide range of simple or complex carbohydrates, organic acids and alcohols or saturated hydrocarbons such as methane or carbon dioxide in the case of photosynthetic or chemoautotrophic hosts or other specific growth conditions, which may include the form and amount of nitrogen, phosphorous, sulfur, oxygen, carbon or any trace micronutrient including small inorganic ions. The regulation of growth rate may be affected by the addition, or not, of specific regulatory molecules to the culture and which are not typically considered nutrient or energy sources.

Microbial expression systems and expression vectors containing regulatory sequences that direct high level expression of foreign proteins are well known to those skilled in the art. Any of these could be used to construct chimeric genes for expression of present genes. These chimeric genes are then be introduced into appropriate microorganisms via known techniques to provide high-level expression of the enzymes. Accordingly, it is expected that introduction of chimeric genes encoding enzymes involved in recombinant resveratrol production are under the control of the appropriate promoters and will demonstrate increased or altered resveratrol production.

Vectors or cassettes useful for the transformation of suitable host cells are well known in the art. Typically the vector or cassette contains sequences directing transcription and translation of the relevant gene, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell and/or native to the production host, although such control regions need not be so derived.

Initiation control regions or promoters, which are useful to drive expression of the instant ORF's in the desired host cell are numerous and familiar to those skilled in the art. Virtually any promoter capable of driving these genes is suitable for the present invention including but not limited to lac, ara, tet, trp, IP_{L}, IP_{R}, T7, tac, and trc (useful for expression in Escherichia coli) as well as the amy, apr, npr promoters and various phage promoters useful for expression in Bacillus, and promoters isolated from the nrtA, gInB, moxF, glyoxlI, htpG, and hps genes useful for expression in Methylomonas (U.S. Ser. No. 10/689,200; hereby incorporated by reference). Additionally, promoters such as the chloramphenicol resistance gene promoter may also be useful for expression in Methylomonas.

Termination control regions may also be derived from various genes native to the preferred hosts. A termination site may be unnecessary, but is preferred.

Knowledge of the sequence of the present gene will be useful in manipulating the overall growth characteristics and/or carotenoid production in any organism having such a pathway and particularly in Escherichia coli. Methods of manipulating genetic pathways are common and well known in the art. Selected genes in a particular pathway may be upregulated or down regulated by a variety of methods. Additionally, competing pathways may be eliminated or sublimated by gene disruption and similar techniques.

Once a key genetic pathway has been identified and sequenced, specific genes may be upregulated to increase the output of the pathway. For example, additional copies of the targeted genes may be introduced into the host cell on multicopy plasmids such as pBR322. Optionally, multiple genes encoding polypeptides involved in resveratrol biosynthesis may be chromosomally expressed to increase the transformed host cell's resveratrol production. However, stable chromosomal expression of multiple genes generally requires that the coding sequences of the genes used comprise nucleotide sequences having low to moderate sequence identity to one another.

When it is desired to regulate expression of the target gene, say when a pathway operates at a particular point in a cell cycle or during a fermentation run, regulated or inducible promoters may used to replace the native promoter of the target gene. Or in some cases the native or endogenous promoter may be modified to increase gene expression. For example, endogenous promoters can be altered in vivo by mutation, deletion, and/or substitution (see, Kmiec, U.S. Pat. No. 5,565,350; Zarling et al., PCT/US93/03868).

When it is desired to down-regulate the expression of one or more known genes in the target or competing pathways—which may serve as competing sinks for energy or carbon—one method is gene disruption. This may be accomplished by insertion into the host cell of genetic cassettes, which comprise foreign DNA, often a genetic marker, and are flanked by sequences having a high degree of homology to a portion of the gene. The highly homologous foreign sequences enable native DNA replication mechanisms to insert the cassette into similar host sequences, which results in transcription disruption of the host gene occurs (Hamilton et al., J. Bacteriol., 171:46174622 (1989); Balbas et al., Gene, 136:211-213 (1993); Gueldener et al., Nucleic Acids Res., 24:2519-2524 (1996); and Smith et al., Methods Mol. Cell. Biol., 5:270-277 (1996)).

Another method of down regulating genes where the sequence of the target gene is known is antisense technology. Here, a nucleic acid segment from the target gene is cloned and operably linked to a promoter. This construct is then introduced into the host cell and the antisense strand of RNA is produced. Antisense RNA inhibits gene expression by preventing the accumulation of mRNA that encodes the protein of interest. The person skilled in the art will know that special considerations are associated with the use of antisense technologies in order to reduce expression of particular genes. For example, the proper level of expression of antisense genes may require the use of different chimeric genes utilizing different regulatory elements known to the skilled artisan.

Although targeted gene disruption and antisense technology offer effective means of down regulating genes for known sequences, other less specific methodologies have been developed that do not depend on the target sequence. For example, cells may be exposed to UV radiation and then screened for the desired phenotype. Mutagenesis with chemical agents is also effective for generating mutants and commonly used substances include chemicals that affect nonreplicating DNA such as HNO₂ and NH₂OH, as well as agents that affect replicating DNA such as acridine dyes, which cause frameshift mutations. Specific methods for creating mutants using radiation or chemical agents are well documented in the art. See for example Thomas D. Brock in Biotechnology: A Textbook of Industrial Microbiology, Second Edition (1989) Sinauer Associates, Inc., Sunderland, Mass., or Deshpande, Mukund V., Appl. Biochem. Biotechnol., 36:227 (1992).

Another non-specific method of gene disruption is the use of transposable elements or transposons. Transposons are genetic elements that insert randomly in DNA. They can be later retrieved and/or located within the target DNA on the basis of their sequence. Both in vivo and in vitro transposition methods are known and involve the use of a transposable element in combination with a transposase enzyme. When the transposable element or transposon is contacted with a nucleic acid molecule in the presence of the transposase, the transposable element will randomly insert into the nucleic acid molecule. The technique is useful for random mutageneis and for gene isolation, since the disrupted gene may be identified on the basis of the sequence of the transposable element. Kits for in vitro transposition are commercially available (see for example The Primer Island Transposition Kit, available from Perkin Elmer Applied Biosystems, Branchburg, N.J., based upon the yeast Ty1 element; The Genome Priming System, available from New England Biolabs, Beverly, Mass.; based upon the bacterial transposon Tn7; and the EZ::TN Transposon Insertion Systems, available from Epicentre Technologies, Madison, Wis., based upon the Tn5 bacterial transposable element.

Suitable Coding Regions of Interest

Coding regions of interest to be expressed in the recombinant bacterial host may be either endogenous or foreign to the host. For example, suitable coding regions of interest may include those encoding viral, bacterial, fungal, plant, insect, or vertebrate coding regions of interest, including mammalian polypeptides.

The coding regions of the present invention are those encoding proteins useful for the production of resveratrol and/or resveratrol glucoside. The coding regions of interest may be optionally codon-optimized using the preferred codon usage of the host cell selected. The present methods are exemplified using specific genes as described by the accompanying sequence listing. However, many of the genes used to recombinantly produce resveratrol and/or resveratrol glucoside are available from alternative sources. For example, a non-limited list of alternative, publicly-available genes of the present invention are provided in Table 1. In a further aspect, the genes selected for recombinant expression in Escherichia coli are codon optimized using the preferred codon usage described by Henaut and Danchin (Analysis and Predictions from Escherichia coli sequences. Escherichia coli and Salmonella, Vol. 2, Ch. 114:2047-2066, 1996, Neidhardt FC ed., ASM press, Washington, D.C.).

Components of Vectors/DNA Cassettes

Vectors or DNA cassettes useful for the transformation of suitable host cells are well known in the art. The specific choice of sequences present in the construct is dependent upon the desired expression products, the nature of the host cell, and the proposed means of separating transformed cells versus non-transformed cells. Typically, however, the vector or cassette contains sequences directing transcription and translation of the relevant gene(s), a selectable marker, and sequences allowing autonomous replication or chromosomal integration. Suitable vectors comprise a region 5′ of the gene that controls transcriptional initiation and a region 3′ of the DNA fragment that controls transcriptional termination. It is most preferred when both control regions are derived from genes from the transformed host cell, although it is to be understood that such control regions need not be derived from the genes native to the specific species chosen as a production host.

As one of skill in the art is aware, merely inserting a chimeric gene into a cloning vector does not ensure that it will be successfully expressed at the level needed. In response to needs for high expression rates, many specialized expression vectors have been created by manipulating a number of different genetic elements that control aspects of transcription, translation, protein stability, oxygen limitation and secretion from the host cell. More specifically, some of the molecular features that have been manipulated to control gene expression include: 1.) the nature of the relevant transcriptional promoter and terminator sequences; 2.) the number of copies of the cloned gene and whether the gene is plasmid-borne or integrated into the genome of the host cell; 3.) the final cellular location of the synthesized foreign protein; 4.) the efficiency of translation in the host organism; 5.) the intrinsic stability of the cloned gene protein within the host cell; and 6.) the codon usage within the cloned gene, such that its frequency approaches the frequency of preferred codon usage of the host cell. Each of these types of modifications are encompassed in the present invention as means to further optimize expression of a chimeric gene.

Transformation of Bacterial Host Cells

Once an appropriate chimeric gene has been constructed that is suitable for expression in a yeast cell, it is placed in a plasmid vector capable of autonomous replication in a host cell or it is directly integrated into the genome of the host cell. Integration of expression cassettes can occur randomly within the host genome or can be targeted through the use of constructs containing regions of homology with the host genome sufficient to target recombination with the host locus. Where constructs are targeted to an endogenous locus, all or some of the transcriptional and translational regulatory regions can be provided by the endogenous locus.

Where two or more genes are expressed from separate replicating vectors, it is desirable that each vector has a different means of selection and should lack homology to the other constructs to maintain stable expression and prevent reassortment of elements among constructs. Judicious choice of regulatory regions, selection means and method of propagation of the introduced construct can be experimentally determined so that all introduced genes are expressed at the necessary levels to provide for synthesis of the desired products.

Constructs comprising a coding region of interest may be introduced into a host cell by any standard technique including, but not limited to chemical transformation, biolistic impact, electroporation, microinjection, conjugation or any other method that introduces the gene of interest into the host cell.

For convenience, a host cell that has been manipulated by any method to take up a DNA sequence (e.g., an expression cassette) will be referred to as “transformed” or “recombinant” herein. The transformed host will have at least one copy of the expression construct and may have two or more, depending upon whether the gene is integrated into the genome, amplified, or is present on an extrachromosomal element having multiple copy numbers. The transformed host cell can be identified by selection for a marker contained on the introduced construct. Alternatively, a separate marker construct may be co-transformed with the desired construct, as many transformation techniques introduce many DNA molecules into host cells. Typically, transformed hosts are selected for their ability to grow on selective media. Selective media may incorporate an antibiotic or lack a factor necessary for growth of the untransformed host, such as a nutrient or growth factor. An introduced marker gene may confer antibiotic resistance or encode an essential growth factor or enzyme, thereby permitting growth on selective media when expressed in the transformed host. Selection of a transformed host can also occur when the expressed marker protein can be detected, either directly or indirectly. The marker protein may be expressed alone or as a fusion to another protein. The marker protein can be detected by: 1.) its enzymatic activity (e.g., β-galactosidase can convert the substrate X-gal [5-bromo-4-chloro-3-indolyl-β-D-galactopyranoside] to a colored product; luciferase can convert luciferin to a light-emitting product); or 2.) its light-producing or modifying characteristics (e.g., the green fluorescent protein of Aequorea victoria fluoresces when illuminated with blue light). Alternatively, antibodies can be used to detect the marker protein or a molecular tag on, for example, a protein of interest. Cells expressing the marker protein or tag can be selected, for example, visually, or by techniques such as FACS or panning using antibodies.

Industrial Production

Suitable growth conditions, especially for commonly used bacterial production hosts such as E. coli, are well known in the art. In general, media conditions which may be optimized for high-level expression of a particular coding region of interest include the type and amount of carbon source, the type and amount of nitrogen source, the carbon-to-nitrogen ratio, the oxygen level, growth temperature, pH, length of the biomass production phase and the time of cell harvest.

Fermentation media in the present invention must contain a suitable carbon source for the production of resveratrol. Suitable carbon sources may include, but are not limited to: monosaccharides (e.g., glucose, fructose), disaccharides (e.g., lactose, sucrose), oligosaccharides, polysaccharides (e.g., starch, cellulose or mixtures thereof), sugar alcohols (e.g., glycerol) or mixtures from renewable feedstocks (e.g., cheese whey permeate, cornsteep liquor, sugar beet molasses, and barley malt). Additionally, carbon sources may include alkanes, fatty acids, esters of fatty acids, monoglycerides, diglycerides, triglycerides, phospholipids and various commercial sources of fatty acids including vegetable oils (e.g., soybean oil) and animal fats. Additionally, the carbon source may include one-carbon sources (e.g., carbon dioxide, methanol, formaldehyde, formate, carbon-containing amines) for which metabolic conversion into key biochemical intermediates has been demonstrated. Hence, it is contemplated that the source of carbon utilized in the present invention may encompass a wide variety of carbon-containing sources and will only be limited by the choice of the host organism. Although all of the above mentioned carbon sources and mixtures thereof are expected to be suitable in the present invention, the preferred carbon sources are. Most preferred is glucose.

Nitrogen may be supplied from an inorganic (e.g., (NH₄)₂SO₄) or organic source (e.g., urea or glutamate). In addition to appropriate carbon and nitrogen sources, the fermentation media must also contain suitable minerals, salts, cofactors, buffers, vitamins, and other components known to those skilled in the art suitable for the growth of the microorganism.

Preferred growth media in the present invention are common commercially prepared media. Other defined or synthetic growth media may also be used and the appropriate medium for growth of the particular microorganism will be known by one skilled in the art of microbiology or fermentation science. A suitable pH range for the fermentation is typically between about pH 4.0 to pH 8.0, wherein pH 5.5 to pH 7.0 is preferred as the range for the initial growth conditions. The fermentation may be conducted under aerobic or anaerobic conditions, wherein aerobic conditions are preferred.

Host cells comprising a suitable coding region of interest operably linked to the promoters of the present invention may be cultured using methods known in the art. For example, the cell may be cultivated by shake flask cultivation, small-scale or large-scale fermentation in laboratory or industrial fermentors performed in a suitable medium and under conditions allowing expression of the coding region of interest.

Where commercial production of resveratrol and/or resveratrol glucoside is desired, a variety of fermentation methodologies may be applied. For example, large-scale production of a specific gene product over-expressed from a recombinant host may be produced by a batch, fed-batch or continuous fermentation process.

A batch fermentation process is a closed system wherein the media composition is fixed at the beginning of the process and not subject to further additions beyond those required for maintenance of pH and oxygen level during the process. Thus, at the beginning of the culturing process the media is inoculated with the desired organism and growth or metabolic activity is permitted to occur without adding additional sources (i.e., carbon and nitrogen sources) to the medium. In batch processes the metabolite and biomass compositions of the system change constantly up to the time the culture is terminated. In a typical batch process, cells proceed through a static lag phase to a high growth log phase and finally to a stationary phase, wherein the growth rate is diminished or halted. Left untreated, cells in the stationary phase will eventually die. A variation of the standard batch process is the fed-batch process, wherein the source is continually added to the fermentor over the course of the fermentation process. A fed-batch process is also suitable in the present invention. Fed-batch processes are useful when catabolite repression is apt to inhibit the metabolism of the cells or where it is desirable to have limited amounts of source in the media at any one time. Measurement of the source concentration in fed-batch systems is difficult and therefore may be estimated on the basis of the changes of measurable factors such as pH, dissolved oxygen and the partial pressure of waste gases (e.g., CO₂). Batch and fed-batch culturing methods are common and well known in the art and examples may be found in Brock (supra) and Deshpande (supra).

Commercial production of resveratrol and/or resveratrol glucoside may also be accomplished by a continuous fermentation process, wherein a defined media is continuously added to a bioreactor while an equal amount of culture volume is removed simultaneously for product recovery. Continuous cultures generally maintain the cells in the log phase of growth at a constant cell density. Continuous or semi-continuous culture methods permit the modulation of one factor or any number of factors that affect cell growth or end product concentration. For example, one approach may limit the carbon source and allow all other parameters to moderate metabolism. In other systems, a number of factors affecting growth may be altered continuously while the cell concentration, measured by media turbidity, is kept constant. Continuous systems strive to maintain steady state growth and thus the cell growth rate must be balanced against cell loss due to media being drawn off the culture. Methods of modulating nutrients and growth factors for continuous culture processes, as well as techniques for maximizing the rate of product formation, are well known in the art of industrial microbiology and a variety of methods are detailed by Brock, supra.

Methods to Isolate Resveratrol and/or Resveratrol Glucoside

Resveratrol can be extracted from plant or other sources by extraction with organic solvents, such as methanol or methanol/water (80:20) (Adrian et al., J. Agric. Food Chem., 48:6103-6105 (2000)) and methanol:acetone:water:formic acid (40:40:20:0.1) (Rimando et al., J. Agric. Food Chem., 52:47134719 (2004)). Dried or freeze-dried extracts are dissolved in methanol, or water, or acetone, before reverse phase HPLC analysis. In one study in which resveratrol glucoside is produced in transgenic alfalfa (Hipskind, J. D., and Paiva, N. L, Molecular plant-microbe interactions, 13(5):551-562 (2000)), resveratrol and other metabolites are extracted in 100% acetone, followed by drying completely in nitrogen, and dissolving in 70% methanol in water. The extract is then analyzed by reverse phase HPLC. It is also possible to extract resveratrol using ethanol, dimethylsulfoxide, or other polar solvents. In the study in which resveratrol is produced in the yeast Saccharomyces cerevisiae at ˜1.4 μg/L (Becker et al., supra), resveratrol was extracted by breaking cells open by glass beads in 100% ice cold methanol and incubating at 37° C. for a few hours. Upon glycosidase treatment, the sample was dried and dissolved in 50% acetonitrile and analyzed by HPLC and mass spectroscopy. It is also possible to extract resveratrol using ethanol, dimethylsulfoxide, acetonitrile or other polar solvents. Resveratrol or resveratrol glucoside can also be detected by ¹H-NMR.

Uses of Resveratrol and Resveratrol Glucoside

The invention is useful for the biological production of resveratrol, which may be used alone or as an ingredient is an antioxidant, anti-inflammatory agent, antimicrobial/antifungal agent a dietary supplement, or as a pharmacological agent used to treat such conditions as hypercholesterolemia or cancer, to name a few. The resveratrol or resveratrol glucoside can be used for synthesis of cosmetics, personal care products (e.g., compositions suitable for contact with hair, skin, nails, teeth, etc.), cosmeceuticals, nutritional supplements, one or more components of a pharmaceutical composition, compositions applied fresh foods and or agricultural crops to deter and/or inhibit microbial/fungal growth, and as antioxidant compositions (e.g., to stabilize/protect readily oxidiziable compounds such as ω-3 fatty acids, carotenoids, etc.)

In another embodiment, the isolated resveratrol-producing bacterial biomass is used as an additive in a composition selected from the group consisting of antioxidants, anti-inflammatory agents, antifungal/antimicrobial agents, cosmetics, cosmeceuticals, nutritional/dietary supplements, feed additives, and pharmacological agents, to name a few. The isolated microbial biomass may be in the form of whole cells, homogenized cells, or partially-purified cell extracts. In one embodiment, the isolated recombinant biomass comprises at least 0.1% dry cell weight (dcw), preferably at least 0.2% (dcw), more preferably at least 1% (dcw), and most preferably at least 2% (dcw) resveratrol. As such, and in a preferred embodiment, the invention provides a composition selected from the group consisting of antioxidants, anti-inflammatory agents, antifungal/antimicrobial agents, person care product, cosmetics, cosmeceuticals, nutritional/dietary supplements, feed additives, and medicaments comprising 0.1 to 99 wt % recombinant recombinant bacterial biomass having at least at least 0.1% dry cell weight (dcw), preferably at least 0.2% (dcw), more preferably at least 1% (dcw), and most preferably at least 2% (dcw) resveratrol.

In another embodiment, resveratrol is used as an antioxidant to stabilize other antioxidants such as carotenoids (including xanthophylls) and polyunsaturated fatty acids, especially ω-3 polyunsaturated fatty acids. In one embodiment, the recombinantly produced stilbene is added to compositions comprising at least one ω-3 PUFA. In a preferred embodiment, the microbial microorganism is engineered to produce both resveratrol/resveratrol glucoside and at least one carotenoid (including xanthophylls) whereby either compounds, preferably the carotenoid, exhibits increased stability to oxidation. Methods to engineer microbial production of carontenoids is well known in the art. Of particular interest is methylotrophic and/or methanotrophic bacterial strains engineered to product carotenoids (U.S. Pat. No. 6,969,595 and U.S. 60/780,524; each incorporated herein by reference).

Unless otherwise specified, all referenced United States patents and patent applications described herein are hereby incorporated by reference.

### EXAMPLES

The present invention is further defined in the following Examples. It should be understood that these Examples are given by way of illustration only. From the above discussion and these Examples, one skilled in the art can ascertain the essential characteristics of this invention, and without departing from the spirit and scope thereof, can make various changes and modifications of the invention to adapt it to various usages and conditions.

### GENERAL METHODS

Standard recombinant DNA and molecular cloning techniques used in the Examples are well known in the art and are described by Sambrook, J., Fritsch, E. F. and Maniatis, T. Molecular Cloning: A Laboratory Manual; Cold Spring Harbor Laboratory Press: Cold Spring Harbor, (1989) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc. and Wiley-Interscience (1987).

Materials and methods suitable for the maintenance and growth of bacterial cultures are well known in the art. Techniques suitable for use in the following examples may be found as set out in Manual of Methods for General Bacteriology (Phillipp Gerhardt, R. G. E. Murray, Ralph N. Costilow, Eugene W. Nester, Willis A. Wood, Noel R. Krieg and G. Briggs Phillips, eds), American Society for Microbiology, Washington, D.C. (1994)) or by Thomas D. Brock in Biotechnology: A Textbook of Industrial Microbiology, Second Edition, Sinauer Associates, Inc., Sunderland, Mass. (1989).

All reagents, restriction enzymes and materials used for the growth and maintenance of bacterial cells were obtained from Aldrich Chemicals (Milwaukee, Wis.), DIFCO Laboratories (Detroit, Mich.), GIBCO/BRL (Gaithersburg, Md.), or Sigma Chemical Company (St. Louis, Mo.) unless otherwise specified.

The meaning of abbreviations is as follows: “sec” means second(s), “min” means minute(s), “h” or “hr” means hour(s), “psi” means pounds per square inch, “nm” means nanometers, “d” means day(s), “μL” means microliter, “mL” means milliliters, “L” means liters, “mm” means millimeters, “nm” means nanometers, “μM” means micromolar, “mM” means millimolar, “M” means molar, “mmol” means millimole(s), “μmole” mean micromole”, “g” means gram, “μg” means microgram and “ng” means nanogram, “ppm” means parts-per-million, “U” means units, “mU” means milliunits, “U mg⁻¹” means units per mg, and “rpm” mean revolutions per minute.

### Example 1

### Cloning of Coumaroyl-CoA ligase from Streptomyces coelicolor BAA-471D™

Genomic DNA of Streptomyces coelicolor BAA-471 D™ was obtained from the American Type Culture Collection (ATCC® BAA-471D™). Primer OT452 (5′-GGGAATTCGCCATATGTTCCGCAGCGAGTACGCAGACG-3′; SEQ ID NO: 1) and OT453 (5′-CACGGAATTCAGATCTCATCGCGGCTCCCTGAGCTG-3′; SEQ ID NO: 2) were used to amplify the coumaroyl-CoA ligase open-reading frame (SEQ ID NO: 3) by PCR, using the Advantage GC cDNA kit from ClonTech (Palo Alto, Calif.). The reaction mixture contained 1 μL each of 20 μM OT452 and 20 μM OT453, 1 μL of 0.1 μg/mL genomic DNA, 10 μL GC-melt™ (1M final)(ClonTech), 10 μL 5×PCR buffer, 1 μL Polymerase mix, 4 μL 25 μM dNTP mix, and 23 μL water. The reaction mixture was heated at 94° C. for 2.5 minutes, followed by 30 cycles of 94° C. 0.5 minutes, 55° C. 0.5 minutes, and 72° C. 2 minutes. The mixture was further incubated at 72° C. for 6 minutes, and kept at 4° C. until purification step.

A 1580 bp DNA fragment was obtained from the PCR reaction. This fragment was purified with Qiagen PCR purification kit (Qiagen, Valencia, Calif.). 10 μg of purified PCR product and 5 μg of pET-Duet™-1 vector (Novagen, Madison, Wis.; SEQ ID NO: 5) were each digested with 10 units each of NdeI and Bg/II, in a final volume of 60 μL, for 2 hours at 37° C. The digested DNA samples were purified again with Qiagen PCR purification kit. 50 ng of digested pET-Duet™-1 vector and 100 ng of the digested PCR product were ligated with T4 DNA ligase in a volume of 20 μL overnight at room temperature. The ligation mixture was used to transform E. coli One Shot® Top10 chemical competent cells (Invitrogen, Carlsbad, Calif.). Transformants were plated on LB plate containing 100 μg/mL ampicillin.

Plasmid DNA from 8 transformants was purified using Qiagen Miniprep kit. DNA samples were analyzed by restriction mapping using NdeI and Bg/II, and by DNA sequencing. One of the clones was chosen and named pCCL-ET-D3 (SEQ ID NO: 6).

Based on the codon usage of E. coli as summarized by Henaut and Danchin (Henaut and Danchin: Analysis and Predictions from Escherichia coli sequences. Escherichia coli and Salmonella, Vol. 2, Ch. 114:2047-2066, 1996, Neidhardt FC ed., ASM press, Washington, D.C.), and the sequence of a stilbene synthase (SEQ ID NOs: 7 and 8), a DNA fragment STI-ET, containing the grape stilbene synthase gene codon-optimized for E. coli expression, was synthesized by Genscript Corporation (Piscataway, N.J.). The DNA fragment was then digested with NcoI and NotI, and ligated with pCCL-ET-D3 digested with the same two restriction enzymes. The ligation mixture was used to transform E. coli One Shot® Top10 competent cells (Invitrogen). Plasmid DNA from 12 transformants was isolated and analyzed by restriction mapping. All 12 transformants were found to contain the codon optimized stilbene synthase gene (SEQ ID NO: 9). The plasmid was named pET-ESTS-CCL (SEQ ID NO: 10). DNA from two of the clones was then used to transform E. coli BL21 (DE3) cells (EMD Biosciences, San Diego, Calif.). Transformants were analyzed for resveratrol production in the presence of added pHCA.

### Example 2

### Production of Resveratrol in E. coli Cells Transformed with Plasmid pET-ESTS-CCL

The transformed strains were named Res1 and Res2. For analysis of resveratrol production, each of the two strains was grown in 200 mL LB medium containing 100 μg/mL ampicillin to an O.D.₆₀₀ of ˜0.5 at 37° C. IPTG (isopropyl-beta-D-galactoside) was added to the cultures to a final concentration of 1 mM, and the cultures were grown for 4 hours at 26° C. with shaking at 250 rpm.

Cells from each culture were collected by centrifugation at 5000 rpm for 10 min, and resuspended in MOPS minimal media containing 0.2% glucose. pHCA was added to a final concentration of 3 mM, and the pH of the cultures was confirmed to be neutral. The cultures were grown at 26° C. for 3 days in the dark. Each culture was centrifuged again to collect the cells. Cells were resuspended in 10 mL ice cold methanol and lysed by sonication at 50% power repeating four cycles of 30 seconds (Fisher Sonic Dismembrator Model 300; Fisher Scientific, Hampton, N.H.). Lysed cells were incubated at 37° C. for 4 hours under constant agitation (250 rpm in an environmental shaker) to extract resveratrol.

The extraction mixture was then centrifuged to remove cell debris, and filtered through 0.2 μm filter (Nylon Spin-X® spin filter, CoStar, Corning Life Sciences, Acton, Mass.). Filtered samples were dried in a Savant DNA 110 Speed Vac (Thermo Savant, Holbrook, N.Y.) to near complete dryness. The samples were redissolved with 500 μL each of 50% acetonitrile, followed by filtration through 0.2 μm filter.

The filtered samples were analyzed for the presence of resveratrol by HPLC, using an Agilent 1100 system (Agilent Technologies, Palo Alto, Calif.) with a Zorbax SB-C₁₈ column, 4.6×150 mm, 3.5 micron. The column was eluted with a gradient of 5% to 80% acetonitrile, in 0.5% TFA (trifluroacetic acid) for 8 min, followed by 80% acetonitrile, 0.5% TFA for 2 min. Both pHCA and resveratrol are detected at 312 nm, with typical retention time of 5.4 min (pHCA) and 6.0 min (resveratrol). The amount of pHCA and resveratrol in the samples were calculated based on a comparison of peak area with known amounts of pure pHCA and resveratrol. Resveratrol was detected to be present in both samples. “Res1” sample contained 5 ppm, and Res2 sample 7 ppm resveratrol. This corresponds to resveratrol levels of 0.0125 mg/L and 0.0175 mg/L in each 200-mL culture, respectively.

The presence of resveratrol was further confirmed by Negative Ion Electrospray LCMS, using a Waters LCT Time of Flight mass spectrometer (Waters Corporation, Milford, Mass.) connected to a Waters Alliance 2790 LC system with an Agilent Zorbax SB-C18 column (2.1×150 mm). A gradient from 5% acetonitrile in H₂O to 100% acetonitrile in 30 minutes, at a flow rate of 0.25 mL/min was used to separate components in the samples. Both solvents contained 0.5% formic acid to sharpen the peaks eluding from the LC column. The mass spectrometer was set to scan from 60 to 800 Daltons in 0.9 seconds with a 0.1 second interscan delay.

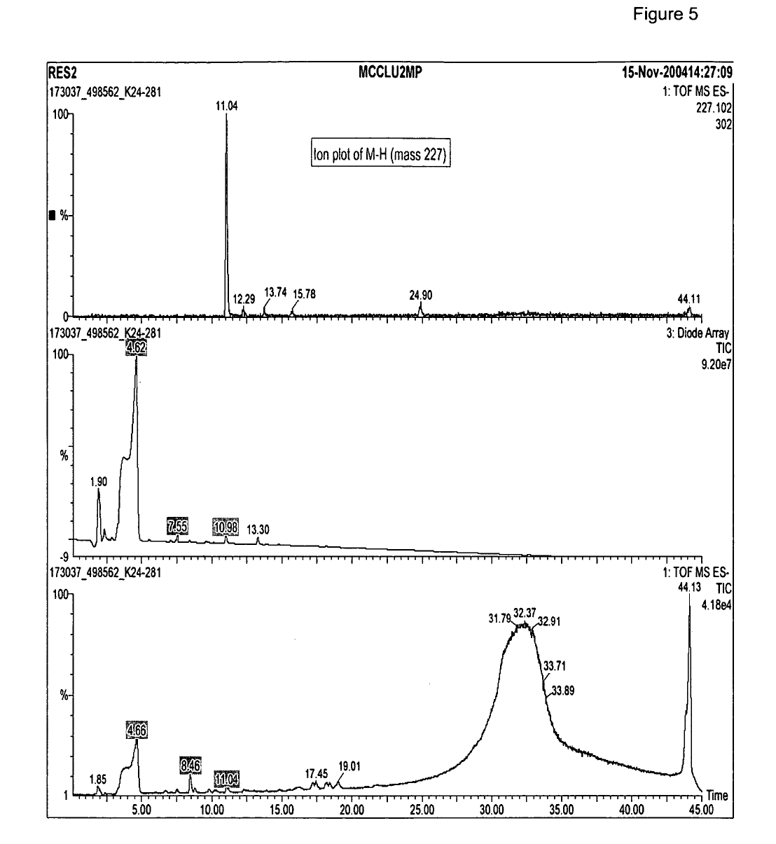

Sample “Res2” was analyzed as described above. The result of the analysis showed that both resveratrol and pHCA were present (FIG. 5). The presence of resveratrol was indicated by the peak at 11.04 min in the negative ion electrospray mass spectra, which contained a molecular ion of 227 Daltons, the same as resveratrol.

### Example 3

### Construction of Expression Vector pACYC.matBC.PCCL

It is quite likely that the supply of malonyl CoA in E. coli is one of the rate limiting factors for resveratrol production. An alternative way of increasing malonyl CoA concentration is the expression of malonyl CoA synthase (MatB) in E. coli and supplementing the growth media with malonate. The malonate could be transported into the E. coli host by a putative malonate transporter protein (MatC), and converted to malonyl CoA by malonyl CoA synthase.

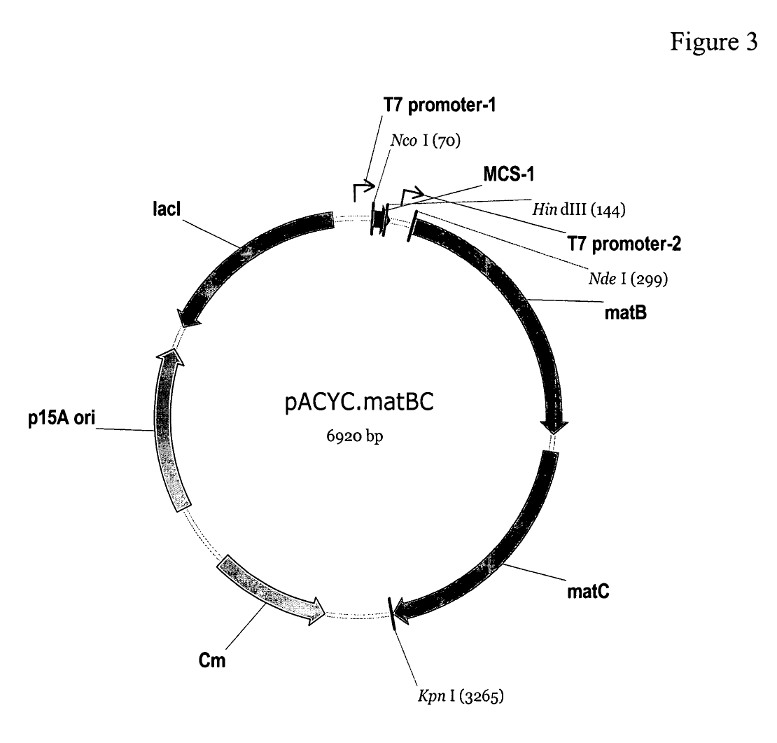

Bacteria strain Rhizobium leguminosarum bv. Trifolii was obtained from the American Type Culture Collection (ATCC strain 14479). Genomic DNA was prepared using standard procedures well known in the art. A PCR reaction was used to amplify the matBC operon, with 5′ primer (OT628: 5′-GGGAATTCGTCAT ATGAGCAACCATCTTTTCGACGCCATGCGG-3′; SEQ ID NO: 11), and 3′ primer (OT648: 5′-ACGGGGTACC TCAAACCAGCCCGGGCACGACGAACACCAA-3′; SE ID NO: 12). The PCR was performed using Phusion PCR enzyme (New England Biolabs, Beverly, Mass.) at 98° C., 30 sec; 35 cycles of 98° C., 10 sec, 55° C., 30 sec, 72° C., 2 min 30 sec; and 72° C., 10 min. The 2.9 kb PCR fragment was digested with NdeI and KpnI restriction enzymes, and ligated into vector pACYC.Duet (Novagen, Madison, Wis.). The resulting plasmid was named as pACYC.matBC (SEQ ID NO: 19), in which matBC gene operon (matB, SEQ ID NOs: 13 and 14; matC, SEQ ID NOs: 15 and 16) is under the control of a T7 promoter (FIG. 3).

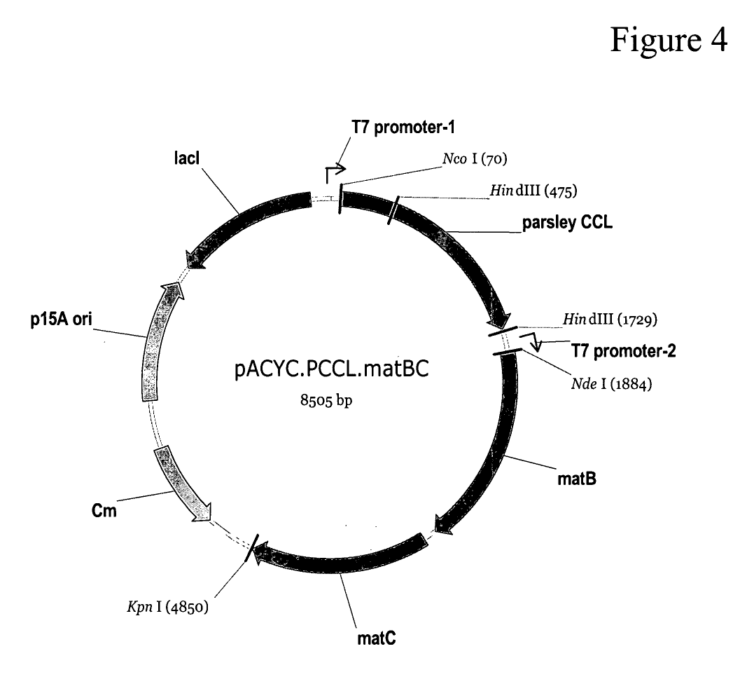

Another likely bottleneck in resveratrol production is the intracellular level of coumaroyl CoA. To increase the level of coumaroyl CoA, the parsley coumaroyl CoA ligase (Pc4cL-2; SEQ ID NOs: 20 and 21) was cloned from Petroselinum crispum (GenBank® accession number X13325) isolated from parsley young leaves by reverse transcription-PCR (RT-PCR) using conditions described in the literature (Lozoya, E. et al., Euro. J. Biochem., 176(3):661-667, 1998), and ligated into pACYC.matBC at NcoI and HindIII restriction sites, thus allowing for expression of parsley coumaroyl CoA ligase directly under the control of another copy of the T7 promoter (FIG. 4).

### Example 4

### Production of Resveratrol in E. coli cells Co-Transformed with Plasmids pET-ESTS-CCL and pACYC.PCCL.matBC

Plasmid pACYC.PCCL.matBC (SEQ ID NO: 22; FIG. 4) was used to transform E. coli BL21AI cells (EMD Biosciences, San Diego, Calif.) to generate strain DPD5157. DPD5157 was in turn transformed with pET-ESTS-CCL to produce strain DPD5158. DPD5158 cells were grown in 80 mL LB medium supplemented with 100 μg/mL ampicillin and 50 μg/mL chloramphenicol at 37° C. to OD₆₀₀ of 0.4, and induced with 0.2% arabinose at 28° C. overnight for 15 hr. The cells were centrifuged at 5000 rpm for 10 min, and resuspended in MOPS minimal media containing 0.2% glucose. pHCA was added to a final concentration of 3 mM. The culture was divided into two equal volume portions. Sample A was used as a control (malonic acid was not added); to sample B, malonic acid was added to 6 mM. The pH of the cultures were confirmed to be neutral. The cultures were grown at 28° C. for 3 days in the dark. Each culture was centrifuged to collect the cells. The levels of resveratrol in both culture supernatants and cell pellets were analyzed as described in previous examples. The level of resveratrol was significantly improved in both cultures (Table 2). The level of resveratrol in sample A was higher compared to sample B. This suggests that the amount of malonic acid supplemented in the growth medium can be further optimized. Overall, the presence of a second plasmid pACYC.PCCL.matBC led to an increase of total resveratrol production from 0.01-0.02 mg/L (0.0015% dcw to 0.003% dcw) to 1.6 to 3.6 mg/L (0.24% dcw to 2.3% dcw).

TABLE 2
Production of resveratrol from DPD5158 strain

| | pHCA | Res | pHCA | Res |
|---|---|---|---|---|
| Sample | (mg/L) | (mg/L) | (% dcw) | (% dcw) |
| DPD 5158 A pellet | 6.2 | 2.3 | 0.93 | 0.35 |
| DPD 5158 B pellet | 8.7 | 0.60 | 1.31 | 0.09 |
| DPD 5158 A supernatant | 29.5 | 1.3 | 4.43 | 1.95 |
| DPD 5158 B supernatant | 28.1 | 1.0 | 4.22 | 0.15 |
| DPD 5158 A total | 35.7 | 3.6 | 5.36 | 2.3 |
| DPD 5158 B total | 36.8 | 1.6 | 5.53 | 0.24 |

What is claimed is:

1. A method for the production of resveratrol comprising:
a) providing a bacterial host cell comprising:
1) at least one nucleic acid molecule encoding an enzyme having resveratrol synthase activity;
2) a source of malonyl CoA and coumaroyl CoA;
b) growing the bacterial host of (a) under conditions where malonyl CoA and coumaroyl CoA are reacted to resveratrol; and
c) optionally recovering the resveratrol of step (b).

2. A method according to claim 1 wherein the bacterial host cell comprises at least one nucleic acid molecule encoding an enzyme having malonyl CoA synthetase activity.

3. A method according to claim 1 wherein the bacterial host cell comprises at least one nucleic acid molecule encoding a polypeptide having malonyl transporter activity.

4. A method according to claim 1 wherein the bacterial host cell additionally comprises:
a) at least one nucleic acid molecule encoding an enzyme having coumaroyl CoA ligase activity; and
b) a source of p-hydroxycinnamic acid.

5. A method according to claim 4 wherein the bacterial host cell additionally comprises:
a) at least one nucleic acid molecule encoding an enzyme having tyrosine ammonium lyase activity; and
b) a source of tyrosine.

6. A method according to claim 4 wherein the bacterial host cell additionally comprises:
a) at least one nucleic acid molecule encoding an enzyme having cinnamate-4-hydroxylase activity; and
b) a source of cinnamic acid.

7. A method according to claim 6 wherein the bacterial host cell additionally comprises:
a) at least one nucleic acid molecule encoding an enzyme having phenylalanine ammonium lyase activity; and
b) a source of phenylalanine.

8. A method according to claim 1 wherein the bacterial host cell is a member of a genus selected from the group consisting of Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus.

9. A method according to claim 8 wherein the bacterial host cell is Escherichia coli.

10. A method according to claim 1 wherein at the least one nucleic acid molecule encoding an enzyme having resveratrol synthase activity is isolated from an organism selected from the group consisting of Vitis sp., Arachis sp., Cissus sp, and Parthenocissus sp.

11. A method according to claim 4 wherein at the least one nucleic acid molecule encoding an enzyme having coumaroyl CoA ligase activity;
is isolated from an organism selected from the group consisting of Streptomyces sp., Allium sp., Populus sp., Oryza sp., Amorpha sp., Nicotiana sp., Pinus sp., Glycine sp., Arabidopsis sp., Rubus sp., Lithospermum sp., and Zea sp.

12. A method according to claim 5 wherein at the least one nucleic acid molecule encoding an enzyme having tyrosine ammonium lyase activity;
is isolated from an organism selected from the group consisting of Rhodotorula sp., Amanita sp., Ustilago sp., Arabidopsis sp., Rubus sp., Medicago sp, Rehmannia sp., Lactuca sp., Petroselinium sp., Prunus sp., Lithospernum sp., Citrus sp., Rhodobacter sp., and Trichosporon sp.,

13. A method according to claim 6 wherein at the least one nucleic acid molecule encoding an enzyme having cinnamate-4-hydroxylase activity;
is isolated from an organism selected from the group consisting of Streptomyces sp., Allium sp., Populus sp., Oryza sp., Amorpha sp., Nicotiana sp., Pinus sp., Glycine sp., Arabidopsis sp., Rubus sp., Lithospermum sp., and Zea sp.

14. A method according to claim 7 wherein at the least one nucleic acid molecule encoding an enzyme having phenylalanine ammonium lyase activity; is isolated from an organism selected from the group consisting of Rhodotorula sp., Amanita sp., Ustilago sp., Arabidopsis sp., Rubus sp., Medicago sp, Rehmannia sp., Lactuca sp., Petroselinium sp., Prunus sp., Lithospernum sp., Citrus sp., Rhodobacter sp., and Trichosporon sp.

15. A method according to claim 1 wherein the source of malonyl CoA is exogenous to the host cell.

16. A method according to claim 4 wherein the source of p-hydroxycinnamic acid is endogenous to the host cell.

17. A method according to claim 4 wherein the source of p-hydroxycinnamic acid is exogenous to the host cell.

18. A method according to claim 5 wherein the source of tyrosine is endogenous to the host cell.

19. A method according to claim 5 wherein the source of tyrosine is exogenous to the host cell.

20. A method according to claim 6 wherein the source of cinnamic acid is endogenous to the host cell.

21. A method according to claim 6 wherein the source of cinnamic acid is exogenous to the host cell.

22. A method according to claim 7 wherein the source of phenylalanine is endogenous to the host cell.

23. A method according to claim 7 wherein the source of phenylalanine is exogenous to the host cell.

24. A method according to claim 1 wherein resveratrol is produced at a concentration of at least 0.2% dry cell weight.

25. A recombinant bacterial host cell comprising at least one nucleic acid molecule encoding an enzyme having resveratrol synthase activity which produces resveratrol.

26. The recombinant bacterial host cell of claim 25 further comprising at least one nucleic acid molecule encoding a polypeptide selected from the group consisting of; malonyl CoA synthetase, malonate transporter protein, coumaroyl CoA ligase, tyrosine ammonium lyase, cinnamate-4-hydroxylase and phenylalanine ammonium lyase.

27. The recombinant bacterial host cell of either of claims 25 or 26 wherein the microorganism is a strain of E. coli

28. An animal feed, pharmaceutical composition, antifungal composition, or a dietary supplement comprising at least 0.1 wt % of the transformed bacterial biomass having at least 0.2% dry cell weight resveratrol.


SEQUENCE LISTING (103 sequences)

SEQ ID NO: 1 - DNA, 38 bp, artificial sequence
  Primer
gggaattcgccatatgttccgcagcgagtacgcagacg

SEQ ID NO: 2 - DNA, 36 bp, artificial sequence
  Primer
cacggaattcagatctcatcgcggctccctgagctg

SEQ ID NO: 3 - DNA, 1569 bp, Streptomyces coelicolor
gtgttccgcagcgagtacgcagacgtcccgcccgtcgacctgcccatccacgacgccgtgctcggcggggccgccgccttcgggagcaccccggcgctgatcgacggcaccgacggcaccaccctcacctacgagcaggtggaccggttccaccggcgcgtcgccgccgccctcgccgagaccggcgtgcgcaagggcgacgtcctcgccctgcacagccccaacaccgtcgccttccccctggccttctacgccgccacccgcgcgggcgcctccgtcaccacggtgcatccgctcgcgacggcggaggagttcgccaagcagctgaaggacagcgcggcccgctggatcgtcaccgtctcaccgctcctgtccaccgcccgccgggccgccgaactcgcgggcggcgtccaggagatcctggtctgcgacagcgcgcccggtcaccgctccctcgtcgacatgctggcctcgaccgcgcccgaaccgtccgtcgccatcgacccggccgaggacgtcgccgccctgccgtactcctcgggcaccaccggcacccccaagggcgtcatgctcacacaccggcagatcgccaccaacctcgcccagctcgaaccgtcgatgccgtccgcgcccggcgaccgcgtcctcgccgtgctgccgttcttccacatctacggcctgaccgccctgatgaacgccccgctccggctcggcgccaccgtcgtggtcctgccccgcttcgacctggagcagttcctcgccgccatccagaaccaccgcatcaccagcctgtacgtcgccccgccgatcgtcctggccctcgccaaacaccccctggtcgccgactacgacctctcctcgctgaggtacatcgtcagcgccgccgccccgctcgacgcgcgtctcgccgccgcctgctcgcagcggctcggcctgccgcccgtcggccaggcctacggcatgaccgaactgtccccgggcacccacgtcgtccccctggacgcgatggccgacgcgccgcccggcaccgtcggcaggctcatcgcgggcaccgagatgcgcatcgtctccctcaccgacccgggcacggacctccccgccggagagtccggggagatcctcatccgcggcccccagatcatgaagggctacctgggccgccccgacgccaccgccgccatgatcgacgaggagggctggctgcacaccggggacgtcggacacgtcgacgccgacggctggctgttcgtcgtcgaccgcgtcaaggaactgatcaagtacaagggcttccaggtggcccccgccgaactggaggcccacctgctcacccaccccggcgtcgccgacgcggccgtcgtcggcgcctacgacgacgacggcaacgaggtaccgcacgccttcgtcgtccgccagccggccgcacccggcctcgcggagagcgagatcatgatgtacgtcgccgaacgcgtcgccccctacaaacgcgtccgccgggtcaccttcgtcgacgccgtcccccgcgccgcctccggcaagatcctccgccgacagctcagggagccgcgatga

SEQ ID NO: 4 - protein, 522 aa, Streptomyces coelicolor
MFRSEYADVPPVDLPIHDAVLGGAAAFGSTPALIDGTDGTTLTYEQVDRFHRRVAAALAETGVRKGDVLALHSPNTVAFPLAFYAATRAGASVTTVHPLATAEEFAKQLKDSAARWIVTVSPLLSTARRAAELAGGVQEILVCDSAPGHRSLVDMLASTAPEPSVAIDPAEDVAALPYSSGTTGTPKGVMLTHRQIATNLAQLEPSMPSAPGDRVLAVLPFFHIYGLTALMNAPLRLGATVVVLPRFDLEQFLAAIQNHRITSLYVAPPIVLALAKHPLVADYDLSSLRYIVSAAAPLDARLAAACSQRLGLPPVGQAYGMTELSPGTHVVPLDAMADAPPGTVGRLIAGTEMRIVSLTDPGTDLPAGESGEILIRGPQIMKGYLGRPDATAAMIDEEGWLHTGDVGHVDADGWLFVVDRVKELIKYKGFQVAPAELEAHLLTHPGVADAAVVGAYDDDGNEVPHAFVVRQPAAPGLAESEIMMYVAERVAPYKRVRRVTFVDAVPRAASGKILRRQLREPR

SEQ ID NO: 5 - DNA, 5420 bp, artificial sequence
  Plasmid
ggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 6 - DNA, 6982 bp, artificial sequence
  Plasmid
gatctcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgttccgcagcgagtacgcagacgtcccgcccgtcgacctgcccatccacgacgccgtgctcggcggggccgccgccttcgggagcaccccggcgctgatcgacggcaccgacggcaccaccctcacctacgagcaggtggaccggttccaccggcgcgtcgccgccgccctcgccgagaccggcgtgcgcaagggcgacgtcctcgccctgcacagccccaacaccgtcgccttccccctggccttctacgccgccacccgcgcgggcgcctccgtcaccacggtgcatccgctcgcgacggcggaggagttcgccaagcagctgaaggacagcgcggcccgctggatcgtcaccgtctcaccgctcctgtccaccgcccgccgrgccgccgaactcgcgggcggcgtccaggagatcctggtctgcgacagcgcgcccggtcaccgctccctcgtcgacatgctggcctcgaccgcgcccgaaccgtccgtcgccatcgacccggccgaggacgtcgccgccctgccgtactcctcgggcaccaccggcacccccaagggcgtcatgctcacacaccggcagatcgccaccaacctcgcccagctcgaaccgtcgatgccgtccgcgcccggcgaccgcgtcctcgccgtgctgccgttcttccacatctacggcctgaccgccctgatgaacgccccgctccggctcggcgccaccgtcgtggtcctgccccgcttcgacctggagcagttcctcgccgccatccagaaccaccgcatcaccagcctgtacgtcgccccgccgatcgtcctggccctcgccaaacaccccctggtcgccgactacgacctctcctcgctgaggtacatcgtcagcgccgccgccccgctcgacgcgcgtctcgccgccgcctgctcgcagcggctcggcctgccgcccgtcggccaggcctacggcatgaccgaactgtccccgggcacccacgtcgtccccctggacgcgatggccgacgcgccgcccggcaccgtcggcaggctcatcgcgggcaccgagatgcgcatcgtctccctcaccgacccgggcacggacctccccgccggagagtccggggagatcctcatccgcggcccccagatcatgaagggctacctgggccgccccgacgccaccgccgccatgatcgacgaggagggctggctgcacaccggggacgtcggacacgtcgacgccgacggctggctgttcgtcgtcgaccgcgtcaaggaactgatcaagtacaagggcttccaggtggcccccgccgaactggaggcccacctgctcacccaccccggcgtcgccgacgcggccgtcgtcggcgcctacgacgacgacggcaacgaggtaccgcacgccttcgtcgtccgccagccggccgcacccggcctcgcggagagcgagatcatgatgtacgtcgccgaacgcgtcgccccctacaaacgcgtccgccgggtcaccttcgtcgacgccgtcccccgcgccgcctccggcaagatcctccgccgacagctcagggagccgcgtga

SEQ ID NO: 7 - DNA, 1179 bp, Vitis sp.
atggcttcagttgaggaatttagaaacgctcaacgtgccaagggtccggccactatcctagccattggcacagctactcctgaccactgtgtctaccagtctgattatgctgattactatttcagggtcactaagagcgagcacatgactgagttgaagaagaagttcaatcgcatatgtgacaaatcaatgatcaagaagcgttacattcacttgaccgaagaaatgcttgaggagcacccaaacattggtgcttatatggctccatctcttaacatacgccaagagattatcactgctgaggtacctagacttggtagggatgcagcattgaaggctcttaaagagtggggccaaccaaagtccaagatcacccatcttgtattttgtacaacctccggtgtagaaatgcccggtgcggattacaaactcgctaatctcttaggtcttgaaacatcggttagaagggtgatgttgtaccatcaagggtgctatgcaggtggaactgtccttcgaactgctaaggatcttgcagaaaataatgcaggagcacgagttcttgtggtgtgctctgagatcactgttgttacattccgtggcccttccgaagatgctttggactctttagttggccaagccctttttggtgatgggtcttcagctgtgattgttggatcagatccagatgtctcgattgaacgaccactcttccaacttgtttcagcagcccaaacatttattcctaattcagcaggagccattgccggaaacttacgtgaggtggggctcacctttcatttgtggcccaatgtgcctactttgatttctgagaacatagagaaatgcttgacccaggcttttgacccacttggtattagcgattggaactcgttattttggattgctcacccaggtggccctgcaattctcgatgcagttgaagcaaaactcaatttagagaaaaagaaactcgaagcaactaggcatgtgttaagtgagtacggtaacatgtcaagtgcatgtgtgttgtttattctggatgagatgagaaagaaatccttgaagggggaaaaggctaccacaggtgaaggattggattggggagtattatttggttttgggccgggcttgaccatcgaaactgttgtgctgcatagcgttcctacagttacaaattaa

SEQ ID NO: 8 - protein, 392 aa, Vitis sp.
MASVEEFRNAQRAKGPATILAIGTATPDHCVYQSDYADYYFRVTKSEHMTELKKKFNRICDKSMIKKRYIHLTEEMLEEHPNIGAYMAPSLNIRQEIITAEVPRLGRDAALKALKEWGQPKSKITHLVFCTTSGVEMPGADYKLANLLGLETSVRRVMLYHQGCYAGGTVLRTAKDLAENNAGARVLVVCSEITVVTFRGPSEDALDSLVGQALFGDGSSAVIVGSDPDVSIERPLFQLVSAAQTFIPNSAGAIAGNLREVGLTFHLWPNVPTLISENIEKCLTQAFDPLGISDWNSLFWIAHPGGPAILDAVEAKLNLEKKKLEATRHVLSEYGNMSSACVLFILDEMRKKSLKGEKATTGEGLDWGVLFGFGPGLTIETVVLHSVPTVTN

SEQ ID NO: 9 - DNA, 1189 bp, artificial sequence
  Codon optimized gene
ccatggcttcagttgaggaatttcgtaacgctcaacgtgccaagggtccggccactatcctggccattggcacagctactcctgaccactgtgtctaccagtctgattatgctgattactatttccgcgtcactaagagcgagcacatgactgagttgaagaagaagttcaatcgcatttgtgacaaatcaatgatcaagaagcgttacattcacttgaccgaagaaatgcttgaggagcacccaaacattggtgcttatatggctccatctcttaacattcgccaagagattatcactgctgaggtacctcgtcttggtcgcgatgcagcattgaaggctcttaaagagtggggccaaccaaagtccaagatcacccatcttgtattttgtacaacctccggtgtagaaatgcccggtgcggattacaaactcgctaatctcttaggtcttgaaacatcggttcgtcgcgtgatgttgtaccatcaagggtgctatgcaggtggaactgtccttcgtactgctaaggatcttgcagaaaataatgcaggtgcacgtgttcttgtggtgtgctctgagatcactgttgttacattccgtggcccttccgaagatgctttggactctttagttggccaagccctttttggtgatgggtcttcagctgtgattgttggatcagatccagatgtctcgattgaacgtccactcttccaacttgtttcagcagcccaaacatttattcctaattcagcaggagccattgccggaaacttacgtgaggtggggctcacctttcatttgtggccgaatgtgcctactttgatttctgagaacatagagaaatgcttgacccaggcttttgacccacttggtattagcgattggaactcgttattttggattgctcacccaggtggccctgcaattctcgatgcagttgaagcaaaactcaatttagagaaaaagaaactcgaagcaactcgccatgtgttaagtgagtacggtaacatgtcaagtgcatgtgtgttgtttattctggatgagatgcgtaagaaatccttgaagggggaaaaggctaccacaggtgaaggattggattggggagtattatttggttttgggccgggcttgaccatcgaaactgttgtgctgcatagcgttcctacagttacaaattaagcggccgc

SEQ ID NO: 10 - DNA, 8085 bp, artificial sequence
  Plasmid
catggcctccgttgaggaattccgaaacgctcagcgagccaagggtcccgctaccatcctggccattggcactgctacccctgaccactgtgtctaccagtctgactatgccgattactacttccgagtgaccaagtccgagcacatgaccgagctcaagaagaagttcaaccggatctgtgacaaatccatgattaagaagcgatacatccacctgactgaagagatgctcgaagagcatcccaacattggcgcttacatggctccttctctgaacatccgacaggagattatcaccgctgaggttccccgactcggtcgggatgctgccctgaaggctctcaaagagtggggacagcccaagtccaagatcacccatctggtcttctgtactacctctggtgtggaaatgcctggagccgactacaagctcgctaacctgctcggccttgaaacctccgtccgacgagtcatgctgtaccaccaaggctgctacgctggtggcaccgtgctccgaactgccaaggacctggccgagaacaacgctggagcacgagtcctcgttgtgtgctccgaaatcactgtcgtgaccttccgaggtccctctgaagatgccctggactccctcgtcggccaggctctgtttggtgatggctcctctgccgtgattgttggatccgatcccgatgtctctatcgagcgacccctcttccagcttgtctccgctgcccaaacctttatccccaactctgctggtgccattgccggaaacctgcgagaggttggcctcaccttccacctgtggcctaatgtgcccactctcatctccgagaacattgagaagtgcctgacccaggctttcgaccctctcggtatctccgactggaactctctgttctggattgctcatcccggaggtcctgccatcctcgacgcagttgaggctaagctcaacctggagaagaagaagctcgaagccactcgacacgtgctgagcgagtacggcaacatgtcctctgcttgtgtgctcttcattctggacgagatgcgaaagaaatccctcaagggagagaaggccaccactggtgaaggcctggactggggagtcctcttcggctttggtcctggactgaccatcgaaactgtcgtgctccactctgttcccaccgtcactaactaagcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgttccgcagcgagtacgcagacgtcccgcccgtcgacctgcccatccacgacgccgtgctcggcggggccgccgccttcgggagcaccccggcgctgatcgacggcaccgacggcaccaccctcacctacgagcaggtggaccggttccaccggcgcgtcgccgccgccctcgccgagaccggcgtgcgcaagggcgacgtcctcgccctgcacagccccaacaccgtcgccttccccctggccttctacgccgccacccgcgcgggcgcctccgtcaccacggtgcatccgctcgcgacggcggaggagttcgccaagcagctgaaggacagcgcggcccgctggatcgtcaccgtctcaccgctcctgtccaccgcccgccgggccgccgaactcgcgggcggcgtccaggagatcctggtctgcgacagcgcgcccggtcaccgctccctcgtcgacatgctggcctcgaccgcgcccgaaccgtccgtcgccatcgacccggccgaggacgtcgccgccctgccgtactcctcgggcaccaccggcacccccaagggcgtcatgctcacacaccggcagatcgccaccaacctcgcccagctcgaaccgtcgatgccgtccgcgcccggcgaccgcgtcctcgccgtgctgccgttcttccacatctacggcctgaccgccctgatgaacgccccgctccggctcggcgccaccgtcgtggtcctgccccgcttcgacctggagcagttcctcgccgccatccagaaccaccgcatcaccagcctgtacgtcgccccgccgatcgtcctggccctcgccaaacaccccctggtcgccgactacgacctctcctcgctgaggtacatcgtcagcgccgccgccccgctcgacgcgcgtctcgccgccgcctgctcgcagcggctcggcctgccgcccgtcggccaggcctacggcatgaccgaactgtccccgggcacccacgtcgtccccctggacgcgatggccgacgcgccgcccggcaccgtcggcaggctcatcgcgggcaccgagatgcgcatcgtctccctcaccgacccgggcacggacctccccgccggagagtccggggagatcctcatccgcggcccccagatcatgaagggctacctgggccgccccgacgccaccgccgccatgatcgacgaggagggctggctgcacaccggggacgtcggacacgtcgacgccgacggctggctgttcgtcgtcgaccgcgtcaaggaactgatcaagtacaagggcttccaggtggcccccgccgaactggaggcccacctgctcacccaccccggcgtcgccgacgcggccgtcgtcggcgcctacgacgacgacggcaacgaggtaccgcacgccttcgtcgtccgccagccggccgcacccggcctcgcggagagcgagatcatgatgtacgtcgccgaacgcgtcgccccctacaaacgcgtccgccgggtcaccttcgtcgacgccgtcccccgcgccgcctccggcaagatcctccgccgacagctcagggagccgcgatgagatctgcaattggatatcggccggccacgcgatcgctgacgtcggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatac

SEQ ID NO: 11 - DNA, 2148 bp, Rhodosporidium toruloides
atgccaccctcgctcgactcgatctcgcactcgttcgcaaacggcgtcgcatccgcaaagcaggctgtcaatggcgcctcgaccaacctcgcagtcgcaggctcgcacctgcccacaacccaggtcacgcaggtcgacatcgtcgagaagatgctcgccgcgccgaccgactcgacgctcgaactcgacggctactcgctcaacctcggagacgtcgtctcggccgcgaggaagggcaggcctgtccgcgtcaaggacagcgacgagatccgctcaaagattgacaaatcggtcgagttcttgcgctcgcaactctccatgagcgtctacggcgtcacgactggatttggcggatccgcagacacccgcaccgaggacgccatctcgctccagaaggctctcctcgagcaccagctctgcggtgttctcccttcgtcgttcgactcgttccgcctcggccgcggtctcgagaactcgcttcccctcgaggttgttcgcggcgccatgacaatccgcgtcaacagcttgacccgcggccactcggctgtccgcctcgtcgtcctcgaggcgctcaccaacttcctcaaccacggcatcacccccatcgtccccctccgcggcaccatctctgcgtcgggcgacctgtctcctctctcctacattgcagcggccatcagcggtcacccggacagcaaggtgcacgtcgtccacgagggcaaggagaagatcctgtacgcccgcgaggcgatggcgctcttcaacctcgagcccgtcgtcctcggcccgaaggaaggtctcggtctcgtcaacggcaccgccgtctcagcatcgatggccaccctcgctctgcacgacgctcacatgctctcgctcctctcgcagtcgctcacggccatgacggtcgaagcgatggtcggccacgccggctcgttccaccccttccttcacgacgtcacgcgccctcacccgacgcagatcgaagtcgcgggaaacatccgcaagctcctcgagggaagccgctttgctgtccaccatgaggaggaggtcaaggtcaaggacgacgagggcattctccgccaggaccgctaccccttgcgcacgtctcctcagtggctcggcccgctcgtcagcgacctcattcacgcccacgccgtcctcaccatcgaggccggccagtcgacgaccgacaaccctctcatcgacgtcgagaacaagacttcgcaccacggcggcaatttccaggctgccgctgtggccaacaccatggagaagactcgcctcgggctcgcccagatcggcaagctcaacttcacgcagctcaccgagatgctcaacgccggcatgaaccgcggcctcccctcctgcctcgcggccgaagacccctcgctctcctaccactgcaagggcctcgacatcgccgctgcggcgtacacctcggagttgggacacctcgccaaccctgtgacgacgcatgtccagccggctgagatggcgaaccaggcggtcaactcgcttgcgctcatctcggctcgtcgcacgaccgagtccaacgacgtcctttctctcctcctcgccacccacctctactgcgttctccaagccatcgacttgcgcgcgatcgagttcgagttcaagaagcagttcggcccagccatcgtctcgctcatcgaccagcactttggctccgccatgaccggctcgaacctgcgcgacgagctcgtcgagaaggtgaacaagacgctcgccaagcgcctcgagcagaccaactcgtacgacctcgtcccgcgctggcacgacgccttctccttcgccgccggcaccgtcgtcgaggtcctctcgtcgacgtcgctctcgctcgccgccgtcaacgcctggaaggtcgccgccgccgagtcggccatctcgctcacccgccaagtccgcgagaccttctggtccgccgcgtcgacctcgtcgcccgcgctctcgtacctctcgccgcgcactcagatcctctacgccttcgtccgcgaggagcttggcgtcaaggcccgccgcggagacgtcttcctcggcaagcaagaggtgacgatcggctcgaacgtctccaagatctacgaggccatcaagtcgggcaggatcaacaacgtcctcctcaagatgctcgct

SEQ ID NO: 12 - protein, 716 aa, Rhodosporidium toruloides
MAPSLDSISHSFANGVASAKQAVNGASTNLAVAGSHLPTTQVTQVDIVEKMLAAPTDSTLELDGYSLNLGDVVSAARKGRPVRVKDSDEIRSKIDKSVEFLRSQLSMSVYGVTTGFGGSADTRTEDAISLQKALLEHQLCGVLPSSFDSFRLGRGLENSLPLEVVRGAMTIRVNSLTRGHSAVRLVVLEALTNFLNHGITPIVPLRGTISASGDLSPLSYIAAAISGHPDSKVHVVHEGKEKILYAREAMALFNLEPVVLGPKEGLGLVNGTAVSASMATLALHDAHMLSLLSQSLTAMTVEAMVGHAGSFHPFLHDVTRPHPTQIEVAGNIRKLLEGSRFAVHHEEEVKVKDDEGILRQDRYPLRTSPQWLGPLVSDLIHAHAVLTIEAGQSTTDNPLIDVENKTSHHGGNFQAAAVANTMEKTRLGLAQIGKLNFTQLTEMLNAGMNRGLPSCLAAEDPSLSYHCKGLDIAAAAYTSELGHLANPVTTHVQPAEMANQAVNSLALISARRTTESNDVLSLLLATHLYCVLQAIDLRAIEFEFKKQFGPAIVSLIDQHFGSAMTGSNLRDELVEKVNKTLAKRLEQTNSYDLVPRWHDAFSFAAGTVVEVLSSTSLSLAAVNAWKVAAAESAISLTRQVRETFWSAASTSSPALSYLSPRTQILYAFVREELGVKARRGDVFLGKQEVTIGSNVSKIYEAIKSGRINNVLLKMLA

SEQ ID NO: 13 - DNA, 1515 bp, Rhizobium leguminosarum bv. Trifolii
gtgagcaaccatcttttcgacgccatgcgggccgccgcgcccggtaacgcaccattcatccggatcgataacacgcgcacatggacctatgacgacgccttcgctctttccggccgcattgccagcgcgatggacgcgctcggcattcgccccggcgaccgcgttgcggtgcaggtcgagaaaagtgccgaggcattgatcctctatctcgcctgtcttcgaagcggcgccgtctacctgccgctcaacaccgcctatacgctggctgagctcgattattttatcggcgatgcggagccgcgtttggtggttgtcgcatcgtcggctcgagcgggcgtggagacaatcgccaagccccgcggtgcgatcgtcgaaactctcgacgctgctggcagcggctcgttgctggatctcgcccgcgacgagccggccgactttgtcgatgcctcgcgctccgccgatgatctggcggcgatcctctacacgtccggaacgacgggacgctccaagggggcgatgctcacgcatgggaacctgctctcgaacgccctgaccttgcgagatttttggcgcgtcaccgccggcgatcgactgatccatgccttgccgatcttccacacgcatggactgttcgtcgccacgaacgtcacactgctcgccggcgcctcgatgttcctgctgtcgaagttcgacccggaggagatcctgtcgctgatgccgcaggcaacgatgctgatgggcgtgccgaccttctacgtgcgcctcctgcagagcccgcgcctcgacaagcaagcggtcgccaacatccgcctcttcatttccggttcggctccactgcttgcagaaacacataccgagttccaggcacgtaccggtcacgccattctcgagcgctacggcatgacggaaaccaatatgaacacgtccaacccttatgaggggaaacggattgccggaacggtcggcttcccgctgcctgatgtgacggtgcgcgtcaccgatcccgccaccgggctcgcgctgccgcccgaacaaaccggcatgatcgagatcaaggggccgaacgttttcaagggctattggcgcatgcccgaaaaaaccgcggccgaattcaccgccgacggtttcttcatcagcggcgatctcggcaagatcgaccgcgacggttatgtccacatcgtcggccgcggcaaggatctggtgatttcgggtggatacaacatctatccgaaagaggttgagggcgagatcgaccagatcgagggtgtggttgagagcgctgtgatcggcgtgccgcatcccgatttcggagaaggcgtaacggccgtcgtcgtgcgcaagcccggcgctgccctcgatgaaaaggccatcgtcagcgccctccaggaccggctcgcgcgctacaaacaacccaagcgcatcatctttgcagaggacttgccgcgcaacacgatgggtaaggttcagaaaaacatcctgcggcagcaatacgccgatctttataccaggacgtaa

SEQ ID NO: 14 - protein, 504 aa, Rhizobium leguminosarum bv. Trifolii
MSNHLFDAMRAAAPGNAPFIRIDNTRTWTYDDAFALSGRIASAMDALGIRPGDRVAVQVEKSAEALILYLACLRSGAVYLPLNTAYTLAELDYFIGDAEPRLVVVASSARAGVETIAKPRGAIVETLDAAGSGSLLDLARDEPADFVDASRSADDLAAILYTSGTTGRSKGAMLTHGNLLSNALTLRDFWRVTAGDRLIHALPIFHTHGLFVATNVTLLAGASMFLLSKFDPEEILSLMPQATMLMGVPTFYVRLLQSPRLDKQAVANIRLFISGSAPLLAETHTEFQARTGHAILERYGMTETNMNTSNPYEGKRIAGTVGFPLPDVTVRVTDPATGLALPPEQTGMIEIKGPNVFKGYWRMPEKTAAEFTADGFFISGDLGKIDRDGYVHIVGRGKDLVISGGYNIYPKEVEGEIDQIEGVVESAVIGVPHPDFGEGVTAVVVRKPGAALDEKAIVSALQDRLARYKQPKRIIFAEDLPRNTMGKVQKNILRQQYADLYTRT

SEQ ID NO: 15 - DNA, 1347 bp, Rhizobium leguminosarum bv trifolii
atgggtattgaattactgtccataggcctgctgatcgccatgttcatcattgcgacgatccagccaatcaacatgggtgcgctcgcctttgccggcgccttcgtgctcggctcgatgatcatcgggatgaaaaccaacgaaatatttgccggctttccgagtgatctgttcctgacgctcgtcgccgtcacctacctcttcgccatagcgcagatcaacggcacgatcgactggctcgtcgaatgtgccgtccgcctggtacgcgggcggatcggcttgattccctgggtgatgttccttgtcgccgccatcattactggcttcggtgcacttgggcctgctgcggtcgccattctcgcacccgtcgcgttgagctttgccgtgcagtaccgcattcatccggtgatgatgggtctgatggtgatccacggcgcgcaggcaggcggcttctcgccgatcagcatctatggcggaatcaccaaccagatcgttgcgaaggccggcctgcctttcgctccgacctcgctgtttctttccagcttcttctttaacctggcgatcgcggtgctggtgttcttcgtgttcggcggcgcgagggtgatgaagcacgatcccgcatcacttggccccttgcccgaactccatcccgagggcgtatcggcgtcgatcagaggccacggcggcacgccggcaaaaccgatcagagagcatgcctatggtacggcggccgataccgcgacgacgttgcgtctgaacaatgagagaattaccaccttgatcggcctgacggcgctcggcatcggcgccctggttttcaagttcaatgttggcctcgtcgccatgaccgtcgccgtcgtcctcgcgctgctgtcaccgaagacccagaaggccgcaatcgacaaggtcagttggtcgaccgtgctgctgattgccggcatcatcacctatgtcggcgtcatggagaaggccggtacggtcgactacgtggcgaatggcatatccagtctcggcatgccgctactggtagcgctcctgctttgctttacgggcgccatcgtctcggcctttgcttcctcgaccgcgctgctcggcgcgatcatcccgcttgccgttccattcctcctgcaagggcacatcagcgccatcggtgtggtcgcggcgatcgccatctcgacgacgatcgtcgacaccagcccattctccaccaacggcgcccttgtcgtcgccaatgcgccggacgacagccgtgagcaggtgttgcgacagctactgatctacagcgccttgatcgctatcatcggtccgatcgttgcctggttggtgttcgtcgtgcccgggctggtttga

SEQ ID NO: 16 - protein, 448 aa, Rhizobium leguminosarum bv. Trifolii
MGIELLSIGLLIAMFIIATIQPINMGALAFAGAFVLGSMIIGMKTNEIFAGFPSDLFLTLVAVTYLFAIAQINGTIDWLVECAVRLVRGRIGLIPWVMFLVAAIITGFGALGPAAVAILAPVALSFAVQYRIHPVMMGLMVIHGAQAGGFSPISIYGGITNQIVAKAGLPFAPTSLFLSSFFFNLAIAVLVFFVFGGARVMKHDPASLGPLPELHPEGVSASIRGHGGTPAKPIREHAYGTAADTATTLRLNNERITTLIGLTALGIGALVFKFNVGLVAMTVAVVLALLSPKTQKAAIDKVSWSTVLLIAGIITYVGVMEKAGTVDYVANGISSLGMPLLVALLLCFTGAIVSAFASSTALLGAIIPLAVPFLLQGHISAIGVVAAIAISTTIVDTSPFSTNGALVVANAPDDSREQVLRQLLIYSALIAIIGPIVAWLVFVVPGLV

SEQ ID NO: 17 - DNA, 43 bp, Artificial Sequence
  Primer
gggaattcgtcatatgagcaaccatcttttcgacgccatgcgg

SEQ ID NO: 18 - DNA, 40 bp, artificial sequence
  Primer
acggggtacctcaaaccagcccgggcacgacgaacaccaa

SEQ ID NO: 19 - DNA, 6920 bp, artificial sequence
  Plasmid
ggggaattgtgagcggataacaattcccctgtagaaataattttgtttaactttaataaggagatataccatgggcagcagccatcaccatcatcaccacagccaggatccgaattcgagctcggcgcgcctgcaggtcgacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggtgagcaaccatcttttcgacgccatgcgggccgccgcgcccggtaacgcaccattcatccggatcgataacacgcgcacatggacctatgacgacgccttcgctctttccggccgcattgccagcgcgatggacgcgctcggcattcgccccggcgaccgcgttgcggtgcaggtcgagaaaagtgccgaggcattgatcctctatctcgcctgtcttcgaagcggcgccgtctacctgccgctcaacaccgcctatacgctggctgagctcgattattttatcggcgatgcggagccgcgtttggtggttgtcgcatcgtcggctcgagcgggcgtggagacaatcgccaagccccgcggtgcgatcgtcgaaactctcgacgctgctggcagcggctcgttgctggatctcgcccgcgacgagccggccgactttgtcgatgcctcgcgctccgccgatgatctggcggcgatcctctacacgtccggaacgacgggacgctccaagggggcgatgctcacgcatgggaacctgctctcgaacgccctgaccttgcgagatttttggcgcgtcaccgccggcgatcgactgatccatgccttgccgatcttccacacgcatggactgttcgtcgccacgaacgtcacactgctcgccggcgcctcgatgttcctgctgtcgaagttcgacccggaggagatcctgtcgctgatgccgcaggcaacgatgctgatgggcgtgccgaccttctacgtgcgcctcctgcagagcccgcgcctcgacaagcaagcggtcgccaacatccgcctcttcatttccggttcggctccactgcttgcagaaacacataccgagttccaggcacgtaccggtcacgccattctcgagcgctacggcatgacggaaaccaatatgaacacgtccaacccttatgaggggaaacggattgccggaacggtcggcttcccgctgcctgatgtgacggtgcgcgtcaccgatcccgccaccgggctcgcgctgccgcccgaacaaaccggcatgatcgagatcaaggggccgaacgttttcaagggctattggcgcatgcccgaaaaaaccgcggccgaattcaccgccgacggtttcttcatcagcggcgatctcggcaagatcgaccgcgacggttatgtccacatcgtcggccgcggcaaggatctggtgatttcgggtggatacaacatctatccgaaagaggttgagggcgagatcgaccagatcgagggtgtggttgagagcgctgtgatcggcgtgccgcatcccgatttcggagaaggcgtaacggccgtcgtcgtgcgcaagcccggcgctgccctcgatgaaaaggccatcgtcagcgccctccaggaccggctcgcgcgctacaaacaacccaagcgcatcatctttgcagaggacttgccgcgcaacacgatgggtaaggttcagaaaaacatcctgcggcagcaatacgccgatctttataccaggacgtaaggcgaccgcgctctctgggaggagagtgcgtcgacatcccgcatcaatcttgaaaacagcaactgcgacgcggaggcgtcggagggaggggaatcatgggtattgaattactgtccataggcctgctgatcgccatgttcatcattgcgacgatccagccaatcaacatgggtgcgctcgcctttgccggcgccttcgtgctcggctcgatgatcatcgggatgaaaaccaacgaaatatttgccggctttccgagtgatctgttcctgacgctcgtcgccgtcacctacctcttcgccatagcgcagatcaacggcacgatcgactggctcgtcgaatgtgccgtccgcctggtacgcgggcggatcggcttgattccctgggtgatgttccttgtcgccgccatcattactggcttcggtgcacttgggcctgctgcggtcgccattctcgcacccgtcgcgttgagctttgccgtgcagtaccgcattcatccggtgatgatgggtctgatggtgatccacggcgcgcaggcaggcggcttctcgccgatcagcatctatggcggaatcaccaaccagatcgttgcgaaggccggcctgcctttcgctccgacctcgctgtttctttccagcttcttctttaacctggcgatcgcggtgctggtgttcttcgtgttcggcggcgcgagggtgatgaagcacgatcccgcatcacttggccccttgcccgaactccatcccgagggcgtatcggcgtcgatcagaggccacggcggcacgccggcaaaaccgatcagagagcatgcctatggtacggcggccgataccgcgacgacgttgcgtctgaacaatgagagaattaccaccttgatcggcctgacggcgctcggcatcggcgccctggttttcaagttcaatgttggcctcgtcgccatgaccgtcgccgtcgtcctcgcgctgctgtcaccgaagacccagaaggccgcaatcgacaaggtcagttggtcgaccgtgctgctgattgccggcatcatcacctatgtcggcgtcatggagaaggccggtacggtcgactacgtggcgaatggcatatccagtctcggcatgccgctactggtagcgctcctgctttgctttacgggcgccatcgtctcggcctttgcttcctcgaccgcgctgctcggcgcgatcatcccgcttgccgttccattcctcctgcaagggcacatcagcgccatcggtgtggtcgcggcgatcgccatctcgacgacgatcgtcgacaccagcccattctccaccaacggcgcccttgtcgtcgccaatgcgccggacgacagccgtgagcaggtgttgcgacagctactgatctacagcgccttgatcgctatcatcggtccgatcgttgcctggttggtgttcgtcgtgcccgggctggtttgaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 20 - DNA, 1635 bp, Petroselineum crispum
  CDS (1)..(1635)
atgggagactgtgtagcacccaaagaagaccttattttccgatcgaaactccctgatatttacatcccgaaacaccttccgttacatacttattgtttcgaaaacatctcgaaagttggcgacaagtcctgtttaataaatggcgctacaggcgaaacgttcacttattctcaagttgagctcctttccaggaaagttgcatcagggttaaacaaactcggcattcaacagggcgataccatcatgcttttgctccccaactcccctgagtattttttcgctttcttaggcgcatcgtatcgtggtgcaatttctactatggccaatccgtttttcacttctgctgaggtgatcaaacagctcaaagcatccctagctaagctcataattacgcaagcttgttacgtagacaaagtgaaagactacgcagcagagaaaaatatacagatcatttgcatcgatgatgctcctcaggattgtttacatttctccaaacttatggaagctgatgaatcagaaatgcccgaggtagtgatcgattcagacgatgtcgtcgcgttaccttactcatcgggtactacaggactaccgaaaggtgttatgttgacccacaaaggacttgttactagcgtggcacaacaagttgatggagacaatccgaatttatatatgcatagcgaggatgtgatgatctgcatattgcctttgtttcatatttattcgcttaacgcggtgttgtgctgtggactcagagcaggggtgacgatcttgattatgcagaaatttgatattgtgccatttttggaactgatacagaaatataaagttacaattggaccgtttgtgccaccaattgtgttggcaattgcgaaaagtccagtggtggataaatatgacttgtcgtcggtgaggacggttatgtctggagctgctccgttagggaaggagcttgaagatgctgttagagctaagtttcctaatgccaaacttggtcagggatatggaatgacagaggcagggccagttttagcaatgtgcctggcgtttgcaaaggaaccatacgagatcaaatcgggtgcctgtggaactgttgtgaggaatgctgaaatgaaaattgtggatcctgagaccaacgcctctcttccacgaaaccaacgcggagagatttgcattcgaggtgaccaaattatgaaaggctacctcaatgatcctgaatcaacaaggacaacaatagacgaagaaggctggttgcacacaggagatataggcttcattgacgacgatgatgagctatttattgttgatagacttaaggaaataatcaaatacaaaggcttccaggttgcccctgctgaacttgaagctctgctacttactcatcctaccatttccgatgctgcagttgttcccatgatagatgagaaagcaggagaggtgcctgtggcttttgttgtgagaacaaacggtttcaccaccactgaggaagaaatcaagcaattcgtctcgaaacaggtggtgttctacaagagaatatttcgtgtattttttgttgatgcaattccgaaatcaccatctggaaagattcttcgaaaggacttgagagcaaaaatagcatccggtgatcttcccaaataa

SEQ ID NO: 21 - protein, 544 aa, Petroselineum crispum
MGDCVAPKEDLIFRSKLPDIYIPKHLPLHTYCFENISKVGDKSCLINGATGETFTYSQVELLSRKVASGLNKLGIQQGDTIMLLLPNSPEYFFAFLGASYRGAISTMANPFFTSAEVIKQLKASLAKLIITQACYVDKVKDYAAEKNIQIICIDDAPQDCLHFSKLMEADESEMPEVVIDSDDVVALPYSSGTTGLPKGVMLTHKGLVTSVAQQVDGDNPNLYMHSEDVMICILPLFHIYSLNAVLCCGLRAGVTILIMQKFDIVPFLELIQKYKVTIGPFVPPIVLAIAKSPVVDKYDLSSVRTVMSGAAPLGKELEDAVRAKFPNAKLGQGYGMTEAGPVLAMCLAFAKEPYEIKSGACGTVVRNAEMKIVDPETNASLPRNQRGEICIRGDQIMKGYLNDPESTRTTIDEEGWLHTGDIGFIDDDDELFIVDRLKEIIKYKGFQVAPAELEALLLTHPTISDAAVVPMIDEKAGEVPVAFVVRTNGFTTTEEEIKQFVSKQVVFYKRIFRVFFVDAIPKSPSGKILRKDLRAKIASGDLPK

SEQ ID NO: 22 - DNA, 8505 bp, artificial sequence
  Plasmid
ggggaattgtgagcggataacaattcccctgtagaaataattttgtttaactttaataaggagatataccatggcccttatgggagactgtgtagcacccaaagaagaccttattttccgatcgaaactccctgatatttacatcccgaaacaccttccgttacatacttattgtttcgaaaacatctcgaaagttggcgacaagtcctgtttaataaatggcgctacaggcgaaacgttcacttattctcaagttgagctcctttccaggaaagttgcatcagggttaaacaaactcggcattcaacagggcgataccatcatgcttttgctccccaactcccctgagtattttttcgctttcttaggcgcatcgtatcgtggtgcaatttctactatggccaatccgtttttcacttctgctgaggtgatcaaacagctcaaagcatccctagctaagctcataattacgcaagcttgttacgtagacaaagtgaaagactacgcagcagagaaaaatatacagatcatttgcatcgatgatgctcctcaggattgtttacatttctccaaacttatggaagctgatgaatcagaaatgcccgaggtagtgatcgattcagacgatgtcgtcgcgttaccttactcatcgggtactacaggactaccgaaaggtgttatgttgacccacaaaggacttgttactagcgtggcacaacaagttgatggagacaatccgaatttatatatgcatagcgaggatgtgatgatctgcatattgcctttgtttcatatttattcgcttaacgcggtgttgtgctgtggactcagagcaggggtgacgatcttgattatgcagaaatttgatattgtgccatttttggaactgatacagaaatataaagttacaattggaccgtttgtgccaccaattgtgttggcaattgcgaaaagtccagtggtggataaatatgacttgtcgtcggtgaggacggttatgtctggagctgctccgttagggaaggagcttgaagatgctgttagagctaagtttcctaatgccaaacttggtcagggatatggaatgacagaggcagggccagttttagcaatgtgcctggcgtttgcaaaggaaccatacgagatcaaatcgggtgcctgtggaactgttgtgaggaatgctgaaatgaaaattgtggatcctgagaccaacgcctctcttccacgaaaccaacgcggagagatttgcattcgaggtgaccaaattatgaaaggctacctcaatgatcctgaatcaacaaggacaacaatagacgaagaaggctggttgcacacaggagatataggcttcattgacgacgatgatgagctatttattgttgatagacttaaggaaataatcaaatacaaaggcttccaggttgcccctgctgaacttgaagctctgctacttactcatcctaccatttccgatgctgcagttgttcccatgatagatgagaaagcaggagaggtgcctgtggcttttgttgtgagaacaaacggtttcaccaccactgaggaagaaatcaagcaattcgtctcgaaacaggtggtgttctacaagagaatatttcgtgtattttttgttgatgcaattccgaaatcaccatctggaaagattcttcgaaaggacttgagagcaaaaatagcatccggtgatcttcccaaataaaagggcgaattcgaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggtgagcaaccatcttttcgacgccatgcgggccgccgcgcccggtaacgcaccattcatccggatcgataacacgcgcacatggacctatgacgacgccttcgctctttccggccgcattgccagcgcgatggacgcgctcggcattcgccccggcgaccgcgttgcggtgcaggtcgagaaaagtgccgaggcattgatcctctatctcgcctgtcttcgaagcggcgccgtctacctgccgctcaacaccgcctatacgctggctgagctcgattattttatcggcgatgcggagccgcgtttggtggttgtcgcatcgtcggctcgagcgggcgtggagacaatcgccaagccccgcggtgcgatcgtcgaaactctcgacgctgctggcagcggctcgttgctggatctcgcccgcgacgagccggccgactttgtcgatgcctcgcgctccgccgatgatctggcggcgatcctctacacgtccggaacgacgggacgctccaagggggcgatgctcacgcatgggaacctgctctcgaacgccctgaccttgcgagatttttggcgcgtcaccgccggcgatcgactgatccatgccttgccgatcttccacacgcatggactgttcgtcgccacgaacgtcacactgctcgccggcgcctcgatgttcctgctgtcgaagttcgacccggaggagatcctgtcgctgatgccgcaggcaacgatgctgatgggcgtgccgaccttctacgtgcgcctcctgcagagcccgcgcctcgacaagcaagcggtcgccaacatccgcctcttcatttccggttcggctccactgcttgcagaaacacataccgagttccaggcacgtaccggtcacgccattctcgagcgctacggcatgacggaaaccaatatgaacacgtccaacccttatgaggggaaacggattgccggaacggtcggcttcccgctgcctgatgtgacggtgcgcgtcaccgatcccgccaccgggctcgcgctgccgcccgaacaaaccggcatgatcgagatcaaggggccgaacgttttcaagggctattggcgcatgcccgaaaaaaccgcggccgaattcaccgccgacggtttcttcatcagcggcgatctcggcaagatcgaccgcgacggttatgtccacatcgtcggccgcggcaaggatctggtgatttcgggtggatacaacatctatccgaaagaggttgagggcgagatcgaccagatcgagggtgtggttgagagcgctgtgatcggcgtgccgcatcccgatttcggagaaggcgtaacggccgtcgtcgtgcgcaagcccggcgctgccctcgatgaaaaggccatcgtcagcgccctccaggaccggctcgcgcgctacaaacaacccaagcgcatcatctttgcagaggacttgccgcgcaacacgatgggtaaggttcagaaaaacatcctgcggcagcaatacgccgatctttataccaggacgtaaggcgaccgcgctctctgggaggagagtgcgtcgacatcccgcatcaatcttgaaaacagcaactgcgacgcggaggcgtcggagggaggggaatcatgggtattgaattactgtccataggcctgctgatcgccatgttcatcattgcgacgatccagccaatcaacatgggtgcgctcgcctttgccggcgccttcgtgctcggctcgatgatcatcgggatgaaaaccaacgaaatatttgccggctttccgagtgatctgttcctgacgctcgtcgccgtcacctacctcttcgccatagcgcagatcaacggcacgatcgactggctcgtcgaatgtgccgtccgcctggtacgcgggcggatcggcttgattccctgggtgatgttccttgtcgccgccatcattactggcttcggtgcacttgggcctgctgcggtcgccattctcgcacccgtcgcgttgagctttgccgtgcagtaccgcattcatccggtgatgatgggtctgatggtgatccacggcgcgcaggcaggcggcttctcgccgatcagcatctatggcggaatcaccaaccagatcgttgcgaaggccggcctgcctttcgctccgacctcgctgtttctttccagcttcttctttaacctggcgatcgcggtgctggtgttcttcgtgttcggcggcgcgagggtgatgaagcacgatcccgcatcacttggccccttgcccgaactccatcccgagggcgtatcggcgtcgatcagaggccacggcggcacgccggcaaaaccgatcagagagcatgcctatggtacggcggccgataccgcgacgacgttgcgtctgaacaatgagagaattaccaccttgatcggcctgacggcgctcggcatcggcgccctggttttcaagttcaatgttggcctcgtcgccatgaccgtcgccgtcgtcctcgcgctgctgtcaccgaagacccagaaggccgcaatcgacaaggtcagttggtcgaccgtgctgctgattgccggcatcatcacctatgtcggcgtcatggagaaggccggtacggtcgactacgtggcgaatggcatatccagtctcggcatgccgctactggtagcgctcctgctttgctttacgggcgccatcgtctcggcctttgcttcctcgaccgcgctgctcggcgcgatcatcccgcttgccgttccattcctcctgcaagggcacatcagcgccatcggtgtggtcgcggcgatcgccatctcgacgacgatcgtcgacaccagcccattctccaccaacggcgcccttgtcgtcgccaatgcgccggacgacagccgtgagcaggtgttgcgacagctactgatctacagcgccttgatcgctatcatcggtccgatcgttgcctggttggtgttcgtcgtgcccgggctggtttgaggtaccctcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 23 - DNA, 2142 bp, Rhodotorula mucilaginosa
atggccccctccgtcgactcgatcgcgacttcggttgccaactccctctcgaacgggttgcacgccgccgccgccgccaacggtggcgacgtccacaagaagacggccggtgctggctccctcctcccgaccaccgagacgacccagctcgacatcgttgagcgcatcttggccgacgccggcgcgacggaccagatcaaactcgatgggtacaccctcacgctcggcgacgtcgtcggcgctgctcgccgtggccgctccgtcaaggtcgcagacagcccgcacatccgcgagaagatcgatgccagtgtcgagttcctccgtactcagctcgacaacagtgtctacggtgtcacgactggtttcggcggctcggccgacacccggactgaggatgcgatctcgctccaaaaggccctgctcgagcaccagctctgcggtgtcctccccacctcgatggatggctttgcgctcggtcgcggcctcgagaactcgcttccgctcgaagtcgtccgaggcgcgatgaccatccgtgtcaactcgctcactcgcggtcactcggcggtccgcatcgtcgtcctcgaagccctcaccaacttcctcaaccacggcatcaccccgatcgtcccgcttcgaggcaccatctcggcgtcgggcgacctttcccccctctcttacatcgccgcctcgatcaccggccacccggactcgaaggtccacgtcgacggcaagatcatgtccgcccaggaggcgatcgcgctcaagggtcttcagcccgtcgtcctcggtccgaaggagggtctcggtctcgtcaacggcacggccgtctccgcctcgatggcgacgctggccctcaccgacgcacacgtcctctcgctcctcgcacaggcgctcactgctcttactgtcgaggccatggtcggacacgccggctcgttccacccattcctccacgacgtcacgcgccctcacccgacccagatcgaggtggcgcgcaacatccggactcttctcgagggcagcaagtacgccgtccaccacgagactgaagtcaaggtcaaggacgacgagggcatcctcaggcaggaccggtacccgctccgctgctcgccgcagtggctcggtccccttgtcagcgacatgattcacgctcacgctgtcctctcgctcgaggctggtcagtcgaccaccgacaacccgctgatcgacctcgagaacaagatgacccaccatggcggagccttcatggcgagcagcgtcggaaacacgatggagaagactcgcctcgccgtcgcgctgatgggcaaggtcagctttactcagctcaccgagatgctcaacgccggcatgaaccgggcccttccgtcctgcctcgctgccgaggacccttccctctcttatcactgcaagggtctcgacattgctgcggccgcctacacttccgagctcggtcaccttgccaacccggtttcgacccacgtccagccggccgagatgggcaaccaggccatcaactcgctcgccctcatctcggcccgccgcaccgccgaggcgaacgacgttctctccctcctcctcgccacccacctctactgcgtcctccaggccgtcgacctccgcgcgatggagtttgagcacaccaaggcgttcgagccgatggtcactgagctgttgaagcagcactttggcgcgctcgcgacggccgaagtcgaggacaaggtccgcaagtcgatctacaagcggttgcagcagaacaactcgtacgacctcgagcagcggtggcacgacacgttctcggtcgcgaccggtgccgtcgtcgaggcgctcgccggccaggaggtctcgctcgcgagcctcaacgcctggaaggtcgcctgcgccgagaaggctatcgcgctcacgcgctccgtccgcgactcgttctgggcggctccgtcgtcgtcgtcgcccgcgctcaagtacctctccccgcggacgcgcgtcctgtattcgttcgtccgggaggaggtcggcgtcaaggcccgccgcggcgatgtctacctcggcaagcaggaggtcacgatcggcaccaacgtcagccgcatctacgaggcgatcaagagcggttgcatcgcccccgtcctcgtcaagatgatggcatag

SEQ ID NO: 24 - DNA, 2311 bp, Amanita muscaria
gtcgctcgcaaatctaaatgggtctcgataactccaagaacactgccaaattttttgacctaccaaaagccgtccatggtatgaatggtacaacccccgtcaatggttttaaagcgacagcgctttccaaggcctcccgaacaatgaccaagactagcgcactctcgcaattcttagaagcgtaccgtgaactcgagggctacaagaatggtagagccatcaaggttgacggtcaaacgttatctattgcagccgtcgctgcagctgctcgctacaatgcggccgttgagttggacgaatccccacttgttaaggagcgcgtgaggaaaagtcagcttgctatcgcaaacaaagtatcgaccggtgccagcgtatacggactgtcaactggtttcggtggcagtgctgatacacggacggacaaaccgatgttgttggggtttgcccttttgcaacaccaacatgtagggatactgcccacctcgactgagcctttggacgtcctacctctccaagatgcaaataacacaagcatgccagaggcgtggattcgcggggccattttgatccgtatgaattcgctaattcgtggccactctggaatcagatgggagttgatcgaaaagatgagagaactactcgcggccaatgtgatacctgtcgttcccctgagaggcagcatctcctcatccggagatctgtctcccctatcctatatcgcaggcacgattattggcaacccatcaatcaaggtatatcacggtccatcaaagtccggaattcgccaaattggatcctcgaaggatgtcttggctctgcataatatcgaacctttcccactggaatcgaaagaacctcttggtattttgaatgggaccgcattctcggcatctgtggcagctttagccctaaacgaagctatccatcttgtcttgttggctcaagtgtgcacggctatggggaccgaggcattgataggcactcgcgcttctcatgcaccgttcattcatgccaccgcacgaccacatcccggtcaagtagaatgtgctgagaacatttggaatttgctcgatgggagtaaattggctcagttagaagagcacgaagttcgcctagaagacgataaatacacccttcggcaggaccgttatccactccgaacttcgcctcaattccttgggcctcagattgaagacataatctccgctttccagactgtaacgcaggagtgtaattacttaccagctactgacaatccactgattgatggggagactggcgaatctcaccacggtggcaatttccaagcgatggctgtaactaatgcaatggagaagacgcgacttgctttacatcacgttggcaaattactattttcccagagcactgaattagtcaatcctgcgatgaaccgcggtctgccgccttcagtagctgccacagatccatctctcaactaccacgccaaaggactagacatagcaactgcggcctacgtagccgaagcgactcctggccccactcacattcagtcggcagaaatgcacaaccaagctgttaactccctggcgttgatttctgctcgggctaccatcacatcgttggaagtgctaacatctctgatcgcgtcttacttgtatattctatgccaagctctcgacctccgtgcccttcagcgcgagttcttgcccggtctagacatcatcattcgtgaggagttaagatcgtcatttggatctttcctgtcatcagaacagatggagaaattgcaacaaaatctaactagtgcatttgaagatcatcttgacaagaccacgacaatggataatactgatcgaatgactacgatggctgctacatcatcatcagttctacttcaattctttactgattctggcgcgtctgttcctccctcgtcttgcgatcttctctccagtgtctcgtccttccaatcttctgtggcgacacggtcttcagttctcatggatgacctacggaaagaatatatttttggagaccgtggccccacgcccgcaagccaatacatcggaaagacacggccagtataccaattcattagaacaactataggcgttcgtaagcatggttctgagaactacaacaagttttataatgggctgggtgtcgaagacgttaccatcggtcaaaatatatcacgcatatacgagtcaatccgggacggcaaaatgcaatccattattgtctcgttgtttgattaggtcttgaaagcttgtatcttattaataaccatacacttcctcgaggtctaaaaaaaaaaaaaaaaaaaaaa

SEQ ID NO: 25 - DNA, 3047 bp, Ustilago maydis
ggtgctccccaacaaatggcgcgcttttttcggtagcatgcaggataatctgttcatcactgtgagggttcacgttcgtgattaaacaacgcgcacattccctgtttggctgtcatcggatattccgcgacaactcggtatattattagtgtagtttgacagagggagtggacgcggctgagatgggaccgttccgtgtcaggagagtggacaacgcattgcgcggaatgaagtcagaatcgatgcatcaatgattcacgattgttgctctgacgatcggctcgcccgttccgttcgcggtgcgcatcctgattgccagatagccagagacgtggagcctgaaggtgactatagtatgggacagcaatcctagccgactttctcacctcctatccgcccatatctgcgtgccgtgcctcttcgatcgtctctacacgaccataacagctgtcctctcgcgtccataccgttcctcttcccaccgcatctggcatcatggctccaaccgcagacgtgctccctcccgtcgaggcatccacgcgtccaggcttgctcgtccagccttcggataccaaacttcgcaaagcatcgtccttccgaaccgagcaggtcgttatcgacggctacaatctcaagatccagggtctcgtcgcttccgctcgatacggtcacgttacccgtcctcgaccctccgctgagacgcgaaagcgtattgatgactcggtccagtccttaatcgccaagctcgacggtggcgagtcaatctacggcatcaacacggggttcggtgggtccgccgactcgaggaccgccaacacacgtgcgcttcagctggccttgctccagatgcagcagtgtggcgtgctccccgtgccatccacattccccacgggcgaacccagctcggcaccctttgcactccctttgacggacacagagtcttcactgatcatgccggaggcatgggtaaggggtgccatcgtggttaggctcagctctctgatgcgcggtcattcgggtgtgcgttgggaggtgctcgacaagatgcagaagcttttcctccagaacaacgtcactccagtcgtaccagtcaggtcgagtatctcggccagtggtgatcttagcccacttagctacgtagccggtgcgcttgccggtcagcgtggcatctactgctttgtcaccgacggccgtggtcagcgtgtcaaggtgactgcggatgaggcttgtcgcatgcacaagatcacccccgtccagtatgagcccaaggaggcgcttggtctgctcaacggcaccgctttttcagcctctgttgcgggtctcgctacctacgaggccgaaaatctagcctctctgacgcagctcaccaccgctatggccgtcgaagccctcaagggtaccgatgccagctttgctcctttcattcacgaaatcgcccgcccgcatcctggtcagatcaagagcgccaagtttatccgcgcgcatctttccggctctaggctagcagagcatctcgaaaacgaaaagcacgtcctcttctccgaagacaacggaacgctgcgtcaggaccgttacacgctgcaaaccgcctcccagtgggtcggcccgggtctcgaggacatcgaaaacgcaaagcgatccgtcgactttgagattaacagcaccacagataaccccatgatcgacccgtacgacggcgacggtcgcatccaccacggaggcaacttccaggccatggccatgacgaatgccgtcgagaagatccgcctcgccttgtgtgctatgggcaaaatgacgttccagcagatgacagagctcgtcaacccggcaatgaaccgaggattgcccgccaacttggcttccacgcctgatctgtcgctcaacttccacgccaagggaatcaatattgcgcttgccagtgtcacttcggaactcatgttcctcggcaaccccgtttcaacgcatgtacaaagtgcagagatggccaaccaggccttcaactcgctggcgctcatcagcggccgccagacgctgcaggcgatcgagtgcctctcgatgattcaggcttggtcgctctacctcttgtgccaagcactcgatattcgcgctttgcagtataaggttgctgagcagctgcccacgctcatcttggcatcgctgcacagtcactttggcgagtggatggatgagaccaagcagcaagagattgcagcacaggtgctcaagagcatgagcaagcgtctcgacgaaacctcgtccaaggaccttcgcgatcgactggtcgagacgtaccaagacgcgtcgtctgtgcttgtgaggtacttttccgagctgcctagcggtggtggtgcggatccgctgaggaacattgtcaagtggcgcgccaccggtgtagctgacacggaaaagatttacaggcaggtaacgatcgaatttcttgacaacccatacgcttgccatgccagccacctgttgggcaagaccaagcgcgcctacgagtttgtcaggaagacgctgggtgtgcccatgcatggtaaggagaacctcaacgaattcaagggcgaatttgagcaatggaacacgacgggcggttacgtctcggtcatctatgctagtattcgagatggcgagttgtataacatgctgagcgagctcgaaagggatttgtaaaggggtgcaagcagcgtattaatagttagtataaattggccatctacggtgacaaattgcgtgtgagtgccaaaagggccatcgaaatgatcatggacagcgacagactgtgtgttgatttgtcaaagtgatttggcactaccgaatatgaccgtgtgtaccggcaccaaggcgaggtgatgcgaatgcatgtttttgcgtggcgtcaaagggggatgcaggacatggtcgactgcttgtcggagctgatgaggtcgtagcggattcggaatttgggttcgagggctgtgaagggatgttgaggtgtatcaaagggacttggcttgtgctgcgcttgggagtgggagggacatttcaggtgcatctgctttcgggat

SEQ ID NO: 26 - DNA, 2209 bp, Arabidopsis thaliana
atggagattaacggggcacacaagagcaacggaggaggagtggacgctatgttatgcggcggagacatcaagacaaagaacatggtgatcaacgcggaggatcctctcaactggggagctgcagcggagcaaatgaaaggtagccatttggatgaagtgaagagaatggttgctgagtttaggaagccagttgtgaatcttggtggtgagactctgaccattggacaagtggctgcgatctcaactattggtaacagtgtgaaggtggagctatcggagacagctagagccggtgtgaatgctagtagtgattgggttatggagagtatgaacaaaggcactgatagttatggtgttactactggttttggtgctacttctcatcggagaaccaaaaacggtgtcgcacttcagaaggaacttattagattccttaacgccggaatattcggaagcacgaaagaaacaagccacacattgccacactccgccacaagagccgccatgcttgtacgaatcaacactctcctccaaggattttccggtatccgatttgagattctcgaagcaattaccagtttcctcaacaacaacatcactccatctctccccctccgtggtacaatcaccgcctccggagatctcgttcctctctcctacatcgccggacttctcaccggtcgtcccaattccaaagctactggtcccaacggtgaagctttaacagcagaggaagctttcaaattagcaggaatcagctccggattctttgatctccagcctaaggaaggtctcgcgctagtcaatggcacggcggttggatctggaatggcgtcaatggtgttattcgaaacgaatgttctctctgttttggctgagattttgtcggcggttttcgcagaggtgatgagtggtaagcctgagttcaccgatcatctcactcacagacttaaacatcatcccggtcaaatcgaagcggcggcgataatggagcatatcctcgacggaagctcgtacatgaaattagctcagaagcttcacgagatggatccgttacagaaacctaaacaagatcgttacgctcttcgtacttctcctcaatggttaggtcctcaaatcgaagtgatccgttacgcaacgaaatcgatcgagcgtgagattaactccgtcaacgataatccgttgatcgatgtttcgaggaacaaggcgattcacggtggtaacttccaaggaacaccaatcggagtttcaatggataacacgagattggcgatagcagcgattggtaaactcatgtttgctcaattctcagagcttgtgaatgatttctacaacaatggtttaccctcgaatctaaccgcttcgaggaatccaagtttggattatggattcaagggagctgagattgcaatggcttcttattgttcagagcttcaatacttagctaatcctgtgactagccatgttcaatcagcagagcaacataaccaagatgtcaactctttgggactaatctcgtctcgcaaaacttctgaagctgttgatattctcaagcttatgtcaacaacgttcctcgttgcgatttgtcaagctgtggatttgagacatttggaggagaatttgagacagactgtgaagaacactgtctctcaagtggcgaagaaagttcttactactggagtcaatggtgagcttcatccttctcgcttctgcgaaaaggatttactcaaagttgtagaccgtgaacaagtctacacatacgcggatgatccttgtagcgcaacgtacccgttgattcagaagctgagacaagttattgttgaccatgctttgatcaatggtgagagtgagaagaatgcagtgacttcaatcttccataagattggagctttcgaggaggagcttaaggcagtgctaccgaaagaagtggaagcagcaagagcagcctacgataacggaacatcggctatcccgaacaggatcaaggaatgtaggtcgtatccattgtatagattcgtgagggaagagcttggaacagagcttttgaccggagagaaagtgacgtcgcctggagaagagttcgacaaggttttcacggcgatttgtgaaggtaaaatcattgatccgatgatggaatgtctcaacgagtggaacggagctcccattccaatatgttaagagtatagtcctctgtttttttcttaccata

SEQ ID NO: 27 - DNA, 2439 bp, Rubus idaeus
aaacactccataactccataactccatttctgaaattcatttctgggttattttctcacactacaatggagagcataacccagaatggacaccaccaccagaatgggatccaaaacggttcgttggacgacggtctctgcatcaaaacagagtccatcaaaacgggctactctgtttcggacccgcttaactggggagcagccgccgagtcaatgacaggcagccacctcgacgaagttaggcgcatggtggccgagtacaggaaaccggtggtgaagctcggtggagaaaccttgactatttcccaggtggcggccatagccaaccatgactctggtgtcaaggttgaactcgctgagtccgccagggcgggtgtgaaggccagtagtgattgggtcatggattccatgaacaaagggactgatagctatggtgtcaccactgggttcggtgcgacctcccacagacgaaccaaacaaggcgctgcacttcaaaaggagttaattagattcttgaatgctggagtattgcgcaatggaacagagtcagctcacactctgcctcactctgcaacaagagcagccatgctcgtcagaatcaacacactcctccaaggctactccggcataagattcgaaatcttagaagccatctccaaatttctcaaccacaacataactccatgcttgcctcttcgtggcacgatcaccgcctccggagaccttgttccgctgtcctacatcgccggactactaacgggccggcccaattccaaggcggtcgggccaaaaggcgagaccctcaatgccgctgaggcttttgcacaagtcggtatcagctcagggtttttcgagctgcagcctaaagaaggacttgctcttgttaacggcactgctgttggctctggcttggcctccacggttcttttcgagaccaacattttggccttgctgtccgaaatcttgtctgcgattttcgctgaagtgatgcaggggaagcccgaattcacagaccacttgacacataaattgaagcaccacccgggtcaaattgaggctgctgcaattatggaacacattttggatggtagctcttacgtcaaagctgccgagaaacttcatgagcaggaccctcttcagaagcctaaacaagaccgctacgctctccgaacatcaccacaatggctcggtccacaaatcgaagtgatcagattttcgactaaatctattgagagggagattaattctgtcaatgacaaccctttgattgatgtttcgaggaacaaggcattgcatggtggcaacttccagggtaccccaattggagtgtccatggacaacacccgtttggctattgcatccattgggaagctcatgtttgctcagttttctgaacttgtcaatgacttttacaacaacggtttgccatcgaatttatcgggtgggagggaccccagtttggattatggcttcaagggagctgagattgccatggcatcttattgttccgagcttcagtttctagccaatccggtgactaaccatgtccagagcgccgagcagcacaaccaggatgtgaactctttggggctgatttcgtcgcgaaaaaccgcagaagctgttgacatattgaagctcatgtcttccacattcttagttgcgctttgccaagccattgacttgaggcatttggaggagaacttgaagagcacggttaaaaacactgtgagtcaattggctaagagggttttgactactggggttaatggggagcttcacccgtcgaggttctgcgagaaggatttgcttatggttgttgaaagggagtaccttttcgcctacattgacgatccttgcagcgccacatatccattgatgcaaaggctaaggcaagtgcttgttgaacacgctttgacaaacggtgagaatgagaaaaatgcaagcacttctattttccaaaagattacggcttttgaggaggagctgaagaccattttgcctaaggaggttgagagcgctagggctgcgtacgagagcgggaatgctgctattccaaacaggattgtggagtgcaggtcatatcctttgtacaaatttgtgagggaggagttggggggagagttcctgacgggtgaaaaggtcagatcccccggggaggagtgtgacaaagtgttcacagctatgtgccagggcaacattattgatcccattctcgactgcctcagcggttggaacggtgaacctcttccgatctgctagccttaatttcggtacccgttttgagtgatgtgtgtcattccattccacttcgatcttctggctccatagttttaagtttgatgaggattgctagctttaattgtgtgactatatataaaacctaataaaatgtaaaaccatctgtttatttgaaactgtagttcttcttttcttacttacc

SEQ ID NO: 28 - DNA, 2409 bp, Medicago savita
cttctttctttcataatcattagaatttccattctatcaaaattctaggtaccaccacacaacatattaaggaacattaatcaatactattaagatatggaaacaatatcagcagctatcacaaaaaacaatgccaatgaatcattctgcttgattcatgcaaagaataataataacatgaaagtgaatgaagctgatcctttgaattggggggtggcagctgaggcaatgaaaggcagtcaccttgatgaggtgaagcgtatggtggcagagtaccggaaaccggtggtccgtcttggtggcgagacactgacgatttctcaggtggctgccattgctgcacatgaccatggtgtgcaggtggacctgtctgaatctgctagggatggagttaaggccagcagtgaatgggtgatggagagtatgaacaaaggcacggacagttacggtgtcaccaccgggttcggcgccacctcgcacagccgtaccaaacaaggtggtgctttgcagaaagaactcatcaggtttttgaatgcaggaatattcggaaatggaacagagtcaaatcacacactaccaaaaacagcaacaagagcagccatgctagtgaggatcaacacactcctccaaggttattcaggaatagattttgaaatcttggaagccatcactaagccccttaacaaaaccgtcactccatgtttaccgcttcgtggtacaatcacagcttcaggtgatttagttcctctttcatacattgctggtttactcaccggaagaccaaattcaaaagctcatggaccatctggagaagtacttaatgcaaaagaagcttttaatttggctggaatcaatgctgagttctttgaattacaaccaaaagaaggtcttgcccttgttaacggaacagctgttggttccggtttagcttctattgttctctttgaggctaacattttggctgtgttgtctgaagttctatcagctatttttgctgaagttatgcaagggaaacctgaatttaccgatcatttgacacacaagttgaaacaccaccctggtcaaattgaggctgctgcgattatggaacacattttggacggcagctcttatgtcaaagcagctaagaagttgcatgagatagatcctttgcagaagccaaaacaagatagatatgcacttagaacttcaccacaatggcttggtcctttggttgaagtgattagattctctaccaagtcaattgagagagagatcaactctgtcaatgacaaccctttgattgatgtttcaagaaacaaagctttgcacggcggaaactttcaaggaacacctattggagtatccatggataatacacgtttggctctcgcatcaattggcaaacttatgtttgctcaattctctgagcttgttaatgacttttacaacaatggattgccttcaaatctttctgctagtagaaatcctagcttggattatggtttcaagggagctgaaattgccatggcttcctattgttctgagttgcaatatcttgcaaatccggttacaacccacgtccaaagtgctgagcagcacaaccaagatgtgaactctttgggtttgatttctgctagaaaaacaaatgaagccattgagatccttcagctcatgtcttccaccttcttgattgcactatgccaagcaattgatttaagacatttggaggagaacttgaaaaactcagtcaagaacaccgtaagtcaagttgccaaaaagactcttaccatgggtgtcaatggagaacttcacccttcaagattctgcgaaaaagacttgttgaaagtggttgacagggagcatgtatttgcttatattgatgatccttgtagcgctacatacccgttgagtcaaaaactaaggcaagtgttggtagatcatgcactagtaaatggagagagtgagaagaattttaacacttcaatctttcaaaagattgctacttttgaggaagagttgaagaccctcttgccaaaagaggttgaaagtgcaaggaccgcatatgagagtggaaacccaacaatcccaaacaagatcaatggatgcagatcttatccactttacaagtttgtgagagaggagctaggaactggtttactaaccggagaaaatgtcatttcaccaggagaagagtgtgacaaactattttcagctatgtgtcagggaaaaatcatcgatcctcttcttgaatgtttgggagagtggaacggtgctccccttcctatttgttaactttgttggttacttttgaaaatgctttatttgtattttatacaagtgtatcaaaaatcatataggtttttcatgctttaacaaattaatatggaaagctaaaaagctccagttcagtttcctccaaaaaaaaaa

SEQ ID NO: 29 - DNA, 2444 bp, Rehmannia glutinosa
acacaaaaacacacacacaagagcaaaaaaataataacacctatcgtgtgtgtgttctgtgtgaaaaaaaaaaaaaaacaacccaaagtcgtgatatctaaaagcgcgtatcaatggagaatgggcaccaccactcgaacgggttgtgcgtggagactacgcgtgatccgttgaactgggtggcggcggcggagtcgctgaaggggagccacctggacgaggtgaagaggatggtggaggagttcaggaagccggcggtgaagctcggcggtgagagcctgactatagcgcaggtggcggcgatcgcggcgagggataatgcggtggcggtggagctggcggagacggcgcgtgcgggggttaaggcgagtagcgattgggttatggagagtatgaataaagggactgacagttatggagttacaacgggttttggtgccacgtcacataggaggactaaacaaggtggtgctcttcagaaggagctcattaggttcttgaatgccggaatattcggcaacggcacggaatctaaccacgcgctgccacactccgccacgagagccgccatgctcgtccgaatcaacacgctcctccaaggatattccggcatccgatttgaaatcctagaagccctaacaaaattcctcaaccacaacatcaccccctgtttgcccctccgcggcacgatcaccgcctccggcgacctcgtcccgctatcctacattgccgggcttttaacgggccggcccaactccaaggccgtcggcccaaacggcgaagccctcaacgccggcgaggctttcagcctcgccggcgttagcggcttcttcgagctgcagcccaaagaaggcctcgcgctagtcaacgggacagctgtcgggtccggattggcctcgatcgccctgtacgacgcgaacatcctcgccgtcctgtcggaagtgacgtcagtgattttcgctgaggtcatgaatgggaaacctgaatttacggatcatttgacacataagctgaaacatcaccctggccaaattgaggccgctgctataatggaacacattttagatggtagcgcgtacgttaaggctgctcagaaattgcacgaaaccgatccgttgcaaaaaccgaaacaggatcggtacgcgcttagaacgtcgcctcaatggctcggcccccaaatcgaagttatccgaaccgcgacgaaaatgatcgagcgggaaattaattcggttaacgacacacctctaatcgatgtctcgagaaataaagcgttacatggcggtaacttccagggcacgccaatcggggtatcgatggacaacaccagattggcgatagcagctatcggaaaattgatgttcgctcaattttccgagctggttaatgatttctacaacaatggattgccgtctaatctctctggcggtaggaatccgagcttggattacggtttcaaagggtccgaaatcgcgatggcttcgtattgttcggagcttcaatttttagctaatcctgttaccaatcatgtccaaagtgcagagcaacataaccaagatgtgaattcacttggattgatttcttctagaaagaccgtcgaggctctggatattctaaagctgatgtcatccacatatttaatcgcgctatgccaggccgtcgatttgaggcacttggaggagaatttgaggctttcagttaaaaacaccgttagccaagtggcgaagaggactctgacaatgggtattaatggcgaacttcatccgtcaagattctgcgagaaggatcttctccgtgtggtggaccgcgagtacgtgtttgcatacatcgacgatccgtgcagcgggacctaccccttgatgcagaagttgaggcaagttctcgtggaccacgcgttgaacaacggtgagagtgagaaaaacgtgagcacgtctatttttcaaaagatcgaggcgtttgaggtagagttgaaggcgatcttgcctaaagaggtcgagagtgcacggatcgcgttggagagtggaaatccggcgattggtaacaggattacggaatgcagatcgtatccgttgtacaagtttatcagagaggaacttgggacgaactacttgacgggcgaaaaggtcgtttctccgggggaggaatgtgataaggtgttcacagctttgagcaagggtttgattgttgatcctttgttgaagtgtcttgagggttggaatggtgcacctccccctatctgctagttcaattaaaatttgttttgtggttaaggacttttgtgtttgttaatgttttcctctcaatgttggtttaattataatgtgattctgtctagggtgaaataaattgtaaaaaaaattatgagttcttatgtttttttaaaaaaaaaaaaaaaaaaaaaaaaaaaaaaaaaaaaaaaaaaaaaaaaaaaaaaaaaaaaaaaaaaa

SEQ ID NO: 30 - DNA, 2442 bp, Lactuca sativa
gagcaatctgatcaatacccattcacgcacaaagagtgtgagtctagtgtgtgaagaagtacacaattagattgttcttgtttctttgatctatagtctacaatctgtataaataataatggagaacggtaatcacgttaatggagtcgttaatgagttgtgcatcaaggatccattgaactggggagttgcagcggaggcgttgaccggaagtcaccttgatgaggtgaagaagatggttgcggagttcagaaagccggtggtgaagctcggaggagagacgcttacagtttctcaggtggcggggatcgcagctgctaatgacagtgacaccgtgaaggtggagctgtcggaagccgcgagggctggagttaaggcgagtagtgattgggttatggagagcatgaataaaggaactgatagttatggtgtcaccaccggcttcggcgccacctctcaccggagaactaagcaaggcggtgctttacagaaggagctcattagatttttgaacgccggaatattcggcaatggaacggaaacaagccacacacttccacattcagccaccagagccgccatgatcgtcagaatcaacaccctcctccagggttactccggcatccgattcgagatcttggaagccatcaccaagttccttaacaacaacatcaccccttgtttacccctccgtggaaccatcaccgcctccggtgaccttgtcccattatcatacatcgccggcctcttaaccggccgccccaactccaaagccgttggccccaccggagaagtcctcaatgccgaaaaggccttcgctgcagccggagttgaaggtgggttcttcgagttacagccgaaagaagggctagcacttgttaacggcaccgccgtggggtccgggatggcttccatggttctatttgatgctaatgtacttgcgttgttgtcggaagtgttatcggcgatcttcgctgaggttatgcaagggaagccggagtttaccgatcacttgacacacaaattgaagcatcaccctggtcaaatcgaggcggcggcgatcatggagtatattttggacggaagcgattacgtcaaggcggcgcaaaaggtccacgaaatggacccgttacagaaaccaaaacaagatcgttatgctctccgtacatctccccaatggctcggacctcaaatcgaagtaatccgatcatcaaccaaaatgatcgagagggaaatcaattccgtcaacgacaacccattgatcgacgtttccagaaacaaagctttacacggtggtaacttccaaggaaccccaatcggagtttccatggacaacacccgtctcgccattgctgcaatcggaaaactcatgttcgctcaattttctgagctggttaacgatttctacaacaatggattaccatcgaatctctccggtggacgtaaccctagtttggactacgggttcaaaggtggagaaatcgccatggcttcttactgttctgagcttcagtttctcgcaaatccagtcaccaaccatgttcaaagcgccgaacaacacaatcaagacgttaattctctcggattaatttcagcgaggaaaaccgcagaagcagtcgacatcttaaaactcatgtcgtcgacatacttagtcgctctatgccaatccatcgatttacgccatttggaagagaacatgaaatcgacagtgaagaacaccgtaagccaagtcgcgaaaaaggtcctcaccatgggcgtcaacggcgagctccacccgtcgagattctgcgagaaagatctcctccgtgttgttgatcgtgaatacgtcttcgcttacatcgacgacgtttgcagcggcacatacccattaatgcagaagctccgacaggttctggtcgaccacgctctaaacaacggcgaaacggagaagaacactaacacctccatcttccaaaagatcgctaccttcgaagaagaattgaaagtcctgttaccgaaagaagttgaaggtgttagaatcgcttatgagaatgatacattgtcgattccaaacaggattaaagcttgcagatcgtacccgttgtataggtttgtaagggaggagctcggcagagggtttttgaccggagaaaaggtgacgtcgccgggagaggagttcgacagggtgttcacggcgatgtgcaaaggtcaaattattgatccgttgttggagtgtcttggagggtggaatggggaacctcttccaatatgttaggaaagtgagtgtgaaaccgtttgaattgtatttgtaatattctgttttttttttttttttttaaattttatttgcatttaatatctcatcaaagacttccactttcaagtgtggtgtatgtggttgtaaatcatatatattaacttattatttttgctaaaaaaaaaaaaaaaaaaaaaaaaaaaaaa

SEQ ID NO: 31 - DNA, 2403 bp, Petroselinium crispum
tttgctacattgttcttcattcattataagtaagttacatataaaatttgtaacgtaaatagagtttatttaatttattctaaagatcatggcatatgtaaatggtaccaccaacgggcatgcaaacgggaacggattagatttgtgcatgaagaaggaagatcctttgaactggggagtggctgcggaggcattgacagggagtcatttggacgaagttaagaggatggtggctgagtacaggaagccggtggtgaagctggaaggagaaacactgacaatttctcaggtggctgctatttcggctagggatgatagtggtgttaaggtggagctttccgaggaggcgagagctggcgttaaggctagtagtgactgggtgatggatagtatgaataaagggacggatagttatggtgttactactggctttggtgctacttctcataggaggactaaacaaggtggtgctcttcaaaaggagcttattaggttcttgaatgctggaatatttggaagtggagctgaagctggtaacaacacattaccacactccgcaacaagagcagcaatgcttgtgagaatcaacacactcctccaaggctattcaggaatccgattcgagatccttgaagccatcaccaagtttcttaaccacaacattactccttgtttgccactccgtggtacaatcactgcttctggtgatcttgtgccattgtcctacattgctggacttctcactggtcgtcccaactccaaggctgttggaccgactggagtaacactcagccccgaagaagcatttaagcttgctggtgtggaaggtggattttttgagttacagccaaaggaaggcctagcacttgttaatggaacagctgttggttctggaatggcctctatggtactttttgaggctaatatattagcagttttagctgaagttatgtcagcaattttcgctgaagtgatgcaagggaagcctgaatttaccgaccatttgacacataagttgaagcaccatcccggccaaattgaggctgcagctataatggaacacattttggatggaagcgcatacgttaaggctgctcaaaagctacatgaaatggatccattacaaaaaccaaaacaagacagatatgctcttagaacatctcctcaatggcttggtcctcaaattgaagttatcagatcatcaactaaaatgatcgaaagagagattaactctgtcaatgataacccattgattgatgtttccaggaacaaggctattcacggtggaaatttccagggcagcccaattggtgtttcaatggacaatacacgtctggctattgcagccataggaaagcttatgtttgctcaattttcagaacttgtcaacgacttttacaacaatgggttgccatcaaatttgtctggagggcgtaacccgagcttggattatggattcaagggtgctgaaattgccatggcatcatactgctccgaactccagtttttagccaatccagtgactaaccatgtccaaagtgctgaacagcacaaccaagatgtgaactctttgggtttaatatcttcaaggaaaacatcagaagctgttgaaatcttgaaactcatgtctactacgtttttggtgggtctatgccaagctatagacttaaggcatttggaggaaaatttgaagagcactgttaaaaacacagtgagccaagtagctaagcgagtactaaccatgggtgtcaacggtgagctccatccctcaagattctgcgagaaagatttgcttagagttgtagaccgcgaatacatatttgcatacattgatgatccctgcagcgcaacctacccattgatgcaaaaactaagggaaactctagttgagcatgcattgaacaatggtgataaagagaggaacttgagcacttccatcttccaaaagattgcagcattcgaggatgaactaaaggctcttctgcctaaagaagtcgaaactgctagagccgcacttgaaagtggaaatccggcaatccccaacaggattaaggagtgcaggtcttaccctctgtacaagtttgtgagggaagaattgggaactgagtatcttacaggagaaaaagtgcggtcacctggggaagaattcgaaaaggtattcacagcaatgtcgaaaggagagataattgatccattgttggagtgtctcgagtcatggaatggtgctcctcttccaatctgctaaattggcatgcagtccagcaatgtattaggaactgtttatctctctgtcaagatttatcttcttgtttgttgatgtctctccctagtgaatgtctgtaaaattcttttaaaacgctgtaaaatcttttgtaatactaatgtacaagtctacgcggccgc

SEQ ID NO: 32 - DNA, 2448 bp, Prunus avium
ggggagatggtcaggctctttcgtgctcccaaacactttgctctcatcagtttttgtattttcccatctgggtttttggtaattaacatggcaaccaactccatcaagcaaaatggtcacaaaaacggatcggtagagttacctgagctctgcataaagaaggaccctttgaactggggtgtggcagcagagacactaaaagggagccacttggatgaggtgaagcgcatggtggctgagtacaggaagccggtggtgaagctcggtggagagagcctgaccatttcccaagtggcggccatagccactcatgactctggggtcaaggttgagctctctgagtcagcccgggccggggtcaaggccagcagcgactgggtcatggacagcatgagcaaagggactgacagctatggtgtcaccaccgggtttggtgctacctcccacagaagaacaaagcaaggggctgcccttcagaaggagctcattagattcttgaacgctggagtgtttgggagcacgaaagagtcgggccacactttgcctcaccaggcaacaagagcagccatgttggttaggatcaacacactcctccagggctactctggcataagatttgagatcttggaagtcatcaccaagttcctcaacaacaatgtcactccatgcttgcccctacgcggcacgatcacagcctccggtgaccttgtcccgctgtcctacatcgccgggatgctaactggcaggcctaattccaaggctgttggaccagatggccagaccctcagtgctgcagaggcctttgagtttgttggtatcaattccgggttctttgagttgcagcctaaagaaggcctggctcttgttaatggcactgctgttggttctggcttggcttccacggttcttttcgacactaacattttggcattgctgtcagaaattctatcagcaatttttgctgaagttatgcaggggaagcctgaatttactgaccacttgacgcataagttgaagcaccaccctggccaaattgaagctgcagcaattatggaacatattttggatggtagctcttatgttaaagctgctaagaagttgcacgagcaggaccctctgcagaagccaaaacaggatcgatatgctctccgaacttcacctcaatggctcggtccacagatcgaagtgatccggtactccaccaaatccattgagagggagatcgactcagtcaatgacaaccctttgattgatgtgtcaaggaacaaggccttgcatggtggcaacttccaggggaccccaattggtgtctctatggacaatactcgtttggctattgcatccattgggaagctcatgtttgctcaattttctgagcttgtcaatgacttttacaacaacggattgccatcaaatctgtctggaggcaggaacccaagtttggattatggcttcaagggggctgagattgccatggcatcttattgttctgagcttcagtttctcgcgaacccggtcactaaccatgtccagagtgcagagcagcacaaccaagacgtgaactctttggggttgatctcttcaagaaagacagctgaagctgttgatatcttgaagctcatgtcttccacatttttggtggcactttgccaagcaattgatttgaggcatttggaggagaacttgaggaacacagttaagaacacagtgagccaagtggctaagagaactttgacaactggggttaatggagagctccacccatcaagattctgtgagaaggatttgcttaaagtggtcgatagggaatatgttttcgcctacatcgacgacccctgcagtgccacttacccattgatgcaaaaactaaggcaagtgctggttgagcatgctttgacaaatggtgagaatgagaagaatgcaagcacttcaatcttccaaaagattgttgcttttgaggaagagctgaaggtgcttttgcctaaagaggtggatagtgcaagggctgcattggacagtggaagtgctggagttccaaacaggattacggaatgcaggtcttaccccttgtacaaatttgtgagggaggagttgggtgcagagtacctaacaggggaaaaggtcaggtcaccgggcgaagaatgcgacaaggtgttcacagctatctgcgagggaaagattatcgacccgattctggattgcctcgagggctggaacggtgcaccacttccgatctgttagcatttcgttacgctttgagtgctgcattccattccactacttctgtgtccataagtttgattgcattgctggtagactgtgtgactatacctattttacctaataaatgtaaaaccatctacttatgctttttgattctttctaagttctttaccacttctttgttatttagtgagaatagtattcaaatagtcaggaaaaaaaaaa

SEQ ID NO: 33 - DNA, 2441 bp, Lithospermum erythrorhizon
tccacacgcatattcctctctacattctctacatacattttaatttgtctctctttgtgtttgtcacatttcttcaactaccctatagttagttcccatttatttttcttggatcaagaaaacattactacattattagtacacacatatatattacaaaatggaaaccatagtggaaaatggaaatggaaaaactatggagttttgcatgaaagatccattgaactgggaaatggcatctgagtcaatgaaggggagccaccttgatgaagtgaaaaacatggtggctgagttcaggaaaccggtggtgcaacttgccggtaagactttgactatcggtcaggtggcggcgattgctgcccgtgacgacggagtcacggtggagctagcggaagctgcccgggaaggtgttaaggctagtagtgattgggttatggatagtatgaataagggcacggatagttatggtgtaaccactggcttcggtgccacttcacataggaggactaaacaagggggtgcccttcaaaaggaacttattagattcttgaatgctggaatatttggcaatggaacagaaactagccacacattaccacactcagcaacaagagcagccatgcttgttaggatcaatactttgcttcaaggttattcaggcatcaggtttgagatcctggaagctatcaccaagttcctcaacaccaacatcactccatgcctaccccttcgtggcacgatcaccgcctctggtgacctcgtccccctctcatacattgccggactacttactggccgtcccaattccaaggccgttggacctaccggggagaagatcaatgcggaggaagcttttcgtctagctgggatcagtaccgggttcttcgagttgcagcctaaggaaggacttgcccttgttaatggaacagctgttggttctggaatggcttcaatggttctttatgaagccaacattttggctgtcttgtctgaagtgatctcggctattttcgctgaggtgatgaatggaaagcctgaattcaccgaccatttgacacacaaactgaaacaccatccaggacagattgaggctgctgctatcatggagcacattttggatggtagtggatatgttaaggctgctcagaagttacatgagatggatcctctgcagaagcctaagcaagatcgttatgccctccgtacatcgcctcaatggcttggtcctcagatcgaagtgatccgttctgctaccaagatgattgagagggaaatcaactctgttaacgacaacccattgatcgatgtttcgaggaacaaggccttacatggaggaaacttccagggcacacctattggtgtggccatggacaacactcgccttgccatcgcctcaattggaaagcttctatttgctcaattttctgaattggttaatgattactacaacaatgggttgccatcaaatttgacaggcagcagaaatccaagcttggattatggttttaagggagctgaaatcgccatggcttcgtactgctcagaactccagttcttggctaatccagtcaccaaccatgtccagagtgctgaacaacacaaccaagatgtcaactctttgggcttaatctcttcaagaaagacatccgaggctgtcgaaatcctgaagctcatgtcttcatcatttttggttgcactcttccaagctgttgatttgaggcatattgaggagaatgtgagactcgcagtcaagaacacggttagtcaggttgccaagcggacattaaccacaggcgttaatggcgagctccacccatcaagattcagcgaaaaggacttgcttcgcgtggttgatcgcgagtatgtctttgcctacgcagacgacccttgcctcaccacctaccccttgatgcagaagctaagagaaactctcgttggacacgccttagacaatggcgagaatgagaaggatgtgaacacttcaatcttccataaaatagccattttcgaagaagaattgaaggccattctccctaaagaggtggagaatgcacgcgcctcggtcgaaaatggcattccagcaatctccaacaggattgaggaatgtaggtcatatccattgtacaagtttgtgagggaagaattggggactgaattgttgactggtgagaaggttagatcaccaggtgaggaattggacaaagtattcactgcaatgtgtgaaggcaagcttgttgatccacttctggcttgtttggaggcttggaatggtgctcctcttccaatctgttaaataaacagttttgtggactatttcatgtacttaactacttctttttgttcttttctttttctatgttcatattaattcttctgttgatttgtttgtaaggtgttgtcttatcaatattatctaatggaacaactaatagattcctatt

SEQ ID NO: 34 - DNA, 2607 bp, Citrus limon
ccacgcgtccgcttggcttgaaaatttccctttcatcatcgtcacagattcacgtttacatgcaataaatatataattgcccccacaaaagattttcccacccattttctctcccacccatcagtacatttacttcttttaactaaaaaacaacaaggaaaaaaaaaatggagttgtcacatgaaacttgcaatggcatcaagaatgataggaatggtggtacttcgtcattggggttgtgcacaggtactgaccctttgaactggaccgtggcagcggactcattgaaagggagtcaccttgatgaagtgaaacggatgattgacgagtacaggaggccggtggtgaagctcggcggcgagtccttgaccattggccaagtgactgctatcgcggcccacgactctggagtcaaggtggagctagcggaggccgcccgcgccggcgtcaaggccagcagcgattgggtgatggacagcatgatgaaagggactgatagctatggtgtcaccactggctttggtgcaacttctcaccggcgaaccaagcaaggtggtgctttgcaaaaggagctcattagattcttgaattctggaatttttggcaatggcactgaatcaagccacacattgcctcactcggcaacaagggcagcaatgctggtgagagtcaacaccttgttacaaggatactcaggcatcaggtttgagatcctggaaaccattaccaagttccttaaccataacatcaccccttgcttgccgctacgtggcacgatcaccgcgtcgggcgacctggtcccactctcgtacattgctgggcttttgacaggcaggcccaactcgaaggctgttgggtccaacggccaagttctcaaccccaccgaggccttcaacctagctggggtcactagtggattttttgaattgcagcctaaggaaggtcttgccctggtgaatggcacagcggttggctctggtttagctgccacggtactctttgaggctaacatattagccattatgtctgaagttttatctgcaatttttgcggaagtgatgaatgggaaacctgaatttacagaccacttgacacataagttgaagcaccatccgggacaaattgaagctgcagctattatggaacacattttggatggcagctcttatgttaaagcagcacaaaagttacatgaaaccgatcctcttcaaaagccaaagcaagacagatatgctcttcgaacatcgcctcaatggctaggtcctcagattgaagtgatcagggcagctaccaaaatgattgaaagagagattaactccgtgaatgacaatccattgatagatgtatcaaggaacaaggcgcttcatggaggcaatttccaggggaccccaattggtgtttccatggacaacacccgtctagccattgcttcaattggcaagctcatgtttgcacaattctctgagcttgtcaatgatttctacaacaacgggttgccttcaaatcttactgggggacgtaatccaagcttggattacggattcaagggtgccgaaattgcaatggcatcatactgttctgaactccaattcctcgccaatcctgtcaccaatcatgtccaaagtgctgagcaacacaaccaagatgtgaactccttaggcctcaattcttctaggaaaactgctgaagcagtagacatattgaagcttatgtcgtcaactttcttagttgctctatgccaagcgattgacttgaggcatctggaagagaacctcaagaacacagtgaagaacaccgtgagtcaagttgccaagagagtcttgaccatgggagtgaatggagagcttcacccttcaagattctgcgaaaaagacttgatcaaagttgtggacagagaatatgtctttgcatacattgatgatccttgcagtgcaagttcaccattgatgcagaagctgaggcaagtgcttgttgatcatgcgttggacaatggagacagagagaagaattcaaccacttcaatcttccaaaagattggagcctttgaggatgaactaaagacccttttgcctaaagaggtcgaaatcgccagaactgaacttgagagtggaaatgcagccattccaaacaggatcaaggaatgcaggtcctatccgttatacaaaattgtgagagaagatattggaacaagtttgttgactggcgaaaaggttcgatccccaggtgaagaattcgacaaagttttcacagcaatgtgtgaagggaagttgattgatcctatgcttgaatgcttgaaggagtggaatggtgctcctcttcccatttgccagaattaatggtttgattaagtacagttttatgactgtctttttttattcttcttgttcttatgcttttacttgccatgttgtgtagtcaacttgaaacagtcaattcaattgcttttttggtttttctgtatttggaaaagttgggacaataacaaatgtaatgttgataacaagaacagacaggtgttcagcatgttcttcttcactgttgctaaagataagagcatcgtcaatatagataaggctttgttaaaaaaaaaaaaaaaaaaaaaaaagg

SEQ ID NO: 35 - DNA, 2151 bp, Rhodotorula glutinis
atggcaccctcgctcgactcgatctcgcactcgttcgcaaacggcgtcgcatccgcaaagcaggctgtcaatggcgcctcgaccaacctcgcagtcgcaggctcgcacctgcccacaacccaggtcacgcaggtcgacatcgtcgagaagatgctcgccgcgccgaccgactcgacgctcgaactcgacggctactcgctcaacctcggagacgtcgtctcggccgcgaggaagggcaggcctgtccgcgtcaaggacagcgacgagatccgctcaaagattgacaaatcggtcgagttcttgcgctcgcaactctccatgagcgtctacggcgtcacgactggatttggcggatccgcagacacccgcaccgaggacgccatctcgctccagaaggctctcctcgagcaccagctctgcggtgttctcccttcgtcgttcgactcgttccgcctcggccgcggtctcgagaactcgcttcccctcgaggttgttcgcggcgccatgacaatccgcgtcaacagcttgacccgcggccactcggctgtccgcctcgtcgtcctcgaggcgctcaccaacttcctcaaccacggcatcacccccatcgtccccctccgcggcaccatctctgcgtcgggcgacctctctcctctctcctacattgcagcggccatcagcggtcacccggacagcaaggtgcacgtcgtccacgagggcaaggagaagatcctgtacgcccgcgaggcgatggcgctcttcaacctcgagcccgtcgtcctcggcccgaaggagggtctcggtctcgtcaacggcaccgccgtctcagcatcgatggccaccctcgctctgcacgacgcacacatgctctcgctcctctcgcagtcgctcacggccatgacggtcgaagcgatggtcggccacgccggctcgttccaccccttccttcacgacgtcacgcgccctcacccgacgcagatcgaagtcgcgggaaacatccgcaagctcctcgagggaagccgctttgctgtccaccatgaggaggaggtcaaggtcaaggacgacgagggcattctccgccaggaccgctaccccttgcgcacgtctcctcagtggctcggcccgctcgtcagcgacctcattcacgcccacgccgtcctcaccatcgaggccggccagtcgacgaccgacaaccctctcatcgacgtcgagaacaagacttcgcaccacggcggcaatttccaggctgccgctgtggccaacaccatggagaagactcgcctcgggctcgcccagatcggcaagctcaacttcacgcagctcaccgagatgctcaacgccggcatgaaccgcggcctcccctcctgcctcgcggccgaagacccctcgctctcctaccactgcaagggcctcgacatcgccgctgcggcgtacacctcggagttgggacacctcgccaaccctgtgacgacgcatgtccagccggctgagatggcgaaccaggcggtcaactcgcttgcgctcatctcggctcgtcgcacgaccgagtccaacgacgtcctttctctcctcctcgccacccacctctactgcgttctccaagccatcgacttgcgcgcgaccgagttcgagttcaagaagcagttcggcccagccatcgtctcgctcatcgaccagcactttggctccgccatgaccggctcgaacctgcgcgacgagctcgtcgagaaggtgaacaagacgctcgccaagcgcctcgagcagaccaactcgtacgacctcgtcccgcgctggcacgacgccttctccttcgccgccggcaccgtcgtcgaggtcctctcgtcgacgtcgctctcgctcgccgccgtcaacgcctggaaggtcgccgccgccgagtcggccatctcgctcacccgccaagtccgcgagaccttctggtccgccgcgtcgacctcgtcgcccgcgctctcgtacctctcgccgcgcactcagatcctctacgccttcgtccgcgaggagcttggcgtcaaggcccgccgcggagacgtcttcctcggcaagcaagaggtgacgatcggctcgaacgtctccaagatctacgaggccatcaagtcgggcaggatcaacaacgtcctcctcaagatgctcgcttag

SEQ ID NO: 36 - DNA, 1572 bp, Rhodobacter sphaeroides
atgctcgccatgagccccccgaagccggccgtcgagctggatcgccacatcgatctggaccaggcccatgccgtggcgagcggcggcgcgcggattgtccttgcccctccggcgcgcgaccggtgccgtgcgtccgaagcgcggctcggcgctgtcatccgcgaggcgcgccatgtctacggactgacaaccggcttcggtccccttgcgaaccgcctgatctcaggtgagaatgtccgaacgctgcaggccaatcttgtccatcatctggccagcggcgtgggaccggtgcttgactggacgacggcgcgcgccatggttctggcgcgtctggtgtcgatcgctcagggagcctccggtgccagcgaggggaccatcgctcgcctgatcgacctgctcaattccgagctcgctccggccgttcccagccgcggcacggtgggcgcgtcgggtgacctgacaccgcttgcgcatatggtgctctgcctccagggccggggagacttcctggaccgggacgggacgcggcttgacggcgcagaagggctccggcgcggacggctgcaaccgctcgatctctcccatcgcgatgcactggcgctggtcaacgggacctccgccatgaccgggatcgcgctggtgaatgctcacgcctgccgccatctcggcaactgggcggtggcgttgacggccctgcttgcggaatgtctgagaggccggaccgaggcatgggccgcggcactgtccgacctgcggccgcatcccggacagaaggacgccgcagcgaggctgcgcgcccgcgtggacggcagcgcgcgggtggtccggcacgtcattgccgagcggaggctcgacgccggcgatatcgggacggagccggaggcggggcaggatgcctacagcctgcgctgcgctccgcaggttctcggggcgggcttcgacacgctcgcatggcatgaccgggtgctgacgatcgagctgaacgcggtgaccgacaatccggtgtttccgcccgatggcagcgtgcccgccctgcacgggggcaatttcatgggccagcatgtggcgctgacgtccgatgcgctcgccacggccgtcaccgttctggcgggccttgcggagcgccagattgcacgtctgacagatgaaaggctgaaccgtgggctgccccccttcctccaccggggccccgccgggttgaattccggcttcatgggcgcacaggtgacggcgaccgcgctcctggccgagatgcgagccacgggacctgcctcgatccattcgatctccacgaacgccgccaatcaggatgtggtctcgcttgggaccatcgccgcgcgcctctgccgcgagaagatcgaccgttgggcggagatccttgcgatcctcgctctctgtcttgcacaagctgcggagctgcgctgcggcagcggcctagacggggtgtctcccgcggggaagaagctggtgcaggccctgcgcgagcagttcccgccgcttgagacggaccggcccctgggacaggaaattgccgcgcttgctacgcacctcttgcagcaatctcccgtctga

SEQ ID NO: 37 - DNA, 2465 bp, Trichosporon cutaneum
  misc_feature (1607)..(1607): n is a, c, g, or t
tggaatgcatgctccggcgacagcccggcataccacactgtaacacactcgtctcccccctcccaccctctcttatcgcgtcacatggctaactctctgactgctcgcacctaacacgaacacggcgccgagcgaggcgatgaacgctatataacaatccgtggtgttgccacctcctccccaccgatcacactcagctcagctcgctcctcgccagcccctctcgctctaactcgctctacgctatcgcggtaccgcaccccatacaacaaacccctcccgagtggcaatgtttattgagaccaatgtcgccaagcccgcttccaccaaggcgatgaacgccggttcggccaaggccgctcctgtgtgagtacccaccactaactggggagtcaccgctgacatgcagtgagccgttcgctacctatgcccactcccaggctaccaagaccgtcagcatcgacggccacaccatgaaggtcggtgacgtcgtcgccgtcgcccgccacggcgccaaggtcgagctcgcggcctcggtcgccggccccgtccgggcctcggtcgacttcaaggagtccaagaagcacacgtcgatctacggcgtcaccaccggctttggcggctcggccgacacgcgcaccagcgacaccgaggcgctccagatctcgctcctcgagcaccagctctgcggcttcctccccaccgacgccacctacgagggcatgctcctcgccgcgatgccgatccccatcgtccgcggcgccatggccgtccgcgtcaacagctgcgtccgcggccactcgggcgtccgcctcgaggtcctccagtcgtttgccgactttatcaacagaggcctcgtcccctgcgtgcccctccgcggcaccatctcggcctcgggcgacctctcgcccctctcgtacattgccggtgcgatctgcggccaccccgacgtcaaggtgttcgacaccgcggcgtcgccccccacggttctcacctcccccgaggcgatcgccaagtacggcctcaagaccgtcaagctcgcctccaaggagggcctcggcctcgtcaacggcacggccgtctcggcggccgcgggcgcgctcgcgctctacgacgccgagtgcctcgccatcatgagccagaccaacactgtgctcacggtcgaggcgctcgacggccacgtcggctcgtttgcccccttcatccaggagatccgccctcacgccggccagatcgaggccgctagaaacattagacacatgctcggtggctccaagctcgccgtgcacgaggagtccgagctcctcgccgaccaggacgccggcatcctccgccaggaccgctacgcgctccgcacctcggcgcagtggatcggcccgcagctcgaggcgctcggcctcgcccgccagcagatcgagaccgagctcaactcgaccaccgacaacccgctcatcgatgtcgagggcggcatgttccaccacggcggcaacttccaggccatggccgtcacctcggccatggactcggcccgcatcgtcctccagaacctcggcaagctcagctttgcccaggtcaccgagctcatcaactgcgagatgaaccacggnctcccctccaacctcgccggctccgagcctagcaccaactaccactgcaagggtctcgacatccactgcggcgcctactgcgccgagctcggcttcctcgccaaccccatgagcaaccacgtccagagcaccgagatgcacaaccagagcgtgaactcgatggcgttcgcgtccgcccgcaggacgatggaggccaacgaggtcctctcgctcctcctcggctcgcagatgtactgcgcgacccaggccctcgacctccgcgtcatggaggtcaagttcaagatggccatcgtcaagctcctcaacgagaccctcaccaagcactttgcggccttcctcacgcccgagcagctcgccaagctcaacacccacgccgccatcacgctgtacaagcgcctcaaccagacgcccagctgggactcggccccgcgcttcgaggacgccgccaagcacctcgtcggcgtcatcatggacgccctcatggtcaacgacgacatcaccgacctcaccaacctccccaagtggaagaaggagttcgccaaggaggccggcaacctctaccgctcgatcctcgtcgcgaccaccgccgacggccgcaacgacctcgagcccgccgagtacctcggccagacgcgcgccgtctacgaggccgtccgctccgagctcggcgtcaaggtccgccgcggcgacgtcgccgagggcaagagcggcaagagcatcggctcgagcgtcgccaagatcgtcgaggcgatgcgcgacggccgcctcatgggcgctgttggcaagatgttctaagccaccagacatttctctatagggtagcaactgtttcagtagcacatgcatcattgtactatt

SEQ ID NO: 38 - DNA, 1569 bp, Streptomyces coelicolor; identical to SEQ ID NO: 3

SEQ ID NO: 39 - DNA, 1626 bp, Allium cepa
atgggttcaatatcaatggatcaagaaacgatattcaggtcgaaacttccggatatttacatccccgaccatctacctctccactcctactgcttccagcacattcaagagttctccgacaaaccctgcatcatagatggcataactgaaaaggtgtatacttacgcagacgtcgagctaacatcaaaacgtgtggcagtcggtctgcgcgacttgggcatcagaaaaggccatgtcatcatgatcctcctacccaactctccggagttcgccttctccttcctcggagcttcctacctcggcgccatgtccacaacagcgaatccttactacaccccagctgagatcaaaaagcaggcaatgggatccggcgttagggtcataataacagaatcctgctacgtgcccaagatcaaagacttagaacacaacgtaaagatcgtagtcatcgatgagttggtcgatgaacacagtacatgcatccccttttcacaactgtcttccgctgatgaaaggaagctcccggaggtggaaatcagtcctgacgatgtggtggcacttccttattcatcgggaactacagggctaccgaaaggagttatgctgacacatgaaggcttgattacaagcgtggctcagcaggtggatggagagaatccgaatttgtatttcagaagcgacgatgtgcttttgtgtgtattaccgctttttcacatatattcgctgaactcggttttgttgtgtggactgagggcggggtcgacgattttgttgatgaggaagtttgatttgactaaagtggtggagttggttggaaaatacagggtgacgatagcgccatttgtgcctcctatttgtattgaaattgctaagaatgacatggttggaatgtgtaatttgttgaacattaggatggttatgtcgggggcggcacccatggggaaggagttggaggataagttgaaggagaagatgcctaatgccgtacttggccagggttacggaatgactgaagcaggtcctgtaatatcaatgtgtcctggctttgcaaaacatccaactcaagccaaatccggatcatgtggaactatcgttagaaatgcagaactaaaagtgatggatccagaaacaggcttttctcttggccgcaaccttcctggagaaatttgcatccgtggtccccagataatgaaaggttatcttaatgaccctgaggcaacttcttcaactatagacttagaaggttggctacatactggagatattggttatgttgatgatgatgatgaagtattcattgttgacagagttaaggaactgatcaaatttaaagggtttcaggtaccgccggctgagctcgagtctctgcttgttagtcacccttgtattgcagatgcagctgtgattcctcaaaaagatgaagttgccggtgaggttcctgttgcatttgttgttaaagcgagtggttcagacattactgaagacgctgtgaaggaattcatttcaaagcaggtggtgttttacaagagattgcagacggtttattttgttcacgcaattccaaaatctccttcaggaaagatattaaggaaggatctgagagctcgactttcttcgtttacatag

SEQ ID NO: 40 - DNA, 1575 bp, Streptomyces avermitilis
gtgttccgcagcgagtacgcagacgtcccgcccgtcgaactccccatccacgaggcggtgctgggccgggccgcggagttcggggaggcacccgccctcgtcgacgcagtggacggcaccaccctcacgtacgaacaactggaccggttccaccggcggatcgccgcggcgctggccgaggcgggcgtccgcaagggcgacgtcctcgccctgcacagcccgaacaccatcgccttcccgacggcgttctacgccgccacgcgcgcgggcgcgtcggtcaccaccgtgcacccgctcgccacggcggaggagttcgccaagcagctgagcgactgctccgcccgctggatcgtcaccgtgtcgccgctcctggacaccgcccgcagggcggccgaactcgcgggcggcgtccgggagatcttcgtctgcgacagcgcgcccgggcaccgctcactgatcgacatgctggccaccgccgcccccgagccgcgggtcgacatcgaccccgcggaggacgtcgcggccctcccgtactcctcgggcacgaccggcacacccaagggcgtgatgctcacccaccggtccatcgccaccaacctcgcccagctcgaaccggccgtgccgacggggccgggcgagcgcatcctcgccgtcctgcccttcttccacatctacggcctgaccgccctcatgaacgcgcccctcaggctcggcgccacggtcgtcgtactgccccgcttcgacctcgacacgttcctcgcggccatcgagaaacaccggatcacccacctgtacgtcgccccgccgatcgtcctcgcgctggccaagcacccggccgtcgcgcagtacgacctgtcgtccctgaagtacgtcatcagcgccgccgcgcccctggacgccgacaccgccgcggcctgctcgcgacgcctgggggtgcccccggtcggacaggcgtacggcatgacggagctgtcacccggcacccacgtggtcccgctgaacgccgtgaacccgcccccggggaccgtcggcaagctcgtcgcgggcacggagatgcgcatcctctccctcgacgacccggaccaggacctgcccgtcggcgaggccggtgagatcgccatccgcggcccccaggtcatgaagggctacctggggcgcccggaagccaccgccgcgatgatcgacgaggacggctggctgcacaccggggacgtcgggcgcgtggacgccgacggctggctgttcgtcgtcgaccgcgtcaaggaactcatcaagtacaagggcttccaggtcgcccccgccgagctggaggcgctcctgctgacccacccgaagatcgcggacgccgccgtcatcggcgtctacaacgacgacaacaacgaggtcccgcacgcccacgtggtgcgccagccgtccgcggccgacctctccgcgggcgaggtgatgatgtacgtcgccgaacgcgtcgccccctacaaacggatccggcacgtcaccttcctcgacgaggtgccccgggccgcctccgggaagatcctccgacgacagctgcgagacctgcgggagcactcatga

SEQ ID NO: 41 - DNA, 1915 bp, Populus tremuloides
ccctcgcgaaactccgaaaacagagagcacctaaaactcaccatctctccctctgcatctttagcccgcaatggacgccacaatgaatccacaagaattcatctttcgctcaaaattaccagacatctacatcccgaaaaaccttcccctgcattcatacgttcttgagaacttgtctaaacattcatcaaaaccttgcctgataaatggcgcgaatggagatgtctacacctatgctgatgttgagctcacagcaagaagagttgcttctggtctgaacaagattggtattcaacaaggtgacgtgatcatgctcttcctaccaagttcacctgaattcgtgcttgctttcctaggcgcttcacacagaggtgccatgatcactgctgccaatcctttctccacccctgcagagctagcaaaacatgccaaggcctcgagagcaaagcttctgataacacaggcttgttactacgagaaggttaaagattttgcccgagaaagtgatgttaaggtcatgtgcgtggactctgccccggacggtgcttcacttttcagagctcacacacaggcagacgaaaatgaagtgcctcaggtcgacattagtcctgatgatgtcgtagcattgccttattcatcagggactacagggttgccaaaaggggtcatgttaacgcacaaagggctaataaccagtgtggctcaacaggtagatggagacaatcctaacctgtattttcacagtgaagatgtgattctgtgtgtgcttcctatgttccatatctatgctctgaattcaatgatgctctgtggtctgagagttggtgcctcgattttgataatgccaaagtttgagattggttctttgctgggattgattgagaagtacaaggtatctatagcaccagttgttccacctgtgatgatggcaattgctaagtcacctgatcttgacaagcatgacctgtcttctttgaggatgataaaatctggaggggctccattgggcaaggaacttgaagatactgtcagagctaagtttcctcaggctagacttggtcagggatatggaatgaccgaggcaggacctgttctagcaatgtgcttggcatttgccaaggaaccattcgacataaaaccaggtgcatgtggaactgtagtcaggaatgcagagatgaagattgttgacccagaaacaggggtctctctaccgaggaaccagcctggtgagatctgcatccggggtgatcagatcatgaaaggatatcttaatgaccccgaggcaacctcaagaacaatagacaaagaaggatggctgcacacaggcgatatcggctacattgatgatgatgatgagcttttcatcgttgacagattgaaggaattgatcaagtataaagggtttcaggttgctcctactgaactcgaagctttgttaatagcccatccagagatatccgatgctgctgtagtaggattgaaagatgaggatgcgggagaagttcctgttgcatttgtagtgaaatcagaaaagtctcaggccaccgaagatgaaattaagcagtatatttcaaaacaggtgatcttctacaagagaataaaacgagttttcttcattgaagcaattcccaaggcaccatcaggcaagatcctgaggaagaatctgaaagagaagttgccaggcatataactgaagatgttactgaacatttaaccctctgtcttatttctttaatacttgcgaatcattgtagtgttgaaccaagcatgcttggaaaagacacgtacccaacgtaagacagttactgttcctagtatacaagctctttaatgttcgttttgaacttgggaaaacataagttctcctgtcgccatatggagtaattcaattgaatattttggtttctttaatgat

SEQ ID NO: 42 - DNA, 1979 bp, Oryza sativa (japonica cultivar-group)
caggaatcggtagttgtcatcacgcgcacttccattccgctcacctacccaccggagaagaggtagtcgccgccgccgccgctgtcgccgtcgccggagaagaagaatgggttcgttgccggagcagttcgtcttccgctcgaggctccccgacatcgccatcccggaccacctcccgctgcacgactacgtgttcgagcgcctcgccgaccgccgcgaccgggcatgccttatcgatggcgccacgggggagacgctctcgttcggcgacgtcgacgcgctgtcgcgccgcgtggcggctgggttgagctcgattggtgtttgccatggtagtaccgtgatgctgctgctgccgaactccgtcgagttcgcggtggcgttcctcgcgtcgtcacggctcggggcggtcaccaccacggccaacccgctgcacaccccgccggagatcgccaagcaggtggcggcgtccggcgcgacggtggtggtcaccgagccggcgttcgtcgccaaggtgagcggcctcgcgggcgtgaccgtcgtcgccaccgggggcggcgccgagaggtgcgcgtcgttcgcgggcctcgccgccgccgacggctcggcgctgccggaggtcgccatcgacgtcgccaacgacgccgtggcgctgccctactcgtcgggcacgacggggctccccaagggggtgatgctgtcgcaccgcgggctggtgaccagcgtggcgcagctcgtcgacggcgagaacccgaacctccacctccgggaggacgacgtggtgctctgcgtgctccccatgttccacgtctactccctccactccatcctcctctgcgggatgcgcgccggcgccgccatcgtggtcatgaagcggttcgacaccgtcaagatgctgcagctggtggagcgccacggcgtcaccatcgcgccgctcgtccctcccatcgtcgtcgagatggccaagagcgacgccctcgaccgccacgacctctcctccatccgcatggtcatctccggcgccgcccccatgggcaaggagcttcaggacatcgtccacgccaagctccccaacgccgtcctcggacaggggtacgggatgacggaggcagggccggtgctatcaatgtgcatggcgttcgcgaaggagccgacgccggtgaagtccggcgcgtgcggcacggtggtgcggaacgccgagctgaagatcgtcgacccggacaccggcttgtcactcccgcgcaaccagccgggggagatttgcatcaggggaaaacaaatcatgaaaggatacctgaacaacccggaggcgaccgagaagacgatcgacaaggacgggtggctgcacactggcgacatcggcttcgtcgacgacgacgacgagatcttcatcgtggaccggctcaaggagctcatcaagtacaagggcttccaggtcgcccccgccgagctcgaggccatgctcatcgcccacgccgccgtcgccgacgccgccgtcgtcccaatgaaggacgattcctgcggcgagatcccagtggcgttcgtcgtcgcacgcgacggctcggggatcaccgacgacgagatcaagcagtacgtcgcaaagcaggtggtgttctacaagaggctgcacaagatcttcttcgtggacgcaatcccgaaggcgccgtcgggaaagattttgaggaaggatctgagagcaaagttggctgctggaattccggcgtgctgatgaaactggcatgagctagcttgcctgagatatccagctattgtttggtttttgttttgatcatacttaaaaagatatagtgaaatgtaaacatgatgtagtcagcaatgtaaaaggaacacgccctctggatatacatgaaagtttgagaggtgatctcatcggtcatcagtaacacaaatttgtcgcaaagatttttattttttgttgttgcacccgtgtaaaagtgaattgatgccattttatttgttgg

SEQ ID NO: 43 - DNA, 1911 bp, Amorpha fruticosa
tcacaaacacaaaataccattcccgcaatggcatttgagacagaagaaccaaaggaattcatcttcaggtcaaaattaccagaaatcccaatctccaaacaccttccccttcactcttactgctttgagaacctctcagaattcgggtcacgtccatgcttgatcagtgccccaacaggggacgtgtacacctactatgacgtggaactcaccgctagaagagttgcctctggactcaacaaattgggtgtccaacaaggtgatgtcatcatgctccttcttcctaattcaccagaatttgtgttctccttcttgggtgcctcttaccgtggtgccatgatcactgctgccaacccattcttcacatccgctgagattgcaaaacaggccaaagcctccaacaccaagttgcttataacacaagcttcttactacgacaaggttaaggatttggatgtgaagttggtgttcgtggactctccccctgatgggcacatgcactattcagagctgcgtgaggctgatgagagtgacatgcctgaggtgaagaccaaccctgatgatgtggtggcacttccctattcgtcagggacaacagggttgcccaaaggggtgatgttatctcacaaagggttggcgaccagcatagcacaacaagttgatggggaaaaccccaacctctactttcacaatgaggatgtcatattgtgtgtgcttccactctttcatatatattctctcaattctgttctgttgtgtgggttgagagccaaggctgctattttgctgatgccaaagtttgagatcaatgccttgttgggtctcattcagaaacaccgagtaacaattgcccctattgtcccacccattgttttggccattgccaagtcaccggatcttgaaaagtatgatctctcttccattagggtgttgaaatctggaggggcttctctgggcaaagaactcgaagacactgtgagggctaaattccccaaggccaaacttggacagggatacggaatgactgaggcagggccagtgctaacaatgtgcttagcatttgctaaggaaccgatagatgtaaaaccaggtgcatgtggaaccgttgtaagaaatgcagagatgaagattgtggatcctgaaactggtaattcgttgccacgaaaccagtccggtgaaatttgcataagaggcgaccagatcatgaaaggttatctaaatgatcaagaggctacgcagagaaccatagacaaagaagggtggttgcatacaggtgacatcggctacatcgacgatgacgatgagttattcatcgttgacaggcttaaggaattgatcaaatacaaaggatttcaggtggctcctgctgaactcgaagcccttcttctctctcatcccaagatcaccgatgctgctgtggttccaatgaaggatgaagcagctggagaggtacctgttgcatttgtggtgagatcaaatggtcacacagacacaaccgaggatgaaattaagcagtttatctccaaacaggtggtgttttataaaagaataagcagagtattcttcattgatgcaattcccaagtcaccgtcaggtaaaatcttacgaaaggatctaagagcaaagcttgcagcaggtgttccaaattgaaaattctaattccaagatatatgatattaccattatcatacgatgcccgcacaaagctccataaaccttgaaggccagagtgcggacgcgtgcttggagcttgaccgcattacttatattcacacgagggcagacatgattaccttaaaagggggggttgctaattatattttaaaactatattgggtaaaatttgattcgatcaaggactttcatattatataatatcgaagtataatttttcaaaaaaaaaaaaaaaaa

SEQ ID NO: 44 - DNA, 1740 bp, Populus tomentosa
cgcaatggacgccacaatgaatccacaagaagaattcatctttcgctcaaaattaccagacatctacatcccgaaaaaccttcccctgcattcatacgttcttgaaaacttgtctaaccattcatcaaaaccttgcctgataaatggcgcgaatggagatgtctacacctatgctgacgttgagctcacagcaagaagagttgcttctggtctgaacaagattggtattcaacaaggtgacgtgatcatgctcttcctaccaagttcacctgaattcgtgcttgctttcctaggcgcttcacacagaggtgccattatcactgctgccaatcctttctccacccctgcagagctagcaaaacatgccaaggcctcgagagcaaagcttctgataacacaggcttgttactacgagaaggttaaagattttgcccgagaaagtgatgttaaggtcatgtgcgtggactctgccccggatggatgcttgcacttttcagagctaacacaggcagacgaaaatgaagcgcctcaggtcgacattagtcccgatgatgtcgtagcattgccttattcatcagggactacagggttgccaaaaggggtcatgttaacgcacaaagggctaataaccagtgttgctcaacaggtagatggagacaatcctaacctgtattttcacagtgaagatgtgattctgtgtgtgctgcctatgttccatatctatgctctgaattcaataatgctctgcgggctgagagtcggtgccccgattttgataatgccaaagtttgagattggttctttactgggattgattgagaagtacaaggtatctatagcaccggttgttccacctgtgatgatgtcaattgctaagtcacctgatcttgacaagcatgacttgtcttctttgaggatgataaaatctggaggggctccattgggcaaggaacttgaagatactgtcagagctaagtttcctcaggctagacttggtcagggatatggaatgaccgaggcaggacctgttctagcaatgtgcttggcatttgccaaggaaccattcgacataaaaccaggtgcatgtgggactgtagtcaggaatgcagagatgaagattgttgacccagaaacaggggcctctctaccgaggaaccagcctggtgagatctgcatccggggtgatcagatcatgaaaggatatcttaatgaccctgaggcaacctcaagaacaatagacaaagaaggatggctgcacacaggcgatatcggctacattgatgatgatgatgagcttttcatcgttgacagattgaaggaattgatcaagtataaagggtttcaggttgctcctgctgaactcgaagctttgttaatagcccatccagagatatccgatgctgctgtagtaggattgaaagatgaggatgcgggagaagttcctgttgcatttgtagtgaaatcagaaaagtctcaggccaccgaagatgaaattaagcagtatatttcaaaacaggtgatattctacaagagaataaaacgagttttcttcattgaagctattcccaaggcaccatctggcaagatcctgaggaagaatctgaaagaaaagttggcaggcatataactgaagatgttactgaacatttaatcctctgtcttatttctttaatacttgagaatcattgtagtgttgaaccaagcatgcttggaaaagacacgtacccaacgtaagacagt

SEQ ID NO: 45 - DNA, 1888 bp, Nicotiana tabacum
agcgtgtccattttttcaaactacttttaccgatggagaaagatacaaaacatggcgatataattttcagatcaaaactccctgatatttacatccctaatcatcttcctttacactcttactgctttgaaaacatttcagagttcagttctcgaccttgtttaatcaatggagccaacaaacaaatttatacgtatgctgatgttgaactcagttcaagaaaagttgctgctggtcttcacaaacaagggatccaacaaaaggatacaatcatgatcctattgcctaactccccagaatttgtgtttgctttcattggtgcatcgtaccttggagctatttctacaatggccaatcctttgtttacggccgctgaggttgtgaagcaagtcaaggcttctggtgctaagatcattgtcacacaagcgtgtcatgttaacaaagtgaaagattatgctttggagaataatgttaagatcatatgcatcgactcggcaccggagggttgtctccacttctccgtgctaactcaggccgatgagcacgatattcctgaggtagaaatccaacccgatgatgtggtggcgttgccctactcctctgggacgactggattacctaaaggagtgatgttgacacacaagggacttgtcacaagcgtggcacaacaagtcgatggtgaaaatcggaatttgtatatccatagcgaggacgtgttgctttgtgtcttgcccttgtttcatatctattccctcaactccgttttgctttgtggattaagggttggagcagcgattttgattatgcagaaatttgatattgttccattcttggagttgatacaaaattacaaggtgacaatagggccgtttgtaccgcctattgtcttggccattgctaagagtcctatggttgatgattatgatctttcatcagtaagaactgtcatgtctggggctgcaccattaggaaaggagcttgaagacactgttcgagccaaatttcctaatgctaaacttggtcagggttatggtatgacagaagctggaccagtgttggcaatgtgcttggcatttgcaaaagaaccctttgaaataaaatcaggggcatgtgggacagttgtgagaaatgctgagatgaaaattgtggatcctgaaactggtaattctcttcccaggaatcagtctggagaaatttgcataagaggagaccaaatcatgaaaggctacctgaatgatccagaggccacagcaagaacaatagacaaagaaggatggttatatacaggtgacattggctatattgatgatgacgacgagctttttattgtggatcgattgaaggagctgattaaatacaaaggatttcaagttgcacctgctgagctcgaagctctccttctcaaccatccaacattttctgatgctgctgttgtccccatgaaagacgaacaagcggaagaagttccagtggcttttgttgttagatccagtggatccaccattactgaagatgaagtcaaggatttcatctcaaagcaggtgatattttataagaggataaagcgggtatttttcgtggatgctgttcctaaatctccatctggcaaaatccttcgaaaagatttgagagctaaactggctgctgggcttccaaattaatactttcagtttagctttaatagtggcaataactataaccagttcgccattgaagacaatttattttttattaaaatgttacataatatgttcttttgattgtaccttcaactacgtgcctcttcggtcagaattaatttaccgaattggcaaaaggaggaaaatgtatgtaaatttgactgtaagaacttcaattttttaaatgtctttttggtattattttattc

SEQ ID NO: 46 - DNA, 1972 bp, Pinus taeda PT4CL2
ctcattcaattcttcccactgcaggctacatttgtcagacacgttttccgccatttttcgcctgtttctgcggagaatttgatcaggttcggattgggattgaatcaattgaaaggtttttattttcagtatttcgatcgccatggccaacggaatcaagaaggtcgagcatctgtacagatcgaagcttcccgatatcgagatctccgaccatctgcctcttcattcgtattgctttgagagagtagcggaattcgcagacagaccctgtctgatcgatggggcgacagacagaacttattgcttttcagaggtggaactgatttctcgcaaggtcgctgccggtctggcgaagctcgggttgcagcaggggcaggttgtcatgcttctccttccgaattgcatcgaatttgcgtttgtgttcatgggggcctctgtccggggcgccattgtgaccacggccaatcctttctacaagccgggcgagatcgccaaacaggccaaggccgcgggcgcgcgcatcatagttaccctggcagcttatgttgagaaactggccgatctgcagagccacgatgtgctcgtcatcacaatcgatgatgctcccaaggaaggttgccaacatatttccgttctgaccgaagccgacgaaacccaatgcccggccgtgaaaatccacccggacgatgtcgtggcgttgccctattcttccggaaccacggggctccccaagggcgtgatgttaacgcacaaaggcctggtgtccagcgttgcccagcaggtcgatggtgaaaatcccaatctgtatttccattccgatgacgtgatactctgtgtcttgcctcttttccacatctattctctcaattcggttctcctctgcgcgctcagagccggggctgcgaccctgattatgcagaaattcaacctcacgacctgtctggagctgattcagaaatacaaggttaccgttgccccaattgtgcctccaattgtcctggacatcacaaagagccccatcgtttcccagtacgatgtctcgtccgtccggataatcatgtccggcgctgcgcctctcgggaaggaactcgaagatgccctcagagagcgttttcccaaggccatcttcgggcagggctacggcatgacagaagcaggcccggtgctggcaatgaacctagccttcgcaaagaatcctttccccgtcaaatctggctcctgcggaacagtcgtccggaacgctcaaataaagatcctcgatacagaaactggcgaatctctcccgcacaatcaagccggcgaaatctgcatccgcggacccgaaataatgaaaggatatattaacgacccggaatccacggccgctacaatcgatgaagaaggctggcttcacacaggcgacgtcgggtacattgacgatgacgaagaaatcttcatagtcgacagagtaaaggagattatcaaatataagggcttccaggtggctcctgctgagctggaagctttacttgtggctcatccgtcaattgctgacgcagcagtcgttcctcaaaagcacgaggaggcgggcgaggttccggtggcgttcgtggtgaagtcgtcggaaatcagcgagcaggaaatcaaggagttcgtggcaaagcaggtgattttctacaagaaaatacacagagtttactttgtggatgcgattcctaagtcgccgtccggcaagattctgagaaaggatttgagaagcagactggcagcaaaatgaaaatgaatttccatatgattctaagattcctttgccgataattataggattcctttctgttcacttctatttatataataaagtggtgcagagtaagcgccctataaggagagagagagagcttatcaattgtatcatatggattgtcaacgccctacactcttgcgatcgctttcaatatgcatattactataaacgatatatgttttttttttt

SEQ ID NO: 47 - DNA, 1825 bp, Glycine max
aaagtcgcaaaaattctcctcctacaccaacaaaaatggcaccttctccacaagaaatcatcttccgatccccactccccgatattccgatccccacacatctcccattgtactcttactgcttccaaaacttgtcacagttccatgaccgtccatgcctcatcgacggcgacaccggcgagaccctcacctacgccgacgtcgacctcgctgctcgccgcatcgcctccggcctccacaaaatcggcatccgccagggtgacgtcatcatgctcgtcctacgcaactgcccgcagttcgccctcgccttcctcggcgccacccaccgtggcgccgtcgtcaccacagccaaccccttctacacgccggcggagcttgcgaagcaagcgacggccacgaaaaccaggctcgtcataacgcaatccgcgtacgtagagaaaatcaagagtttcgcggacagcagcagcgatgtcatggtgatgtgcattgatgatgatttttcttatgaaaacgacggcgttttgcatttctcaacgctcagtaacgccgacgaaacggaagcccctgccgttaagattaaccctgacgagctcgttgcgcttccgttttcttctggcacgtctgggctccccaagggcgttatgttatcgcataaaaacttggtcaccacgatagcgcagttagttgacggcgaaaacccgcaccaatacactcacagcgaggatgtgctactctgtgtgttgcctatgtttcatatctatgcgctcaattccattttgctctgcgggattcgttccggtgcggccgtgcttattttgcagaagtttgagatcactactctgttggagctcatcgagaagtacaaggtgacggttgcgtcgtttgtgccgcccatcgttttggcgttggttaagagcggagagactcatcgctacgacctgtcgtctattcgcgctgtggtcaccggcgcggcacccttaggaggggaacttcaagaagccgttaaggctaggctaccacacgctacttttggacagggatatgggatgacagaagcaggaccacttgccattagcatggcatttgcaaaagtaccctctaagattaaaccaggtgcatgcggaaccgttgtgagaaacgccgagatgaaaatcgtggatacagaaacgggtgattcacttccaagaaacaaacacggtgaaatttgcataataggcacaaaggtcatgaaaggatatctaaatgacccagaggctacagagagaactgtagacaaagaaggatggttacacacaggagatattggtttcattgatgatgatgatgaactcttcattgttgatcggttaaaggaattgatcaaatacaaaggattccaagtggctcctgctgagcttgaagcattgttgattgcccacccaaacatttctgatgctgccgttgtaggcatgaaagatgaagctgcaggggaaattccagttgcatttgttgtaaggtcaaatggttctgagatagccgaggatgaaatcaagaaatacatttcacaacaggtggttttttacaagagaatatgtagagttttcttcacggactctattcctaaagcaccctcaggcaaaattctgcgaaaggtattaactgcaagacttaacgaaggtttggtggtggccaattaggtccataattgtgacagaggaaaatcgtggctgttttacttaccgtaccacaggcccttcctgttgtggtttttgttccaattttatatctcgttatcaatatatatatataatatgcaagtattgcatgaaaaaaaaaaaaaaaaaaa

SEQ ID NO: 48 - DNA, 1957 bp, Arabidopsis thaliana
caaacgttactttccaaaacaatcttttcagttttagataaaaatttgatattaacttctgattcatgacgacacaagatgtgatagtcaatgatcagaatgatcagaaacagtgtagtaatgacgtcattttccgatcgagattgcctgatatatacatccctaaccacctcccactccacgactacatcttcgaaaatatctcagagttcgccgctaagccatgcttgatcaacggtcccaccggcgaagtatacacctacgccgatgtccacgtaacatctcggaaactcgccgccggtcttcataacctcggcgtgaagcaacacgacgttgtaatgatcctcctcccgaactctcctgaagtagtcctcactttccttgccgcctccttcatcggcgcaatcaccacctccgcgaacccgttcttcactccggcggagatttctaaacaagccaaagcctccgcggcgaaactcatcgtcactcaatcccgttacgtcgataaaatcaagaacctccaaaacgacggcgttttgatcgtcaccaccgactccgacgccatccccgaaaactgcctccgtttctccgagttaactcagtccgaagaaccacgagtggactcaataccggagaagatttcgccagaagacgtcgtggcgcttcctttctcatccggcacgacgggtctccccaaaggagtgatgctaacacacaaaggtctagtcacgagcgtggcgcagcaagtcgacggcgagaatccgaatctttacttcaacagagacgacgtgatcctctgtgtcttgcctatgttccatatatacgctctcaactccatcatgctctgtagtctcagagttggtgccacgatcttgataatgcctaagttcgaaatcactctcttgttagagcagatacaaaggtgtaaagtcacggtggctatggtcgtgccaccgatcgttttagctatcgcgaagtcgccggagacggagaagtatgatctgagctcggttaggatggttaagtctggagcagctcctcttggtaaggagcttgaagatgctattagtgctaagtttcctaacgccaagcttggtcagggctatgggatgacagaagcaggtccggtgctagcaatgtcgttagggtttgctaaagagccgtttccagtgaagtcaggagcatgtggtacggtggtgaggaacgccgagatgaagatacttgatccagacacaggagattctttgcctaggaacaaacccggcgaaatatgcatccgtggcaaccaaatcatgaaaggctatctcaatgaccccttggccacggcatcgacgatcgataaagatggttggcttcacactggagacgtcggatttatcgatgatgacgacgagcttttcattgtggatagattgaaagaactcatcaagtacaaaggatttcaagtggctccagctgagctagagtctctcctcataggtcatccagaaatcaatgatgttgctgtcgtcgccatgaaggaagaagatgctggtgaggttcctgttgcgtttgtggtgagatcgaaagattcaaatatatccgaagatgaaatcaagcaattcgtgtcaaaacaggttgtgttttataagagaatcaacaaagtgttcttcactgactctattcctaaagctccatcagggaagatattgaggaaggatctaagagcaagactagcaaatggattaatgaactaggttttatatgatccacgtatatgaatgcaatcttatcagaaaaatgaaacaaaatttcgttttgtgaacaaaggaattaaacttacacgtaaaagaataatatttgtgctttttcctttatgtgtatgtaatggataaatagttgtatcttttgtttggtgggaatgatgtaacctttccatattgtggcatattgctcgaatataatcaataattgcctt

SEQ ID NO: 49 - DNA, 1689 bp, Arabidopsis thaliana
atgctgacgaaaaccaacgacagccgtttgattgaccggagctccggcttcgatcaacggacaggaatctatcacagtcttcgtccctctctttctctacctcctatagatcaacctctctccgccgccgaattcgcgctttctctcctactcaaatcctcaccacctgccaccgccgggaaaaacattgaagccttaacctacctagttaactcgagctctggtgataacctcacttatggagagcttcttcgtagagttcgttctcttgctgtatctctccgggagcgatttccttctcttgcctccagaaatgtcgcttttatcctctctccttcttcgttggacataccagtgctttacttagctttgatgtcgatcggtgttgttgtttcaccggcgaacccaatcggatctgaatcggaggtgagtcatcaagtcgaagtcagtgaaccagtaattgcgttcgcgacatcgcagacggttaagaagcttcaatcctcttctttgcctctcggaactgttctgatggactcgactgagtttctctcctggttaaatcgatcggattcttcatcggttaatccatttcaggttcaggtcaaccaatcggaccctgccgctatcctcttttcctctggaacaaccgggcgggtcaaaggcgttttgctcactcaccgtaacctaatcgcctcgaccgccgtatctcaccaacggactctccaagatccggttaattacgatcgcgttggactgttctcgcttccgctcttccacgtgtttggtttcatgatgatgattcgagccatctcgcttggagagacattggtgcttttagggagatttgaactcgaggcgatgtttaaggcggtggagaaatataaggttactggtatgcctgtatctcctccgttgattgtagcgttggtcaaatcggagctcacgaagaagtacgatctccggtcgttgcgttcccttggctgcggaggagctccactcggcaaagacatcgcagagaggtttaagcagaaattcccagatgtagatattgtacagggctatggcttgacagagagctcgggaccagctgcctcaacgtttggacctgaagagatggtaaaatatggctcagttggtcgtatctctgagaatatggaagccaaaattgttgatccatccaccggagaatccttgccaccgggaaaaactggtgaactctggctccgaggaccagtcatcatgaaaggttatgtgggaaacgagaaagcgagtgcggagacagtagacaaagaagggtggttaaagactggtgatctctgttattttgattcggaagattttctatatattgttgatcggctaaaggagctaatcaaatacaaggcttatcaggttccaccggtagagttggagcagattcttcactcgaatccagatgtgattgatgctgcagttgttccgttccctgacgaggatgcaggagagattccaatggctttcatagtgagaaaaccaggaagcaatctcaacgaagcgcaaatcattgatttcgtagctaaacaagttactccgtacaagaaggtaagacgagttgcttttataaatgcaatcccaaaaaatcctgctggcaagattctgcgtcgggagcttactaaaatcgctgtggatggcaatgcatcaaaactttga

SEQ ID NO: 50 - DNA, 1849 bp, Rubus idaeus
tcttcgaaatcccatttcgcaatggcggtccaaacacctcaacacaacatcgtctaccgctccaagctcccggacatccatatcccaaaccacctccctctccattcctacatattccaaaacaaatcccacctcacctcaaagccctgcatcatcaatggcactactggcgacatccacacctacgccaaattcaaactcaccgcccggaaagtcgcctccggcctcaacaagctcggcatcgagaaaggcgacgtcttcatgcttttgctccccaacacttccgaattcgtctttgccttcttgggagcctcgttctgcggagccatgatgacagccgccaaccctttcttcactccggcggaaatcgcgaaacaggccaaggcgtcgaaggcgaagctgatcatcactttcgcttgctattacgacaaagtaaaagacttatcatgcgacgaagtgaagttgatgtgcattgactcgccgccacctgactcgtcttgtcttcatttctccgaactgactcagtcagacgagaacgacgtgccggatgtggacatcagcccggacgacgtcgtggcgttaccttattcctccgggacgacgggactgccgaaaggggtgatgttgacgcacaaagggctggtgacgagcgtgtctcagcaggtggacggagagaatccgaatttgtactacagcagcgacgacgtcgttctgtgcgtgctgccgctgtttcatatttactcgctgaactcggtcttgctatgcgggttaagagccggagctgccattctgctgatgcagaagtttgagattgtgtcgcttttggagctgatgcagaagcatagggttagtgttgcgccgattgtgcctccgactgttttggcgatcgccaagtttccagatcttgacaagtatgatttgggatccataagggtgctgaagagtggaggagcaccattggggaaggagcttgaagatacagtcagagctaaatttcccaatgtcacactcggtcagggatatggaatgacagaggcaggtccggtgttgacaatgtcgttggcatttgcaaaggaacccttcgaggtgaaaccaggtgggtgtgggactgtcgtcagaaacgcagagttgaagatcgttgatcctgaaactggtgcctctttgccgcgcaaccaccctggtgagatttgcatcagaggccaccagatcatgaaaggttatcttaatgatccggaggccacaagaacaaccatagacaagcaaggttggctacacacaggtgacataggcttcattgatgacgatgaagagctcttcattgttgatcgattaaaggagctcatcaaatacaaaggctttcaggttgcccctgctgagcttgaagccttgctcgtcacccatcctaacatctctgatgctgccgttgtcccaatgaaggatgacgcagctggcgaggttccggttgcatttgtcgtgagtccaaagggctctcaaatcactgaggatgaaatcaagcaatttatttcaaaacaggttgtattctacaaaagaataaaacgagtatttttcattgaagccattcccaagtccccatcgggcaagatcttgcggaaggagttgagagcaaagcttgctgctggctttgcaaattgaggaatgtttgccctccatttatccctatcatgaaagggctatgtatacttattaaaaggttttttttttccttttttttttctggacttaaaagtttgattaatgtgattcatccttaattaatttgaatccggaatttctacaaacttaatttatgtaaaaatcaattgaaactatatattgcttcgaaaaaaac

SEQ ID NO: 51 - DNA, 2213 bp, Lithospermum erythrorhizon
cttgaactttcctgcattcttgcacatttcttgttctaatttatttatttataaatcatttcaagaaaagaagtaaggtgctttggtatacccaagccaaggagagttggttagaagagaaagagtgagatagagaaccaaatttaaaagaaagctcatcacagaagtgaggtgggaataaatcccagaaaaacacaaaaacaaggcaatattattaccacctatagtattatcaccaaaccaaccgaacaacaacaaataacaatgttgtctgtagcttcccctgaaacccaaaagccagagctttcctccattgctgcccccccttcttccaccccccaaaaccaatcctccatttctggagataacaactccaatgaaaccatcatcttcagatccaaactacctgatatacccatctccaataacctccctctccacacatactgcttccagaatgcttctgaataccccaacagaacatgcatcattgatagcaaaactggaaaacaatacactttttctgaaacagattcaatctgcagaaaagttgcagctggattatcaaatcttggcatccaaaaaggagatgtgatcatggtcctcctccaaaactgtgctgaattcgttttcaccttcatgggtgcttccataataggtgcagtcatcaccacaggaaaccccttctacacaactgcagaaatcttcaaacaagtcaacgtctccaacacaaaactcatcattactcaatccaactacgttgacaagctccgtaacaccaccataaacgaatccgacaacaaatatccaaaacttggagaggattttaaggtgatcacaattgatacccccccagaaaactgcctacccttttcactcctcattgaaaacacccaagaaaaccaagttacatcagtttccatcgactcaaacgacccaatagcattaccattttcctcaggcaccacagggttaccaaaaggagtgatcctaacacacaaaagcctcattacaagcgttgcacaacaagtagatggagacaacccaaacttgtacctaaaacatgatgatgtagtactatgtgtacttcctttgttccatatatactccctaaattcagtacttttgtgttcattaagagctggagcagcagtgttgatcatgcagaaatttgagataggggcattgttggaacttatacaaagccaccgtgtatcggtggcggcggtggtgcctccgctagtattggcgttggcaaagaatccaatggtggataaatatgatctgagttcgataagggtggtgttgtcgggggcggcgccgctggggagggagttggaactagcgttacttaatagagtcccacatgccatttttgggcagggttatggcatgactgaagctggaccagtactatcaatgtccccttcatttgcaaagcacccatacccagcaaaatccgggtcatgtggaactgtagttagaaatgcagacctcaaggtgattgaccccgaaaccggttcctccctcggccgaaaccaacctggagaaatttgcattcgtggcgaacagatcatgaaaggctatctcaacgaccccgaggcaactgccaggaccgttgacatcgaggggtggctccataccggtgacattggctatgtggacgacgatgatgaagtgttcattgttgatagggtgaaggaactcatcaaattcaaggggttccaagttccaccagctgagcttgaggctctcctcatttcccaccccaacattgctgatgctgctgttgtaccgcaaaaagatgctgctgctggagaagtccctgttgcttttgtggttccttctaatgatggctttgaattaacagaagaagctgtcaaagaattcatttctaaacaggttgtgttctacaaaaggttgcacaaggtgtactttgtccactctattccaaagtcgccgtccggcaagattttgaggaaagatctcagagccaaactggccgccgccgcctcctcttgaattcttattgttcgatagttgcataaaagttattattgccatgtattatggctaattaataaataataggaattatttttcaaatgtagtcattattgtttatctatgtgaatgtttgcatgagactgagtaattgaactcattgatgagttcttttgttatgtgtgagaatggaatccaaccattttact

SEQ ID NO: 52 - DNA, 1668 bp, Zea mays
  misc_feature (603)..(603): n is a, c, g, or t
atgggttccgtagacgcggcgatcgcggtgccggtgccggcggcggaggagaaggcggtggaggagaaggcgatggtgttccggtccaagcttcccgacatcgagatcgacagcagcatggcgctgcacacctactgcttcgggaagatgggcgaggtggcggagcgggcgtgcctgatcgacgggctgacgggcgcgtcgtacacgtacgcggaggtggagtccctgtcccggcgcgccgcgtcggggctgcgcgccatgggggtgggcaagggcgacgtggtgatgagcctgctccgcaactgccccgagttcgccttcaccttcctgggcgccgcccgcctgggcgccgccaccaccacggccaacccgttctacaccccgcacgaggtgcaccgccaggcggaggcggccggcgcccggctcatcgtgaccgaggcctgcgccgtggagaaggtgcgggagttcgcggcggagcggggcatccccgtggtcaccgtcgacgggcgcttcgacggctgcgtggagttcgccgagctgatcgcggccgaggagctggaggctgacgccgacatccaccccgacgacgtcgtcgcgctgccntactcctccggcaccaccgggctgcccaagggcgtcatgctcacccaccgcagcctcatcaccagcgtcgcgcagcaggttgatggcgagaacccgaacctgtacttccgcaaggacgacgtggtgctgtgcctgctgccgctgttccacatctactcgctgaactcggtgctgctggccggcctgcgcgcgggctccaccatcgtgatcatgcgcaagttcgacctgggcgcgctggttgacctggtgcgcaggtacgtgatcaccatcgcgcccttcgtgccgcccatcgtggtggagatcgccaagagcccccgcgtgaccgccggcgacctcgcgtccatccgcatggtcatgtccggcgccgcgcccatgggcaaggagctccaggacgccttcatggccaagatccccaatgccgtgctcgggcaggggtacgggatgacggaggcaggccccgtgctggcgatgtgcctggccttcgccaaggagccgtacccggtcaagtccgggtcgtgcggcaccgtggtgcggaacgcggagctgaagatcgtcgaccccgacaccggcgccgccctcggccggaaccagcccggcgagatctgcatccgcggggagcagatcatgaaaggttacctgaacgaccccgagtcgacgaagaacaccatcgaccaggacggctggctgcacaccggcgacatcggctacgtggacgacgacgacgagatcttcatcgtcgacaggctcaaggagatcatcaagtacaagggcttccaggtgccgccggcggagctggaggcgctcctcatcacgcacccggagatcaaggacgccgccgtcgtctcaatgaacgacgaccttgctggtgaaatcccggtcgccttcatcgtgcggaccgaaggttctcaagtcaccgaggatgagatcaagcaattcgtcgccaaggaggtggttttctacaagaagatccacaaggtcttcttcaccgaatccatccccaagaacccgtcgggcaagatcctgaggaaggacttgagagccaggctcgccgccggtgttcactga

SEQ ID NO: 53 - DNA, 1300 bp, Vitis sp. cv. Optima
cttaatcttaagcttcaatttcattacgtatctagcatccatggcttcagttgaggaatttagaaacgctcaacgtgccaagggtccggccactatcctagccattggcacagctactcctgaccactgtgtctaccagtctgattatgctgattactatttcagggtcactaagagcgagcacatgactgagttgaagaagaagttcaatcgcatatgtgacaaatcaatgatcaagaagcgttacattcacttgaccgaagaaatgcttgaggagcacccaaacattggtgcttatatggctccatctcttaacatacgccaagagattatcactgctgaggtacctagacttggtagggatgcagcattgaaggctcttaaagagtggggccaaccaaagtccaagatcacccatcttgtattttgtacaacctccggtgtagaaatgcccggtgcggattacaaactcgctaatctcttaggtcttgaaacatcggttagaagggtgatgttgtaccatcaagggtgctatgcaggtggaactgtccttcgaactgctaaggatcttgcagaaaataatgcaggagcacgagttcttgtggtgtgctctgagatcactgttgttacattccgtggcccttccgaagatgctttggactctttagttggccaagccctttttggtgatgggtcttcagctgtgattgttggatcagatccagatgtctcgattgaacgaccactcttccaacttgtttcagcagcccaaacatttattcctaattcagcaggagccattgccggaaacttacgtgaggtggggctcacctttcatttgtggcccaatgtgcctactttgatttctgagaacatagagaaatgcttgacccaggcttttgacccacttggtattagcgattggaactcgttattttggattgctcacccaggtggccctgcaattctcgatgcagttgaagcaaaactcaatttagagaaaaagaaactcgaagcaactaggcatgtgttaagtgagtacggtaacatgtcaagtgcatgtgtgttgtttattctggatgagatgagaaagaaatccttgaagggggaaaaggctaccacaggtgaaggattggattggggagtattatttggttttgggccgggcttgaccatcgaaactgttgtgctgcatagcgttcctacagttacaaattaagagaaataaaagagaatggttgacccttcaatggcgtaatgtatcaaataggagttagcaaaggtatttatctccgaaatt

SEQ ID NO: 54 - DNA, 1185 bp, Vitis vinifera
atggcttcagtcgaggaatttagaaacgctcaacgtgccaagggtccggccaccatcctagccattggcacagctacccccgaccactgtgtctaccagtctgattatgctgattactatttcagggtcactaagagcgagcacatgactgagttgaagaagaagttcaatcgcatatgtgacaaatcaatgatcaagaagcgttacattcacttgaccgaagaaatgcttgaggagcacccaaacattggtgcttatatggctccatctcttaacatacgccaagagattatcactgctgaggtacctagacttggtagagatgcagcattgaaggctcttaaagagtggggccaaccaaagtccaagatcacccatcttgcattttgtacaacctccggtgtagaaatgcccggtgcggattacaaactcgctaatctcttaggtcttgaaacatcggttagaagggtgatgttgtaccatcaagggtgctatgcaggtggaactgtccggcgaactgctaaggatcttgcagaaaataatgcaggagcacgagttcttgtggtgtgctctgagatcactgttgttacattccgtgggccttccgaagatgctttggaccctttagttggccaagccctttttggtgatgggtcttcagctgtgattgttggatcagatccagatgtctcgattgaacgaccactcttccaacttgtttcagcagcccaaacatttattcctaattcagcaggagccattgccggaaacttacgtgaggtggggctcacctttcatttgtggcccaatgtgcctactttgatttctgagaacatagagaaatgcttgactcaggcttttgacccacttggtattagcgattggaactcgttattttggattgctcacccaggtggccctgcaattctcgatgcagttgaagcaaaactcaatttagagaaaaagaaattggaagcaactaggcatgtgttaagtgagtacggtaacatgtcaagtgcatgtgtgttgtttattttggatgagatgagaaagaaatccctaaagggggaaaaagccaccactggtgaaggattggattggggagtactatttggttttgggccaggcttgaccatcgaaactgttgtgctacatagcattcctatggttacaaattaagtgaag

SEQ ID NO: 55 - DNA, 1547 bp, Vitis vinifera
cttcctcaacttaatcttaggccttaatttgagtacgtagctgggatcaatggcttcagtcgaggaaattagaaacgctcaacgtgccaagggtccggccaccatcctagccattggcacagctacccccgaccactgtgtctaccagtctgattatgctgatttctatttcagggtcactaagagcgagcacatgactgcgttgaagaagaagttcaatcgcatatgtgacaaatccatgatcaagaagcgttacattcatttgaccgaagaaatgcttgaggagcacccaaacattggtgcttatatggctccatctcttaacatacgccaagagattatcactgctgaggtacccaagctcggtaaggaagcagcattgaaggctcttaaagagtggggtcagcctaaatcgaagatcacccaccttgtattttgtaccacctcgggtgtagaaatgcctggtgcagattataaactcgctaatcttttaggtctcgaaccatccgtcagaagagtgatgttgtaccatcaagggtgctatgcaggtggaactgtccttcgaaccgctaaggatcttgcagagaataatgcaggagcacgagttcttgtggtgtgctctgagatcacagttgttacatttcgcggcccttccgaagatgctttggactctttagttggccaagccctttttggtgatgggtctgcagctgtaatcgtaggatcagatccggatatctcaattgaacgaccactcttccagcttgtctcagcagcccaaacatttattcctaattctgcaggtgccattgcaggaaacttacgtgaggtgggactcacctttcatttgtggcccaatgtgcccactttaatttctgaaaacattgagaaatgtttgactcaggcttttgacccacttggtattagcgattggaactccttattttggattgctcacccaggtggccctgcaattcttgatgcagttgaagcaaaactcaatttagataaaaagaaactcgaagcaacgaggcatgtgctaagtgagtatggaaacatgtcaagtgcatgtgtgttgtttattttggatgagatgagaaagaaatcccttaagggggagagggccaccacgggtgaaggattggattggggagtattattcggttttggaccaggcttgactattgaaactgttgtgttgcatagcattcctatggtgacaaattaagtgaaggaaaagagaatggtcccttcaatgtcctattatgttgaataggagtaaggtatttatctccgaaactaaattatactcttatactattttattatttttttctaaatttagattgtaatctagtgattgttagaccctcttggtgagctcaaatgaaacggttgagtttcaagttcagactgttttatcatcttgaagattccctaaacattgtaatgttgtgttcatatgaacattattgaaaagtaaataaaagaaatattggattttgataaaaaaaaaaaaaaaaaaaaaaaaaaaaaaaaaaaaaaaa

SEQ ID NO: 56 - DNA, 1170 bp, Arachis hypogaea
atggtgtctgtgagtggaattcgcaaggttcaaagagcagaaggtcctgcaaccgtattagcgattggcacagcaaatccaccaaactgtgttgatcagagcacatacgcagattactattttagagtaaccaatagcgagcacatgaccgacctcaagaagaaatttcagcgcatttgtgagagaacacagatcaagaacagacatatgtatctaacggaagaaatactgaaggagaatcctaacatgtgcgcatacaaagcaccgtccttggatgcaagggaagacatgatgatcagggaggtaccaagggttggaaaagaggctgcaactaaggcaatcaaggaatggggtcagccaatgtctaagatcacacatttgatcttctgcaccaccagcggtgttgcgttgcctggcgttgattacgaactcatcgtactcttagggctcgacccaagcgtcaagaggtacatgatgtaccaccaaggctgcttcgctggcggcactgtccttcgtttggctaaggacttggctgaaaacaacaaggatgctcgtgtgcttattgtttgttctgaaaatacttcagtcacttttcgtggtcctagtgagacagacatggatagtcttgtaggacaagcattgtttgccgatggagctgctgcaattatcattggttctgatcctgttccagaggttgagaatcctctctttgagattgtttcaactgatcaacaacttgtccctaacagccatggagccatcggtggtctccttcgtgaagttggacttacattctatcttaacaagagtgttccggatattatttcacaaaacatcaatgatgcactcagtaaagcttttgatccactaggtatatctgattataactcaatattttggattgcacatcctggtggacgtgcaattttggaccaagttgaagagaaggtgaacttgaagccagagaagatgaaagccaccagagatgtgcttagcaattatggtaacatgtcaagtgcgtgtgtgttcttcattatggatttgatgagaaagaagtcacttgaagcaggacttaaaaccaccggagaaggacttgattggggtgtactttttggttttggtcctggtctcactattgaaactgttgttctccgcagcatggccatataa

SEQ ID NO: 57 - DNA, 1179 bp, Cissus rhombifolia
atggcttcagttgaggaatttagaaacgctcaacgtgccaagggtccagctaccatcctagccattggcacagctactcccgatcagtgtgtctaccagtctgattatgctgattactatttccgggtcactaagagcgagcacatgactgagttgaagaagaagttcaatcgcatatgtgagaaatcaatgatcaagaagcgttacagtcatttgaccgaagaaatgctcgaggagcacccaaacattggtgcttatatggctccatctcttaacatacgccaagagattatcactgctgaggtacctaagcttggtaaggaagcagcattgaaggctctaaaagagtggggccagccaaagtccaagatcacccatcttgtattttgtacaacctccggtgtagaaatgcctggtgcagattacaaactcgctaatctcttagggcttgaaacatcggtcagaagagtgatgttgtaccatcaagggtgctatgcaggtggaactgtcctccgaactgctaaggatcttgcagagaataatgcaggagcacgagttcttgtggtgtgctctgaaatcactgttgttacattccgtgggccttctgaaaccgctttggactctttagttggtcaagccctttttggtgatgggtctgcagctgtgatcgttggatcagatccagatatcttgattgaacgaccgctcttccaactcgtctcagcagcccaaacatttattcctaattcagcaggtgccattgccgggaacttacgtgaggtgggactcaccttccatttgtggcccaatgtgcctactttaatttctgagaacatagagaaatgcttgactcaggcttttgacccacttggtattagcgattggaactcgttattttggattgctcacccaggtggtccagctattcttgacgcggttgaagcaaaactcagtttagataaacaaaaactcgaagcaacgaggcatgtgctaagtgagtatggcaacatgtcaagtgcatgtgtgttgtttattttggatgagatgagaaaaaaatcccttaagggggagaaggccaccacaggtgaaggattggattggggagtattattcggttttggcccaggtttgactattgagactgttgtgttgcatagcattcctatggttacaaattaa

SEQ ID NO: 58 - DNA, 1179 bp, Parthenocissus henryana
atggcttcagttgaggaatttagaaacgctcaacgtgccaagggtccggccaccatcctagccattggcacagctactcccgaccagtgtgtctaccagtctgattatgctgattactattttagggtcactaagagcgagcacatgactgagttgaagaagaagttcaatcgcatatgtgaaaaatcaatgatcaagaagcgttatattcatttgactgaaaagatgcttgaggagcacccaaacattggtgcttatatggctccatctcttaacatacgccaagagattatcactgccgaggtacccaagcttggtaaagaagcagcattgaaggctcttaaagagtggggtcaacccaaatccaagattacccatcttgtattttgtaccacctctggtgtagaaatgcctggtgccgactataaactcgctaatctcttaggcctcgaaacatctgttagaagagtgatgttgtatcatcaaggttgctatgcaggtggaactgtccttcgaactgctaaggatcttgcagagaataatgcaggagcacgagttcttgtggtgtgctctgagatcactgttgtcacattccgtggaccttccgaaactgctttggactctttagttggccaagccctttttggtgatgggtctgcagctgtgatcgttggatcagatccagatatctcgattgaacaaccactttttcaactcgtctcagcagcccaaacatttattcctaattcagcaggtgccattgccgggaacttacgtgaggtgggactcacatttcatttgtggcccaatgtgccaactttaatttctgagaacatagagaaatgcttgactcaggcttttgacccacttggtattagcgattggaactcgttattttggattgctcacccaggtggccctgcaattcttgatgcggttgaagcaaaactcaatttagacaaaaagaaacttgaagcaacgaggcatgtgttaagtgagtatggcaacatgtcaagtgcatgtgtgttgtttattttggatgagatgagaaagaaatcacttaagggggagaaggccaccacaggtgaaggattggattggggagtattatttggctttggatcaggcttgactattgagactgttgtgttgcatagcattcctatggttacaaattaa

SEQ ID NO: 59 - DNA, 1179 bp, Parthenocissus quinquefolia
atggcttcagttgaggaatttagaaacgctcaacgtgcaaagggtccagccaccatcctagccattggcacagctactcccgacaactgtgtctatcagtcagattatgctgatttctacttcagggtcactaagagcgagcacatgactgagttgaagaagaagttcaatcgcatatgtgagaaatcaatgatcaagaagcgttacagtcatttgaccgaagaaatgctcgaggagcacccaaacattggtgcttatatggctccatctcttaacatacgccaagagattatcactgctgaggtacctaagcttggtaaggaagcagcattgaaggctctaaaagagtggggccagccaaagtccaagatcacccatcttgtattttgtacaacctccggtgtagaaatgcctggtgcagattacaaactcgctaatctcttagggcttgaaacatcggtcagaagagtgatgttgtaccatcaagggtgctatgcaggtggaactgtcctccgaactgctaaggatcttgcagagaataatgcaggagcacgagttcttgtggtgtgctctgtaatcactgttgttacattccgtgggccttctgaaaccgctttggactctttagttggtcaagccctttttggtgatgggtctgcagctgtgatcgttggatcagatccagatatcttgattgaacgaccgctcttccaactcgtctcagcagcccaaacatttattcctaattcagcaggtgccattgccgggaacttacgtgaggtgggactcaccttccatttgtggcccaatgtgcctactttaatttctgagaacgtagagaaatgcttgactcaggcttttgacccacttggtattagcgattggaactcgttattttggattgctcacccaggtggtccagctattcttgacgcggttgaagcaaaactcagtttagataaacaaaaactcgaagcaacgaggcatgtgctaagtgagtatggcaacatgtcaagtgcatgtgtgttgtttattttggatgagatgagaaaaaaatcccttaagggggagaaggccaccacaggtgaaggattggataggggagtattattcggttttggcccaggtttgactattgagactgttgtgttgcatagcattcctatggttacaaattaa

SEQ ID NO: 60 - DNA, 1618 bp, Vitis riparia
gcttcaatttcattacgtatctagcatccatggcttcagttgaggaatttagaaacgctcaacgtgccaagggtccggccactatcctagccattggcacagctactcctgaccactgtatctaccagtctgattatgctgattactatttcagggtcactaagagcgagcacatgactgagttgaagaagaagttcaatcgcatatgtaagtatattcatgcattaattcttatatacataacaattgtatgcatctaagagtgtgagctattaggtgaggctcacctccaagcgaatgaatgttccaacctttctagagtaaagcttttagataaattagttcaggaaacttgaaaatcattttacttcagtaaccaatattcctttcatttgactgtaatggcttgaagagctgttttttgaatcatatagcactgctagctataattaagaatacccttttatactttcttcaatgttaaatgcatgttgatcatcttgaacaatatactatatgacttgtcgattggtaaaactaatgtgttcatgttacttcatttacaggtgagaaatcaatgatcaagaagcgttacattcacttgaccgaagaaatgcttgaggagcacccaaacatcggtgcttatatggctccatctcttaacatacgccaagagattatcaccgctgaggtacctagacttggtagggatgcagcattgaaggctcttaaagagtggggccaaccaaagtccaagatcacccatcttgtgttttgtacaacctccggtgtagaaatgcccggtgcggattacaaactcgctaatctcttaggtcttgaaacatcggttagaagggtgatgttgtaccatcaagggtgctatgcaggtggaactgtccttcgaaccgctaaggatcttgcagaaaataacgcaggagcacgagttcttgtggtgtgctctgagatcactgttgttacattccgtgggccttccgaagatgctttggactctttagttggccaagccctttttggtgatgggtcttcagctgtgattgttggatcagatccagatgtctcgattgaacgaccactcttccgacttgtttcagcagcccaaacatttattcctaattcagcaggagccattgctggaaacttacgtgaggtggggctcacctttcatttgtggcccaatgtgcctactttgatttctgagaacatagagaaatgcttgacccaggcttttgacccacttggtattagcgattggaactcgttattttggattgctcacccaggtggccctgcaattctcgatgcagttgaagcaaaactcaatttagagaaaaagaaacttgaagcaactaggcatgtgttaagtgagtacggtaacatgtcaagtgcatgtgtgttgtttattttggatgagatgagaaagaaatccttgaagggggaaaatgctaccacaggtgaaggattggattggggagtattattcggttttgggccgggcttgaccatcgaaactgttgtgctgcatagcattcctacaattacaaattaagagaaataaaagagaatggtttaccttataatgcactaatgtatcaaatag

SEQ ID NO: 61 - DNA, 1618 bp, Vitis labrusca
gcttcaatttcattacgtatctagcatccatggcttcagttgaggaatttagaaacgctcaacgtgccaagggtccggccactatcctagccattggcacagctactcctgaccactgtatctaccagtctgattatgctgattactatttcagggtcactaagagcgagcacatgactgagttgaagaagaagttcaatcgcatatgtaagtatattcatgcattaattcttatatacatgacaattgtatgcatctaagagtgtgagctattaggtgaggctcacctccaagcgaatgaatgttccaacctttctagagtaaagcttttagataaattagttcaggaaacttgaaaatcattttacttcagtaaccaatattcctttcatttgactgtaatggcttgaagagctgttttttgaatcatatagcactgctagctataattaagaatacccttttatactttcttcaatgttaaatgcatgttgatcatcttgaacaatatactatatgacttgtcgattggtaaaactaatgtgttcatgttacttcatttataggtgagaaatcaatgatcaagaagcgttacattcacttgaccgaagaaatgcttgaggagcacccaaacattggtgcttatatggctccatctcttaacatacgccaagagattatcaccgctgaggtacctagacttggtagggatgcagcattgaaggctcttaaagagtggggccaaccaaagtccaagatcacccatcttgtattttgtacaacctccggtgtagaaatgcccggtgctgattacaaactcgctaatctcttaggtcttgaaacatcggttagaagggtgatgttgtaccatcaagggtgctatgcaggtggaaccgtccttcgagccgctaaggatcttgcagaaaataacgcaggagcacgagttcttgtggtgtgctctgagatcacagttgttacattccgtgggccttccgaagatgctttggactctttagttggccaagccctttttggtgatgggtcttcagctgtgattgttggatcagatccagatgtctcgattgaacgaccactcttccaacttgtttcagcagcccaaacatttattcctaattcagcaggagccattgccggaaacttacgtgaggtggggctcacctttcatttgtggcccaatgtgcctactttgatttctgagaacatagagaaatgcttgacccaggcttttgacccacttggtattagcgattggaactcgttattttggattgctcacccaggtggccctgcaattctcgatgcagttgaagcaaaactcaatttagagaaaaagaaacttgaagcaactaggcatgtcttaagtgagtacggtaacatgtcaagtgcatgtgtgttgtttattttggatgagatgagaaagagatccttgaagggggaaaatgctaccacaggtgaaggattggattggggagtattattcggttttgggccgggcttgaccatcgaaactgttgtgctgcatagcattcctacagttacaaattaagagaaataaaagagaatggtttacccttcaatgcagtaatgtatcaaatag

SEQ ID NO: 62 - DNA, 1739 bp, Vitis sp. cv. ′Norton′
tttgaagccaactaatcattcaaaacccaaattcaaatatctaacattatttattgaccgccaatagatgagagttggtgagacaagctataaaagcccggcacccacaaccagctttctcaagccaactctaagcacttgagttctctttccttcctcaacttaatcttaagcttcaatttcattacgtatctagcatccatggcttcagttgaggaatttagaaacgctcaacgtgccaagggtccggccactatcctaaccattggcacagctactcctgaccactgtatctaccagtctgattatgctgattactatttcagggtcactaagagcgagcacatgactgagttgaagaagaagttcaatcgcatatgtaagtatattcatgcattaattcttatatacatgacaattgtatgcatctaagagtgtgagctattaggtgaggctcacctccaagcgaatgaatgttccaacctttctagagtaaagcttttagataaattagttcaggaaacttgaaaatcattttacttcagtaaccaatattcctttcatttgactgtaatggcttgaagagctgttttttgaatcatatagcactgctagctataattaagaatacccttttatactttcttcaatgttaaatgcatgttgatcatcttgaacaatatactatatgacttgtcgattggtaaaactaatgtgttcatgttacttcatttataggtgagaaatcaatgatcaagaagcgttacattcacttgaccgaagaaatgcttgaggagcacccaaacattggtgcttatatggctccatctcttaacatacgccaagagattatcaccgctgaggtacctagacttggtagggatgcagcattgaaggctcttaaagagtggggccaaccaaagtccaagatcacccatcttgtattttgtacaacctccggtgtagaaatgcccggtgctgattacaaactcgctaatctcttaggtcttgaaacatcggttagaagggtgatgttgtaccatcaagggtgctatgcaggtggaaccgtccttcgagccgctaaggatcttgcagaaaataacacaggagcacgagttcttgtggtgtgctctgagatcacagttgttacattccgtgggccttccgaagatgctttggactctttagttggccaagccctttttggtgatgggtcttcagctgtgattgttggatcagatccagatgtctcgattgaacgaccactcttccaacttgtttcagcagcccaaacatttattcctaattcagcaggagccattgccggaaacttacgtgaggtggggctcacctttcatttgtggcccaatgtgcctactttgatttctgagaacatagagaaatgcttgacccaggcttttgacccacttggtattagcgattggaactcgttattttggattgctcacccaggtggccctgcaattctcgatacagttgaagcaaaactcaatttagagaaaaagaaacttgaagcaactaggcatgtcttaagtgagtacggtaacatgtcaagtgcatgtgtgttgtttattttggatgagatgagaaagaaatccttgaagggggaaaatgctaccacaggtgaaggattggattggggagtattattcggttttgggccgggcttgaccatcgaaactgttgtgctgcatagcattcctacagttacaaattaa

SEQ ID NO: 63 - DNA, 1629 bp, Cicer arietinum
atggatcttctcctattggaaaagacccttttagcccttttcatcgccgccactatcgccatcacaatttcaaaactccgtggcaaaagattcaaacttccaccaggtccaatcccagtccccgtcttcggtaactggcttcaagtcggcgatgatctcaaccaccgtaatctcaccgacttagcaaaacggttcggcgatatcttcctccttcgtatgggtcaaagaaacctcgtcgtagtttcatcacctgaactcgcaaaagaggttctccacactcaaggtgtcgaattcggttcccgaacaaggaacgttgttttcgatatcttcaccggaaaaggacaggatatggttttcaccgtttacggaacattggcggaaatgagaagaatcatgacggtgccgtttttcacaaacaaagttgttcaacagtaccgttttggatgggaatttgaagctcagagtgttgtcgacgatgttaagaaaaatccagaggcgtgttcgagtggaattgttcttcggagaagattgcaacttatgatgtataatattatgtataggattatgtttgatagaagatttgagagtgaagaggatcctttgtttgtgaagcttaaagctttgaatggtgaaagaagtcgtttggctcaaagctttgagtataattatggtgattttattcctattttgagaccctttttgaaaggttatttgaagctttgtaaagaggttaaggatcgtaggttgcagctcttcaaagactatttcgttgatgagagaaagaaacttggaagcaccaagagcaccaccaatgaaggactgaaatgtgctattgatcacattttggatgctcaacagaagggtgagatcaatgatgacaatgttctttacattgttgagaacatcaatgttgctgcaattgaaacaacattatggtcaattgaatggggcattgctgagctagtgaatcaccaaaagatccaaaacaaagtaagggaagaaattgatagagttcttggaccaggacaccaagtaactgaaccagatctccaaaagctaccttacctacaagccgtaatcaaagaaacacttcgtcttcgaatggcgattccactcctcgtcccacacatgaaccttcacgatgcaaagctcagtggttttgacatcccggccgagagcaagatattggtcaatgcgtggtggctcgcaaacaacccggcccaatggaaaaagcccgaggaatttaggcccgagaggttcttagaggaagagtctcatgtcgaggctaatggaaatgactttaggtaccttccgttcggcgttggtagaaggagttgtcctggaattattctcgctttaccgatcctcggtattactttgggacgattggttcagaatttcgagcttttgcctcctccgggacagtctaagatcgacacggctgagaaaggaggacaatttagtttgcatatactcaaacattccaccattgtttgtaagccaagatcatttaattaattagtcctcacatcaataataccctttaatttgttttactttactctactttgtgtaatgcatatttcaatgattatgtgggaatgttggtaataaaaaaaaaaaaaaaaa

SEQ ID NO: 64 - DNA, 1518 bp, Populus tremuloides
atggatctcctactcctggagaagaccctcttgggttctttcgttgccattctcgttgccattctcgtttctaaactacgtggcaaacgttttaaactccctccaggtcctttacctgtccccgtgtttggaaactggcttcaagttggtgatgatttgaaccaccgtaacctcaccgacttagccaagaaattcggtgacatcctcctccttcgcatgggccaacgcaatcttgtagtcgtctcctcacctgagctatccaaagaggttctgcacacacaaggtgttgagttcgggtcgagaacaagaaatgttgtttttgatatctttactggaaagggacaagacatggtgttcactgtctatggtgagcattggaggaagatgaggagaatcatgacagtccctttctttacaaacaaggttgtccaacaatataggtatggatgggaagaggaagcggctcaagttgtcgaggatgttaagaaaaaccccggggctgcaactcatgggattgttttgaggaggagactgcaactgatgatgtataacaacatgtataggattatgtttgataggagatttgagagcgaagaagatcctttgtttaataaacttaaggctttgaatggtgagaggagcagattggctcagagttttgattataattatggtgatttcatccccattttgagacctttcttgagaggttacttgaagatctgccaggaggttaaggagagaaggttgcaactcttcaaggactactttgtcgatgagaggaagaaacttgcaagcacaaagaacatgtgcaatgaagggttgaagtgcgcaatagaccatatcctggatgctcaaaagaagggagagatcaacgaggacaacgtcctttacattgttgagaacatcaacgtcgctgcaattgagacaacactatggtcgatcgagtggggaattgctgagcttgtgaaccatcctgaaatccagaagaagttgcgccatgagctcgataccttgcttggacctggtcaccaaatcaccgagcctgacacctacaagctcccttaccttaacgctgttgtcaaagagaccctccgactcaggatggcaattcctctactcgtcccacacatgaaccttcatgatgccaagcttggaggctttgacattccagctgagagcaagatcttggtcaacgcctggtggctcgccaacaaccctgcccactggaaaaaccctgaagaattcaggccagagaggttcttggaagaggaggccaaggtcgaggccaatggcaatgatttcaggtaccttccatttggagttgggagaaggagctgccctgggattattcttgcattgccaattcttggcattactctgggacgtctggtacagaatttcgagctcttgcctcctcctggacagtcaaagatcgacacctcagagaaaggtggacagttcagtttgcacatattgaagcactccactattgttgcaaagccaaggtccttttaa

SEQ ID NO: 65 - DNA, 1602 bp, Oryza sativa
atggcggcctccgcgatgagggtggccatcgccaccggggcgtcgttggcggtgcatttgttcgtcaagtcgttcgtgcaggcgcagcatcctgctctcaccttgctgctgccagtggctgtgtttgtcggcattgcggtgggcgcgaagggcgggagcggtggtgacgggaaggcgccgccggggccggcggccgtgccggtgttcggcaactggctgcaggtgggcaacgacctgaaccaccggttcctcgcggcgatgtcggcacggtacggtcccgtgttccgtctgcggctgggcgtgcgcaacctggtggtggtgtcggacccgaagctggcgacggaggtgctgcacacgcagggcgtggagttcggctcccgcccgcgcaacgtcgtcttcgacatcttcaccgccaacggcgccgacatggtgttcaccgagtacggcgaccactggcgacgcatgcgccgcgtcatgacgctgccgttcttcacggcgcgcgtcgtgcagcagtacaaggccatgtgggaggccgagatggacgccgtcgtggacgacgtgcgcggcgacgcggtggcgcagggcaccggcttcgtggtgcgacgcaggctgcagctcatgctgtacaacatcatgtaccggatgatgttcgacgcgcggttcgagtcggtggacgaccccatgttcatcgaggccaccaggttcaactccgagcgcagccgcctcgcgcagagcttcgagtacaactacggcgacttcatccccatcctccgtcccttcttgcggggctacctcaacaagtgccgtgacctccagagcaggaggctcgccttcttcaacaacaactacgtcgagaagagaaggaaggtgatggacactccgggagacaggaacaagctccggtgcgcgatcgaccatatccttgaggcggagaagaacggcgagctgacggcggagaacgtgatctacatcgtggagaacatcaacgtggccgccatcgagacgacgctctggtccatcgagtgggcgctggccgaggtcgtcaaccacccggcggtgcagagcaaggtccgcgccgagatcaacgacgtgctcggcgacgacgagcccatcaccgagtccagcatccacaagctgacttacctgcaggccgtgatcaaggagacgctgcggctgcactccccgatcccgctgctggtgccgcacatgaacctggaggaggccaagctcggcgggtacaccatccccaagggatccaaggtggtggtgaacgcgtggtggctggccaacaacccggcgctgtgggagaaccccgaggagttccggcctgagcggttcttggagaaggagagcggcgtggacgccaccgtcgccgggaaggtggacttcaggttcctgcccttcggcgtgggccgccgcagctgcccggggatcatcctggcgctgcccatcctggcgctcatcgtcgggaagctggtgaggagcttcgagatggtgccgccgccgggcgtggagaagctggacgtgagcgagaaaggcgggcagttcagcctccacatcgccaagcactccgtcgtcgccttccaccccatctctgcctga

SEQ ID NO: 66 - DNA, 1173 bp, Camellia sinensis
atggtgttcactgtatacggtgagcactggaggaagatgaggaggatcatgacggttcctttttttaccaacaaggtggtgcagcagtacaggttcgggtgggaggacgaggcgggtcgggtcgtggaggatgtgaagaagaacccggaagcgaagaccaatgggatcgtgctgaggaggcggttgcagctgatgatgtacaataacatgtacaggattatgtttgattcgaggttcgagagcgaggaggacccgttgttcgtgaaattgaaggcgttgaatggagagaggagtaggttggctcagagctttgagtataactacggcgattttattccgattttgaggccgttcttgagagggtacttgaagatctgcaaagaagttaaagagaggaggttgcagcttttcaaggactattttgtcgatgaaaggaagaagttagccaagccacgaagagccatggacacagttactctaaaatgtgcgattgatcatattttggatgctcaacaaaagggagagatcaacgaggacaacgttctttacatcgtggagaacattaacgtcgctgcaattgagacaacattatggtcgatagaatggggcatagcagaacttgtaaaccacccccaaatccagaaaaagcttcggcacgaacttgacaccatgcttggccttggagtccaaatcaccgagccagacacctacaaactcccctacctccaagctgtagtcaaagagaccctccgcctccggatggcaattcccctcttagtcccccacatgaacctccacgatgcaaagctctctggctatgacatccctgctgagagcaaaatcttggtaaacgcgtggtggcttgcaaacaaccccgacaactggaagaacccagaagagttcaggcccgagaggttcttggaagaggaggctaaggttgaggccaatggcaatgactttaggtaccttccgtttggtgtcggaaggaggagttgccctggaattatccttgctctgccaattctcggcatcactttgggaaggttggttcagaatttcgagctcttgcctcctccgggacaggccaagattgatactgctgagaaggggggacagttcagcttgcatattttgaagcactcgaccattgttctgaaaccaagatcgttctga

SEQ ID NO: 67 - DNA, 1766 bp, Vigna radiata var. radiata
aatttaccaccgtcacgtcaccaaaatggatctcctcctcctggagaagaccctcctcggcctcttcctagcggcggtggtagccattgttgtctccaagctccgcggcaagcgtttcaagctcccgccgggcccactccccgtccccatcttcggcaactggctccaggttggcgacgacctcaaccaccgcaacctcactcaactcgccaagcgcttcggcgacatcttcctcctccgcatggggcagcgcaacctggtcgtggtttcctcgccggacctcgccaaggaggtgctgcacacgcagggcgtggagttcggctcccgcacccgcaacgttgtcttcgacatcttcaccggcgagggccaggacatggtcttcaccgtctacggcgagcactggcgcaagatgcgacgcatcatgaccgtgcccttcttcaccaacaaggtcgtccagcagtaccgccacggttgggaggccgaggccgccgccgtcgtggacgacgtcaggaagaatcccgacgcggccgtctccggcctggtcatccgccgaaggctacagctcatgatgtacaacaacatgtaccgcatcatgttcgaccggagattcgagagcgaagaagaccctctgttccagcgtctgaaagcgctgaacggcgagaggagtcgcttggctcagagctttgagtataactatggcgatttcattcccatcttgagacccttcttgaagggttacttgaagatttgcaaggaagtgaaagaaaccaggttgaagcttttcaaggattacttcgtcgacgagaggaagaatattggaagcacgaagagcactaacaacgaaggacttaaatgtgctattgatcacattttggatgctgagaaaaagggtgagatcaacgaagacaacgtgctttacattgttgagaacatcaacgttgctgcaattgaaacaactctctggtcaattgaatggggtattgctgagcttgtgaaccatccagagatccagcagaaagtgagggatgaaattgacagagttcttggagtagggcatcaggtgactgagccagatatccaaaagcttccataccttcaagcagtggtgaaggaaacccttcgcctcagaatggcaatccctctccttgtcccacacatgaacctccatgatgctaagcttggtggctatgacatcccagctgaaagcaagattttggtgaatgcatggtggctggcgaacaaccctgcacactggaagaagccagaagagttcaggcctgagaggttcttcgaagaggaatcgcatgtggaagcgaatggcaatgacttcaggtaccttccctttggtgttgggagaagaagctgccccggaatcattcttgcattgcccattcttggcatcactttgggacgcttggtccaaaactttgagctcttgcctccccctgggcagtcccagattgacaccagtgagaaaggaggacagtttagcttgcacatactaaagcattccaccgttgttgcaaagccaaggtccttttagacttcaccacatcatcgttaccaatcccctttattattttttctttcttattctccctgtattatcgatgtttcaaaatggggttgctccatgccatgtataatgggcctcctaatgggtaggtggtgatgtatctcttggtcccattgtaattctctcacaacttcaactcatgaatgatcttgagatggttttgtaataaacttacacttttgtctctaat

SEQ ID NO: 68 - DNA, 1620 bp, Helianthus tuberosus
  misc_feature (1588)..(1588): n is a, c, g, or t
  misc_feature (1591)..(1591): n is a, c, g, or t
  misc_feature (1593)..(1594): n is a, c, g, or t
  misc_feature (1597)..(1597): n is a, c, g, or t
  misc_feature (1599)..(1600): n is a, c, g, or t
  misc_feature (1602)..(1602): n is a, c, g, or t
  misc_feature (1604)..(1604): n is a, c, g, or t
  misc_feature (1607)..(1608): n is a, c, g, or t
aaatcacacaacaccaccaccaccgtaaccatggacctcctcctcatagaaaaaaccctcgtcgccttattcgccgccattatcggcgcaatactaatctccaaactccgcggtaaaaaattcaagctcccacctggcccaatcccggttccaattttcggcaactggctacaagttggcgatgatttgaaccaccggaacttaaccgatctggctaagaggtttggtgagatcttgctgctacgcatggggcagaggaatctggtagttgtgtcttcgcctgagcttgctaaagaggtgttgcatacacaaggagtggagtttggttcgagaacaaggaatgttgtgttcgatatttttactgggaagggtcaggatatggtgtttacggtttatggtgagcattggaggaagatgaggaggatcatgaccgtaccctttttcaccaacaaagttgttcagcaatacaggtatgggtgggaggctgaggccgcggcggttgtggacgatgtgaagaagaatccggctgcagcaactgaaggaatcgtgatccgaagacggttacaactcatgatgtataacaacatgttcagaatcatgttcgacagacgattcgaaagtgaagatgatcccttgtttttgaaactcaaggcgttgaacggtgagaggagtcgattggcgcagagctttgagtacaactatggcgatttcatccctattttgcggccgtttttgagaaattatttgaagttgtgcaaggaagttaaagataaaaggattcagctcttcaaggattacttcgttgacgaaaggaagaagattggaagcactaagaaaatggacaacaatcagttgaaatgtgccattgatcacattcttgaagctaaagagaagggtgagatcaatgaagacaatgttctttacattgttgaaaacatcaatgttgcagcaatcgagacaactctatggtcgatcgaatggggaattgcggagctagttaaccatcccgagatccaagccaaactcaggcacgagctcgacaccaagctcgggcccggtgtccagatcaccgagcccgacgtccaaaacctcccttacctccaagccgtggtcaaggaaaccctccgtctccgtatggcgatcccgcttctagtcccacacatgaacctccatgacgctaagctcggcgggtttgacatcccggccgaaagcaagatcttggtcaacgcgtggtggttagcaaacaaccccgaccaatggaagaaacccgaggagtttaggccagagaggtttttggaagaggaagcgaaggttgaggctaacgggaatgattttaggtacttgccgtttggagtcgggagaaggagttgccccgggattattcttgcattgccgatacttggtattacaatcgggcgtttggtgcagaatttcgagctgttgcctccaccgggacagtctaagatcgataccgatgagaagggtgggcagtttagtttgcatatcttgaagcactctactatcgtagctaaacctaggtcattttaaggattcttgtttatgttctttattgtatgataaaccaagnggngnnggngnngngngannaaaaaaaaaaaa

SEQ ID NO: 69 - DNA, 1518 bp, Camptotheca acuminata
atggatcttctcctggtagagaagaccctcttggcactatttgctgccattgttcttgctatcaccatctctaaactgcgtggtaagcgctttaaactccctccgggtccactacccgtacccgtttttggcaactggctccaagtcggcgatgacttgaaccatcgaaacctcacggatttggctaagaagttcggtgacatgttcttgctccgtatgggccaacgcaaccttgttgtggtttcgtctccagaccttgccaaagaggtgttacacactcagggtgtcgagttcgggtcccgaacccgaaacgttgtattcgatattttcaccggaaaggggcaggacatggtgttcaccgtttatggtgagcactggaggaaaatgagacgcatcatgaccgtccctttcttcaccaacaaggtggtccagcagtaccgttatgggtgggaggaagaggcggcgcgcgtggtcgaggatgtgaagaagatgccggaggcattgacgacggggattgttttaagaaggcggttgcaactaatgatgtacaacaacatgtaccggatcatgttcgataggaggttcgagagtgaggacgacccgttgtttgtgaagcttaaggctttgaacggagagaggagtcgattggctcagagctttgagtacaattatggtgatttcattcccattctgaggcctttcttgagaggttatttgaagatctgtaaggatatcaaggagagaaggcttcagctctttaaggactattttcttgacgaaaggaagaagctgacaagcacgaaaggcatggacaactatggcctaaaatgtgccattgatcatattcttgaggcccaacagaagggagagatcaacgaggacaatgttctttacattgttgagaacatcaacgttgccgcaattgaaacaacattgtggtcgatcgaatggggcattgcagaactcgtcaaccacccagaaatccagcagaagctgcggcatgagattcaaactgtgctgggacctggaacccaagtcaccgagcctgaagtccaaaaattgccttatctccaagcagtagtcaaagaaacccttcgactccggatggcaattcctcttctggtgcctcacatgaaccttcatgatgcaaagctcggagggtatgacgtgccagccgagagcaaaatcttagtcaatgcctggtggctcgccaacaaccctgctcactggcagaagccagaagaatttaggcccgagaggttcttggaagaggagtctaaggttgatgccaatggcaatgacttccgataccttccatttggtgtcggaagacgaagctgcccgggaattatcctagccctgccaattcttggcattactttgggacgtttggtgcagaatttcgagctcttgcctccacccgggcagtcaaagatcgatacctcggagaagggtgggcagttcagtctgcacattttgaagcattccaccattgttgcaaaaccaatatcattttga

SEQ ID NO: 70 - DNA, 1727 bp, Arabidopsis thaliana
gccgacgattttctcaccggaaaaaaacaatatcattgcggatacacaaactataatggacctcctcttgctggagaagtctttaatcgccgtcttcgtggcggtgattctcgccacggtgatttcaaagctccgcggcaagaaattgaagctacctccaggtcctataccaattccgatcttcggaaactggcttcaagtcggagatgatctcaaccaccgtaatctcgtcgattacgctaagaaattcggcgatctcttcctcctccgtatgggtcagcgaaacctagtcgtcgtctcctcaccggatctaacaaaggaagtgctcctcactcaaggcgttgagtttggatccagaacgagaaacgtcgtgttcgacattttcaccgggaaaggtcaagatatggtgttcactgtttacggcgagcattggaggaagatgagaagaatcatgacggttcctttcttcaccaacaaagttgttcaacagaatcgtgaaggttgggagtttgaagcagctagtgttgttgaagatgttaagaagaatccagattctgctacgaaaggaatcgtgttgaggaaacgtttgcaattgatgatgtataacaatatgttccgtatcatgttcgatagaagatttgagagtgaggatgatcctcttttccttaggcttaaggctttgaatggtgagagaagtcgattagctcagagctttgagtataactatggagatttcattcctatccttagaccattcctcagaggctatttgaagatttgtcaagatgtgaaagatcgaagaatcgctcttttcaagaagtactttgttgatgagaggaagcaaattgcgagttctaagcctacaggtagtgaaggattgaaatgtgccattgatcacatccttgaagctgagcagaagggagaaatcaacgaggacaatgttctttacatcgtcgagaacatcaatgtcgccgcgattgagacaacattgtggtctatcgagtggggaattgcagagctagtgaaccatcctgaaatccagagtaagctaaggaacgaactcgacacagttcttggaccgggtgtgcaagtcaccgagcctgatcttcacaaacttccataccttcaagctgtggttaaggagactcttcgtctgagaatggcgattcctctcctcgtgcctcacatgaacctccatgatgcgaagctcgctggctacgatatcccagcagaaagcaaaatccttgttaatgcttggtggctagcaaacaaccccaacagctggaagaagcctgaagagtttagaccagagaggttctttgaagaagaatcgcacgtggaagctaacggtaatgacttcaggtatgtgccatttggtgttggacgtcgaagctgtcccgggattatattggcattgcctattttggggatcaccattggtaggatggtccagaacttcgagcttcttcctcctccaggacagtctaaagtggatactagtgagaaaggtggacaattcagcttgcacatccttaaccactccataatcgttatgaaaccaaggaactgttaaactttctgcacaaaaaaaaggatgaagatgactttataaatgtttgtgaaatctgttgaaatattcccttgttttgcttttgtgagatgtttttgtgtaaaatgtctttaaatggttcgttctacgattgcaataataattagtggtgctcatt

SEQ ID NO: 71 - DNA, 1521 bp, Ruta graveolens
atggatctcctcttactggagaaggccctcctaggcctcttcgccgccgcggtcgtagcgattgctgtttctaaactccgaggcaagcgcttcaaactcccgccggggcccttcgggttcccggtttttggaaactggcttcaagtcggcgatgacttgaaccaacggaaacttgccaatttatccaagaaattcggagatgtataccttctccgcatgggccagcgcaatctcgtcgtcgtttcgtcgccggaaatggccaaggaggtgttgcatactcagggagtggagttcggctctcggacgagaaacgtcgtcttcgatatcttcaccgggaaaggccaggacatggtgttcacggtttacagtgagcactggcggaagatgcggaggatcatgaccgtccctttcttcacaaacaaagtcgtccagcagcagagatttaactgggaagacgaggcggccagggtcgtcgaggatgtgaagaaagacccccaggcggcgaccactgggatcgttctgaggcggcggctgcagctcctgatgtacaacaacatgtacagaatcatgttcgataggagattcgagagcgtcgacgatcctttgttcaacaaattgaaggccttgaatggcgagaggagccgattggctcagagcttcgagtacaactacggtgatttcattcctattttgaggcctttcttgagaggttatttgaagctggtgaaggaagttaaggaaagaagactcaagcttttcaaggactattttgttgaagagagaaagaaattaacaagcacaaagagcatgaccgaggaaaacttcaaatgcgccattgatcatgtcttggacgctcagcagaagggagaaatcaacgaggacaacgttctgtacattgtcgagaacattaatgttgcagcaattgagacaactttgtggtccatcgagtggggtattgcagagttggtgaatcatccagacatccagaagaagctccgtgctgaaattgacagagtcctcggtcctgaccatcaaatcaccgagcctgacacccacaagctcccctaccttcaggctgtgatcaaggagactctccgcctcaggatggcaattcctcttcttgtaccacacatgaaccttaacgatgctaagcttgcaggctacgacattccagctgagagcaagatactggtaaacgcatggtggctggccaacaaccccgctcactggaaagacccgcaagtattcaggccggagaggttccttgaggaggagtctggggttgaggctaatggaaatgacttccgatacattccttttggtgtcgggagaagaagctgtcctggaattatacttgctttgccgattctcggaatcactattgggcgtatggtgcagaactttgagctgttgcctcctccaggacagtcgaagattgatacttcagagaaaggtgggcagttcagtttgttcattctgaaccactccacgattgtgctcaagcctagatcttctgtctaa

SEQ ID NO: 72 - DNA, 1521 bp, Glycine max
atggatctcctccttctggaaaagaccctcataggtctcttcctcgctgcggtggtcgccatcgccgtctccaccctccgcggccggaaattcaagctcccaccgggcccactccccgtcccaatcttcggcaactggctccaagtcggcgacgacctcaaccaccgcaacctcaccgatttggccaaaaaattcggtgacatcttcctcctccgcatggggcagcgcaacctcgtcgtggtttcttcccctgagctcgccaaagaggttctccacacgcagggcgtggagttcggctcccgcacccgcaacgtcgtcttcgacatcttcaccggaaagggccaagacatggtcttcaccgtctacggcgagcactggcgcaaaatgcgccgcatcatgaccgtccccttcttcaccaacaaggttgtgcaacaataccgccatggatgggaatcggaggctgccgccgtcgtcgaggacgtcaagaaaaaccccgacgccgccgtctccggcaccgtcatccgccgccgccttcagctcatgatgtacaacaacatgtaccgcataatgttcgaccggaggttcgagagcgaggaggatcccatcttccagaggctaagagccttgaacggagagaggagtcgcttggcgcagagctttgagtataactatggtgattttattcccatcttgagacccttcttgaagggttacttgaagatttgcaaggaggtgaaggagacgaggttgaagcttttcaaggattacttcgttgacgagaggaagaagcttggaagcaccaagagcaccaacaacaataatgaacttaaatgcgctattgaccacattttggatgcccagagaaaaggcgagatcaacgaagacaacgtcctctacattgttgaaaacatcaacgttgctgcaattgaaacaactctatggtcgattgagtggggcattgctgagcttgtgaaccacccagagatccagcaaaagttaagggatgagattgacagagttcttggagcagggcaccaagtgactgagccagacatccaaaagctcccatacctccaagcagtggtcaaggaaactcttcgtcttagaatggcaatccctctccttgtaccacacatgaacctccacgacgcaaagcttgggggctatgatatcccagctgagagcaagatcttggtgaatgcatggtggctggccaacaaccctgcacactggaagaagccagaggagttccggcctgagaggttcttcgaggaggagtcgcttgttgaagccaatggcaatgactttaggtaccttccctttggtgttggcagaagaagctgccctggaatcattcttgcattgccaattcttggcatcactttgggacgtttggtccaaaactttgagctcttgcctccccctggccagtcacagattgacactagtgagaaaggagggcaatttagcttgcacatactcaagcattccaccattgtggcaaagccaaggtcattttag

SEQ ID NO: 73 - DNA, 1611 bp, Citrus sinensis
atggcaaatcttgttacaatttcattctttagcatccttctcacaatctcactgctttcgttcaacaaatctttaaatcttatatcaatcactctccctcttgttcctcttattgcatacgttttgaaatcctttttaaaatcttcgaaagccttttaccctccaactcctatctctatcccaatatttggcaattggctccaagttggcaatgaccttaaccacaggttactagcatcaatggcacaaatttacggccccgtattccgtctaaaacttggttcaaaaaatttaatagtggtatcagagccagacctagctacccaagtactacacacgcaaggtgtagaattcggatcccgcccacgcaacgtggttttcgatattttcacgggcaatggacaggacatggtgttcactgtttatggtgagcattggcgcaaaatgcgtaggattatgacactgccatttttcaccaataaagttgtgcacaattacagtgacatgtgggagcaggaaatggacctagtggtgcatgacttgaaaaatgattatgagagtgtgagcacaaaagggattgttattaggaagcgtttgcagctcatgctatacaatattatgtataggatgatgtttgatgcaaaatttgagtcacaagaggatcctttgttcattgaagcaactaggtttaattctgaaaggagtcggttggctcaaagttttgagtacaattatggagattttattcctttgctcaggccatttttaagagggtacttgaacaagtgcagagacttgcagtgtaggaggttggctttctttaacaacaattttgttgagaaaagaaggaaaatcatggctgccaatggagagaagcacaagataagctgcgccattgatcacataattgatgctcaaatgaaaggggagatcactgaagaaaatgttatttacattgttgagaacataaatgtggcggcaatagaaacaacactatggtccatggaatgggcaatagctgagttagtcaatcacccagaggttcaacagaagatccgtcgtgaaatctcgacagtccttaaaggaaatccggtcacagaatcaaacctgcatgaattaccgtacctgcaagccgcagtaaaagaggtactaagattacacactccaattccgttgttggtgccacatatgaatctagaagaagcaaaacttggaggcttcacaattcctaaagagtccaaaattgtggtgaatgcatggtggctagcaaacaaccccaaatggtgggaaaaacctgaggagtttcggccagagagattcttggaagaggaatgtaatattgatgctgttgctggtggtggcaaagttgacttcaggtacttgccttttggcgtgggaaggcgaagctgccctggaatcatacttgcattaccaatcttggggcttgtgattgcaaaactggtgacatcttttgagatgaaagctccacaagggatagataagattgacgtgagtgaaaaaggaggccaattcagcttgcacattgcaaatcattcaactgttgtcttcgatccgataatggaatcactttcccaaccaatgccacagtaa

SEQ ID NO: 74 - DNA, 891 bp, Chromobacterium violaceum
atgaacgaccgcgccgactttgtggtgcccgacatcaccacccgcaagaatgtcggactgagccacgacgccaacgacttcaccttgccgcagccgttggatcgctactctgcggaagatcacgccacctgggccacgttgtaccagcgccaatgcaagctgctgcccggccgcgcctgcgacgagtttctggaaggcctggagcgcctggaagtggacgccgacagggtgccggacttcaataagctcaacgagaagctgatggccgccaccggctggaagatcgtcgcggtgccgggcctgattcccgacgacgtgttcttcgagcacctggccaaccgccgcttcccggtcacctggtggctgcgcgagccgcaccagctcgactacctgcaggagccggacgtgttccacgacctgttcggccacgtgccgctgctgatcaatccggtgttcgccgattacctggaggcctacggcaagggcggggtgaaggcgaaggcgctgggcgctgccgatgctggcgcggctgtactggtacacggtggaattcggcctgatcaatactccggccggcatgcgcatctacggcgccggcatcttgtccagcaagtcggaatccatctactgcctggacagcgccagccccaaccgcgtcggcttcgacctgatgcgcatcatgaacacgcgctaccggatcgacaccttccagaaaacctacttcgtcatcgacagcttcaagcagctgttcgacgccaccgcgccggatttcgctccgctatacttgcagctggccgacgcgcaaccgtggggcgcggcgacatcgcgccggacgacctggtgctgaatgccggcgaccgccaaggatgggcggataccgaagacgtctga

SEQ ID NO: 75 - DNA, 1200 bp, Pseudomonas aeruginosa
atgagtcatttcgccaaggtcgcccgcgtaccgggcgacccgatcctgggcctgctcgacgcctaccgcaacgatccgcgcgcggacaagctggacctcggcgtcggtgtctacaaggatgcccagggcctgaccccgatcctgcgctcggtgaaactcgccgagcagcgcctggtcgagcaggaaaccaccaagagctacgtcggcggccacggcgatgcgctgttcgccgcgcgcctggcggaactggcgctcggcgccgcctcgccgctgttgctggagcaacgcgccgacgccacccagacgcccggcggcaccggcgccttgcgcctggccggcgacttcatcgcccattgcctgcccggccgcggcatctggctgagcgacccgacctggccgatccacgagaccctgttcgccgccgccggcctgaaggtttcccactacccctacgtcagcgccgacaaccgcctggatgtcgaggcgatgcttgctggcctggagcgcattccccagggagacgtggtgctgctgcatgcctgctgccacaacccgaccggtttcgacctgagccacgacgactggcgcagggtgctcgacgtggtgcgtcgccgcgagctgctgccgctgatcgacttcgcctaccagggcttcggcgacggtctcgaggaagacgcctgggcggtacgcctgttcgccggcgaactgccggaggtgctggtcaccagttcctgctcgaagaacttcggcctgtaccgcgaccgcgtcggtgcgctgatcgtctgcgcgcagaacgccgagaagctcaccgacctgcgtagccaactggccttcctcgcccgcaacctctggtcgaccccgccggcgcatggtgccgaggtggtcgcggcaatcctcggcgacagcgagttgaagggactctggcaggaagaggtcgaaggcatgcgctcgcgcatcgccagcctgcgcatcggcctggtcgaagccctggcgccgcacggcctggccgagcgcttcgcccatgtcggcgcgcaacgcgggatgttttcctataccggactgagcccgcagcaggtcgctcggctgcgcgacgagcacgccgtttacctggtctccagcggccgagccaacgtcgccggtctccacgcgcgccgcctcggccgcctggcgcaagccatcgcccaggtctgcgcggactga

SEQ ID NO: 76 - DNA, 1353 bp, Godia cydonium
atggatattgagcctccagctacaaagaagagcaaaatggacagcaatggagaagcctcctacattcctgtacagactccaacaggcgaaaacagtgcaaacctgtctttgatattttctctcaaagatgagcaaggatctctagtcacgtcattgaagcctttccaggatatgggtatcaacatgacccacttggagtcgagaccttccaagtctaacccaggctctgagtatgacttctatgtcgactgtgtgtgccctccagacaagaaagaagatcttctctcttctctcagagccaactcactcactgtcaatatcctctccagggaccctggagaggatgaagtgccttggtttcctcgtaagattgctgaaattgaccggtttgccaaccaagttttgtcctatggagctgagttggactctgaccaccctggtttcactgatgcagtgtatagagcaaggaggaagcagtttgcagacattgcatttcactgcaagcatggtcaacccataccaagagtggagtacacacctcaagagattgacacatggcgtacgatattcacgaaccttgtggacctctttccaacgcatgcctgcaaagaacacaaccatgtgttccctctcttgcaagagaactgtggatacagggaagacaacatacctcaattggaggaagtgtcccagtacctccaatcctgtactggattcagactgagacctgtggcaggtcttctgtcctcacgagacttcttggctggtctggcctttagagtgtttcactccacacagtacatacgtcactactctcagccaaactacacaccagaacctgatgtgtgtcacgagctcattggacatgtccggtgttctgtgatcctctttgcacagttttctcaggagatcggattggcttccctcggagcaccagaggagtacgtacaacaactggccacgctgtactggttcacgatagagtttggcctttgtaaacaagatggacagacaaaggcttacggagctggtctaatctcatcttttggagagttacagtactgtctgtcagacaaacctgaagtccgtcctctagatcctttcaaaacttctcttcaaacataccccatcacagagatgcaacctgtctactttttggccaacagttttgaggatgccaagcagaagctcatggagtttgcccgtaccattcctcgtcctttctctgtgcgttacaacccgtacactcagagtgtggacattataaaggacaagagctccgtacagaccttggtcaatgacatcagatatgaggtggacatactccaggacgccctacgtaaacttgactaa

SEQ ID NO: 77 - DNA, 891 bp, Xanthomonas axonopodis
atgactaccgctccgcaacgcgtcgaaaaccagctcaccgacaagggctatgtcccggtctacaccactgcggtggtggagcagccgtgggacggctacagcgccgacgaccacgccacctggggcaccttgtatcggcgccagcgcgagctgctggtcggtcgcgcctgcgaggagttcctgcaggcgcaggatgcgatgggcatgggccagacgcacatcccgcgcttcgatgcgctcaatcgcgtgctgcaggcagccaccggctggaccctggtcggcgtgcagggcctgctgccggagctggatttcttcgatcacctggccaacaggcgcttcccggtgacctggtggatccgccggcccgatcagatcgattacatcgccgaacccgatctgttccatgacctgttcgggcatgtgccgttgttgatgaacccgctgtttgccgacttcatgcaggcctatggccgcggcggggtcaaggcgcacggcatcggcccggacgcgctgcagaacctcacccggctgtattggtacacggtggaattcggtctgatcgacacgccccagggcctgcgcatctacggtgccggcatcgtgtcgtccaagggcgaatcgctgtattcgctggaatcgccggcacccaaccgcatcgggttcgacctgcaacgcatcatgcgcacgcgctaccgcatcgacagctttcagaagacctatttcgtcatcgacagctttgcgcagttgatggaagccactgcgccggacttcaccccgatctatgccgagcttgcacaacagccgcagatgccggcgggtgacgtgctgccgggcgatcgggtgatccaacgcggcagcggcgagggttggagccgcgacggcgacgtgtga

SEQ ID NO: 78 - DNA, 891 bp, Xanthomonas campestris
atgaacacagcgccgcgccgcgtcgagaaccagctcaccgacaagggctatgtgccggtctacaccaccgcggtggtggagcagccgtgggatggttacagcgccgacgaccatgccacctggggcacgctgtaccggcggcagcgcgcgctgctggtcgggcgggcctgcgatgagttcctgcaggcgcaggacgcaatgggcatggacgacacccagattccgcgcttcgacgcgctcaacgcggtgctgcaggcgaccaccggctggacgctggtcggtgtggaagggctgctgccggagctggatttcttcgatcatctggccaaccggcgcttcccggtgacctggtggatccgccgcccggaccagatcgactacatcgccgaaccggacctgttccatgatctgttcgggcacgtgccgctgctgatgaatccgctgtttgccgacttcatgcaggcctatgggcgcggtggcgtcaaggcgcacggaattggcccggacgcgttgcaaaatctcacccggctgtactggtacacggtggaattcggcctgattgccacgccgcaggggttgcgcatctacggtgcgggcatcgtctcgtccaagggcgaatcgctgcattcgctggaatcggcggcgccgaaccgggtgggcttcgatctgcagcgggtgatgcgcacgcgctaccgcatcgacagtttccaaaagacctacttcgtcatcgacagctttacgcagctgatggacgccaccgccccggatttcaccccgatctatgccgcgctggcgcaacagccgcaggtgccggccggcgaggtgctggcaaccgaccacgtcctgcagcgcggcagcggcgaaggctggagccgcgacggcgacgtgtga

SEQ ID NO: 79 - DNA, 900 bp, Nocardia farcinica
ctagcctcgcgagccggacccgacgcgccgcgcgcgcagcggggtctcgtcgtccatctccgcgaagaacccgccgatcacgtcctcgatctggccgatcgactccgcgcagtacagcaccggctgatagtgcgtgatgtcgtagggttcggtgcccatcgcgaccacgtccagcggacgcagccgggcccgccggaactcctcgatctcgccgaacgaggacagcagacccgctccgtaacagcggatctcgccgcgttcccggaccaccccgaactccatcgagaaccagaacacgtccgccaggaacttcagcgccgcctcggtacgcaggcgcgccaccgccgcgccgacggtccggtagatcgcggcgaaccgcgggctggcgatctggttcgcgtgcccgatgatctcgtggatggcgtccggctcgggggtgtacagcggtgcggaatggtggcggatgtactgggtggagtggaacaccgactcggcgaaggagccgaagaactcccgcagcggcaccagaccggcggcggggacgtaacggaacccgctgagcggggccagcgccgcgctcacctcgtcgagctgcgggatgtggtcggtgggcagggcgaggcgctcggcggcggccagcacctcggcgctggcgtaggtgcggtgcttgcgcgccagctcggtcgagaccatccgccagacccgctgctcctcctcggtgtaatcgacccggggaagggccgcgccgggcgtgtaacccagcgccagcgcggcgatggcgttgcgccgcgcccggtagtccggatcgcgcacgccggggtgctcgtcgctcaggtgcaccgtcaccgcaccgtctcggtcacgggtcaccggcgagtacagctgcgcttcggtgaacat

SEQ ID NO: 80 - DNA, 1341 bp, Gallus gallus
atggatgcacagcactgcaagatgaatggagactccttccaggaatccacatacactgaagagccctcgaacaaaaatggtgtgatttctttgattttctctttgaaagaagaagttggggcactggctaaagttctgcgcacatttgaggaaaaaggcataaatctgactcacattgaatctcgaccttctcgtctcaataaagatgagtatgaattcttcattaacttggaaggcaagaatgtcccagcactggacaagatcatcaagtccttaagaaatgatattggagtaacagtgcatgagctttcacgaacaaaaaagaaggacactgttccctggttcccaagaagtatccaggagctggacagatttgccaatcagatcctaagctatggagcggagctggatgctgaccatcctgggttcaaagatcctgtgtaccgggcccggaggaaggagtttgcagacatcgcctacaactacagacatggtcaacctattcctcgagttacctatacagaagaagaaaagaaaacttggggcaccgtattcagagagctgaagaatctctatccaactcatgcttgttatgaacacaaccatgtgttcccactgctggagaagtactgtggctaccgggaggataacattccccagcttgaagatgtttcaaagttcttgcagacctgcactggatttcgcctgcgtcctgttgcaggcttgctctcctctcgggatttcttggctggactggcattccgagtatttcactctacacagtatattcgccatgcatccaaacctatgtacacaccagagcctgatatttgccatgagctactaggacatgtgcctctttttgctgatcccagttttgctcagttttcccaggaaattggactggcatctctgggagctccagatgatttcatcgagaaacttgctacggtttattggtttactgtggaatttggactgtgtaaggaaggagattcactaaaggcatatggtgctgggctgctgtcttcatttggagagctgcagtactgtttatcaggtaagcctgagattcggcctctcgttctcgaaaacacttctgtgcagaagtactctgttactgagttccagcctacctactttgttgctgaaagttttaatgatgcaaaagaaaagctaaggaaatttgctcaaacaattcctcgtcctttctctgttcggtacaatccctacacccagaggatcgaagtcttggacaatgcaaagcagctgaagaacttagctgacactatcaacagtgagatggggatcctctgcaatgccctccagaagatcaaatga

SEQ ID NO: 81 - DNA, 6702 bp, Saccharomyces cerevisiae
ttatttcaaagtcttcaacaatttttctttatcatcggtagataacatcttgataacttcagataatccatcaatagcattgtcatggtcgcttctgatctttttagctaagtcttgagcgaatgactctaatttcaaaccctttagtttatcgtccaaagttttgtagttttcttcaatccatgttgcgacttgcctatcatcttcatggtccactgaagcagggtaccacgatctaattcttgcgatcttttctaatcttgatgcttcgcctacctgatggctcaaccttttaatcaaatattcttcgttcaatcttcttctcaatctccagaagaagaaacgacgtgcctcggtccattccagttccttagaaataacacccttggccaccatacgtgaagacctatcgtgcaaatcagcaaattgaagactgatttgtccgtaaattggcaatagttctctctcacgatcagctaattgcttggatatttgctgatgtacttctggagccaaactcttgttggataattgagatctcaattctctgtacttgtcatccaatctgttcatggtgtccagcaatttttctctacggaacttgataccaaccataccttgtggttccaaaacaccagctctagcgttgacgtcggcatacatttccatttggtcagcgttgatagttggatcgacaacaacccatgaaccacctcttagttcaccggtaggtgggatatagataataattggttgtttgtaatccaccaatgcgtcaacaataaacgaaccatacttcaagacttcgttgaacatatcacgttgaccaccagagaaacctctccagttggccaaaatcatcattggcaattgttcaccgttgttaaagtcattgatagcttgagcagtcttgaaggcggagtttggatgccaaacttgaccaggttcttgaattaatgtttcagcactatttggattagctggatcagcaggaatcaagttctcgacagttcttgtttcaacaccaataacacccagtggaataccaccaagacgggctctaccaacgacaacacctttggcccatcctgacaaagtttcaaagaaagaccctttatcaaacaaaccatattcaaatccactttcagtctcacgaccttcaatcatccatcttacatcgtaagtttcatcattagttggagtgaaatcaactggtctatcccatgtgtctttagtttccaagataggaactggcatattacgcttggctggaacataagacatccattcaacaatcttctctacaccagctaaatcgtcaacagcagtcaaatgtgaaacaccgttgttatacatgatttgagtaccacccaattgtaagttagaagtataaacttctctacccagcattttgttgattgcaggagcaccagttaaaataattggctggccttcgacctgaatagctctttgacccaaacgaaccaaataagcaccgataccgacggatctacaagtgactaaggtgatagtgaagatatcgtggtaagcccttgacgttgcaccagcaattaaaccagatccacgtagacattcgacacctaacccatcttcagaaccaataattgtcttgatgacaaatctttcttcaccgtttataacagtacgttcagtgagaacagaattttctttgtcaaatttctttaaagtttccataccttcacttgttaagtataagtattggaagcccttgtccggattggcagcatcattccatgcaacttgaaatagtggaacaatctcttcagccataccaattctggcacctgagtttgcagccaagtaaattcttgggataccacgctttctagcatattcagtaaccttattgaagaattcgtcttcttgtggaccaaaggaaccgatcttgaatgtgatatcgttagcaacaacaacaaattgacggcctcttggatattcaggagtctttacagtaatcttaaaggcaaccataccaatagcgttggcaccaggttctctttccacctcagttaattcgccgttttcatcttcaatcaactcgttggaaataaagaaatcatctgttaacttaacatctgcagagaaatttttccattgggatgacgatgcttggcggaataattctgggaagtcatagacatatgtggtacccatcaagtgtgccttataacgttttggttgcaaccattccttaacagggtaaggagtagcaataggtcttaaatgcatggatccaggtttacccaaagacttaaatacccattcaccttttgcgttcttgacttcggtgtacatttctgttttgataacataaccagaaacgttattgatcaaggcacgcaatggtactggggcacctgtttgaggatctttgatgatgattctaatttcggcagaagaaacacgcaatctcaacaatctcttaccaaatctttctaagaaaccaccgaaggcggcttcgacatcttctggagagatatcaaacaccgcaatgaagttgatgaagatatgattcaaatcagaatttgaagtgtcggtgacttctaaattatccaatatatcactcatcaatctgttagcttcagaagtcagatattcttgaatagaaatgtcatcacggatatgacccgttctaataatacctcttgtaaagaatctcttatccaatggagaagtcttactaacagcttcgtagacatggatgtttctattatcagtgaaaattggtttaatgttgaagttggacaatcttcctaattccagttggaaggccaaagccggctcaatgtgacgaattgtttcattttcgttataatttggaccgttaaaagtataatactttggataagacccatctttaaaaccgaacataaatgtgatacgacggatagaagcattgattaattcctgcttattcaaatccaaaatttctctcaaccttaccaaaatttcctcttcagattcgaaaccttctgtagaagcaacacaaacattagcaacattactcaacgatgcggagctaccagaacgatcaggagcaggtccgttagaagaagattggtgacgaggaataacttccaaactttgtgacaaaatttcatcaacatcatctaaatgatccacagccatcaaaataccttctcttaacggagatgactgactgtttgcaacatatgacaaatctgaaacagaaacagccctgttcatacccattttagatttaacagttggaaaggtggagaacgcagctgaaggtagttggaatttccattcaacaattggaactgtgacaccttcgtgaactctaatatctcctatggtgtaagcacgataagcacgacgaatatagacttgagcagctgcagcagtcacaactgggtcttgatgggttaggaattgaagtaaaacatcgaacacaacgtaattagaatcgatcaagtccttcaagatattcaaatctggttcagagcgctttggattggatgagccataggcaaccttcacaacagaggattttaagatatgttcaatttgttcagttctttccttgaccgaaggtaaagcgccttgaatcaaaatttctcttgcttgtagagcgaccttagcggtagccttagattctagttcaacaatatgttgtagaggagtagagaaaatggcagaaactttagaagataacttgcacaatggttgataatgtttcaagatagctaggatcaggttattcttcgctgaaactttcgaatgagacaaaacagttagcgcaactttatctagatctttagggttttcatcacgcaatttcagaatgatattttcctcacgaacatttggaccattgaataacttttcaacttcgtaatattcttccaagaaatggacaaatatagaatgttcatgggcttctaacccgttagagtacttatgagcaatatccgccaatggttccacgacggcgcccagcaatttgtcggggttgtattcaggattcttcacggccatatcaatcaatttacttaattgtctagctgggaaaacagcaccacgtctcaaagaacgtgcaactaactcttccatttgttcatctagcttagcaggcaatcttgaatgtaaagcagagatgtgtagtttccattctgagtaaggcagttttggatttctcaaaacctctatcaattgttgcaaggaagcgttcataataacttggttgtcataacccttcaaaatgttttccaaagtagacactaatgacttgaatttataggcaggtttggttccttcgataactggagaaccaaaatctggcagcataccttcaaatggtagagcgtgcttgaccttggatggatcgtcaagagtcataatagccatgatatcacctgcaacaatggtagaaccaggttgctttaataactggacgataccattttcttgagaaaccaaaggcatttgcattttcataacttcaatttctgcatatggttggcccttgataatgtgttcaccattttccaccaagaatttaaccaatttaccaggggatggagtacgcaactgggttggatcgttttcaacttccaacaaagtagtcatagagtcaacggataatcttgtagcagcaacttcttctttccaatagatggtatgcgatttaccgcctatggcaatcaaaagaccaccatcagatagttgacgcagtatgatatcacatttagaaccattgataaataatgtgtaacggtcattaccggatttagctacggtgaacttgtatcttttaccctcatggataaaatctacagggaacatagtttgcagtaggtctttagatagaacttgtcccttttgtaaggattcgatatacttgtggcgggcttcttcagatgctaagaaagcctttgtagcggcaccgcaaatgacggcaagagttggatcaggcttttcagcggtcattttatgagtaatcaaatcgtccaaccaaccggtggtaatagtgttatcctcgaaatcttcagtttccaaaagtttgatcaagtattccacagtagttctgaaatcacccctaatggacaattccttcagggcaacaaccatgtgtttcctggaagcttgtctattttcaccaaaagcaaaaatatggccgaactgagagtccgaaaaggagtgaatattaccattgttacccacggagaagtaaccccaaacattagaggaagaacggaagtttagttcatgcaaagtaccacccgatggcttgaatccatcgtttggatcttctgatgtgatacgacaagcggtacaatgaccctttggaataggtcttctttgtttcttggtggcatcttgagttttgaattcgaaatcgatttctgaggcagaatgaggattcataccatataaagttctaatgtcacttattctatgcatagggatacccatagcgatttgtaattgagctgcaggtaagttaacaccggagaccatttccgttgttggatgctcgacttgtaatcttgggttcaattctaaaaagtagaattttccatcatcatgagaatatagatactccacggtaccggcagagacataaccgactagtttccccagtctgacggcagccttttccatctcgtgaaatgtttcagccttggcaattgtaactggtgcttcttcgataattttttgatgacgtctctgaacggaacagtctctaccgaacaaggaaatatttgtaccgtactgatctgctagcagttgaacttccaagtgacgcgctctaccggccaacttcatgatgaaaatgggggagcctggaatttcgttggctgcctggtggtataaagcgatgaaatcttcttcacgttcaacttgtctgatacctttaccaccaccaccttcggatgccttaatcatgacaggaaaaccaatacgcttggccttttgtaaaccatcttcaggagaggtacaacaacccttttgatagatgtcatcgtcgacagagaccagaccggttttctcgtccacgtgaacggtgtcaacaccggtaccagaccatggaatacatgggactttagcactttgagcgacaatggtagaggagattttatcacctaaagacctcatggcgttacctggaggcccaataaagatgactttcctcttagactgggacaatttttcaggcaatagtggattctcggaggcgtgaccccagccagcccatacggcgtctacgtctgctctttcggcgatgtctacgatcaagtctacgttagcgtagttgttattattagtaccacctggcacttcaatgtattgatcggccatacggatatattctgcgttggcctccagatcttctggggtggccatggcgacgaattggacggttctgtcatcgccgaacgtctcgtatgcccattttctgacggatctaatttctttcacggcggcaataccattatttgctatcaggatcttggatatgaccgtgtgaccaccgtgactcttaacaaagtcccttaacggggactcctctagtttatctactgtattgaggccaatgaaatgacctggaagttctgtatgtctttctgagtagtttgtaatttcgtactccatcttctgtggagaagactcgaataagctttcttcgctcat

SEQ ID NO: 82 - DNA, 8413 bp, Saccharomyces cerevisiae
gtcgacatgcattccaccaggacctgatattatgttattgatgtttggctcgaaacctaaactctgcttcaacgatagttccttaggcttggttgtatcttggccgcccacggcagcattcgcatcatcggtaggtgtcttatctccaaaaagccacgacatgattgtgtgtgatctgttaaacaagtatacctatattatcttggttattttttttttttctatctgcttttgttaacgctataacgtgtagtatgtacaggcaaagagagtagaagaggaaaatggtctttttttttttttttttctgcaatggagggcgaatgcaataacctattatttctattaattaaacgcaacaaatgtttcccttttgctctacgtaaaggttcctttctctcttttttttgtcggtgtcttttttttttcagtattttctctttttttcaatgaatcgtcgatttccttttcttccttttgcgattaaattatttttacccagctttagcaagccagttcgtacgcagcgactagcaaacagccgggtaactcacattttgtttgcacacttaaaatacccatacagaaccattatatatgttgggttgaattgggacctaatgtgctgctcaggtgccgcgtatatcatgacacttatacttggtggggaatcgcccgtcaggcctgaacgcaacgaacccgcgcatgcatcgacgtcacagtgagctcaggccgcatcacggctgtacgccctccagagtcaccacgactgcgactagtatcatccgtcaagaagaacaagaacaagaacaagaacaagaacaacaaactccgggcacatctctcggcttcagtcgctttcgctcattgcctgtaggttggcccgatatgcgttgacgttatccaaaggggaatgcttcatcttgttgaacaacgcccaacaatttccactgcccaccgaatcgttgcgcccgttaaaatcttcacatggcccggccggcgcgcgcgttgtgccaacaagtcgcagtcgaaattcaaccgctcattgccactctctctactgcttggtgaactaggctatacgctcaatcagcgccaagatatataagaagaacagcactccagtcgtatctggcacagtatagcctagcacaatcactgtcacaattgttatcggttctacaattgttctgctctcttcaattttcctttccttattctactctttttatccctttcgtacagtttacctgaagataaaaaacaacaaagccaattccctaatttgcaatcgccatttgcatctatatatatatatttgttgtgccatttttttatcctctgtgagtgatcggtgcatgtgtttataaaagtttattcattctactatacgaacttttccctctgcccttccctcccgcttcatccttatttttggacaataaactagagaacaatttgaacttgaattggaattcagattcagagcaagagacaagaaacttccctttttcttctccacatattattatttattcgtgtattttcttttaacgatacgatacgatacgacacgatacgatacgacacgctactatactatacaaatataatagtataataaccgattcgtcttctagcttaatttttttccgttcccgaaacagcgcagaaaattagaaaaaatcaagtttctaccatgagcgaagaaagcttattcgagtcttctccacagaagatggagtacgaaattacaaactactcagaaagacatacagaacttccaggtcatttcattggcctcaatacagtagataaactagaggagtccccgttaagggactttgttaagagtcacggtggtcacacggtcatatccaagatcctgatagcaaataatggtattgccgccgtgaaagaaattagatccgtcagaaaatgggcatacgagacgttcggcgatgacagaaccgtccaattcgtcgccatggccaccccagaagatctggaggccaacgcagaatatatccgtatggccgatcaatacattgaagtgccaggtggtactaataataacaactacgctaacgtagacttgatcgtagacatcgccgaaagagcagacgtagacgccgtatgggctggctggggtcacgcctccgagaatccactattgcctgaaaaattgtcccagtctaagaggaaagtcatctttattgggcctccaggtaacgccatgaggtctttaggtgataaaatctcctctaccattgtcgctcaaagtgctaaagtcccatgtattccatggtctggtaccggtgttgacaccgttcacgtggacgagaaaaccggtctggtctctgtcgacgatgacatctatcaaaagggttgttgtacctctcctgaagatggtttacaaaaggccaagcgtattggttttcctgtcatgattaaggcatccgaaggtggtggtggtaaaggtatcagacaagttgaacgtgaagaagatttcatcgctttataccaccaggcagccaacgaaattccaggctcccccattttcatcatgaagttggccggtagagcgcgtcacttggaagttcaactgctagcagatcagtacggtacaaatatttccttgttcggtagagactgttccgttcagagacgtcatcaaaaaattatcgaagaagcaccagttacaattgccaaggctgaaacatttcacgagatggaaaaggctgccgtcagactggggaaactagtcggttatgtctctgccggtaccgtggagtatctatattctcatgatgatggaaaattctactttttagaattgaacccaagattacaagtcgagcatccaacaacggaaatggtctccggtgttaacttacctgcagctcaattacaaatcgctatgggtatccctatgcatagaataagtgacattagaactttatatggtatgaatcctcattctgcctcagaaatcgatttcgaattcaaaactcaagatgccaccaagaaacaaagaagacctattccaaagggtcattgtaccgcttgtcgtatcacatcagaagatccaaacgatggattcaagccatcgggtggtactttgcatgaactaaacttccgttcttcctctaatgtttggggttacttctccgtgggtaacaatggtaatattcactccttttcggactctcagttcggccatatttttgcttttggtgaaaatagacaagcttccaggaaacacatggttgttgccctgaaggaattgtccattaggggtgatttcagaactactgtggaatacttgatcaaacttttggaaactgaagatttcgaggataacactattaccaccggttggttggacgatttgattactcataaaatgaccgctgaaaagcctgatccaactcttgccgtcatttgcggtgccgctacaaaggctttcttagcatctgaagaagcccgccacaagtatatcgaatccttacaaaagggacaagttctatctaaagacctactgcaaactatgttccctgtagattttatccatgagggtaaaagatacaagttcaccgtagctaaatccggtaatgaccgttacacattatttatcaatggttctaaatgtgatatcatactgcgtcaactatctgatggtggtcttttgattgccataggcggtaaatcgcataccatctattggaaagaagaagttgctgctacaagattatccgttgactctatgactactttgttggaagttgaaaacgatccaacccagttgcgtactccatcccctggtaaattggttaaattcttggtggaaaatggtgaacacattatcaagggccaaccatatgcagaaattgaagttatgaaaatgcaaatgcctttggtttctcaagaaaatggtatcgtccagttattaaagcaacctggttctaccattgttgcaggtgatatcatggctattatgactcttgacgatccatccaaggtcaagcacgctctaccatttgaaggtatgctgccagattttggttctccagttatcgaaggaaccaaacctgcctataaattcaagtcattagtgtctactttggaaaacattttgaagggttatgacaaccaagttattatgaacgcttccttgcaacaattgatagaggttttgagaaatccaaaactgccttactcagaatggaaactacacatctctgctttacattcaagattgcctgctaagctagatgaacaaatggaagagttagttgcacgttctttgagacgtggtgctgttttcccagctagacaattaagtaaattgattgatatggccgtgaagaatcctgaatacaaccccgacaaattgctgggcgccgtcgtggaaccattggcggatattgctcataagtactctaacgggttagaagcccatgaacattctatatttgtccatttcttggaagaatattacgaagttgaaaagttattcaatggtccaaatgttcgtgaggaaaatatcattctgaaattgcgtgatgaaaaccctaaagatctagataaagttgcgctaactgttttgtctcattcgaaagtttcagcgaagaataacctgatcctagctatcttgaaacattatcaaccattgtgcaagttatcttctaaagtttctgccattttctctactcctctacaacatattgttgaactagaatctaaggctaccgctaaggtcgctctacaagcaagagaaattttgattcaaggcgctttaccttcggtcaaggaaagaactgaacaaattgaacatatcttaaaatcctctgttgtgaaggttgcctatggctcatccaatccaaagcgctctgaaccagatttgaatatcttgaaggacttgatcgattctaattacgttgtgttcgatgttttacttcaattcctaacccatcaagacccagttgtgactgctgcagctgctcaagtctatattcgtcgtgcttatcgtgcttacaccataggagatattagagttcacgaaggtgtcacagttccaattgttgaatggaaattccaactaccttcagctgcgttctccacctttccaactgttaaatctaaaatgggtatgaacagggctgtttctgtttcagatttgtcatatgttgcaaacagtcagtcatctccgttaagagaaggtattttgatggctgtggatcatttagatgatgttgatgaaattttgtcacaaagtttggaagttattcctcgtcaccaatcttcttctaacggacctgctcctgatcgttctggtagctccgcatcgttgagtaatgttgctaatgtttgtgttgcttctacagaaggtttcgaatctgaagaggaaattttggtaaggttgagagaaattttggatttgaataagcaggaattaatcaatgcttctatccgtcgtatcacatttatgttcggttttaaagatgggtcttatccaaagtattatacttttaacggtccaaattataacgaaaatgaaacaattcgtcacattgagccggctttggccttccaactggaattaggaagattgtccaacttcaacattaaaccaattttcactgataatagaaacatccatgtctacgaagctgttagtaagacttctccattggataagagattctttacaagaggtattattagaacgggtcatatccgtgatgacatttctattcaagaatatctgacttctgaagctaacagattgatgagtgatatattggataatttagaagtcaccgacacttcaaattctgatttgaatcatatcttcatcaacttcattgcggtgtttgatatctctccagaagatgtcgaagccgccttcggtggtttcttagaaagatttggtaagagattgttgagattgcgtgtttcttctgccgaaattagaatcatcatcaaagatcctcaaacaggtgccccagtaccattgcgtgccttgatcaataacgtttctggttatgttatcaaaacagaaatgtacaccgaagtcaagaacgcaaaaggtgaatgggtatttaagtctttgggtaaacctggatccatgcatttaagacctattgctactccttaccctgttaaggaatggttgcaaccaaaacgttataaggcacacttgatgggtaccacatatgtctatgacttcccagaattattccgccaagcatcgtcatcccaaggaaaaaatttctctgcagatgttaagttaacagatgatttctttatttccaacgagttgattgaagatgaaaacggcgaattaactgaggtggaaagagaacctggtgccaacgctattggtatggttgcctttaagattactgtaaagactcctgaatatccaagaggccgtcaatttgttgttgttgctaacgatatcacattcaagatcggttcctttggtccacaagaagacgaattcttcaataaggttactgaatatgctagaaagcgtggtatcccaagaatttacttggctgcaaactcaggtgccagaattggtatggctgaagagattgttccactatttcaagttgcatggaatgatgctgccaatccggacaagggcttccaatacttatacttaacaagtgaaggtatggaaactttaaagaaatttgacaaagaaaattctgttctcactgaacgtactgttataaacggtgaagaaagatttgtcatcaagacaattattggttctgaagatgggttaggtgtcgaatgtctacgtggatctggtttaattgctggtgcaacgtcaagggcttaccacgatatcttcactatcaccttagtcacttgtagatccgtcggtatcggtgcttatttggttcgtttgggtcaaagagctattcaggtcgaaggccagccaattatttggtatcggtgcttattaactggtgctcctgaatcaacaaatgctggtagagaagtttatacttctaacttacaattgggtggtactcaaatcatgtataacaacggtgtttcacatttgactgctgttgacgatttagctggtgtagagaagattgttgaatggatgtcttatgttccagccaagcgtaatatgccagttcctatcttggaaactaaagacacatgggatagaccagttgatttcactccaactaatgatgaaacttacgatgtaagatggatgattgaaggtcgtgagactgaaagtggatttgaatatggtttgtttgataaagggtctttctttgaaactttgtcaggatgggccaaaggtgttgtcgttggtagagcccgtcttggtggtattccactgggtgttattggtgttgaaacaagaactgtcgagaacttgattcctgctgatccagctaatccaaatagtgctgaaacattaattcaagaacctggtcaagtttggcatccaaactccgccttcaagactgctcaagctatcaatgactttaacaacggtgaacaattgccaatgatgattttggccaactggagaggtttctctggtggtcaacgtgatatgttcaacgaagtcttgaagtatggttcgtttattgttgacgcattggtggattacaaacaaccaattattatctatatcccacctaccggtgaactaagaggtggttcatgggttgttgtcgatccaactatcaacgctgaccaaatggaaatgtatgccgacgtcaacgctagagctggtgttttggaaccacaaggtatggttggtatcaagttccgtagagaaaaattgctggacaccatgaacagattggatgacaagtacagagaattgagatctcaattatccaacaagagtttggctccagaagtacatcagcaaatatccaagcaattagctgatcgtgagagagaactattgccaatttacggacaaatcagtcttcaatttgctgatttgcacgataggtcttcacgtatggtggccaagggtgttatttctaaggaactggaatggaccgaggcacgtcgtttcttcttctggagattgagaagaagattgaacgaagaatatttgattaaaaggttgagccatcaggtaggcgaagcatcaagattagaaaagatcgcaagaattagatcgtggtaccctgcttcagtggaccatgaagatgataggcaagtcgcaacatggattgaagaaaactacaaaactttggacgataaactaaagggtttgaaattagagtcattcgctcaagacttagctaaaaagatcagaagcgaccatgacaatgctattgatggattatctgaagttatcaagatgttatctaccgatgataaagaaaaattgttgaagactttgaaataa

SEQ ID NO: 83 - DNA, 6696 bp, Kluyveromyces lactis
atgagtgaggaaaatctttctgaggtttcaatctctcaaagtaaacaatacgaaattactgaatatagcgatagacattccaagttggcttctcatttcattggtctgaacactgtggataaggcagatgattctccattgaaagagtttgtcaaatcacatggtggtcatactgtgatctcaaaggttttgatcgctaacaatggtatcgcagccgttaaagaaatcagatcggttcgtaaatgggcctatgaaaccttcggcgatgaaagaactgttcaattcgtggccatggccactccagaagatcttgaagccaacgcagaatacattcgtatggctgatcaatatatcgaagttcccggtggtaccaacaataacaattatgcaaacgttgacctaattgttgaagttgccgaaagagctgatgtagatgcagtttgggcaggttggggtcatgcttcagaaaacccactactccctgaaaggctagccgcttctcacagaaagattatatttattggtccaccaggaaatgccatgagatctctcggtgataagatctcgtccactatcgttgcccaacacgctaaggttccttgtatcccatggtctggtactggtgtcgatgaagttcatgttgataaagaaactaacttggtctctgtcgaagataaagtataccaagaaggttgttgttcgtctccagaagacggtctaaagaaagccaaggaaattggtttcccaattatggtcaaggcttccgaaggtggtggtggtaaaggtatcagaaaagtcgaaaatgaagatgagttcctgtctttgtaccaacaagctgctaatgaaattcctggttctccaatttttattatgaagttggctggtaaggctcgtcatttggaagttcaacttttggctgatcaatatggtaccaacatctctctatttggtcgtgattgttctgttcaaagacgtcatcaaaagattatcgaagaagctcctgtaactatcgctaagccagataccttcactgaaatggaaaaagcagccgtcagattaggtcaattggttggttacgtttctgctggtaccgtcgaatatttatattctcatgatgaagacaagttctacttcttggagttgaacccaagattacaagttgaacatccaaccacagaaatggttactggtgttaacttgccgtctgcccagttacaaatcgctatgggtattccaatgcacagaatcagagatattagattgttatacggtgtcgatccaaaatctgcatccgaaattgactttaacttctctacacctgagtctgctaaaactcaaagaaaaccaactcctaaaggtcactgtactgcctgccgtatcacatccgaagatccaaatgagggtttcaaaccatctggcggtgctttacacgaattgaacttccgttcttcttccaacgtttggggttatttctctgttggtaataatggtggtatccattcattctctgactctcaattcggtcatatcttcgccttcggtgaaaacagacaagcttcaaggaaacatatggttgttgctttgaaggaattatctatcagaggtgatttcagaactacggttgaatatttaatcaaattattggaaaccgaagacttcgaagacaataccatcacgactgggtggttggatgatttgatttctcagaaaatgacagctgaaaagcctgatagaaccctatctgtcatttgtggtgccgctaccaaggctcatattgcctcccaaaaagccagagaagattacatctcatctttgaagagaggccaagttccaaacaaatcattactacaaacaatgtacccaattgaatttattcatgatggtatgagatatagatttactgttgctaaatcagccgacgatcgttatactctattcattaacggttccaagtgcgaagttggcgtaaggaagttatctgatggtggtttgttgattgccgttggtggtaaatcacacaccatttactggaaggaagaagttgctgctaccagattatcaatcgactctaagacaactctactagaggttgaaaatgatccaacacaactcagaactccatctcctggtaaattggtcaagtttttggtcgaaaacggtgatcatgttattgctggccaaccatatgccgaagttgaagttatgaagatgcaaatgccattgatttctcaagaaaatggtgtcgttcagttattgaaacaaccaggctctactctggccgccggtgacattctagccatcttaactctagatgaccctagtaaagtcaaacatgctaagccttacgaaggcatgctaccagaattgggtgctccaatcgttgaaggtaccaagcctgcatacaaatttaaatctttggtcactactttggaaaacatcttgaagggatacgacaatcaagttattatgaatgcttcattgcagcaattaattgaagtgttgagacaaccagaattaccatactctgaatggaaattacaagtttctgctttacattcaagattaccacctaagttagacgaaatgcaagaacaattggtcacccgttcattcaagagaaatgcggatttcccagcaagacaactagaaaagatgttagaagctgccttaaatgatcctaacgttgacccattgtttagcactaccattgaaccacttgttgatattactacccgttactctaagggacttgctgctcatgaacattttgtctttgccactttcttagaaaactattacaatgtcgaaaaattgttctctgggccaaacattcgggaagaagacgtcatcttaaaattgcgtgatgagaaccctgacgacttggagaaggttgttttaaccgtcctagctcactccagagtatcagccagaaacaacctgatccttgccattttgaagcattatcaaccgctatgcaaattgagctctgaggtagccgctgctatcgaacaaccattgaaacacatcgtcgaattagaatctaaggccaccgctaaggttgctctacaagccagagaaattttgattcaaggtgctttgccatctatcaaggagagaacagatcaagttcaatacattcttaagtcatctgttttaagcacttcatatggttcatccgaaacgaagcgcacaaaacctgatttagaagttttaaaggacttgatcgattccaactatgttgttttcgatgtgttggcccaattcttgacaaatccagatgatgccgtttctgctgctgctgccgaggtctacattagaagagcatacagagcgtacactattggtgatttgaagcatcaaaagtcttctggatcacctgtagttgagtggaagttccaacttccatctgctgcattcacctcattgccacaggttaagagtaaattgggtatgaacagagctatttctgtctctgatttgacttatgtctctgacggtgaaaaccaaccattaagaactggtttgttgattcctgctagacatctagatgatgttgatggtattttgtcgtcagctctatctttaattccttctcatcatatgtctactggccctgtcccagacagatctggctcttcagccagcttgtctaatgttgccaatgttgttgtgtcttcaactgaaggatttgaatctgagtcggatgttttaaagagactcagagagatactcgatttaaacaagcaatcattagttgactctgctattcgtcgtattaccttcgtgtttggatacagtgatggtacatatccaaagtactataccttccgtggtccaaattacaatgaagatgaaacaattcgtcacattgaaccagctctagctttccaacttgaactaggtaagatgtcgaacttcaatatcagacaaatatttactgagaacagaaacattcatgtctatgaggccgttggtaaaaactctccggttgacaagagattctttaccagaggtattatcagaacaggtcgtattagtgacgacatttccatccatgaatatttgacttcagaagctaacagattaatgagtgacattttggacaacttagagatcattgacacttctaactcagatcttaaccatattttcattaacttctctgctgtatttgacatttcgccagaagctgttgaagctgcctttggcggtttcttggaaagatttggcagaagattgctcagattacgtgttgccgctgctgaaatcagaattattatcaaggaccctcaaactggcaccccggttccaatcagagcgttgatcaacaacgtctcgggctttgttgtgaagactgaattgtatacagagatcaagaatgcacaaggtgaatggattttcaaatctttagataaaccaggtgctatgcatttgagacctattgccactccttatcctgcaaaggagtggttacagccaaaacgttacaaggctcatttgatgggaaccacatacgtttacgatttcccagagctattccgtcaagccaccgtggcacaatggaagaaacactctccaaagaccaagttgtcagacgattttttcattgcaaatgaattgattgaagatgaaaatggtgaattaactgaagttgatcgtgaacttggtgctaataacatcggtatggttgcattcaaggttactgcaaaaactccagaatactctcatggccgtcaatttgtcatagtcgccaatgatatcactttcaaaattggttcgttcggtccacaggaagatgccttcttcaacaaggttactgaatatgcaagaaagcgtggtatcccaagaatatacttatctgccaattcaggtgcaagaattggtattgccgaagagcttgttccattgttccagattgcttggaatgatgaaaaagatccagcaaagggtttccaatacttatggttgtcagatgagtctcttgaagaactcaaatctaagggtaaagacaatgctgttgttaccgaatgtgttgttgaagaaggtaaggtcagaaacgtcattactgctattatcggttcggaagatggtcttggtgttgagtgtttgaagggatccggtttaattgcaggtgccacttcaagagcgtacaaggatatcttcacgatcaccttagttacttgtaggtctgtgggtatcggtgcttatctagtcagattaggtcaaagagccattcaaatcgaagcacagccaatcattttaaccggtgctcctgccatcaataagcttcttggtagagaagtttactcttcaaacttgcaattgggtggtactcagatcatgtacaacaatggtgtttcacacttaactgctccagatgatttggctggtgttgaaaagatcatggactggttatcttacattcctgccaaacgtgatctaccggttcctattttggaatctgaagataaatgggacagaaaaattgactatgctccatctttaaacgaacagtacgatgttaggtggatgattgcaggtcgtgaatctgccgatggtttcgaatatggtcttttcgataaaggttccttccaagaaaccttgtctggttgggccaagggtgttgttacaggtagagcccgtttaggtggtattccattaggtgttattgccgttgaaacaagaattgtcgaaaatttgattcctgctgatcctgctaatcctgattctaccgaaatgttgattcaagaagccggccaagtttggtatccaaattccgcgttcaagacagcccaggctatcaatgacttcaatcatggtgaacaattgccattaatgatcctagccaactggagaggtttctctggtggacaacgtgatatgtacaacgaagttttgaagtatggttctttcatcgttgatgcattggttgattacaaacagcctataattacatacattcctccaactggtgaactaagaggtggttcttgggttgttgttgatccaactatcaatgctgaccaaatggaaatgtatgctgatataaactcaagagctggtgttttggaaccagaaggtatggtcggtatcaaataccgtagagaaaaattgttggctactatggcaagattagatgacaagtacagagagttgaaagccaagttggccgattccactttgactccagaagaacatcaagaagtatcaaagcagcttgctatccgtgagaagcaattgttgccaatttaccatcaaattacagtacagtttgctgacttgcatgatagatccggtcgtatgttggcaaagggtgtgatcaaaaaggaattggactggccagaagctcgtcgcttctttttctggagattaagaagaagattaaacgaagaatatttgatgagaagattaaataacgagctaggatctgcttctagactagagaaaatggcaagaatcatatcatggtacccagcttctgtgagccaagataacgacagagaagttgctacttggatcgaagaaaactaccaattcttggatgaacaagttaagagtctgaagttggaagctttcgcacaaaatttggcaaaatctatcagaaacgaccgtgaaaattccatcaatggtttggcggaagttttgaaattattgtctgccaaagacaaagaaaagcttcaaaaagctttggaatga

SEQ ID NO: 84 - DNA, 6894 bp, Debaryomyces hansenii
atgagcgccatttgtgccaaattgaaaaaacttacaactaacatttcactcaataggttactatttccaaatcttaatttacgtcatcaatatagaatattaaagtatattccagttaaacgacttaatagatacatcaactacaacagtgtcaacactactaataacgacaccaacgataacaaactaaagaataccattgataataaatataggatgtctgacgtaacaacagaggtgagaaattacactcaaatgcatcagaaattagctgaccactttaaagggttgaactcggcagataatgctgagccaggtaaggtgacggactttgtaagatcgcacgaaggtcacacggtaatttcgagagttttaattgcaaataacggtattgctgccgtcaaggagatcagatcggttagaaaatgggcctacgagacattcggtgatgaaagggccattcaattcactgtgatggctaccccagaagatttggaagctaatgcggagtacattcgtatggcggaccaatttatcgaggttccaggtggaactaacaataataattatgccaacgttgagttgatcgtggaaattgccgaaagaaccaatgttgacgctgtctgggccggatggggtcatgcttcagaaaacccattattaccagaaatgttagctgcctctccaaagaaaatcttgtttatagggcctccgggatctgctatgagatctttaggagataagatttcttctactatcgtagctcaacacgcagatgttccatgtattccatggtccggtactggcgttagggaagttaagattgacgaagaaactaacttggtttcggtttccgacgctgtttacgccaagggttgttgcacaagtccagaagatggtcttgttaaggcaaaagaaattggtttcccagtcatgatcaaagcttctgaaggtggtggtggtaaaggtattagaaaagtcgataacgaaaaagactttattgccttatacaagcaagcttcgaacgaaattccaggatctcctattttcattatgaagttagccggtgatgctagacatttggaagttcaattattagccgatcaatacggtacgaatatttctctttttggaagagattgttccgttcaaagaagacatcaaaagatcattgaagaggctccagtcactattgccaaaaaagaaagtttccacgccatggaaaacgctgccgtaaggttaggtaaattagttggctatgtttctgcaggtacagttgaatacctttattcgcatagtgaagataaattctatttcttagaattgaatccaagattacaagttgaacatccgactactgaaatggtcactggggttaatttaccagctgcccaattacaaattgctatgggtattccaatgcaccgtattagggatattaggtcattatacggtgtagaccctcacacttctaccgaaattgattttgaatttaagactgaaagttcattagttagtcagcgtcgtcctgttccaaagggccacactactgcatgtcgtattacttcagaagatccaggtgaaggatttaagccttctgggggttcgttacatgaattgaattttagatcatcttctaatgtctggggttatttctctgtcggtaatcaatcttctattcattctttctccgattcgcaattcggtcatatttttgcctttggtgaaaaccgttctgcttcaagaaaacatatggttgttgctttgaaagaattatctattagaggtgattttagaactaccgtggaatatttaattaaattattagaaacaccagattttgaagacaatacaatcactactggatggttagacgaattaatttcaaagaaattaacgtccgaaagacctgatcatattgttgcagttgtgtgtggtgccgctaccaaagcccatattcaatccgaagaagatagaaaagagtatatccaatcattggagaaaggtcaagttccaaacaaagctttattaaggactatttatccaattgaattcatttatgaaggatatagatataaattcaccgcaaccaagtcatctaatgattcttatactttattcttaaatggtacaagaggagttgttggtgttcgttcattatctgatggtgggttattatgtgcaattgacgggaaatctcattctatttattggaaagaagagcctgctgcaaccagattatcagtgaatggtaaaacctgcttattagaagctgaaaacgatccaacacaactaagaacaccatctccaggtaagttagtgaagtacttaattgaaagtggtgaacatgttaattccggagaagtttatgctgaggttgaagttatgaaaatgtgtatgccattaattgcccaagacaatggtgttgttcaattaataaaacagcctggctctacagtgaatgctggtgatattttagctattttagagttagatgacccatctaaggttaaacatgctatgccttacgaaggaaccttacctccattaggtgatcctgtcgttagaggtaccaaatcagcacatgctttccaacattatactaatattttgagaaatatcttagcagggtttgataatcaagttatcatgaattcaactttgaagagcttaattgagatcttaaaaaacaaagacttgccctactcagaatggaatcagtatgcctctgcattacactcaagattaccaattaagttagatgaagcattgtcagctttgattgaaagaaaccaatcgagaggtgctgagttcccagctcgtcaaatcttgaagcagattcaaaaattcactaccgatccatcgatcgatgcaagtgttaatgaagtggttaaaccattaattgatattgcgactagatactctaacggtcttgttgagcacgagtatgaatttttctcaaatttgatcaatgaatactttgaaattgaaaacttattctctggtacaaatgttcgtgaggatgatgttgttttgaaattaagggatgaaaacaaagctgatttaaataaagttattagtattgtattatctcattccagagtcagctcaaaaaacaatttagttttggctattttagatgaataccaaccactattacagtcatcttccaatacagctaacggaattagaaatgcattgaaggatattgttgaattggatactagaggcgcggctaaggtggctttgaaagcaagggaaatgttaattcaatgctctttgccatcaattcaagaaagatcagatcaattagaacatatcttgagatcctccgtgcttcaaacttcttatggtgagatctatgctaatcaccgtactccaagattagatattattcgcgaggttgtcgattccaaacacacagtctttgacgtgttgcctcaatttttagtcaaccaagatgaatgggtttctattgcggctgcagaagtctatgttcgtcgttcatatagggcatactccttgggaccgatcacttatgacttccatgacaaattaccgatcattgaatggaaattccaattaccaagtcttaactcatcccagttaactggtgttcaacaaactcagaatccagatcaacctgctatgaaccgtgcggcatctgtttctgatttgtcttttgtcgtcgatcaaaacaaagaacaaaagacaagaattggtgtcttagtaccttgtagacatcttgatgatgtggatgaaatgattactgcagcattagaaaagatccaaccttctgacggtattacgtttaaggctaaagagtcggaggaatctaaagcttcttatttaaatgttttcaacatcgtcgtaacgaatattgatggttacaataatgaagaggaagtattggcccgggttcatgaaattctcgatgaatttaaggaagaccttaagtcagcttctattcgtcgtatcactttcgtatttgctaataagattggtgtttatcctaaatactttacttttaccgcaccagattatgttgaaaacaaggttatccgtcatattgagcctgcattggcattccaattggaattgggaagattaaataactttgacattaagccgatatttaccgacaacagaaatattcatgtttatgaagctgttggtaagaactctccatctgataagagattctttacaagaggtatcattaggactggaattattcgtaatgatataagtattagtgaatacttgattgccgaatctaatcgtttgatgtcaagcattttggatgcacttgaggttattgatacttctaattcagatcttaatcatatcttcatcaacttttctgctgtatttaatgtcttgcctgaggaggttgaagccgcttttggctcatttttagagagattcggtagaagattgtggagattacgtgtgactggggctgaaattagaattgcatgtactgatccaaatactggtaattctttcccattgcgcgcaattatcaccaatgtctcaggttacgttgttaaatctgagttgtatatggaagttaaaaacactaagggtgaatgggttttcaaatccattggttctacgggttccatgcacttgagaccaatttcaactccttatccagcgaaggaatcgttgcaaccaaaacgttataaggctcataatatgggtactacgtacgtttatgatttcccagaattattccgccaggctactctttctcaatggaaaaatcatccgaaagaaaaagttcctaaggaaatctttacgtctttagaattaatttctgacgagaatggagatttgacggcagtagaacgtgatcctggcagcaacaagattggtatggttggattcaaggtaactgccaaaaccccagaatatcctcgcggccgtcaatttattattgttgccaatgatattacccataaaattggttcatttggtccagaagaagatgaattcttcaacaaatgtactcaattagctagaaaattaggaattccaagaatttatctttcagctaattccggtgccagaattggtattgctgatgaattggttccacttttcaatgttgcttggaatgttgaaggttctccagataagggtttcagatacttattcttgacccctgaagataagaagagcattgatgaagctggaaaatctgatacaattgtcactgaaagaatcgttgaagaaggccaggaaagatatgtcatcaagtcgatcgttggagaagaagatggtttaggtgttgaatgtcttaaaggatctggtttgattgctggtagtacctcgagggcctataaggatattttcactattaccttagtgacttgtagatcagttggtattggtgcttacttggttagattaggtcaaagagccattcaagttgaaggtcaaccaatcattttaactggtgctcccgctattaataagttattaggtagagatgtctattcgtctaacttgcaattaggtggtactcaaattatgtatcgtaatggtgtttcccatcttacagcttcagatgatttagcgggagttgagaagattatggaatggatgtcttatgttccggctaagcgtgatatgccaattccaattttggaaagtgaagacagctgggatagagaggttgaatatgttccacctaaggatgaaccatatgatgttcgttggatgatagaaggaaaacagttagataatggtgaattcgaatcaggtttatttgataagaattctttccaagaaacattatccggttgggccaaaggtgttgttgttggtagagcacgtcttggtggtataccaattggtgtcattggtgtcgaaacaagaactatagacaacttagtacctgctgatcctgctaatccagagtccactgaaatgatgattcaagaagctggtcaagtttggtatccaaactctgctttcaagactgctcaagcgattaacgatttcaaccatggtgaacaattgccattaatgattttggctaactggagaggtttctctggtggtcaacgtgatatgttcaatgaagttcttaaatacggttcttttattgttgatgcgttagttgactttaagcaaccaattttcacttatattccaccaaatggtgaattaagaggtggttcatgggttgttgttgacccaaccattaatgctgatatgatggaaatgtatgctgatgtcaattccagagctggtgttttggaacccgagggaatggttggtattaaatacagacgtgacaagttattatctactatagaaagattagatccaacatacagggaccttaaaaagcaattaaacgaaagcaaattatcaccagaagaacatgcccaaatttctgctaagttgactactcgtgaaaaggcattgttaccaatttatgcccaggtttcagttcaatttgctgacctccacgatagatctggtcgtatgttagccaagggtgtcattagaaaagaaatcaactggccagaagcacgtcgtacctttttctggcgtttacgtcgtcgtttgaatgaagaatacttgttgaaacttattggtgaacaaatcaaatcagacaacaaattagaaaaggttgccaggttgaagagttggatgccaacagttgactatgacgacgatatggctgtcagcaattggatcgaacagaaccactctaagttgcagaagagaattgaagaattgaaacacgaatccgctcgtcaaaacttagttaatatcttgagagaggaccctaaaagctcggtttccgttattaaagatttcttatctaaccttccagaggaccaaagatcagaatttgcggcatccttaaaatag

SEQ ID NO: 85 - DNA, 6801 bp, Yarrowia lipolytica
atgcgactgcaattgaggacactaacacgtcggtttttcagtatggcttcaggatcttcaacgccagatgtggctcccttggtggaccccaacattcacaaaggtctcgcctctcatttctttggactcaattctgtccacacagccaagccctcaaaagtcaaggagtttgtggcttctcacggaggtcatacagttatcaacaaggtcctcatcgctaacaacggtattgccgcagtaaaggagatccgttcagtacgaaaatgggcctacgagacctttggcgacgagcgagcaatctcgttcaccgtcatggccacccccgaagatctcgctgccaacgccgactacattagaatggccgatcagtacgtcgaggtgcccggaggaaccaacaacaacaactacgccaacgtcgagctgattgtcgacgtggctgagcgattcggcgtcgatgccgtgtgggccggatggggccatgccagtgaaaatcccctgctccccgagtcgctagcggcctctccccgcaagattgtcttcatcggccctcccggagctgccatgagatctctgggagacaaaatttcttctaccattgtggcccagcacgcaaaggtcccgtgtatcccgtggtctggaaccggagtggacgaggttgtggttgacaagagcaccaacctcgtgtccgtgtccgaggaggtgtacaccaagggctgcaccaccggtcccaagcagggtctggagaaggctaagcagattggattccccgtgatgatcaaggcttccgagggaggaggaggaaagggtattcgaaaggttgagcgagaggaggacttcgaggctgcttaccaccaggtcgagggagagatccccggctcgcccatcttcattatgcagcttgcaggcaatgcccggcatttggaggtgcagcttctggctgatcagtacggcaacaatatttcactgtttggtcgagattgttcggttcagcgacggcatcaaaagattattgaggaggctcctgtgactgtggctggccagcagaccttcactgccatggagaaggctgccgtgcgactcggtaagcttgtcggatatgtctctgcaggtaccgttgaatatctgtattcccatgaggacgacaagttctacttcttggagctgaatcctcgtcttcaggtcgaacatcctaccaccgagatggtcaccggtgtcaacctgcccgctgcccagcttcagatcgccatgggtatccccctcgatcgaatcaaggacattcgtctcttttacggtgttaaccctcacaccaccactccaattgatttcgacttctcgggcgaggatgctgataagacacagcgacgtcccgtcccccgaggtcacaccactgcttgccgaatcacatccgaggaccctggagagggtttcaagccctccggaggtactatgcacgagctcaacttccgatcctcgtccaacgtgtggggttacttctccgttggtaaccagggaggtatccattcgttctcggattcgcagtttggtcacatcttcgccttcggtgagaaccgaagtgcgtctcgaaagcacatggttgttgctttgaaggaactatctattcgaggtgacttccgaaccaccgtcgagtacctcatcaagctgctggagacaccggacttcgaggacaacaccatcaccaccggctggctggatgagcttatctccaacaagctgactgccgagcgacccgactcgttcctcgctgttgtttgtggtgctgctaccaaggcccatcgagcttccgaggactctattgccacctacatggcttcgctagagaagggccaggtccctgctcgagacattctcaagacccttttccccgttgacttcatctacgagggccagcggtacaagttcaccgccacccggtcgtctgaggactcttacacgctgttcatcaacggttctcgatgcgacattggagttagacctctttctgacggtggtattctgtgtcttgtaggtgggagatcccacaatgtctactggaaggaggaggttggagccacgcgactgtctgttgactccaagacctgccttctcgaggtggagaacgaccccactcagcttcgatctccctctcccggtaagctggttaagttcctggtcgagaacggcgaccacgtgcgagccaaccagccctatgccgagattgaggtcatgaagatgtacatgactctcactgctcaggaggacggtattgtccagctgatgaagcagcccggttccaccatcgaggctggcgacatcctcggtatcttggcccttgatgatccttccaaggtcaagcatgccaagccctttgagggccagcttcccgagcttggaccccccactctcagcggtaacaagcctcatcagcgatacgagcactgccagaacgtgctccataacattctgcttggtttcgataaccaggtggtgatgaagtccactcttcaggagatggttggtctgctccgaaaccctgagcttccttatctccagtgggctcatcaggtgtcttctctgcacacccgaatgagcgccaagctggatgctactcttgctggtctcattgacaaggccaagcagcgaggtggcgagtttcctgccaagcagcttctgcgagcccttgagaaggaggcgagctctggcgaggtcgatgcgctcttccagcaaactcttgctcctctgtttgaccttgctcgagagtaccaggacggtcttgctatccacgagcttcaggttgctgcaggccttctgcaggcctactacgactctgaggcccggttctgcggacccaacgtacgtgacgaggatgtcattctcaagcttcgagaggagaaccgagattctcttcgaaaggttgtgatggcccagctgtctcattctcgagtcggagccaagaacaaccttgtgctggcccttctcgatgaatacaaggtggccgaccaggctggcaccgactctcctgcctccaacgtgcacgttgcaaagtacttgcgacctgtgctgcgaaagattgtggagctggaatctcgagcttctgccaaggtatctctgaaagcccgagagattctcatccagtgcgctctgccctctctaaaggagcgaactgaccagcttgagcacattctgcgatcttctgtcgtcgagtctcgatacggagaggttggtctggagcaccgaactccccgagccgatattctcaaggaggttgtcgactccaagtacattgtctttgatgtgcttgcccagttctttgcccacgatgatccctggatcgtccttgctgccctggagctgtacatccgacgagcttgcaaggcctactccatcctggacatcaactaccaccaggactcggacctgcctcccgtcatctcgtggcgatttagactgcctaccatgtcgtctgctttgtacaactcagtagtgtcttctggctccaaaacccccacttccccctcggtgtctcgagctgattccgtctccgacttttcgtacaccgttgagcgagactctgctcccgctcgaaccggagcgattgttgccgtgcctcatctggatgatctggaggatgctctgactcgtgttctggagaacctgcccaaacggggcgctggtcttgccatctctgttggtgctagcaacaagagtgccgctgcttctgctcgtgacgctgctgctgctgccgcttcatccgttgacactggcctgtccaacatttgcaacgttatgattggtcgggttgatgagtctgatgacgacgacactctgattgcccgaatctcccaggtcattgaggactttaaggaggactttgaggcctgttctctgcgacgaatcaccttctccttcggcaactcccgaggtacttatcccaagtatttcacgttccgaggccccgcatacgaggaggaccccactatccgacacattgagcctgctctggccttccagctggagctcgcccgtctgtccaacttcgacatcaagcctgtccacaccgacaaccgaaacatccacgtgtacgaggctactggcaagaacgctgcttccgacaagcggttcttcacccgaggtatcgtacgacctggtcgtcttcgagagaacatccccacctcggagtatctcatttccgaggctgaccggctcatgagcgatattttggacgctctagaggtgattggaaccaccaactcggatctcaaccacattttcatcaacttctcagccgtctttgctctgaagcccgaggaggttgaagctgcctttggcggtttcctggagcgatttggccgacgtctgtggcgacttcgagtcaccggtgccgagatccgaatgatggtatccgaccccgaaactggctctgctttccctctgcgagcaatgatcaacaacgtctctggttacgttgtgcagtctgagctgtacgctgaggccaagaacgacaagggccagtggattttcaagtctctgggcaagcccggctccatgcacatgcggtctatcaacactccctaccccaccaaggagtggctgcagcccaagcggtacaaggcccatctgatgggtaccacctactgctatgacttccccgagctgttccgacagtccattgagtcggactggaagaagtatgacggcaaggctcccgacgatctcatgacttgcaacgagctgattctcgatgaggactctggcgagctgcaggaggtgaaccgagagcccggcgccaacaacgtcggtatggttgcgtggaagtttgaggccaagacccccgagtaccctcgaggccgatctttcatcgtggtggccaacgatatcaccttccagattggttcgtttggccctgctgaggaccagttcttcttcaaggtgacggagctggctcgaaagctcggtattcctcgaatctatctgtctgccaactctggtgctcgaatcggcattgctgacgagctcgttggcaagtacaaggttgcgtggaacgacgagactgacccctccaagggcttcaagtacctttacttcacccctgagtctcttgccaccctcaagcccgacactgttgtcaccactgagattgaggaggagggtcccaacggcgtggagaagcgtcatgtgatcgactacattgtcggagagaaggacggtctcggagtcgagtgtctgcggggctctggtctcattgcaggcgccacttctcgagcctacaaggatatcttcactctcactcttgtcacctgtcgatccgttggtatcggtgcttaccttgttcgtcttggtcaacgagccatccagattgagggccagcccatcattctcactggtgcccccgccatcaacaagctgcttggtcgagaggtctactcttccaacttgcagcttggtggtactcagatcatgtacaacaacggtgtgtctcatctgactgcccgagatgatctcaacggtgtccacaagatcatgcagtggctgtcatacatccctgcttctcgaggtcttccagtgcctgttctccctcacaagaccgatgtgtgggatcgagacgtgacgttccagcctgtccgaggcgagcagtacgatgttagatggcttatttctggccgaactctcgaggatggtgctttcgagtctggtctctttgacaaggactctttccaggagactctgtctggctgggccaagggtgttgttgttggtcgagctcgtcttggcggcattcccttcggtgtcattggtgtcgagactgcgaccgtcgacaatactacccctgccgatcccgccaacccggactctattgagatgagcacctctgaagccggccaggtttggtaccccaactcggccttcaagacctctcaggccatcaacgacttcaaccatggtgaggcgcttcctctcatgattcttgctaactggcgaggcttttctggtggtcagcgagacatgtacaatgaggttctcaagtacggatctttcattgttgatgctctggttgactacaagcagcccatcatggtgtacatccctcccaccggtgagctgcgaggtggttcttgggttgtggttgaccccaccatcaactcggacatgatggagatgtacgctgacgtcgagtctcgaggtggtgtgctggagcccgagggaatggtcggtatcaagtaccgacgagacaagctactggacaccatggctcgtctggatcccgagtactcctctctcaagaagcagcttgaggagtctcccgattctgaggagctcaaggtcaagctcagcgtgcgagagaagtctctcatgcccatctaccagcagatctccgtgcagtttgccgacttgcatgaccgagctggccgaatggaggccaagggtgtcattcgtgaggctcttgtgtggaaggatgctcgtcgattcttcttctggcgaatccgacgacgattagtcgaggagtacctcattaccaagatcaatagcattctgccctcttgcactcggcttgagtgtctggctcgaatcaagtcgtggaagcctgccactcttgatcagggctctgaccggggtgttgccgagtggtttgacgagaactctgatgccgtctctgctcgactcagcgagctcaagaaggacgcttctgcccagtcgtttgcttctcaactgagaaaggaccgacagggtactctccagggcatgaagcaggctctcgcttctctttctgaggctgagcgggctgagctgctcaaggggttgtga

SEQ ID NO: 86 - DNA, 9251 bp, Aspergillus nidulans
gcagcggtaaagccgacaatcttattggcagattggtgcgtaagtcaaatcagactttattcggtactgactagctcgaccttcccccccggtctagactgtctagccgacggaaaacgggcgctacgtgcttcaagagagatcgatcgtctctgcagaatgtctccgcgagccttggggctagggcattgcaaaaaatcgaatttcctgctactcaagcccaactaagtgcaaaagacatccgttcaatgatactagagacggcggaaaaagctaccgtctcatttgaagtcaaagggggctccctaccgtggggcagaggctgccacatcctgattttctgctgatcaagcaagaaccagcgcccgattgttacttactacagtttatagtcgtgacctcgtgggtttcaagacttgcacctcagaatacccgccataccacatatggttagttgccaggcgatttgctgaggccgatcctctcccgaggaccaactccccaacgtgagcttcatatccgaagattgtgattggtgggatacttgaccggtgccaacatgtccgaaggcggctagtatatttctgctctcgataccaccaccgtttattggttatcgtacgtattgatcttacaggttgaaacctcgatggctggactacggaggattttaagagtatcaacaggccatggttgctttcctgatgtataccattgggaacttgacaagaaagttattgagatgttacttttgaacgaattgccggcggcggtgcaaaaccggacttcttggaatcctttgaggacagacttgtaggaataaaactcccgagccgacacttactccggaacaggttccgtacaaacttggcctatgaaatactatcgaaatcttactgtactccgcatactccggccaacaagcagtcagcttatactccggagaggtaagcagataagatgaagagactcctgtagcgatataaaggttgccataaattcccagctgaatgatccattgatacgatccacgcgtggtagaggtcgttcgacgcagctgagattcaatctgtctatgcggatatttcaaacgcagccttatactccgtaaaaatactgtactctgcgtaattaccgaacaccacctgactggaaaaccaaaaaagccaactccagctttcggagcggagtattaatattttggggccaaatggacgtcattgggagttggcacgctatatgagacactaaggattctgaaattgcataggcaggcatacacagtaaaacggggcaaaaaagtggtgggaagagtgcgggcggcccaacaatgcagtcaatggggtgggaatcctggacccggactccgaagaagattcattactgccgcgtatccagattacgttcctgatccagctcggtctttttctcacgttctcctcgcctctgtatcatattcttttccccctagggataaaagaagaagaatattaggattcatttttcctcttgttcatttcagatttcttcttctgactctctttgaccggtggtggtaagtactgcgaatccttccgttcctggcgcgctgtaccgccgtttgcgactgaggcgagtacagtagctttcgatttttctgggacccttcaggttaacgttgcgttctgtcagcccagctctttctctcttcatcctccttccggcacgaatgctctctcctgcctaattgacttatcctggctcttctcttctgattctccaacccgggcttatctcacacccttgtcgtttcacgaattgaacgaagcccgtattctccccttctctctggaccttcgggctgtttccgccgactttcctactttccccccgaacttcttttcgagctgcgcattaatatatatcgcatgggaagcgtttaatacataatactcaaacagccactgcaaatatgggcgtcccagacggtacaacaaacggccacggaggctctcgagccgccaaacacaacctcccctcacatttcattggtggcaaccacttagacgctgctgccccaagcagcgtcaaggactttgtcgctaaccatgaaggtcactccgtcatcacctcggtgagtttagcctggcgactattgaagaataatttagaggcggtcggaccggcgactaactagaactctcactttcaggtccttatcgcgaataacggtattgcggccgtcaaggagattcgatctgtccgaaaatgggcctacgagacattcggcaacgagcgtgccattcaattcacagtgatggcaaccccagaagatctggcggcgaacgccgactatatccgtatggctgatcaatatgttgaggtatggaaacgcctttcggatgattcggagtgtatataatataggtcaaatttgttaaatctcctcgcaggtccctggtggtacgaataacaacaactacgccaacgtcgagctgattgtggatgtggctgaacggatggacgttcacgccgtctgggccggttggggtcacgcctctgagaacccccggttaccagaatctctagccgcttctcccaaaaagatcatctttattggacctcccgcctctgcgatgcgatctcttggtgacaagatttcctctactatcgtcgctcagcacgctcaggtaccgtgcattccgtggtctggaaccggtgtagatgaggtgaaggttgatgagaacggcatcgttacggtggaggaagaggtttacaacaagggatgcacattctctccggaagagggtctagagaaagccaagcagattggattccccgtcatgattaaagcctccgagggtggcggtggtaagggtatccgtaaggttgagaaggaagaggactttatcaacctgtacaatgctgcggcgaatgagattcctgggtcacctatcttcatcatgaagcttgccggtaacgcccgccacttggaagtgcagttgttggctgaccagtacggtaacaatatttcgcttttcggtcgtgactgttccgtgcagcgacggcaccagaagattattgaggaggcgccagtaaccattgcgaaccctacaactttccaggccatggaacgtgccgccgtgagcttgggtaagcttgtcggttacgtctccgccggtacggttgagtacctgtactctcacgctgatgacaaattttacttcctggagctcaacccgcgtctgcaggtcgagcatcccaccactgaaatggtcactggtgtcaacttgcccgctgcccagctccagattgccatgggtatccctctgcaccgtatccgtgacattcgtctgctttatggcgttgaccccaatacatcggcggagatagacttcgacttttccagcgaagagagcttcaagactcagcgccgtcctcagcccaagggacacaccaccgcttgccgtatcacttccgaagatcctggtgagggtttcaagccctctagcggaaccatgcacgagttgaacttccgaagttcatctaacgtttggggttacttctctgtcggaacagcgggtggtatccacagtttctccgacagccagttcggtcacatcttcgcgtacggagagaaccgctccgcctcgcgaaagcacatggtcattgccctgaaagaattgagcattcgtggtgatttccggacgacaattgagtacctgatcaagctcttggagacgccagcttttgaggaaaacaagatcaccactggttggttggatcagctgatttccaacaagctgactgcagagcgtcccgatacaacgatcgctgtgctctgcggtgctgtcactaaagcccatcaggctagcgaggcgcgccttgaagagtaccgtaacggcattcagaagggtcaggttccctctaaggatgtcctgaaaaccgtcttccccgtggacttcatctacgagggtaagcggtacaagttcactgccacccgtgccggtcttgacagctatcacctcttcatcaacggttctaagtgctcgattggtgtgcgtgccttggctgacggtggactactcgtcctcctcaacggtcggagccataacgtatactggaaggaggaggccgctgctacccgtattagtgtggacggcaagacttgcttgctcgagcaggagaatgatcctactcaacttcgtactccctctcccggaaagttggtcaagttcaccgtcgagaacggagagcatgtccgcgccggtcagccttttgctgaagttgaagtcatgaagatgtacatgcctctgatcgcccaggaggacggtattgtccagctcatcaagcagcccggtgccacccttgaggctggtgacattcttggtatccttgcccttgacgatccatcccgtgtcaagcatgctcagccgttcaccgagcagcttcccccaattggaccccctcaggtcgttggtaataagcctgctcaacgatttttcctcttgcacagcattttggagaacatcttgaagggtttcgacaaccaggttattatgaactctactctcaaggagctcatcgaggtccttcgcgaccccgagttgccttacagcgaatggaacgcccagtcttccgccctccactcccgcatgccccagaaattggatgctcagcttcaaaacattgttgaccgcgctcggtcacgcaaggccgagtttccggccaggcagctgcagaagactatggtccgattcattgaagagaatgtcaaccctgctgacgccgagatcctgaagactacacttcttcctttggttcaggttattaataactacatcgaaggcttgaaggcgcacgaatacaaggtgttcgttggacttctcgagcagtactacgctgtggagaagctgttctctggcagcaaagctcgatatgaggatggtatcctcgccctccgtgaggagcacaaggatgatgttgccactattgtgcagatcgccctgtctcacagccgcatcggcgccaagaacgacctcatcctcgcgatcctgtcgatctaccgtcccaaccagcctggaatggccaatgtgggccagtacttcaagtcgattctgaagaaactgactgaaattgagtcgcgtgctgcggccaaggtcaccctgaaggctcgtgaagtcctcattcagtgcgctctgccttcgctggaggagcgtctttctcagatggagctcattctgcgctcctctgttgcggagtctcagtacggcgagaccggctgggcccaccgtgagcccgatctcggtgccctcaaggaggttgtcgattccaaatacaccgtgttcgacgttctgccacgcttctttgttcacaaggatgcgtgggtcactttggcggctctcgaagtctatgtgcgccgcgcctaccgtgcttactcaattcagggtatccagtatcaccacgagggcgagccagcattcctgtcttgggacttcacaatgggcaagctgggtcagcctgagttcggttccatgactgctgtcacccacccctccacgccaagcacgcctaccactgaatcaaaccccttcaagcgcgtctcctcaatcagtgacatgtccaacttgctaaatgacagccccaacgggactcccagaaagggtgtcatccttcctgtacagtacctcgaagatgccgaagagtacctcaccaaggctttggaagtgttcccaagggctggcactaggaagcctagcgaccatggcctaattgcctctcttgaggggaagcgccgtccggctccccgtgctgacagtgagtctactgagctgaccggagtcttaaacatcgccatccgtgacatcgaggagcttgatgatgcccagatcgttgcccagatcagtaagctcgtttctagcttcaaggacgagttccttgcgcgccgcattcgtcgtgtgacgttcatctgcggcaaggatggtgtctaccccagctactacaccttcagaggtcccaactacgaagaggatgagagtatccgccacagcgagcctgccctggccttccagctcgaactcaaccgtctttccaagttcaagatcaagcccgtattcacagagaacaggaacatccatgtctacgaggcgattggcaaggggcctgagaacgataaggctttggacaaacggtacttcgttcgcgctgtcgtccgtcccggccgactccgtgacgatatccccactgcggagtaccttacctcggaagctgaccgtttgatgaacgacattcttgatgcccttgaggtcattggcaacaacaactccgatctcaaccacatcttcatcaacttctcccccgtcttcaacttacagcccaaagatgtggaagaggcattagcaggcttcttggatcgcttcggccttcgcctttggcgccttcgtgtcactggtgccgagatccgcattctatgcaccgatcccgccactggcatgccataccctctgcgtgtgatcattagcaacactgttggctatatcatccaggttgagctttacattgagaaaaagtccgagaagggcgagtggcttcttcacagcattggtggcactaacaagcttggatccaaccacttgcgtccggtttccaccccttaccctaccaaggagtggctgcagcctaagcgctacaaggctcacgtcatgggtactcaatatgtgtacgacttccctgagctcttccgtgaagctttccagaactcgtggaccaaggccatagagaagagcccgagcttgatcgagcgtcgtcctcctcttggcgagtgcatggaatacagcgagcttgtcttagacgatactgacaacctggttgagatttctcgcggccctggcaccaacacccacggtatggttggatggatagttactgctcgcacccccgagtatcccgagggcagacgcttcatcattgttgcgaatgacattaccttccagatcggttcgttcggtccattggaagacaagttcttccacaaatgtaccgaattggctcgtaagctcggaatccctcgtgtctacctttctgccaactctggtgctcgcattggtatggcggatgagctcatcccatacttctccgttgcttggaacgaccctgctaagcctgaggctggcttcaagtacctttacctcacacctgaggtgaagaagaagttcgacgcaagcaagcagaaggaggttatcactgaactgattcacgatgagggtgaggagcgccacaagatcaccaccattatcggtgccaaggatggtcttggagtcgagtgtcttaagggttctggtctcattgctggtgccacttcccgcgcttacgaagacatttttaccattactctcgtcacctgccgttcagtcggtattggtgcctaccttgtccgtcttggacaaagagctattcaggttgagggccagcctatcattcttactggtgctcctgccatcaacaagctgctaggaagagaggtctatacttccaacctgcagcttggtggtactcagatcatgtacaggaacggtgtttctcacatgaccgctgctaacgacttcgatggtgtcgagaaaattgtcgactggcttgccttcgtccccgaaaagaagggctctctgccacccatccgaccactcgccgacccttgggatcgtgacgtttcttaccaccctcctgcaaagcaagcctacgatgtccgttggctcatcaatggtaaggaagacgaggaaggcttcctccctggtctttttgacgccggctccttcgaggaggctcttggtggctgggctcgcactgtcgttgttggtcgtgctagacttggtggcatccccatgggtgttatcgctgtcgagacacgctctgtggagaacgttactccggctgatcctgctaaccccgactctatggaaatgatcacccaggaagcgggcggtgtctggtaccctaactcgtccttcaagactgcccaggccctccgggacttcaacaacggcgagcagcttcccgttatgatattggctaactggagaggtttctctggtggacaacgtgacatgtacaacgaggttctgaagtacggttcctacatcgtggatgctctcgtcaaatacgagcagcctattttcgtgtacattcctccgttcggtgaactccgtggtggttcttgggtcgttgtcgaccctaccatcaaccccgaccagatggaaatgtacgccgatgaggaggcccgtggtggtgtcctggagcccgagggtatcgttaacatcaaattccgccgcgacaagcagttggagaccatggctcgtttggaccctacttacggagaacttcgccgcgctcttcaggacaagaacctcagcaaggagaaactttccgacatcaaggacaaaatggcggcacgcgaggagcaactccttcctgtttacatgcagattgcattgcagtttgccgatctgcacgatcgtgctggccgcatgcaagccaagaacaccatccgccaagccctctcctggaagaacgctcgtcgcttcttctactggcgtgttcgccgccgtattagcgaggagtacattatcaagcgcatgctcaccgcatgccctgctcctgttcagggtgaaggcagcggagctgtcgcccagggtgtgtcgcctgcccctagcgactcccctcgcaccacccatctccgcactttgcactcatggactcccttccttgagaacgaggttgagaatgacgaccgtcgcgtcgccgtctggtatgaggagaacaaggagcttatccaggagaagattgaagctctcaagtctcaagccatcgcttcccagatctccgacgtcctcttcagcaaccgcgaaagcggcctcaagggcattcagcaggctctcagcttcctccctgttgaagagaaagagtccattctcaaatacctcggatccaactagattcacggagtcccccattgtctctacgaagaacaaacctactccttgtgaagaattgatttattgcattactactatcttcttttaaagcgccttgttcttttctttacattcttcagatccagactcctttaaggcgacgattactgattgcttgacggtggcttgttatgtttgctttgactgggttagaaggcacatgatatggaatggtttggattttgcatatactgttgcgtctttgttatttagcttttacgtctcattgaatggaacatttcatag

SEQ ID NO: 87 - DNA, 7795 bp, Schizosaccharomyces pombe
gtcgacatttgagattaagagattttaaatttacaagacatcaaattagaatacaattattaaaatctatgtatttttagaaaagttggatgcgtgggaactcaaaaacacgggacttaccatgcgccagagcgttacctcttcctcttcctgtagcaagctctacgcgataaaagcaaccatttcccttccacgactcttttaccgtagactgagaactatggctcctcgtgtagcctcccattttctgggtatgttatattaatcatttgatgtaggaattgcttgtagaaagttttgagatattgctgagcgtctgcggatgaaatgggttgttgtcgaacggtcagaagactagcttttttcgttgataatttggcaaaaacgagttagataaacttctttactatgtatacagtaattgctagtctcatttcctctaaaatgacactgtgtgcaaaatcgaatgtttcttcatgcggaacttgctgcccatgtttatcacttttcaagcactagctgtttgttttttccttagaaaccattctttcacgattattcatagaggatacattgtttctttacgcgtagatttcaaacatggatttgtgtgtctctgctgttgactggcatgattttactcgctcaatttttaactgttcgttaagcatgtttacccacgatacataattacttatattcttacaactttttcctcatttcctttgcaatcagtcgtctctgcttttccttctctcccaatcaaggggttctttttttaacggttcatttttattgacgttcttcttatctcgcaactttcgatttcaagcttttcttttttcattttgtactttattaaccatattttaggaggcaattccttagataaagcacctgcaggaaaggtgaaagattatattgcatcacacggaggacacactgttatcacgtctattcttattgctaataatggtattgcggccgtgaaagaaatccgaagcattcgaaaatgggcttatgaaaccttcaataatgaaagagctatcaagtttactgttatggcaacgccagatgatttaaaagttaatgccgattatattcgtatggccgatcagtatgtcgaagtacctggcggctcaaataacaataattatgctaatgtcgaacttatcgttgacattgctgaacgtatgaacgtccatgctgtttgggctggttggggacacgcatctgaaaaccctaaattgcctgagatgctttctgccagtagtaagaaaatcgtttttattggtcctccaggtagcgcaatgcgtagtcttggtgacaaaattagttctacaatcgttgctcaaagtgctcgtgtaccttgtatgtcttggtccggtaatgaactcgaccaagtacgtattgatgaagagacaaacattgttactgttgacgatgatgtttatcaaaaagcctgtattcgctctgcagaagaaggtattgccgtagccgagaagattggttattccgtcatgattaaggcctctgaagggggtggtggtaaaggtattcgtcaagttacttcaaccgaaaagtttgctcaagcattccaacaagtacttgatgaactccctggatctcccgtttttgttatgaaacttgctggacaagcacgccatttggaagttcaaattttagctgatcaatatggtaataatatttctctttttggtcgtgattgttccgttcaacgccgtcatcaaaaaatattagaggctcctgttaccatcgcacctgccgctaccttccatgaaatggagcgtgccgccgtgcgtttaggtgaattggtcggttacgcttctgctggtaccattgagtatctttatgagccagagaatgacaggttctatttccttgaactgaaccctcgtttacaggtcgagcatccaactaccgaaatggtttctggcgttaatttacccgctgcacaacttcaagttgctatgggtttgcctcttagtcgtattccacacattcgtgagctctatggcttaccacgtgatggtgactctgaaatcgattttttctttcaaaatcccgaatcttttaaagtacagaaggtccctactcctaaaggccattgtgttgcctgtcgtattacgtctgaagatcccggcgaaggatttaaaccatcgagcggtatgattaaagatctcaactttcgttcttctagcaatgtgtggggttatttctctgttggtactgctggtggaattcatgagttttcggattcccaattcggtcatattttttcatttacagaatctcgtgaatcctctcgcaaatcgatggtggttgcgttaaaagaattatctattcgtggtgattttagaactactgtcgaatatctcgtgcgtctccttgaaactaaggagttttctgaaaatgagtttaccacaggatggctagatcggcttattgcacaaaaagttacatctgctcgtcccgacaagatgcttgctgttgtatgtggtgctcttgtccgtgctcatgctactgccgatactcagtaccgtgctttcaaatcctaccttgaacgcggtcaagtaccgtcccgtgaatttttgaaaaatgtgtatgatattgaatttatttatgataacactcgctatcgttttaccgcatctcgttcttctccaggctcttatcatttgtttttaaatggttctcgttgtactgctggtgtccgttctttgactgatggtggattgttagttttgctaaacggacattcctatacagtatactatcgtgatgaggtaactggtactcgtatatctatcgataacctttcttgtatgctggaacaagaaaatgatcctactcaattaagaactccttcccctggcaagttggttcgtttcttggttgaaacaggtgagcatattaaagccggtgaagcgtatgcagaggtagaagttatgaaaatgattatgcctttagtagcaaccgaagatggtgttgttcaattgataaagcaacccggtgcatctttagacgccggtgatattcttggaatactcacgcttgatgatcctagccgtgtcacccatgcattaccatttgatggtcagcttcctaattggggtgagcctcaaattgcgggaaataagccttgtcaacgctatcatgctcttttgtgtattcttttggacattctaaagggatatgataaccaaatcattctcaacagtacctacaatgaatttgttgaagtccttcgtaatcatgaattgccctatagcgaatggagtgctcattattcagcattggttaatagaatctctcctgtacttgataagctttttgtatctataatcgaaaaagccagatctcgtaaagctgaatttcctgccaaacagcttgaggttgctattcagacttattgtgatggtcaaaatttggcgacgactcaacaattaaaggtccaaattgcacctctccttaaaatcatatctgactacaaagacggcctcaaagttcatgaatacaatgttattaaaggtttgctcgaagaatattataatgttgaaaagttgttctctggaattaataagcgagaagaagatgttattcttcgtctacgtgacgaaaataaagatgatgttgataaagttattgcgttggctttatctcattctcgtataggatctaagaataacttgttaattacaattcttgatctaatgaagtccgaaccatcaacttttgtttctctgtactttaatgacattttgaggaagcttacagatttggattcaagggttacttctaaagtgtctctaaaggctcgtgagttgttaattacatgtgctatgccttctcttaatgagcgattctctcaaatggagcacatattgaaatcgtctgtagttgaaagtcattatggtgatgctaaattctcacaccgtacaccatctttagacattctgaaagaattgattgattctaaatatacagtctttgatgttttacctgctttcttttgtcacaccgacccatggtattctttagctgctcttgaggtatatgttagacgtgcttatcgtgcctactctgttcttgaaatcaactatcataccgaggccggaactccgtatgtactcacgtggcgttttcagcttcattcaagtggtgctccgggtttgggtgccaactcaactaatggttccaatttccctgcaagcactactccttcatacgaaaacagcaatcgacgcctgcagtctgttagtgatctttcttggtatgtcaataaaacagactctgagccattccgttttggtacaatgattgccgcagaaactttcgatgaattggaaaataaccttgcccttgcaatcgaccgtttaccactttctcgtaattactttaatgctggtttaacgttggatggcaattcttcttcagctaacgataacactcaagaattaactaatgtagtgaacgttgcgttaacctcaactggtgatttggatgattctgctattgttagcaagcttaaccaaatccttagtgatttccgtgatgatttgcttgagcataatgttagaagagtgacaattgttggtggcagaattaacaagtctgcttatccttcctactatacttatcgtgtttccgctgaacaaaaagacggcaatcttgtacactataacgaagatgagcgtattcgtcatattgaacctgcattggcattccaattagaattgggtcgtctatcgaacttcaatattgaacccgttttcaccgataatcataacattcatgtttattcggctaccgccaaaaatatggatacagataagcgattctttactcgtgcattagttagaccaggaagattacgtgacgagatacctactgctgagtatcttatatccgaaacccatcgtttaattaatgatattttggatgctttggaagttatcggtcatgaacaaacagacttgaatcatattttcattaactttacaccagcctttggtcttgctcctaagcaagttgaagctgccctcggagaatttttggaacgttttggcagtcgtttatggcgcttgagagtaactgcagctgaaattcgtattatttgcacggacccatcaactaacactttgtttcctcttcgtgtcattatttctaatgtttctggatttgttgtgaacgttgaaatttattctgaagtcaagactgagaataattcttggatatttaagagtatcggacaacctggatccatgcatcttcgccccatcagtacaccttatcctaccaaagaatggcttcaacctcgtcgttacaaagctcaattaatgggcactactttcgtttatgacttcccagaattattccgtcgcgccttcaccgatagctggaaaaaggttccaaatgggcgatccaaagttactataccccagaatatgtttgaatgtaaggagcttgttgctgacgaacatggtgtattacaggaagtcaatagggagcctggaactaactcctgtggtatggtagcatggtgcattactgttaagacgcctgaatatcctaatggacgaaaaattatcgtagtggctaacgacatcactttccaaattggttcttttgggccccaagaggatgaatacttttataaagttactcaattggcacgtcaacgcggtattcctcgtatttacctcgctgccaattccggtgcacgtattggagttgctgatgaaatcgtccctcttttcaatattgcttgggtcgatcccgatagtccagaaaagggttttgattatatctatcttactccagaggcatatgagcgtcttcagaaagaaaatcccaatattctcaccactgaggaggttgttactgaaactggggaacttcgccataagattaccacaatcattggctcaagcgagggtcttggtgttgaatgtttgcgtggatccggtctgattgctggtgtcacatctcgcgcatacaatgacatttttacatgtactttggtcacttgtcgtgctgttggtattggcgcgtacttggttcgtctcggccaaagagctgtacaaatcgaaggccaaccaattattctaacaggtgcacccgcccttaacaaggttttaggccgtgaggtctatacctccaacttgcaattaggtggtactcaagttatgcatagaaatggtatatcccatcttactagtcaagatgattttgatggcatttcgaaaattgtaaactggatttcctatatccccgataaacgtaacaatccagtaccaatttcaccatcatcagatacatgggatcgtgatgtggagttctatccttctcaaaatggttacgatcctcgttggttaattgccggaaaggaagatgaagattctttcttgtatggtttatttgacaaaggatctttccaggaaactttgaatggctgggccaagactgttgttgttggtcgtgctagaatgggcggaattcctactggtgtgattgctgtcgagactcgtactattgaaaacactgtaccggctgatccagctaaccctgactctactgaacaagtattaatggaggctggtcaagtttggtatcccaactcagccttcaagactgctcaagcaatcaatgacttcaaccatggtgaacagttacctctttttattcttgctaattggcgtggattttctggtggtcaacgtgatatgtttaatgaggtactcaaatatggttcttatatcgtagatgctttggcttcttataaacaacctgtatttgtatacattcctccattcagtgaacttagaggtggctcttgggttgtagtagatccaaccatcaatgaggatcaaatggaaatgtacgcagatgaagagagtagagctggtgttttggaacctgaaggtatggtcagtattaaattcagacgtgaaaagttgctttctttgatgcgacgctgtgatcataaatatgcatcattgtgcaatgagcttaaaagagatgatttgagtgctgatgacctttcaactataaaggtcaagttgatggaacgcgaacagaagcttatgccaatttatcaacaaattagtattcattttgccgacttgcatgatcgtgttggtcgtatggttgcaaagaaggttgtccgtaaaccgttgaaatggacagaagctagacgtttcttttactggcgtctccgcagacgtttgaatgaacattatgctcttcaaaagattacccagctcattccttccttgactatccgtgaatctcgtgagtatctccagaaatggtatgaagagtggtgtggaaagcaagattgggatgaatctgataagtctgttgtttgttggattgaggaacataacgacgatttgagtaagagaactcaggaacttaagagtacttattacagtgagcgtctttctaaactccttcgttcagataggaagggaatgatagacagccttgcacaagttttgaccgagctcgacgaaaatgaaaagaaagaattggccggaaaactcgcgtcggttaattaagagtgcgatgatgatttttattcttcattctataacatctacatatcggtcttcacatgcttgaaaaaatgagattaatagatatgtttttagataactaagtgctatgagccttaatagtaaaagcccagtcttgcgttacccagcttttgatttttaggatggaggtacgttcctttccttttgatatatactaggtatttgaactgcag

SEQ ID NO: 88 - DNA, 7874 bp, Ustilago maydis
ggatccaaactgcgctccagccaagtcggaaaatctctcatgctccaagctggaagtctgggagtgtgccgctagttccgcctggccggtgcatgggtattcgcgtgtgtagaggtgtgtgtgtgtgctttcttccaagttttttggttttgcctcgaccatctcccatcccatcggtcgtccagcacttgatctcacaaaccttgactgtgttggctcttccgagaaacgtcgggtcttactgttatcattcctggcggtgcgtgcccttcttgcttcctcaccatcactatcgtcatcttcatcctcatccctccttcctgtgacctctcagtccaacatctgtccgccaacaccatcctctggccttccgactacggctctccgcacctctttccagccgcatcgttctcaaggtttccctcacccttgactatttgttgcagctcctacaccacctctctctccccgctttactttcgagctgtcagtgttagtcgagcagacgttactctcgacctactcttcgactcaccagagaatcacgtctaaatccctctcgggctcacttttctcggacacgctctcgcttccttcgtcgtcttcgagctccctctctcgaacgccgatcgagttgccacgtaacactcgttcaatcctcgatcgagaagttttgttctaaagacccaagcgttctgtcttgacctattccccgaatcctctgcaagcgcagctcttatttttacgcacgtaaagaatcagacaaccgtcagaatgccgcctccggatcacaaggcagtcagccagtttatcggtaagtttgaatgtaaaagtcttgtatttaccctacaagttggcgctgacccaactcccaactgcgctatgcgactacaggcggcaacccgcttgaaaccgctcccgccagccctgttgccgactttattcgcaaacagggtggtcacagtgtcatcaccaaggtcctcatttgcaacaacggtatcgccgccgtcaaggagattcgctccatccgaaaatgggcctacgagacctttggcgatgagcgtgccattgaatttaccgtcatggccacccctgaggacctcaaagtcaatgccgactacatccgcatggccgaccaatacgtcgaggtacccggtggctctaacaacaacaactacgctaacgtcgacctcatcgtcgatgtcgctgagcgagccggcgttcacgccgtatgggctggctggggtcacgcctccgagaacccacgcctacctgaatcgctcgccgcctccaagcacaagatcatctttatcggtccccccggctccgccatgcgctcgcttggtgacaagatctcgtccaccatcgtcgcacagcacgccgacgtgccatgcatgccctggtccggtaccggcatcaaggagaccatgatgagcgatcagggtttcctgaccgtctcggacgacgtctaccaacaggcctgcatccacaccgctgaagaaggtcttgagaaggccgaaaagatcggctaccccgtcatgatcaaggcctccgaaggtggaggaggaaagggtatccgaaagtgtaccaacggcgaagaattcaagcagctctacaacgccgttctcggtgaagtgcccggctcgcccgttttcgttatgaaactcgccggccaggcgcgtcatctcgaggtgcagctgctggccgatcagtacggcaacgccatcagcatctttggtcgtgactgctctgtccagcgtcgtcaccaaaagatcatcgaggaggctcctgtcactatcgctcctgaggatgcccgcgagtccatggagaaggctgccgtgcgtctcgccaaactggtcggctacgtctctgccggtaccgtcgaatggctctactctcccgagtcgggcgagtttgccttcctcgagctcaacccccgtcttcaggtcgagcaccctactaccgagatggtctcgggtgtcaacattcccgctgcccagcttcaggtcgccatgggtatccctctctactcgatccgcgacatccgaaccctttacggcatggaccctcgcggtaatgaggtcatcgactttgacttctctagccccgagtcgttcaagacccagcgcaagcctcagccccagggccacgtagtcgcctgccgtatcactgccgaaaaccccgacaccggcttcaagcctggcatgggtgccctcactgagctcaacttccgctccagcacctccacctggggttacttctccgtcgcagccagcggtgctctccacgagtacgccgattcgcagttcggacacatctttgcctatggtgccgaccgatccgaggcgcgaaaacagatggtcatctcgctcaaggagctctccattcgcggtgacttccgtaccaccgtcgaatacctcatcaagttgctcgagaccgacgccttcgagtccaacaagatcaccactggatggctcgatggtctcattcaggaccgtctcactgccgaacgacctcctgcggacctcgctgtcatttgcggtgctgccgtcaaggctcatctccttgcgcgtgagtgcgaggacgagtacaagcgcatcttgaatagaggtcaggtccctcctcgcgacaccatcaagaccgtcttctcgatcgacttcatctacgagaacgtcaagtacaactttactgccacgcgcagctccgtctccggctgggtcctctacctcaacggtggacgtacgctggtgcagctccgaccccttaccgacggaggtctgctcattggtctttcgggcaagtcgcaccccgtctactggcgtgaggaggtcggcatgacccgtctcatgatcgactccaagacctgcctcatcgagcaggagaatgaccccacccagatccgctcgccctcgcccggtaagctcgttcgcttcttggtggattcgggcgaccacgtcaaggccaaccaggccattgcagagatcgaggtcatgaagatgtacttgcctctcgttgccgccgaggacggcgtcgtctcgtttgtcaagaccgccggtgttgctctcagccctggagacattatcggtattctctcgcttgatgaccctagccgtgtccagcacgctaaaccctttgctggccagctgcccgactttggaatgcccgtcatcgttggcaacaagcctcaccagcgttacacggcccttgtcgaggtactcaacgatatcctcgatggttacgaccagagcttccgcatgcaggcggtcatcaaggagctcatcgagacgctccgcaaccccgagctgccctacggtcaggcctcccagattctgtccagcttgggcggccgtatccctgccaggctcgaggatgtggtgcgcaacacaattgagatgggccactcgaagaacattgagttccccgctgctcgtctgcgcaagctcaccgagaacttcctccgtgacagcgtcgaccctgctatccgcggacaggtgcaaatcaccattgctcctctctaccagctcttcgagacctacgctggcggcctcaaggctcatgagggcaacgtgcttgcttcgttcctccaaaagtactacgaagttgagtcccagtttaccggtgaggctgacgtcgttctcgagcttcgtctccaggccgacggcgacctcgacaaggttgtggccctgcagacttcgcgcaatggcatcaaccgcaaaaacgctctgctgctcaccttgcttgacaagcacatcaagggcacctcgcccgtctcgcgtactagcggtgctaccatgatcgaggctctgcgcaagcttgcctcgcttcagggcaagtcgactgcccccatcgccctcaaggctcgtgaggtctcgctcgacgccgacatgcccagtcttgccgaccgatcagctcagatgcaggccattcttcgtggctccgtcacctcgtccaagtatggtggtgatgatgagtaccatgctccctcgcttgaggttctccgcgagctcagcgactcacagtacagcgtgtacgatgtgctgcacagcttcttcggtcaccgcgagcaccatgtcgcctttgccgcgctctgcacctacgtcgtccgcgcctaccgagcttacgagattgtcaacttcgactatgccgttgaggactttgacgtcgaagaacgcgctgtgctcacctggcagttccagctgcctcgaagcgcttcttcgctcaaggagcgtgagcgtcaggtgtctatcagcgacctcagcatgatggataacaacaggagggctcgccccatccgcgagctgcgcactggtgccatgaccagctgcgccgatgtggccgacattcctgaacttctccctaaggttctcaagttcttcaagtcttctgccggtgccagtggagcgcccatcaatgtgctaaacgttgctgttgtcgaccagactgactttgtcgacgccgaagtgcgaagccagcttgccctgtacaccaatgcctgcagcaaggagttttccgctgctcgtgtccgccgtgtcacctacctcctttgccagcccggcttgtatcccttcttcgccaccttccgtcccaacgagcagggcatctggtccgaagagaaggcgattcgcaacatcgaacccgcgcttgcctaccagcttgagctcgacagggtcagcaagaactttgagctcacccccgttccggtctcgtcgtccacgatccatctctactttgctcgtggtatccagaactcggccgatacccgattctttgttcgctcactcgtccgtcccggccgcgtgcagggcgacatggctgcatacctcatctccgaatcggaccgcattgtcaacgatattctcaacgtcatcgaggtagctcttggccagcccgagtaccgcaccgccgatgcttcgcacatcttcatgtctttcatctaccagctggatgtcagcctcgtggatgtgcagaaggctattgccggcttccttgagcgacacggcacccgcttcttccgtctccgcatcacaggtgccgagatccgcatgattctaaacggtcccaacggcgagccccgcccgatccgagcctttgtcaccaacgagaccggtctggtcgtccgatacgagacatacgaggagactgtcgccgatgacggctctgtgattctgcgcggcatcgagccccagggcaaggatgccacgctcaatgcccagagcgcacacttcccttacacaaccaaggtggcactgcagtcgcgacgatctcgtgcccacgctttgcagaccaccttcgtctacgactttatcgatgtgcttggtcaggccgtgcgtgcgtcgtggagaaaggttgctgccagcaagattcccggtgatgtcatcaagtcggccgtcgagttggtctttgacgagcaggagaacctgcgtgaggtcaagcgtgctcctggtatgaacaacatcggcatggttgcttggctcgtcgaggtgctcacccccgagtaccccgctggccgtaagctcgttgtcatcgggaacgacgtcaccatccaggctggctcgttcggccccgttgaggaccgcttcttcgctgctgcctccaagctcgcccgtgagcttggtgtgccgcgcctctacatctcggccaattcgggtgcccgtatcggcttggcaactgaggcgctcgacctgttcaaggtcaagttcgtcggcgacgaccctgccaagggtttcgagtacatctacctcgacgacgagtcgctccaagccgtccaggccaaggcgcccaacagtgtcatgaccaagcccgtccaggccgctgatggcagcgtccataacatcatcaccgatatcatcggcaagcctcaggggggtctcggtgtcgagtgtctgtcgggcagtggtctcattgccggtgagaccagccgtgcaaaggaccagatcttcactgccaccatcatcacgggacgaagtgtcggtatcggtgcctatcttgctcgtctgggcgagcgtgtaatccaggtcgagggctcgcccttgatcctcactggttatcaggcactcaacaagctgctgggtcgtgaggtctatacctcgaacctacagctcggtggtcctcagatcatgtacaagaacggtgtttctcacctcactgctcaggacgacctcgacgctgtcaggtcgtttgtcaactggatatcatacgttcctgctcagcgtggtggacctctgccgatcatgcccaccaccgatagctgggaccgagcggtcacataccagcctcctcgtggtccttacgacccacgatggctcatcaacggtaccaaggccgaagacggcaccaagctcaccggtcttttcgatgaaggctcatttgtcgagacgcttggcggctgggccacttcggtagtcactggtcgtgctcgcctgggcggcatccctgtcggtgtgatcgctgtcgagacgcgcacgctcgagcgtgttgttccggccgaccctgcgaaccccaactcgaccgagcagcgcatcatggaagccggccaggtgtggtaccccaactcagcgtacaagactgcccaagccatctgggactttgacaaagagggtctgcctttggtcatccttgccaactggcgtggattttcgggtggccagcaggacatgtacgacgagatcctcaagcagggctccaagatcgtcgacggtctgtcgtcgtacaagcagcccgtgtttgttcacattccacctatgggtgagcttcgcggtggttcgtgggtcgtggtcgactctgcgatcaacgacaacggtatgatcgagatgtcggccgatgtcaacagcgcacgaggtggtgtgctggaagcctcaggtctggtcgagatcaagtaccgtgccgacaagcaacgtgctaccatggagcgactcgacagcgtctatgccaagttgagcaaggaagctgccgaagcgaccgacttcaccgcgcagaccaccgctcgtaaggcgttggcagagcgagagaagcagctcgcacctatctttacggcgatcgctaccgagtatgcagatgcacacgaccgtgcaggacgcatgcttgcgactggagtgctgcgatcggcgctgccatgggagaacgcgcgtcgatacttctactggcgtctcaggagaaggttgaccgaggtcgctgctgaacgcacggttggcgaggccaacccgacgctgaagcatgttgagaggctggctgtattgcgacagtttgttggtgctgctgcgagcgatgacgacaaggcggtggctgagcacttggaggcttcggccgaccagctgttggccgcatccaaacagttgaaggcacagtacatcttggctcagatctcgacattggaccctgaactgcgcgctcaactagccgcttcgctcaagtaatggacgtgactcttgcaagaattcgttctccaggcgccaggcgatcgttggcgcgattgacatcggattgagggatccacatgccattctccttcacgagcacctggctttacgttgaacgaatttttacgatgcagacctcattacgctctgcatgagcgctcttctggaatcagatctctcaaagaccactgaggggtggtctgcgaacctcttagaagtagcgcacgcactgggcgatggtccctgtcaattgttcttgttcttgttcttgttttggtttttgattatgttcttttcagcgtttagaatgtcccgtggctgcgcaatcgtgaaggttgattttcggctgatgtgggcgcgacgtgtggccgaactagcataattctctcttgcacacgagagcttggtctgcagagtgctggcggcgattggagagagtcacgagatcaagtagtcgac

SEQ ID NO: 89 - DNA, 7367 bp, Gallus gallus
catcgccgcgcccccagcaccgccgctgccctgcaccaggccgggggcccgcggcgcctccaccgcgcccggcaccctgagttcattttggaagtggataactgctcagattgcaagaataacaagagtgctgagagctcaatttggggagccatggaagagtcttcccaacctgctaaacccctggagatgaaccctcactctcgctttattattggttccgtgtcagaggataactcagaagatgagacgagctccttggtgaaacttgacctgctggaggagaaagagaggtctctgtcccctgtttctgtctgctcggattccctttcggatttgggacttcctagtgctcaagatggtttggcaaaccatatgaggcccagcatgtctggtttgcacctcgtaaagcaaggccgggacaggaagaaagttgacgtgcagcgggatttcactgtggcttctccagcagaatttgttactcgttttggagggaacagagttattgagaaggtcctgatagccaacaatgggattgcagcagtgaaatgcatgaggtcgatccggcgctggtcctatgagatgttccgaaacgagcgggcaatcagatttgttgtcatggtgactcctgaggacctgaaagcaaatgcagagtacattaaaatggcagatcactacgtgccagttccaggaggaccaaacaacaacaactatgcaaatgtggaactcattctcgatattgcaaaacgcattccagtgcaggctgtttgggctggctggggccatgcctccgagaacccaaaactaccagaacttctccacaaaaatgggattgctttcatgggtcctccaagccaagcaatgtgggctttaggagataaaattgcgtcgtcaatagtggctcagactgctggcatcccaactcttccttggaatggcagtggtcttcgagtggattggcaggagaatgatcttcagaagcgtatcctgaatgttcctcaggagctgtatgaaaaaggctatgtgaaagatgcagacgatggcctgcgggctgctgaggaagttggctaccctgtcatgatcaaggcctctgaaggaggaggagggaagggaattaggaaagtcaataatgcggatgacttccccaacctatttagacaggttcaggctgaagtcccaggctctccgatctttgtaatgaggctagccaaacagtcccgccacttggaggtgcagatcctggcagaccagtatggcaatgccatctctctctttggtcgggattgctccgtgcaacgcaggcatcagaagattattgaagaagcacctgcttctattgcaacttcggtggtatttgagcacatggaacagtgtgcagtgaagcttgcaaaaatggtggggtatgtgagtgcgggcactgtggaatacctgtacagccaggatggcagcttctactttctggagttgaatccccgtctgcaagtggagcacccctgcaccgagatggtagctgatgttaatcttcctgcagcacagctccagattgccatggggattccactccaccgtatcaaggatatccgagtgatgtatggtgtttccccatggggagatggatctattgattttgagaattcagcccatgtcccctgtccacgtggccatgttattgctgcacgtatcaccagtgagaatcctgatgagggatttaagcccagttctggtacagtccaggaactgaatttccgcagcaataagaatgtttggggctatttcagtgttgctgctgcaggagggctgcatgaatttgctgattctcagtttggtcactgcttctcttggggagagaatcgtgaagaagccatctcaaacatggtggtggctttgaaggagctgtccatccgaggggatttccgaaccactgttgaatacttgataaaactgttggaaacagaaagcttccagcagaaccgcattgacactggctggttggatcggcttattgctgagaaagtgcaggctgaaaggcctgataccatgctaggagtggtatgtggagctcttcatgtggctgatgtgagctttcgaaacagcgtctcaaacttcctgcactctttagaaaggggccaagtcctgcctgctcatactttgctaaacactgtggatgtggaactcatctatgaaggacggaaatatgtgttgaaggtgacccgacagtctcccaattcctacgtggtcatcatgaacagctcttgtgtggaagttgatgtgcacagactgagcgatggagggctgctcctatcttacgatggtagcagctacaccacctacatgaaagaagaagtggacaggtatcgcatcactataggtaacaagacctgtgtgtttgaaaaggaaaatgatccttctattctgcgctcaccttcggctgggaagcttatccagtatgtggtggaggatgggggacacgtgtttgcaggccaatgctttgcagaaatagaggtgatgaaaatggtgatgacactaacagcaggagagtcaggctgcatccattatgtcaaacgccccggggcagtgctggatccaggctgtgtgattgccaaactccagctggatgatcccagcagggttcagcaggctgaactgcacacaggcaccttgccacagatccagagcacagcacttcgaggcgaaaaactccatcgcatcttccattatgtcctggataacctggtcaacgtgatgaatgggtactgcctgccagagccctactttagcagcaaggtgaagggctgggttgagcgactaatgaagacactgagagatccatctttgcctctgctggaacttcaggacatcatgaccagtgtttctggacggattccacccaatgtggagaagtccatcaagaaggagatggcccaatatgccagcaacatcacgtcagtcctttgccagtttcccagccaacagattgccaatatcttggatagccatgcagccaccttgaaccgcaaatcagagcgtgaggtctttttcatgaacactcagagtattgtgcagcttgtacagaggtaccggagtggtattcggggtcacatgaaagcagtggtcatggatttgctccgtcaatatctgaaggtggagactcagtttcagcatggtcactatgacaagtgtgtctttgcccttcgggaagagaataaaagcgacatgaatgctgtattgaactacatcttctcacatgctcaggtcaccaagaagaacctgcttgtcacaatgctcattgaccagctctgtggccgtgaccccaccctgacagatgagctgatcaatattctgacagagctgacccagctcagcaagacaaccaacgccaaagtggcgctgcgggcacggcaggttctcattgcttcccatttgccgtcctacgagctgcgtcacaaccaggtggagtccatcttcctatctgctattgacatgtatggacaccagttctgcattgagaacctgcagaaactcattttgtcagagacatccatctttgatgtgctacccaactttttctaccacagtaatcaggtggtgagaatggcagctttggaggtgtacgttcgaagggcgtacattgcctacgagctgaacagcgtccagcaccgccagctgaaggacaacacctgcgtggtggagttccagttcatgctgcccacctcccacccaaacagaatgtccttctcttccaacctcaatcactacgggatggtccacgtagccagtgtgagtgacgtgctgctggacaactcgttcactcccccgtgccagcggatgggagggatggtctctttccgcacgtttgaagattttgtcagaatctttgatgaagtgatgagttgtttttgcgactctcctccccagagcccaaccttccctgaagctggccatgcttccctctatgatgaagacaaggctgcccgtgaggagcccattcacattcttaatgtcgctattaaaactgatggggacgtggatgatgatgggctggcagccatgttcagagagttcacacaaagcaagaaatcagtcctgattgagcatggcatccggaggctgacattccttgtggcacagaagagggaatttccaaagttcttcacgttccgtgccagggataagtttgaagaagacagaatctaccgccatctggagccagctctggctttccagctggagctgaaccgaatgcggaactttgacctcactgccattccatgtgccaaccataaaatgcatctctacctgggagcagctaaagttgaagtaggaacagaagtgacagactacaggttctttgtgagggccattataaggcattcagaccttgttaccaaggaagcctccttcgagtacctgcaaaacgagggagagcgattgcttttggaagccatggatgagttggaggtggcatttaataataccaacgtgcgcacggactgcaatcacatcttcttaaattttgtgcctactgtcatcatggacccatccaagatcgaggaatccgtgcggagcatggtgatgcgctacgggagccgcctgtggaagctccgcgtcctccaggccgagctgaagatcaacattcggctgacaccgacaggaaaggccatccccattcgtctcttcctgaccaacgagtcgggctactacctggacatcagcctgtacaaagaggtgacggattccaggacagggcagattatgttccaggcctatggggataaacagggaccacttcacgggatgctgataaataccccatacgtgaccaaggaccttcttcagtccaagagattccaggcacagtctttagggacatcctatgtctatgacattcctgagatgtttcggcagtctttaattaaactctgggattctatgaatgaacatgcattcctgccaacaccgccgctgccgtctgacatactgacatacactgaattggtgctggatgatcagggccagctcgtgcacatgaacaggctgccaggaggaaacgagattgggatggtagcctggaaaatgaccctcaagaccccggagtatcccgaaggccgtgatatcatcgtcattggcaatgacattacgtaccggataggttcttttgggcctcaggaggacgtgctgttcctgagggcttcagagcttgctcgaactcatggcatcccccgcatctacgtggctgccaacagcggagccaggattggtttggctgaggagatccggcacatgttccatgttgcgtgggaagatccagatgacccatacaaaggatacaagtacttgtatctgacacctcaagactataagaaagtcagcgctctgaactcagttcactgtgaacacgtggaggacaacggagagtccaggtataagataacagatattatcggaaaggaagacggacttggaatagagaacctcagaggatctggcatgattgctggagaatcatctttagcctacgagagtattatcaccatcaacttggttacgtgtcgggcaattggaattggagcttacctcgttcggttagggcagaggactatccaggttgagaactctcacataatcctgactggctgtggagccctcaacaaggtgctgggacgggaggtgtacacctccaacaaccagctgggcgggatccagatcatgcacaacaacggggtgacccacggcaccgtgtgcgacgattttgaaggagtctacactatcctgctgtggctttcctacatgcccaagagcgtatacagccctgttcctatcctcaaggtcaaggatcctatagacagaaccatagacttcgttcctaccaagactccctatgatcctcgctggatgctggctggacgcccaaatccaagtcaaaaagggcaatggcagagcggtttctttgacaatggctcgttcctggagatcatgcagccctgggcacagacggttgtggttggcagagcaaggctgggaggaatacctgtaggagtagttgccgtagaaaccagaacagtggagctgagcatccctgctgatcccgccaacctggactcggaggccaagataatccagcaggctggtcaggtgtggttccccgactctgcctttaagacagcccaggccatcaacgacttcaacagagaagggctgcctctgatggtctttgccaactggagaggcttctctggtggcatgaaagacatgtacgaccaggtgctcaagtttggtgcctacatcgtggacggcctgcgggagtaccggcagcccgtgctcatctacatcccaccgcaggcggagctcaggggcggctcctgggctgtcatcgaccccaccatcaaccccaggcacatggagatgtacgcggaccgtgaaagcagagggggaatcctggagccggaggggacggtggaaatcaagttccgcaggaaggacctggtgaagacaatgaggagagtggaccccgtctacatgcggctggccgagcggctgggtacccctgagctgagtgctgccgaccgaaaagacctggagagcaaactgaaggagcgggaggaattcctgattcccatttaccaccaggtggccatgcagtttgctgacctgcacgacacacccggccgcatgcaggagaagggtgccatcacggacattctggactggaaaacgtctcggaccttcttctactggaggctgagacgtcttcttctggaagatgtggtcaaaaagaagatccatgatgccaaccctgagctgaccgacgggcagatccaggccatgctgcgacgctggtttgtggaagtggaggggacggtaaaggcgtacctgtgggacagcaataaggacctggtggagtggctggagaagcagctgatggaggaggagggggttcgctcggttgtggatgagaacattaagtacatctccagggattacatcctgaagcagatccgcagcctggtccaggccaatcccgaggttgccatggattcgatcgtgcacatgacccagcatatatcacccacccagcgagccgagatcgtgcggatcctctccacaatggactctccttcttcaacgtaagagcatcgatttcctgtactcccccctgctcggtacagtggaggggaagaaaaaaagaaaaaaagctcagaattgccctttgtctgctcaactgcgaccgctgtaccgagacggggaggctcagggaaacgctggaagagtgacagttttagttttttcaaaccagactgaccagaggaagtcgctttggccggagacacgaggaagatgtataaacacgggccctgcaggattgagtt

SEQ ID NO: 90 - DNA, 1359 bp, Mesoplasma florum
atgttaaaatttcctaaaaattttcacatcggtgcttcaatgagtgctatgcaaacagaaggaaaaggaattactgaaataggtgatttaacttttgatgcatatttcaaagaaaatccggaattgttttaccatggtgttgggccagatctgacaagtgatattacaagacactataaagatgatattgaaaaatttaaatacatcggattagattcagttagaacaggtttttcttgagctagattatttccagatggtattaatctaaacaaagaagcagtaaagttctatcatgactatatcgatgagtatttaaaaaatgatattgaaattattatgactttatttcactttgacatgcctttatgagcacatgaattaggtggttgagagagcagagaagttattgaaaaatttataagttattgtgaatttgtattcaaggaatatggatcaaaaataaattattttgttaccttcaatgaaccacttgttcctgtatttgaaggatatgtaggtaaaatgcactatcccgcaaaggatagtcccaaagaagctgtagctcaagcatatggaattttcctagctcatgctaaagcagtaaagttatttaaagaattaaaaattgattcaaaaataggagttgtttataactgaaactttacattcccattttcagattcagcagaagataaaatttcagctgaaatctatgatgcttatgtaaatagaggaccattaaacattatgtataatggaaatattaacccaattattataaaaaccttagaagaatataacataactccatttcacacaagcgaagaaattgaaataattaaacaaactgaaattgattttttaggagttaattattatttcccttgtagagttaaaacaaatgaaaatgtaaaaaatagatgagctttagatcaaatgcatattgaaattcctgcagatgcaaaaattaatccttttagagggtgagaaatttatcctgaagggctatatgatatatctatagcaattaaaaaagagttaaataacattccatgatacattgctgaaaatggtatgggtgttgaaaatgaagatagatttagaaatgaaaatggacaaatagatgatgattacagaattgagtttttagaaactcatatgtctgaattaaaaagaggtttagatgctggatcaaattgttttggttaccacatttgagctgccattgactgctgaagctttagaaatgcttataaaaatagatatggtttaattgaagttgatttaaaagaccaatctagaaagtttaaaaaatcagcttactgatataaagaactaatagaaaataaggagtaa

SEQ ID NO: 91 - DNA, 1152 bp, Oryza sativa
atgataaatgagctagtaaaagcaggtattcagattcatgctgttctgtaccatatagatcttccacagagccttcaagatgagtacggtggatgggttagccccaaagttgtggatgacttcgcagcatacgctgatgtgtgcttccgcgagttcggtgacagagtcgcgcattggacaacttccattgagccaaatgtcatggctcaatctggctatgacgatgggtatctcccgccaaatcgttgctcgtatccgtttggcagaagcaactgcacactaggaaattccacggttgagccatacttgttcatacaccacaccctgctagctcatgcttcagctgttagactttacagggaaaagcaccaagctgcacagaagggcgttgtcggcatgaacatatactccatgtggttctacccactcacagagtcaactgaagatattgctgccactgaaagagtaaaggatttcatgtatggatggatcttgcatcctttggtgtttggagattacccagagaccatgaagaaggccgccggttcccgtcttccattattctctgactacgaatctgagctggttactaatgcgttcgacttcattgggttgaatcattatacctcaaattatgtgagcgataatagtaacgcagtaaaggcgccgctacaggatgtcactgacgatatttcttctttgttctgggccagcaagaatagcacacctactcgagagtttctaccagggacctcattagatcctcgggggctagagctcgcgcttgaatatcttcaggaaaagtatggaaatttgctgttttatatccaggaaaacggtagtggatcaaatgcaaccctggatgatgtggggaggattgactgcttgacacaatacattgcagccacgctgcgatccatcaggaatggcgccaacgtgaagggatactgcgtgtggtcattcatggatcagtacgagatgtttggcgattacaaggcgcatttcggcattgttgccgtcgattttggcagcgaagaactgacaaggcagcccaggcgctctgctagatggtactcggacttcttgaagaacaatgctgtcatcaaggtggatgatggttctgtctccacagccttccatgctcagctttga

SEQ ID NO: 92 - DNA, 2292 bp, Pseudomonas putida
tcaaagcaattcaaagctctgctgctgcaccgcctgggaatccagccctacctggacattgaactcccctggttcggcaacccgctgcaactggccattgtagaacttcaggtcgtcctcgctgatgcggaaggtcaacgtgcgggtttcacccggttcaagcatcagcttctggaagtttttcagctctttcactgggcggctcatcgacgccgacacatcctgcaggtacagctgcaccaccgtctcgcctgcaaccttgccggtgttcttgaccgttaccttggcatccagcgtatcgccgcgcttgagatccttgctcgacaggttcaggcctgacagctcgaagctgctgtagctcaagccatagccgaacggataaagcggcccgttgggttcttcgaagtattgcgaggtgtagttgcccggcttgcccggcgtgaacggccggccgatgcgggtgtggttgtagtacatcgggatctgccccaccgaccgtgggaaggtaatggccagcttgccggaggggttgtagtcgccaaacagcacgtcggcgatggcattgccgccttcggtgccggcgaaccaggtttccaggatggcgtcggcctgctcgcgctcccagctgatcgacagcggccggccgttcatcagcaccagcaccagcggtttgccggtggccttgagcgccttgatcagctcgcgctggctggccgggatctccagggtggtgcggctcgacgactcgtgggacatgccgcgggactcgccgaccactgcgaccacgacgtcggattgcttggctgccttgattgcttcatcgatcagcaccgcaggcgggcgtgggtcgtcgacgatttccggggcatcgaagttgaggaagttcaggtaatcgaagatcgccttgtcgcccgtgacgttggagcctttggcgtagaccagcttggccttgccttccacggcgcggcgcaagccttcgcgcacggtcaccgagtggacgggtttaccgtcggcggcccagctgcccatcatgtcgatcggagcatcggccagcgggccgaccaaggcaatggtgccggcctttttcagcggcagggtctggttgcggttttccagcagcaccaggctgcggcgtgccacgtcgcgcgcagcttcgcggtgcaggcggtcgttgccgtagtagtccttcagatcggtttcggccttgccgatgcgcacgtacgggtccttgaacaggcccatgtcgtacttggcacccagcacttcacgcaccgcctggtccagctcgcgctgggtcacctcgccggacttcagcaggcccggcagctcttcgccgtacagggtatcgttcatgctcatgtcgatgccggccttgatcgccagcttggcggcttcacggccgtcacgggcgacgccgtggcgaatcagctcctggatggcaccgtggtcgctgatggtcacgcccttgaagccccactccttgcgcagcaggtcgttcatcagccaggtgttggaggtggccggtacgccgttgatcgagttcagcgccaccatcacgccaccggcacccgcatcgagcgcggcgcgatagggtggcaggtagtcgttgtacattttcggcaggctcatatcgaccgtgttgtagtcgcgcccgccttccacggcgccatacaaggcgaaatgcttgacgatggccatgatgctgtcggggttggccgggctgctgccctggaacgagcgcaccattacctggccgattctcgaagtcaggtaggtgtcttcgccgaaaccttcgcttgtgcggccccagcgcgggtcacgggcgatatccaccattggcgcgaacgtcatgtccagggcgtcggccgaggcttcgatcgcggcggtgcggccgaccttggcgacggcctccatgtcccaggtcgcggccatgcccaggccgatcgggaagatggtgcgctcgccgtggacggtgtcgtaggcgaagaacatcgggatcttcaggcggctgcgcatggccgcgtcctgcatcggccggttttcgggggcagtgcgtgagttgaaggtgccaccgatacggccggcggcgatttcctcgcggatcttgtcgcggggcatttccgggccgatgctgatcaggcgcaactggccgattttttcggcttcggtcatctggctgatcagatgctcgataaaggcctgcttgtcctgtaggggcggggcggtgggggcggcgagggccgcctgactggcaaggcccatggccaggcccagcaaagacagtttcatcat

SEQ ID NO: 93 - DNA, 2742 bp, Pseudomonas syringae
atgaacaagcgaaagatgataggcgcgcactcggcgctggccttgctggcactggccgtttctcaggtgcacgccgctgatccgactgtgcagcagggacgtgaagaccgcgctgaaaaagccgcgcaaaagaccctggcgaaaatgaccatggaagaaaagctggcctacatcggcggcaccggcggctgggatgtgaagccgctgaccaactacggcgttccgcagattcacggcgctgacggcggcgtgggcgtgcgttacaccagcgaaggcaacgatcagggcgtggtctatccgtccggccctaacctggcagccaccttcaacccgcgtcgcgccatcgacctgggccgtgctctgggttatgacactgcgaccggcggctaccagttcatcacaggtccaggtgtaaacctgtaccgcatgccttacggcggccgtgcattcgaatacctttccggtgaagatccgttcctgggcgccagcctggcaccgggtgtcatcaacggtatccagtcccgtggtgtgtgggccaacgccaagcactacgcggccaacgaccaggaaagcaaccgtttcaatcttgaccagaagatgcccgagcgcgtactgcgtgaaatgtcgctgcctgcgttcgagtcgtcgtcgaaaaacggcaatgttgcgatgatgatgtgcgccttccagaaagtgaacggtgatttcgcctgcgaaagcgagcacctgatcgcccagatcctgaagaaggaatggggctacaaaggcttcgtgcagagcgactacaacgctgtggtgcacggctttgaagctgcccgcgccggtaccgatctggacatgatgggctaccagatgaacagctccgtgctcaagccgcacctggacgccggtgacctgagcgctgcgaccatcgatgacaaggtgcgccgcatcctcaagcagatctacctgtacaagttcgacagcaaggcaccgctgaccacccacaacatgaacagctcgaccagcaacaaggtcgctctgaatgctgcgcgtgaaggcatcgtgctgctgaaaaaccagggcgatctgttgccactggacaagcagaaggtcaagaaaatcgccgtcgtcggcaccctggccaaatatgcaccaccgaccggtttcggtagcgccaatgtcatggccagccattacgtcagcgagttgagcggcctgcagcaaatggcacccaacgccaaggtcgagttcatcgatggcctgtcgctggacccaagcacctctgcctggaacaccactgacgccgctggcaacagtgttcagggcatgaaggtcgaatacttcagcaacaccaactggtctggcgatgcagcggtcacccgtaccgagcagcacgttgacctggactgggccaacgacaagaacctgccgttcgagagcaacacctcaacgtccgatccgtacaccaccaaaggctcgaccgctggtgagctgaacggtgacacgtcttcgacctcgatccgctacaccggcaagatcaccccgacccagagcggcgaacaggtgttcaaggtgcgtgccgacggcgctgtgcgcctgtgggtcaacggcaagaaaatcatcgacaacggtgacggcaagccattgccgggcaacagcatcccgccgaccattccagagttcgccaagatcaatctggaagcaggccagtcctacgacgtgaagcttgagtactcgcgtcgcgccgggtacctgtcgaccatgggtggtctggtcggtgtgcagttgagctccgcttcgctgaacgcgcctcaggacctgtccggttatgacgcggttgtggttgcagtgggtaacagcaacgaatacgaaggtgaaggtttcgaccacagcttcgatctgcccgagttccagaacgaactgatccagaacatcgccaaggtcaacccgaacaccgtggtgaccatgtatggcggtaccggcctgaagatgagcgactggatcgaacaggttccggtagcgctgcacgccttctacccgggtcagaacggtggtcaggccctggccgaaattctgttcggcaagatcaacccgtcgggcaagctgccgatcagtatcgaacgcaacatcgaagacaacccggcctacgcctccttcccgaaattcgacaaccagaacacgctggctgaaatggattacaaggatgacctgatgctgggttatcgcggttacgagaagaaaggcatcaagccgctttacccgttcggttacggcctgtcgtacaccacgttcggctacagcaacatcaaagtcacgccaggcgtcgcggtgggcaatacgccgatcaaggtgtccttcgacctgaccaacaccggcaaggtcggcggttcggaagttgcacagctgtacgttggccagcagaacccgaaagtcgagcgtccgatcaaggaactcaaaggctacaagaaggtgttcctcaagccgggtgaaagcaagcgcgtgaccatcgagctcaatgaccgctcgctggcctacttcgacgtgaaaaccaaccagtgggtggttgacgccgacaccttcaacctgtcgctgggtggctcgtcgcaggacattcgcctgaacgccaagctggtcaactcgttccgccaggaactgtcgaccactaccagcaacccgctgccacgttcggcgctgaactcggtgctggtcgagaagccaccggtcaagaccggtggtgtgttccagcagactgtcgagtaa

SEQ ID NO: 94 - DNA, 2580 bp, Streptomyces coelicolor
gtggggacgagtgacgaagagatcgaccggctgctcggcaagctgacaccacgcgcccgcgcactgctgctgaacggcgccacgacctggcgcacgagggcggaaccagcggtggagctgagggagttggtgatgtcggacggtccggcgggcgtacggggcgaggcctgggacgagcggagcacctctctcctgctgccctccgcctcggcgctcgccgccacctgggacgaggcgctggtcgaagacctcggtggcctgctcgccgccgaggcccggcgcaagggcgtggacgtcctcctcgccccgaccctcaacctgcaccgcagcccgctgggcggccggcacttcgagtgcctctccgaggaccccgagctgaccggccggatcggcgccgcgctggtccgcgggatccaggcgcacggcgtggccgccaccgccaagcactacgtggccaacgactccgagaccgaccgcctcaccgtcgacgtgcgggtgggcgaacgggcgctgcgagaggtctacctcgcccccttcgaggcggcggtggccgccggggtccggctcgtcatggcggggtacaacgcggtcaacggcaccacgatgaccgcgaacgccctcctcaccgacccgctgaagagcgagtggggcttcgacggcgtcgtcgtgtccgactggggcgcggtgcgcggcacgaccggcaccgcccgcgccggtctcgacctcgccatgccgggtcccgacggcccctggggcgaggcgctggcccgcgcggtggccgagggcgcggtgcccgaaccggccgtcgacgacaaggcacggcgcctgctgcgactcgcggcgtggctgggcgcgctgggcgggcgcgacgtgtcccggtcgccggtcccgggcaggccggccgactcgccgggtgcggagggtgcggacggtggggcgggcgctggcccgtcgtccggtgcggagggcctcccgggccggggcccggcgcacggtgcgaagccgtccgggccccgaccgcggcgtgccggggacgggcgggcgctggcccgtcgtgccgtcgccgccggggccgtgctgctggccaacaaggacgtcctgccactcgaccccgagcacctcgggacggtcgccgtgatcggcgcgcacgccgcgcggacccgtacccagggcggcggcagcgcgggcgtcttcccgcggggcgaggtgtccgtcctcgacggcatccgggccgaactgcgcggccgggcccgcgtcgtgcacgtcccgggcccccggccggacggccccgcgcccccactggacccggacacatgcaccgacccgcgctcggggctgcccggcgtcctgctgcggatgctcgacgcggacggccgcgagctgtacgccgaacggcgccgcggcgggcgcctgctggagccccgcctggtgccgggcgcgcacaccgtcgagatccgcgcccggctgtgtccccgcaccggcggctcctggtccctgggcgtggccgggttcggccggatgagcctgacgacggacggacgcaccctgctggagggggacttcccgccgtccaccgacgatccggcggtgatgcacgtcaacccgcccgcccagtacgccaccgccgacctcaccgccggccgggacaccctgctggtggcccggcgcgagctggcacccggcaccggccgggcgaccgtcctcgtcgcggccccgcccgccccggacgtgaccgcgtcgctcgccgaggccgtccgcgcggccggtgcggcggacgccgcggtcgtggtcgtcggcaccaccgagcacggggagtcggagggctacgaccgtacggacctggcgctcggcgccacccaggacgcgctggtccgcgccgtcgcggccgccaacccgcgcaccgtcgccgtcgtcaacagcggcggcccggtggaactgccgtggcgggagcaggcgggtgcggtgctgctggcctggtttcccggacaggagggcggcggtggactggccgacgtgctcttcgggcacgccgagccgggcggacgactgcccaccacctggccggccgtcctcgccgacgccccggtcacccgcacccgccccgacggcggccgcctcgactacgacgagggactgcacctcggtcaccggggctggctgcgccatcaccgcacgcccgcctactggttcggacacgggctcggctacacgacgtggcggtacgaggagctgaccgtcccgccggtgacccgggcgggcgacggcctcaccgtgcgcgtgcgggtgcgcaacaccggtgcgcgagcgggccgggaggtcgtccaggtgtacctggcccggcccgcgtcggccctcgaccgtcccgcgcgttggctcgccgggtacacggcggtgcgggcgcgcccgggggagacggtgacggcgacggtgcgcgtcccggcgcgggccctgcgccactggtcggtggcggagcacgcgtggcgtaccgaggcggggccctgccgggtgctggccgggcggtcggcgggggacgtgccgctggccgcggaggtggaggtggtgcctacggcttccgcgtga

SEQ ID NO: 95 - DNA, 1410 bp, Caulobacter crescentus
tcagggggaggacggcaaggtcccggtctgcgccaaggccttgaaccagccgtaggacgccttcgggatcctacggccgctggcggcttccatcgtcgtgatcccaaacttcgaggtgtagcccaggtcccactcgaaattgtcgatcagggtccactcgaaatacccccggacgtcacaacccgcctcgcgggccgccagcaccgcctcaaggtggcggcgcaagtacttgatgcggaaggtgtcatccaagatcgctggaccgctgctgaacgggtcggaacatccgttctcggtcaccagcatcttgggcgcgccgtactcgcggcgcacacgatccagaacctcgaacaggccggacggatctatgtgccggccgaacgcgtcctgctcggcgctattgggcgcggcggcggcggcgatcttgctgggcgccgacagatccagccgcacgtaggccggcgcgtagtagttcacccccaggaaatcaacaggctggcgcgtggtcttcaagtcgccgtcgcgaaccacgcccttgagcggctcctccatcgccttggggtaggttcccttgaacagcgggtcgagccaggcaaggttccagatctcgtccagaccatccgacgccagccggttccagaacgccaatggcccacccgccggccggcacggctgaagcgccatcgtggtgcctaccgaaaggtcgctccgcgccgcgcgcagagcctggatcgccagaccctggcccagattcatatggtgcgtgactggacccagcagcgcagcgtccttgagccccggcgcatgatcgcccagcacgtggccgaacacggtatggaccgccgcctcattgagaatgatgtagttcttcagccggtcgcccagccgctcgaccacggcgcgcgcatagtccgccagacgctgggccgtgtcgcggttcgcccagccgcccttgtcctgcaggccttgcggcaggtcccaatgaaacagggtggcgtagggcgtgatccccttggccagcagcgcgtcgaccagccgcgaatagtgatcgaggcctgcggcgttcaccgcgcccgccccggtcggcagaatccgcgaccagctcatcgagaaccgataagcgctcaggctggcgccggcgatcaggtcgacatcgtcctggtagcgccgatagctgtcggtggcgtccgcagcggtgtcgccattcttgacgtggcccggaactctctcgaacacgtcccagatgctgggcccgcgcccgtcggcggtttgcgaaccttcggtctggaaagcggccgtagccacgccccagacaaagtccttcggaaactgccgacccttgggggtcaggtccgtttcgcccggcccctcgcacgccgatagccccagagccgcgccgcccagggccaa

SEQ ID NO: 96 - DNA, 2158 bp, Candida wickerhamii
gaattcaattgaagtcatgatgtttgcattttgccaatttgtcagtttacttagaatttcattatttaaatgatactttttgcctttgtggaagtatttgaaattttatcaattaaaaactgttaagaaaagatgttctcacaaaagtatcttttatcattagctgcaataattgcaatcgctaaagcagctccagctgatgacgcttctaaaccaggtattgggaaatttgcaccaggtcaattaggtttccgttattatatcgacaccaccaccgagtacgcaactcctgccactgctactgctcctgcaagttccactacgtacgctgcaccatatgctgaattgtcatccttggttggaaacttgtccacgacgacatggggtaattggtatcctgacgctaccgaggctgccacggatactgatgacccatatggacaatacgcatggtctcaattatgggaagctaccactttcccaaattttactcgtggtatttacagtaccacggtggatccaacaccgatcccaaccgagagtttagttgtgccaccagatgacccagtcaagagggcattccaagatttgggaatcaaattccctctgggtttcattcaaggtgttgccggttccgctgctcaaattgaaggtgccgtcgccgatgaaggtagatcaccaactaatttagaagttagttccgctagtagacatttacctgaagatttcgtcactaatgaaaattattatctttacaaacaagatatcaccagattagcagctatcggcgttgaatactattcgttcactatcccatggactagaatcttaccattcgcctatcccggttctcctgtgaatcaacaaggtttagatcattatgacgacttgatcaacactgtcttagcatatggaatgaaaccaattgtcacattgatccatttcgattcaccattacaacttgtcgacttcaatgccacattggaattgggactgccaggtggatacgaaggtgaagatttcgtcgaggcatttgtcaattacggtaaaatcgtcatgacccatttcgctgatcgtgtcccattatggatcatctttaatgaacctgtccaattcgccactaatggactcggtgtcaaacatgtcgtccaagccacggctcaattatacgatttctaccataacgagatcaacgggtccggtaagattggtatgaagttcagtcacatcttcgggttccctgaggatccaactaacccagaacatgttgctgccgcagacagatcaaatgaattgcaattaggtctctttgctgatccattgttcttaggtgaagactacccagacagtttcaagaccacattattgaaaacgcagccagcactggcttggacactggatgaattagccgctgttaagggtaaatgtgatttcttcggtgttgatccatacacttataacactatcaagccattggataacggtactgcatcatgtgaagccaacgtcaccgacacttactggccaacgtgtgtcaatgtcaccgttactgaagctgataactggagtatcggttaccgttcccaatcctatgtctacatcacaccaagacaattaagagtctcgttgaactacatctggcaacactggcacgttcctatcttcatcacggaatttggtttccctgaatggagagaaggtgagaaactcttagttgaccaagtccaagatttggacagatccatttactacagatctttcttgactgcagcattagaggcatctcagtacgacggtgtcgagataatgggtgccttggcttggagttttgccgataattgggaattcggtgattataaccaacaattcggtttacaagtcgttaatagaactactcaggagagattctataagaagagtttctttgattttgtcggttttattaatgataatagagcttgagatccctaatccatttatgattatattttttaaaaaactttctatgatgactattatttctttaatgacattactacactaaatgtcaatttctttacttacgcttcttttattttataacccagaaaagttgatatacaatttacttgtcttttaccaatttaaataaaaaattaaataaataaaattcaagctt

SEQ ID NO: 97 - DNA, 1527 bp, Bradyrhizobium japonicum
atgaaccgagctgccaacgccaacctgttttcccgcctgttcgacggcctggacgatcccaaacgcctcgcgatcgagacgcatgacggcgcccgtatcagctatggcgatctgatcgcgcgggccgggcaaatggcgaacgtgctggtcgcgcgcggcgtgaagcctggcgaccgcgtcgcggtgcaggtcgagaaatcggtcgcgaacatcgtgctttatctcgccacggtgcgggccggcgcggtctacctgccgctgaacaccgcctatacgctcaacgagctcgattacttcatcggcgacgccgagccgtcgctggtggtctgcgatccctccaaggccgagggcctcgccccgatcgccgcgaaggtgaaggccggggtcgagacgctcggccccgacggcaagggctctctgacggaggccgccgacaaggcgagcagcgcgtttacgacggtgccgcgcgaaaacgacgatctggccgcgatcctctacacgtcgggcaccaccggccgctccaagggggcgatgctcacccacgataatctcgcgtcgaactcgctctcgctcgtcggctactggcgcttcaccgacaaggacgtgctgatccacgcgctgccgatctaccatacgcacggtctgttcgtggcgaccaacgtgacgctgttttcgcgggcgtcgatgatcttcctgccgaagctcgaccctgacctgatcatcaagctgatggcgcgcgccaccgtgctgatgggcgtgccgaccttctacacccgcctcctgcaaaatgccgcgctgtcgcgcgagaccaccaggcacatgcggctgttcatttcgggctcggcgccgctgctcgccgaaacccatcgcgaatggtcggcgcgcaccggacatgccgtgctcgagcgttacggcatgaccgagaccaacatgaacacgtcgaacccctatgacggcgagcgcgtgcccggcgcggtcggttttcccctccccggcgtctccctgcgcgtgaccgaccccgagaccggcaaggagctgccgcgcgaggagatcggcatgatcgaggtcaagggcccgaacgtgttcaagggctactggcgcatgccggagaagaccaaggcggaattccggcccgacggcttcttcatcaccggcgacctcggcaagatcgacggcaagggctacgtccacatcctcggccgcggcaaggatctcgtgatctccggcggcttcaacgtctatccgaaggaaatcgagagcgagatcgacgcgatgcccggcgtggtcgagtccgccgtgatcggcgtgccgcatgccgatttcggcgagggcgtcaccgccgtgctggtctgcaacaagggtgccgaggtcagcgaagcctccgtgctgaaagcactcgacggccggctcgccaaattcaagatgccgaagcgcgtcttcgtggtcgacgagctgccgcgcaacaccatggggaaggtgcagaagaacgtgctgcgggatacctacaaggatatctacgcgaagaaa

SEQ ID NO: 98 - DNA, 1527 bp, Bradyrhizobium sp. BTAi1
atgaaccagaccgccaacgccaatctgttcgcccgcttgttcgacggtctcgacgatcccagccgcctcgcgatcgagacccatgacggtcagcgcatcacctatggcgacctgattgcgcgcgcgggccagatggcgaatgtgctggtcagcaggggcgtgaagccgggcgaccgcgtcgcggcgcagaccgagaaatcagtgtcgggcctcgtgctctatctcgccacggtccgcgccggcggcgtctatctgccgctcaacacggcctacacgctcaacgagctcgactatttcatcggcgacgccgagccgaccgtcgtcgtgtgcgatcccgccaaggccgagggcatccgcacgctggctgccaaggtcggcgccacggtggacacgctcgatgcgtcaggcaagggctcgctgaccgaggccgccgacaaggccgccaccgcgttcaccaccgtgccgcgcggcgccgacgatctcgccgcgatcctctatacgtcaggcaccaccggccgctccaaaggcgcgatgctgtcccacgacaatctcgcctcgaactcgctgaccctgatcgactactggcgcttcaccaaggacgacgtgctgatccacgcgctgccgatctatcatacgcatggcctgttcgtggccagcaacgtgacgctgttcgcgcgcgcgtcgatgatcttcctgcccaagctcgatcccgacctgatcatcaatctgatggcacgcgccaccgtgctgatgggcgtgccgaccttctacacccggctgctgcagaacccgcggctgaacaaagagacgaccagccacatgcgtctcttcatctccggctctgcgcctttgctcgccgacacccatcgcgaatggttcgcgcgcaccggccacgccgtgctcgagcgctacggcatgaccgagaccaacatgaacacgtcgaacccgtatgatggcgagcgcgtgcccggcgcggtcggcttcccgctgcctggcgtgtccgtgcgcgtgaccgatcccgagaccggcaaggagctcgcgcgcgacgagatcggcatgatcgaggtgaagggcccgaacgtgttcaagggctattggcggatgccggagaagaccaagtcggagttccgtcctgacggcttcttcatcaccggcgacctcggcaagatcgacacgcagggctacgtccacatcgtcgggcgcggcaaggatctggtcatctccggcggcttcaacgtctatccgaaggagatcgagagcgagatcgacgccatgccgggcgtggtcgagtccgccgtcatcggcgtgccgcatgccgatttcggcgagggcgtcacggcggtcgtcgtgcgtcaccccggtgctgacgtcaatgaagccagcgtgctcaaggggctcgacggccggctggccaagttcaagatgccgaagcgcgtgttcgtggtcgacgagctgccgcgcaacaccatgggcaaggtgcagaagaacgtgctgcgtgatcagtacaaggacatctatacgaaatga

SEQ ID NO: 99 - DNA, 1512 bp, Rhodopseudomonas palustris
atgaacgccaacctgttcgcccgcctgttcgataagctcgacgacccccacaagctcgcgatcgaaaccgcggccggggacaagatcagctacgccgagctggtggcgcgggcgggccgcgtcgccaacgtgctggtggcacgcggcctgcaggtcggcgaccgcgttgcggcgcaaaccgagaagtcggtggaagcgctggtgctgtatctcgccacggtgcgggccggcggcgtgtatctgccgctcaacaccgcctatacgctgcacgagctcgattacttcatcaccgatgccgagccgaagatcgtggtgtgcgatccgtccaagcgcgacgggatcgcggcgattgccgccaaggtcggcgccacggtggagacgcttggccccgacggtcggggctcgctcaccgatgcggcagctggagccagcgaggcgttcgccacgatcgaccgcggcgccgatgatctggcggcgatcctctacacctcagggacgaccggccgctccaagggcgcgatgctcagccacgacaatttggcgtcgaactcgctgacgctggtcgattactggcgcttcacgccggatgacgtgctgatccacgcgctgccgatctatcacacccatggattgttcgtggccagcaacgtcacgctgttcgcgcgcggatcgatgatcttcctgccgaagttcgatcccgacaagatcctcgacctgatggcgcgcgccaccgtgctgatgggtgtgccgacgttctacacgcggctcttgcagagcccgcggctgaccaaggagacgacgggccacatgaggctgttcatctccgggtcggcgccgctgctcgccgatacgcatcgcgaatggtcggcgaagaccggtcacgccgtgctcgagcgctacggcatgaccgagaccaacatgaacacctcgaacccgtatgacggcgaccgcgtccccggcgcggtcggcccggcgctgcccggcgtttcggcgcgcgtgaccgatccggaaaccggcaaggaactgccgcgcggcgacatcgggatgatcgaggtgaagggcccgaacgtgttcaagggctactggcggatgccggagaagaccaagtctgaattccgcgacgacggcttcttcatcaccggcgacctcggcaagatcgacgagcgcggctacgtccacatcctcggccgcggcaaggatctggtgatcaccggcggcttcaacgtctatccgaaggaaatcgagagcgagatcgacgccatgccgggcgtggtcgaatccgcggtgatcggcgtgccgcacgccgatttcggcgagggcgtcactgccgtggtggtgcgcgacaagggtgccacgatcgacgaagcgcaggtgctgcacggcctcgacggtcagctcgccaagttcaagatgccgaagaaagtgatcttcgtcgacgacctgccgcgcaacaccatgggcaaggtccagaagaacgtcctgcgcgagacctacaaggacatctacaagtaa

SEQ ID NO: 100 - DNA, 1515 bp, Mesorhizobium loti
atgagcaatcatctgttcgatgcgttccggtcccggatgccggcgcccgggcatctgttgatggaaaccgatgacgggcgctcgctgagctatggcgacatgctggcgcggtcggcacagttcgcgcacgcgctgctgcaattggatgtcgagcccggcgaccgggtggccgtgcaggtcgagaagagcccggaggcgctgctgctctatctcgcctgcgtgcgcgccggtgccgtcttcctgccgctcaacaccgcctacacgctgaccgaactcggctatttcttcggggatgcagcgccgcgcgtcatcgtctgcgatccggcaagggcggccgatatcgggcgcatggtcgagccatccggcgccgtcgtcgtcacgctcgaccgcaacggccggggatcgctggcggaccaggcctcgcgtctgccgtcggattttcacgatgtggcgcgcgggccggatgatctcgcggcaatcctctacacgtcgggaacgaccggccgctcgaaaggcgccatgctcagccacgagaacctggcctcgaatgcacgggtgctggtcgagcaatggcgcttcacatcaggcgacgtgctgatccacgcactgccgatctttcacacccatggcctgttcgtcgccaccaacgtcgtcctgatggcgggcgccgcgatgctgttcgagcagaaattcgatccggcccgcatcgtcgcgctgctgccgcgcggcaccgcgctgatgggcgtgccgaccttttatgtgcgcctgctgcagcaggatggactggaccggcaggcggcgaagactatccgcctgttcgtgtccggctccgcgccgctgctggccgacacgcacaaggcctggcgcgagcgcaccggccacgccatcctcgaacgctacggcatgaccgagaccaacatgaacacctcgaacccctatgagggcgaacggcgcgccggcacggtcggcttccccttgccgggtgtcgctttgcgcatcgccgatcccgacacgggcaagccgctggcccaaggcgaagtcggcatgatcgaggtcaagggtccgaacgttttcggcggctattggcgcatgccggaaaagaccaaggcggaattccgcgccgacggtttcttcatcaccggcgatctcggcatggtcgacaccgacggctatgtccatatcgtcggccgcggcaaggatctgatcatttcgggcggctacaacatctatccgaaggagctcgaaagtgagatcgacgcgcttgatggagtgagcgaaagcgccgtcatcggcgtcgcccatccggatttcggcgaaggcgtcaccgcggtcgtcgtgcgagcgccgggggcagcgatcaccggagccgaagtgcttggcgcaatcgccggacgcctggctaggtacaagcatccgaagcaggtgatcttcgtcgacgaattgccgcgcaacacgatgggcaaggtccagaagaatcttctgcgagacgcctacaaggatctctacacatcctag

SEQ ID NO: 101 - DNA, 1350 bp, Rhizobium etli
atgggtattgaaatactggccataggcctgctcatcgctatgttcatcgttgcgacgatccagccgatcaacatgggcgcgctcgctttcgccggcgccttcgtgctcggctccatgaccatcggcatgaaaaccaccgatatttttgcgggctttccgagcgatctgttcctgacgctggtcgccgtcacctacctcttcgccatcgcgcagatcaacggcaccatcgactggctggtcgaatgcgccgtgcgcctggtgcgtggacgcatcggcctgatcccctgggtgatgttcctcgtcgccgccgtcattaccggtttcggcgcgcttggacctgctgccgtcgccattctcgcgccggtcgcgctgagctttgccatccagtaccgcattcaccccgtcatgatggggctgatggtgatccacggcgcgcaggcgggcggcttttcgccgatcagcatctatggcggcattaccaaccagatcgttgccaaggccggcctgcccttcgcgccgacatccctgtttctctccagctttttcttcaatctggcgattgcggtgctggtcttcttcgttttcggcggcgcgaaggtcatgaagcaagtgccggcagcatcgcttggccccttgcccgaactgcaccccgagggcgtgtcggcctcgatcagaggtcacggcggcacgccggcaaaaccgatccgggaacatgcctacggcacggcggccgatactgctgcgacgctgcgtctgaacaatgagcgaattaccacgctggtcggactgatcgcgctcggtatcggggcgctcgttttcaagttcaatgtcggcctcgtcgccatgaccgtcgccgtcgccctcgcgctgctgtcgccgaaaacccagaaggcggcaatcgacaaggttagctggtcgaccgtgcttttgatcgccggcatcatcacctatgtcggcgtcatggagaaagccggtaccgtcgattacgtcgcaaacggcatatccagcctcggcatgccgctgctggtggcgcttctgctttgctttaccggcgccatcgtttcggccttcgcatcctcgaccgcgctgctcggcgccatcatcccgctggccgtccccttcctcatgcagggacatgtcagcgccatcggcgtcgtcgccgccatcgcgatctcgacgacgatcgtcgataccagcccattttccaccaacggtgcgcttgtggtcgccaatgcgccggatgaaaggcgcgagcaggtcctcaagcagctgctgatctacagcgcactgattgccatcatcggcccgatcgtcgcctggctggtgttcgtcgtgccggggctggtttga

SEQ ID NO: 102 - DNA, 1356 bp, Xanthomonas campestris pv. Vesicatoria
atgaccccgcaaattgcaacgattcttggcttggtgatcatgttcatcgtcgccaccgcattgcccatcaacatgggggcggtggcgttcgcgctggccttcatcattggcggcgtgtgggtggggctggagggcaaggaagtgctggccggctttcccggcgacctgttcctgaccctggtgggcattacctatctgttcgccatcgcgcagaaaaacggcaccatcgatttgctggtgcactgggcagtgcgcgcggtgcgggggcgcatcgtcgccatcccctgggtgatgttcgtcatcaccgggctgctgaccgcattcggtgcgctggggccggcagcggtggccatcatcgggccggtggcgctgcgcttcgccaagcagtaccgcatcaacccgttgatgatgggcctgctggtgatccacggtgcacagggcggcggcttctcgccgatcagcgtgtatggcggcatcaccaacaaggtggtcgaaaaggccgggctggacgtcagcgagatggcggtgttcctcaccagcctgggcttcaacctgatgatggcgatcatctgcttctgcgccttcggtgggttgaagctgatgcgccggcaggagctgtcgctggccgatgcgcagtacgtggcggttgccggcgaccaggcatcgcaccgcggcttcgcgatcgaagggcatggcgcgctggtggcgccgggcggcggcaccctgtccaccgacccgctggcattggaggcggtgttcatcacccgcgaccgcgtgatcaccttgatcggcctgctgggcctgggcgttgctgcgctgatctacaacctcaacgtcggcctggtctcgatcacggtggcggtggccctggcgctgatctcgcccaacgcacagaagggcgcggtggacggcatcagttggtcgacggtgctgctgatcagcggcgtggtgacctatatcgccgtgctggaaaaatccggtgcggtggactacatcggcaccggcgtgtcgcacatcggcatgccgctgctgggcgcgctgctggtctgctacgtcggcggtatcgtctctgcattcgcctcctcggcagcggtactgggcgccaccattccgttggcggtgccattcctgatgcagggccagttgggcgcggccggggtgatttgcgcgctggccgtgtcctccaccatcgtcgacgtcagcccgttttccaccaacggcgcgctggtggtggcctcggcggccaaggaagagcgcgacggcctgttccgccgcttcctgatctacagcggcctggtggtgttgctggggccgctggcggcgtggctggtcttcgtggtgccgggctggctgtga

SEQ ID NO: 103 - DNA, 1356 bp, Xanthomonas campestris pv. Campestris
atgaccccgcaaattgcaacgattcttggcttggtgatcatgttcatcgtggccaccgcattgcccatcaacatgggtgcggtggccttcgcgctggccttcatcatcggtggcgtgtgggtggggctggacggcaaggaggtcctggccggcttcccgggcgatctgttcctcaccctggtcggcatcacctatctgttcgccatcgcgcagaagaacggcaccatcgatctgctggtgcactgggcggtccgcgcagtgcgtgggcgcattgtggccatcccgtgggtgatgttcgtcatcaccggcctgctcaccgcgtttggtgcgttggggccggcggcagtggccatcatcggcccggtggcactgcgctttgccaagcaataccgcatcaatcccttgatgatgggcctgctggtgatccacggcgcgcagggcggcgggttttcgcccatcagcgtgtatggcggcatcaccaacaaggtggtggaaaaggccgggctcgatgtcaccgagatggccgtattcctcaccagcctgggcttcaacctgatgatggcgatcatttgcttctgcgcctttggtggcctgcagttgatgcgccgcaacgagctgtcgctggccgatgcgcagtacgtgccggtggccgacgaccaggcctccaagcgcccgttcgccatcgaaggccacggtgcgttggtggcggccggcggcggcaccttgtcgaccaacccgctcgcgctggaagcggtggccatcacccgcgaccgcgtgatcaccctggtcggcctgctcgggctgggcgtggctgcgctggtctacaacctcaacgtcggcctggtgtcgatcaccgttgcggtggcactggcgctgatttcgcccagcgcgcagaaaggcgcagtggatggcatcagctggtccacggtgttgttgatcagcggcgtggtgacctatgtggcggtgctggaagaggctggtgcggtggattacatcggcaccggcgtctcgcacatcggcatgccgctgctgggcgcgttgctggtctgctacgtgggcggcatcgtgtcggcgtttgcctcgtcggcggcggtgctcggtgccaccattccgctggcggtgccgttcctgatgcaagggcatctgggcgcggccggtgtgatctgcgcattggcggtgtcgtccaccatcgtcgatgtcagcccgttttccaccaatggcgcgctggtggtggcctcggccgccaaggaagagcgcgacgcgctgttccgccgtttcctggtctatagcggcctggtagtggtgctcggccctctggcggcgtggctgctgttcgtggtgccgggttggctgtga